SEQUENCE LISTING (39 sequences)

SEQ ID NO: 1 - DNA, 2137 bp, Homo sapiens
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 2 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 3 - DNA, 1723 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgg

SEQ ID NO: 4 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 5 - DNA, 5598 bp, Homo sapiens
ggcccctcagccctgctgcccagcacgagcctgtgctcgccctgcccaacgcagacagccagacccagggcggcccctctggcggctctgctcctcccgaaggatgcttggggagtgaggcgaagctgggcgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcactttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagccccaagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatggtaccccaagaaggatgtgagaggtggccgcttggagtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcac

SEQ ID NO: 6 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 7 - DNA, 4017 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 8 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 9 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 10 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 11 - DNA, 2305 bp, Homo sapiens
ttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 12 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 13 - DNA, 6633 bp, Homo sapiens
ttctccccgccccccagttgttgtcgaagtctgggggttgggactggaccccctgattgcgtaagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacaggaggagactgcaagagatcatttgggaaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccggatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctctcgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttcttcatgcctgatgaaagctttggcaaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgggtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtt

SEQ ID NO: 14 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 15 - RNA, 10 nt, Artificial Sequence
  Synthetic anti-VEGF aptamer
gaagaauugg

SEQ ID NO: 16 - RNA, 8 nt, Artificial Sequence
  Synthetic anti-VEGF aptamer
uuggacgc

SEQ ID NO: 17 - DNA, 3007 bp, Homo sapiens
gcccggagagccgcatctattggcagctttgttattgatcagaaactgctcgccgccgacttggcttccagtctggctgcgggcaacccttgagttttcgcctctgtcctgtcccccgaactgacaggtgctcccagcaacttgctggggacttctcgccgctcccccgcgtccccaccccctcattcctccctcgccttcacccccacccccaccacttcgccacagctcaggatttgtttaaaccttgggaaactggttcaggtccaggttttgctttgatccttttcaaaaactggagacacagaagagggctctaggaaaaagttttggatgggattatgtggaaactaccctgcgattctctgctgccagagcaggctcggcgcttccaccccagtgcagccttcccctggcggtggtgaaagagactcgggagtcgctgcttccaaagtgcccgccgtgagtgagctctcaccccagtcagccaaatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcgtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatgatataaaatattgttgctctgacaaaaatacatgtatttcattctcgtatggtgctagagttagattaatctgcattttaaaaaactgaattggaatagaattggtaagttgcaaagactttttgaaaataattaaattatcatatcttccattcctgttattggagatgaaaataaaaagcaacttatgaaagtagacattcagatccagccattactaacctattccttttttggggaaatctgagcctagctcagaaaaacataaagcaccttgaaaaagacttggcagcttcctgataaagcgtgctgtgctgtgcagtaggaacacatcctatttattgtgatgttgtggttttattatcttaaactctgttccatacacttgtataaatacatggatatttttatgtacagaagtatgtctcttaaccagttcacttattgtactctggcaatttaaaagaaaatcagtaaaatattttgcttgtaaaatgcttaatatcgtgcctaggttatgtggtgactatttgaatcaaaaatgtattgaatcatcaaataaaagaatgtggctattttggggagaaaatt

SEQ ID NO: 18 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 19 - DNA, 3997 bp, Homo sapiens
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 20 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 21 - DNA, 3979 bp, Homo sapiens
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 22 - protein, 364 aa, Homo sapiens
MHRLIFVYTLICANFCSCRDTSATPQSASIKALRNANLRRDDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 23 - DNA, 29 bp, Artificial Sequence
  Synthetic anti-PDGF aptamer
caggcuacgcgtagagcaucatgatccug

SEQ ID NO: 24 - protein, 118 aa, Artificial Sequence
  Synthetic anti-VEGF antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTL

SEQ ID NO: 25 - protein, 110 aa, Artificial Sequence
  Synthetic anti-VEGF antibody
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 26 - protein, 118 aa, Artificial Sequence
  Synthetic anti-VEGF antibody
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTL

SEQ ID NO: 27 - protein, 110 aa, Artificial Sequence
  Synthetic anti-VEGF antibody
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 28 - protein, 44 aa, Homo sapiens
RKSRVVDLNLLTEEVRLYSCTPRNFSVSIREELKRTDTIFWPGC

SEQ ID NO: 29 - protein, 20 aa, Homo sapiens
RKSRVVDLNLLTEEVRLYSC

SEQ ID NO: 30 - protein, 43 aa, Homo sapiens
RKSKVDLDRLNDDAKRYSCTPRNYSVNIREELKLANVVFFPRC

SEQ ID NO: 31 - protein, 19 aa, Homo sapiens
CKSKVDLDRLNDDAKRYSC

SEQ ID NO: 32 - protein, 16 aa, Homo sapiens
CNDEGLECVPTEESNI

SEQ ID NO: 33 - protein, 16 aa, Homo sapiens
CPDDGLECVPTGQHQV

SEQ ID NO: 34 - RNA, 8 nt, Artificial Sequence
  Synthetic anti-VEGF aptamer
gugaaugc

SEQ ID NO: 35 - RNA, 27 nt, Artificial Sequence
  Synthetic anti-VEGF aptamer
cggaaucagugaaugcuuauacauccg

SEQ ID NO: 36 - RNA, 27 nt, Artificial Sequence
  Synthetic anti-VEGF aptamer
cggaaucagugaaugcuuauacauccg

SEQ ID NO: 37 - DNA, 30 bp, Artificial Sequence
  Synthetic anti-PDGF aptamer
caggcuacgcgtagagcaucatgatccugt

SEQ ID NO: 38 - DNA, 30 bp, Artificial Sequence
  Synthetic anti-PDGF aptamer
caggctacgcgtagagcatcatgatcctgt

SEQ ID NO: 39 - DNA, 32 bp, Artificial Sequence
  Synthetic anti-PDGF aptamer
caggcuacgncgtagagcaucantgatccugt

(19) United States
(12) Patent Application Publication
(10) Pub. No.: US 2014/0179621 A1
(43) Pub. Date: Jun. 26, 2014

(54) Methods for Treating or Preventing Ophthalmological Diseases

(71) Applicant: Ophthotech Corporation, Princeton, NJ (US)

(72) Inventors: Samir Patel, Princeton, NJ (US); Harvey Masonson, Princeton, NJ (US)

(73) Assignee: Ophthotech Corporation, Princeton, NJ (US)

(21) Appl. No.: 13/963,872

(22) Filed: Aug. 9, 2013

Related U.S. Application Data
(63) Continuation of application No. 13/284,221, filed on Oct. 28, 2011, now abandoned.
Application No. 13/284,221 is a continuation of application No. PCT/US2010/032816, filed on Apr. 28, 2010.
(60) Provisional application No. 61/174,746, filed on May 1, 2009.
(60) Provisional application No. 61/178,010, filed on May 13, 2009.
(60) Provisional application No. 61/245,784, filed on Sep. 25, 2009.

(51) Int. Cl. A61K 47/48; A61K 38/17

(52) U.S. Cl. CPC A61K 47/48215; A61K 38/17
USPC 514/20.8

(57) ABSTRACT

This invention relates to methods and compositions useful for the treatment or prevention of an ophthalmological disease, comprising administration of an effective amount of a PDGF antagonist and a VEGF antagonist to a mammal in need thereof.

### 1. RELATED APPLICATIONS

This application is a continuation of U.S. application Ser. No. 13/284,221, filed Oct. 28, 2011, which is a continuation of International Application No. PCT/US2010/032816, filed Apr. 28, 2010, which claims the benefit of U.S. Provisional Application No. 61/174,746, filed May 1, 2009, U.S. Provisional Application No. 61/178,010, filed May 13, 2009, and U.S. Provisional Application No. 61/245,784, filed Sep. 25, 2009, each of which is incorporated by reference herein in its entirety.

### 2. DESCRIPTION OF THE TEXT FILE SUBMITTED ELECTRONICALLY

The contents of the text file submitted electronically herewith are incorporated here by reference in their entirety: A computer readable format copy of the Sequence Listing (filename: OPHT_{—}007_{—}05US_SeqList_ST25.txt, date recorded: Aug. 9, 2013, file size 116 kb).

### 3. FIELD OF THE INVENTION

This invention relates to methods and compositions useful for the treatment or prevention of an ophthalmological disease, comprising administration of an effective amount of (a) ARC-127, Antagonist A, Antagonist B, Antagonist C, Antagonist D, 1B3 antibody, CDP860, IMC-3G3, imatinib, 162.62 antibody, 163.31 antibody, 169.14 antibody, 169.31 antibody, αR1 antibody, 2A1E2 antibody, M4TS.11 antibody, M4TS.22 antibody, A10, brefeldin A, sunitinib, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, 5-methyl-7-diethylamino-s-triazolo (1,5-a) pyrimidine, interferon, protamine, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, 4-(2-(N-(-2-carboxamidoindole)aminoethyl)-benzenesulfonamide, 4-(2-(N-(-2-carboxamidoindole)aminoethyl)-sulfonylurea, CGP 53716, human antibody g162, pyrazolo[3,4-g]quinoxaline, 6-[2-(methylcarbamoyl)phenylsulphanyl]-3-E-[2-(pyridine-2-yl)ethenyl]-indazole, 1-{2-[5-(2-methoxy-ethoxy)-benzoimidazole-1-yl]-quinoline-8-yl}-piperidine-4-ylamine, 4-[4-[N-(4-nitrophenyl)carbamoyl]-1-piperazinyl]-6,7-dimethoxyquinazoline, 4-amino-5-fluoro-3-(6-(4-methyl-piperazine-1-yl)-1H-benzimidazole-2-yl)-1H-quinoline-2-one, (4-tert-butylphenyl){4-[(6,7-dimethoxy-4-quinolyl)oxy]phenyl}methaneone, 5-methyl-N-[4-(trifluoromethyl)phenyl]-4-isoxazolecarboxamide, trans-4-[(6,7-dimethoxyquinoxaline-2-yl)amino]cyclohexanol, (Z)-3-[(2,4-dimethyl-5-(2-oxo-1,2-dihydroindole-3-ylidenemethyl)-1H-pyrrole-3-yl)-propionic acid, 5-(5-fluoro-2-oxo-1,2-dihydroindole-3-ylidenemethyl)-2,4-dimethyl-1H-pyrrole-3-carboxylic acid, 1-(4-chloroanilino)-4-(4-pyridylmethyl)phthalazine, N-[4-(3-amino-1H-indazole-4-yl)phenyl-N′-(2-fluoro-5-methylphenyl)urea, 1,2-dimethyl-7-(2-thiophene)imidazolo[5,4-g]quinoxaline, 1,2-dimethyl-6-phenyl imidazolo[5,4-g]quinoxaline, 1,2-dimethyl-6-(2-thiophene)imidazolo[5,4-g]quinoxaline, AG1295, AG1296, 3-arylquinoline, 4-pyridyl-2-arylpyrimidine, sorafenib, MLN518, PKC412, AMN107, suramin, or neomycin, or a pharmaceutically acceptable salt thereof and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof, to a mammal in need thereof.

### 4. BACKGROUND OF THE INVENTION

Various disorders of the eye are characterized, caused by, or result in choroidal, retinal or iris neovascularization or retinal edema. One of these disorders is macular degeneration. Age-related macular degeneration (AMD) is a disease that affects approximately one in ten Americans over the age of 65. One type of AMD, “wet-AMD” accounts for only 10% of age-related macular degeneration cases but results in 90% of cases of legal blindness from macular degeneration in the elderly. Another disorder of the eye is diabetic retinopathy. Diabetic retinopathy can affect up to 80% of all patients having diabetes for 10 years or more and is the third leading cause of adult blindness, accounting for almost 7% of blindness in the USA. Other disorders include hypertensive retinopathy, central serous chorioretinopathy, cystoid macular edema, Coats disease and ocular or adnexal neoplasms such as choroidal hemangioma, retinal pigment epithelial carcinoma and intraocular lymphoma.

Therefore, although advances in the understanding of the molecular events accompanying neovascularization have been made, there exists a need to utilize this understanding to develop improved methods for treating or preventing neovascular diseases disorders, including ocular neovascular diseases and disorders such as the neovascularization that occurs with AMD and diabetic retinopathy.

### 5. SUMMARY OF THE INVENTION

In one aspect the invention provides methods for treating or preventing an ophthalmological disease, comprising administering to a mammal in need thereof an effective amount of (a) ARC-127 or imatinib, or a pharmaceutically acceptable salt thereof; and (b) ranibizumab, bevacizumab, aflibercept, KH902 VEGF receptor-Fc fusion protein, ORA102, bevasiranib, SIRNA-027, decursin, decursinol, picropodophyllin, guggulsterone, PLG01, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) 1B3 antibody, CDP860, IMC-3G3, 162.62 antibody, 163.31 antibody, 169.14 antibody, 169.31 antibody, αR1 antibody, 2A1E2 antibody, M4TS.11 antibody, M4TS.22 antibody, A10, brefeldin A, sunitinib, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, 5-methyl-7-diethylamino-s-triazolo (1,5-a) pyrimidine, interferon, protamine, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, 4-(2-(N-(-2-carboxamidoindole)aminoethyl)-benzenesulfonamide, 4-(2-(N-(-2-carboxamidoindole)aminoethyl)-sulfonylurea, CGP 53716, human antibody g162, pyrazolo[3,4-g]quinoxaline, 6-[2-(methylcarbamoyl)phenylsulphanyl]-3-E-[2-(pyridine-2-yl)ethenyl]-indazole, 1-{2-[5-(2-methoxy-ethoxy)-benzoimidazole-1-yl]-quinoline-8-yl}-piperidine-4-ylamine, 4-[4-[N-(4-nitrophenyl)carbamoyl]-1-piperazinyl]-6,7-dimethoxyquinazoline, 4-amino-5-fluoro-3-(6-(4-methyl-piperazine-1-yl)-1H-benzimidazole-2-yl)-1H-quinoline-2-one, (4-tert-butylphenyl) {4-[(6,7-dimethoxy-4-quinolyl)oxy]phenyl}methaneone, 5-methyl-N-[4-(trifluoromethyl)phenyl]-4-isoxazolecarboxamide, trans-4-[(6,7-dimethoxyquinoxaline-2-yl)amino]cyclohexanol, (Z)-3-[(2,4-dimethyl-5-(2-oxo-1,2-dihydroindole-3-ylidenemethyl)-1H-pyrrole-3-yl)-propionic acid, 5-(5-fluoro-2-oxo-1,2-dihydroindole-3-ylidenemethyl)-2,4-dimethyl-1H-pyrrole-3-carboxylic acid, 1-(4-chloroanilino)-4-(4-pyridylmethyl)phthalazine, N-[4-(3-amino-1H-indazole-4-yl)phenyl-N′-(2-fluoro-5-methylphenyl)urea, 1,2-dimethyl-7-(2-thiophene)imidazolo[5,4-g]quinoxaline, 1,2-dimethyl-6-phenyl imidazolo[5,4-g]quinoxaline, 1,2-dimethyl-6-(2-thiophene)imidazolo[5,4-g]quinoxaline, AG1295, AG1296, 3-arylquinoline, 4-pyridyl-2-arylpyrimidine, sorafenib, MLN518, PKC412, AMN107, suramin, or neomycin, or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) ARC-127 or imatinib, or a pharmaceutically acceptable salt thereof; and (b) 2C3 antibody or pegaptanib, or a pharmaceutically acceptable salt thereof, wherein the ophthalmological disease is choroidal vasculopathy, condition associated with choroidal neovascularization, hypertensive retinopathy, sickle cell retinopathy, condition associated with peripheral retinal neovascularization, retinopathy of prematurity, venous occlusive disease, arterial occlusive disease, central serous chorioretinopathy, cystoid macular edema, retinal telangiectasia, arterial macroaneurysm, retinal angiomatosis, radiation-induced retinopathy, or a neoplasm.

In another aspect the invention provides methods for treating or preventing an ophthalmological disease, comprising administering to a mammal in need thereof an effective amount of (a) Antagonist A, a compound of Formula A or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) Antagonist B, a compound of Formula B or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) Antagonist C, a compound of Formula C or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) Antagonist D, a compound of Formula E or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

In another aspect the invention provides methods for treating or preventing an ophthalmological disease, comprising administering to a mammal in need thereof an effective amount of (a) 1B3 antibody, CDP860, IMC-3G3, 162.62 antibody, 163.31 antibody, 169.14 antibody, 169.31 antibody, αR1 antibody, 2A1E2 antibody, M4TS.11 antibody, M4TS.22 antibody, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, or HYB 9613 monoclonal antibody, or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof.

The invention provides compositions comprising an effective amount of (a) ARC-127, Antagonist A, Antagonist B, Antagonist C, Antagonist D, 1B3 antibody, CDP860, IMC-3G3, imatinib, 162.62 antibody, 163.31 antibody, 169.14 antibody, 169.31 antibody, αR1 antibody, 2A1E2 antibody, M4TS.11 antibody, M4TS.22 antibody, A10, brefeldin A, sunitinib, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, 5-methyl-7-diethylamino-s-triazolo (1,5-a) pyrimidine, interferon, protamine, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, 4-(2-(N-(-2-carboxamidoindole)aminoethyl)-benzenesulfonamide, 4-(2-(N-(-2-carboxamidoindole)aminoethyl)-sulfonylurea, CGP 53716, human antibody g162, pyrazolo[3,4-g]quinoxaline, 6-[2-(methylcarbamoyl)phenylsulphanyl]-3-E-[2-(pyridine-2-yl)ethenyl]-indazole, 1-{2-[5-(2-methoxy-ethoxy)-benzoimidazole-1-yl]-quinoline-8-yl}-piperidine-4-ylamine, 4-[4-[N-(4-nitrophenyl)carbamoyl]-1-piperazinyl]-6,7-dimethoxyquinazoline, 4-amino-5-fluoro-3-(6-(4-methyl-piperazine-1-yl)-1H-benzimidazole-2-yl)-1H-quinoline-2-one, (4-tert-butylphenyl) {4-[(6,7-dimethoxy-4-quinolyl)oxy]phenyl}methaneone, 5-methyl-N-[4-(trifluoromethyl)phenyl]-4-isoxazolecarboxamide, trans-4-[(6,7-dimethoxyquinoxaline-2-yl)amino]cyclohexanol, (Z)-3-[(2,4-dimethyl-5-(2-oxo-1,2-dihydroindole-3-ylidenemethyl)-1H-pyrrole-3-yl)-propionic acid, 5-(5-fluoro-2-oxo-1,2-dihydroindole-3-ylidenemethyl)-2,4-dimethyl-1H-pyrrole-3-carboxylic acid, 1-(4-chloroanilino)-4-(4-pyridylmethyl)phthalazine, N-[4-(3-amino-1H-indazole-4-yl)phenyl-N′-(2-fluoro-5-methylphenyl)urea, 1,2-dimethyl-7-(2-thiophene)imidazolo[5,4-g]quinoxaline, 1,2-dimethyl-6-phenyl imidazolo[5,4-g]quinoxaline, 1,2-dimethyl-6-(2-thiophene)imidazolo[5,4-g]quinoxaline, AG1295, AG1296, 3-arylquinoline, 4-pyridyl-2-arylpyrimidine, sorafenib, MLN518, PKC412, AMN107, suramin, or neomycin, or a pharmaceutically acceptable salt thereof; (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof; and (c) a pharmaceutically acceptable carrier or vehicle.

The invention provides compositions comprising an effective amount of (a) Antagonist A or a pharmaceutically acceptable salt thereof; (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof; and (c) a pharmaceutically acceptable carrier or vehicle.

The invention provides compositions comprising an effective amount of (a) Antagonist B or a pharmaceutically acceptable salt thereof; (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG01, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof; and (c) a pharmaceutically acceptable carrier or vehicle.

The invention provides compositions comprising an effective amount of (a) Antagonist C or a pharmaceutically acceptable salt thereof; (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof; and (c) a pharmaceutically acceptable carrier or vehicle.

The invention provides compositions comprising an effective amount of (a) Antagonist D or a pharmaceutically acceptable salt thereof; (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof; and (c) a pharmaceutically acceptable carrier or vehicle.

In another aspect the invention provides Antagonist A or a pharmaceutically acceptable salt thereof.

In another aspect the invention provides compositions comprising Antagonist A or a pharmaceutically acceptable salt thereof.

In another aspect the invention provides compositions comprising: (a) an effective amount of Antagonist A or a pharmaceutically acceptable salt thereof; and (b) a pharmaceutically acceptable carrier or vehicle.

In another aspect the invention provides compounds of Formula B and a pharmaceutically acceptable salt thereof.

In another aspect the invention provides compositions comprising a compound of Formula B or a pharmaceutically acceptable salt thereof.

In another aspect the invention provides compositions comprising: (a) an effective amount of a compound of Formula B or a pharmaceutically acceptable salt thereof; and (b) a pharmaceutically acceptable carrier or vehicle.

In another aspect the invention provides a compound of Formula C or a pharmaceutically acceptable salt thereof.

In another aspect the invention provides compositions comprising a compound of Formula C or a pharmaceutically acceptable salt thereof.

In another aspect the invention provides compositions comprising: (a) an effective amount of a compound of Formula C or a pharmaceutically acceptable salt thereof; and (b) a pharmaceutically acceptable carrier or vehicle.

In another aspect the invention provides methods and compositions as described above, wherein Antagonist A, Antagonist B, Antagonist C or Antagonist D is linked with one or more nonphysiologically active groups, lipophilic groups or high-molecular weight compounds.

### 6. BRIEF DESCRIPTION OF THE DRAWINGS

Reference is made to the following detailed description, which sets forth illustrative embodiments and the accompanying drawings of which:

FIG. 1 (A) is a schematic representation of the nucleic acid sequence of a human PDGF-B (GenBank Accession No. X02811) (SEQ ID NO: 1).

FIG. 1 (B) is a schematic representation of the amino acid sequence of a human PDGF-B (GenBank Accession No. CAA26579) (SEQ ID NO: 2).

FIG. 1 (C) is a schematic representation of the nucleic acid sequence of a human PDGF-A (GenBank Accession No. X06374) (SEQ ID NO: 11).

FIG. 1 (D) is a schematic representation of the polypeptide sequence of a human PDGF-A (GenBank Accession No. CAA29677) (SEQ ID NO: 12).

FIG. 1 (E) is a schematic representation of the nucleic acid sequence of a human PDGF-C (GenBank Accession No. NM_{—}016205) (SEQ ID NO: 17).

FIG. 1 (F) is a schematic representation of the polypeptide sequence of a human PDGF-C (GenBank Accession No. NP_{—}057289) (SEQ ID NO: 18).

FIG. 1 (G) is a schematic representation of the nucleic acid sequence of a human PDGF-D, variant 1 (GenBank Accession No. NM_{—}025208) (SEQ ID NO: 19).

FIG. 1 (H) is a schematic representation of the polypeptide sequence of a human PDGF-D, variant 1 (GenBank Accession No. NP_{—}079484) (SEQ ID NO: 20).

FIG. 1 (I) is a schematic representation of the nucleic acid sequence of a human PDGF-D, variant 2 (GenBank Accession No. NM_{—}033135) (SEQ ID NO: 21).

FIG. 1 (J) is a schematic representation of the polypeptide sequence of a human PDGF-D, variant 2 (GenBank Accession No. NP_{—}149126) (SEQ ID NO: 22).

FIG. 2 (A) is a schematic representation of the nucleic acid sequence of a human VEGF (GenBank Accession No: NM_{—}003376) (SEQ ID NO: 3).

FIG. 2 (B) is a schematic representation of the amino acid sequence of a human VEGF polypeptide (GenBank Accession No. NP_{—}003367) (SEQ ID NO: 4).

FIG. 3 (A) is a schematic representation of the nucleic acid sequence of a human PDGFR-B (GenBank Accession No. NM_{—}002609) (SEQ ID NO: 5).

FIG. 3 (B) is a schematic representation of the polypeptide sequence of a human PDGFR-B (GenBank Accession No. NP_{—}002600) (SEQ ID NO: 6).

FIG. 3 (C) is a schematic representation of the nucleic acid sequence of a human PDGFR-A (GenBank Accession No. NM_{—}006206) (SEQ ID NO: 13).

FIG. 3 (D) is a schematic representation of the polypeptide sequence of a human PDGFR-A (GenBank Accession No. NP_{—}006197) (SEQ ID NO: 14).

FIG. 4 (A) is a schematic representation of the nucleic acid sequence of a human VEGFR-1 (Flt-1) (GenBank Accession No. AF063657) (SEQ ID NO: 7).

FIG. 4 (B) is schematic a representation of the polypeptide sequence of a human VEGFR-1 (Flt-1) (GenBank Accession No.) (SEQ ID NO: 8).

FIG. 4 (C) is a schematic representation of the nucleic acid sequence of a human VEGFR-2 (KDR/Flk-1) (GenBank Accession No. AF035121) (SEQ ID NO: 9).

FIG. 4 (D) is a schematic representation of the polypeptide sequence of a human VEGFR-2 (KDR/Flk-1) (GenBank Accession No. AAB88005) (SEQ ID NO: 10).

FIG. 5 is a graph of change in mean foveal thickness from a baseline over a 12 week period when treated with Antagonist A and ranibizumab (as the commercially available composition Lucentis®). The square symbol represents foveal thickness in the central subfield and diamond symbol represents foveal thickness in the central point.

FIGS. 6A-F show Formula A, wherein the 5′ end of its aptamer (SEQ ID NO: 23) is modified with Me(OCH₂CH₂)ₙOC(O)NH(CH₂)₄CH(NHC(O)O(CH₂CH₂O)ₙMe)C(O)NH(CH₂)_{w}—, wherein w is an integer from 2 to 12 and n is about 450

FIGS. 7A-F show the chemical structure of Antagonist A, wherein the 5′ end of its aptamer (SEQ ID NO: 23) is modified with Me(OCH₂CH₂)ₙOC(O)NH(CH₂)₄CH(NHC(O)O(CH₂CH₂O)ₙMe)C(O)NH(CH₂)₆—, where n is about 450.

FIGS. 8A-F show Formula B, wherein the 5′ end of its aptamer (SEQ ID NO: 23) is modified with Me(OCH₂CH₂)ₙOCH₂CH(O(CH₂CH₂O)ₙMe)CH₂OC(O)NH(CH₂)_{w}—, wherein w is an integer from 2 to 12 and n is about 450.

FIGS. 9A-F show the chemical structure of Antagonist B, wherein the 5′ end of its aptamer (SEQ ID NO: 23) is modified with Me(OCH₂CH₂)ₙOCH₂CH(O(CH₂CH₂O)ₙMe)CH₂OC(O)NH(CH₂)₆—, where n is about 450.

FIGS. 10A-F show Formula C, wherein the 5′ end of its aptamer (SEQ ID NO: 23) is modified with H₂N(CH₂)_{w}— and w is an integer from 2 to 12.

FIGS. 11A-F show the chemical structure of Antagonist C, wherein the 5′ end of its aptamer (SEQ ID NO: 23) is modified with H₂N(CH₂)₆—.

FIGS. 12A-F show the chemical structure of Antagonist D (SEQ ID NO: 23).

FIGS. 13A-F show Formula E, wherein the 5′ end of its aptamer (SEQ ID NO: 23) is modified with (R)ₓ(L)-, where L is a linker, y is 0 or 1, R is a nonphysiologically active group, lipophilic group or High Molecular Weight Compound, and x is an integer ranging from 1 to 4.

### 7. DETAILED DESCRIPTION OF THE INVENTION

### 7.1 Definitions and Abbreviations

As used herein, the following terms and phrases shall have the meanings set forth below. Unless defined otherwise, all technical and scientific terms used herein have the same meaning as commonly understood to one of skill in the art to which this invention belongs.

The term “about” a referenced numeric indication means the referenced numeric indication plus or minus up to 10% of that referenced numeric indication. For example, “about 100” means from 90 to 110.

The term “antagonist” refers to an agent that inhibits, either partially or fully, the activity or production of a target molecule. In particular, the term “antagonist,” as applied selectively herein, means an agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the antagonist may directly or indirectly inhibit the target molecule.

The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the Vi_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those in the art, and the fragments can be screened for utility in the same manner as whole antibodies.

The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein.

A nucleotide sequence is “complementary” to another nucleotide sequence if each of the bases of the two sequences matches, i.e., are capable of forming Watson Crick base pairs. The complement of a nucleic acid strand can be the complement of a coding strand or the complement of a non-coding strand.

The phrase “conserved residue” refers to an amino acid of a group of amino acids having particular common properties. A functional way to define common properties among individual amino acids is to analyze the normalized frequencies of amino acid changes among corresponding proteins of homologous organisms. According to such analyses, groups of amino acids may be characterized where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gin and Pro,

(ix) an aliphatic group consisting of Val, Leu, Ile, Met and Cys, and

(x) a small hydroxyl group consisting of Ser and Thr.

Members of each of the above groups are conserved residues.

The term “label” includes, but is not limited to, a radioactive isotope, a fluorophore, a chemiluminescent moiety, an enzyme, an enzyme substrate, an enzyme cofactor, an enzyme inhibitor, a dye, a metal ion, a ligand (e.g., biotin or a hapten) and the like. Examples of fluorophore labels include fluorescein, rhodamine, dansyl, umbelliferone, Texas red, luminol, NADPH, alpha-beta-galactosidase and horseradish peroxidase.

The term “nucleic acid” refers to a polynucleotide such as deoxyribonucleic acid (DNA) or ribonucleic acid (RNA). The term also includes analogs of RNA or DNA made from nucleotide analogs, and, as applicable to the embodiment being described, single (sense or antisense) and double-stranded polynucleotides, ESTs, chromosomes, cDNAs, mRNAs, and rRNAs.

The terms “RNA interference,” “RNAi,” “miRNA,” and “siRNA” refer to any method by which expression of a gene or gene product is decreased by introducing into a target cell one or more double-stranded RNAs, which are homologous to a gene of interest (particularly to the messenger RNA of the gene of interest, e.g., PDGF or VEGF).

The term “neovascularization” refers to new blood vessel formation in abnormal tissue or in abnormal positions.

The term “angiogenesis” refers to formation of new blood vessels in normal or in abnormal tissue or positions.

The term “ophthalmological disease” includes diseases of the eye and the ocular adnexa.

The term “ocular neovascular disorder” refers to an ocular disorder characterized by neovascularization. In one embodiment, the ocular neovascular disorder is a disorder other than cancer. Examples of ocular neovascular disorders include diabetic retinopathy and age-related macular degeneration.

The term “mammal” includes a human, monkey, cow, hog, sheep, horse, dog, and cat.

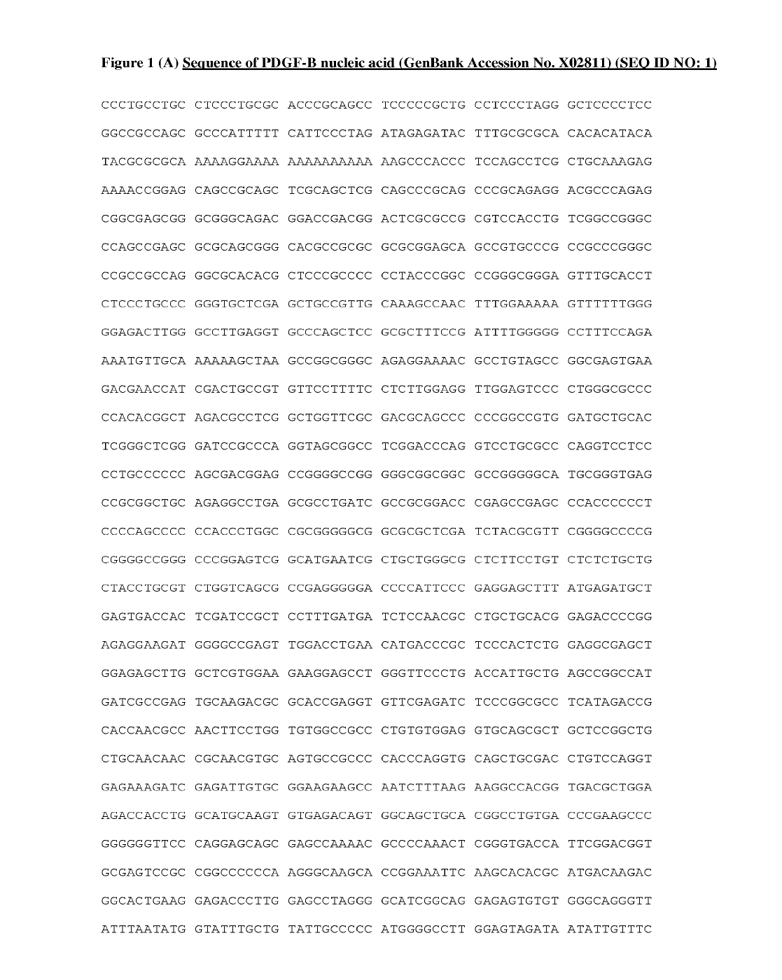

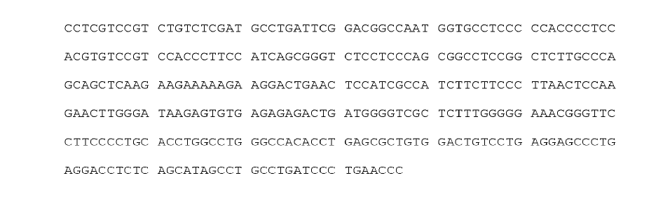

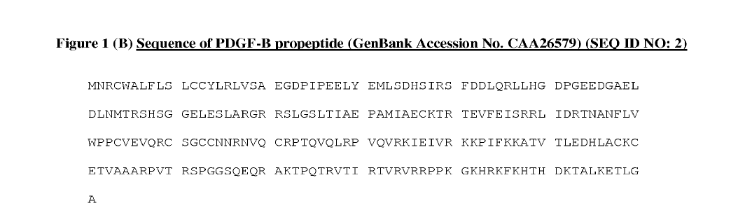

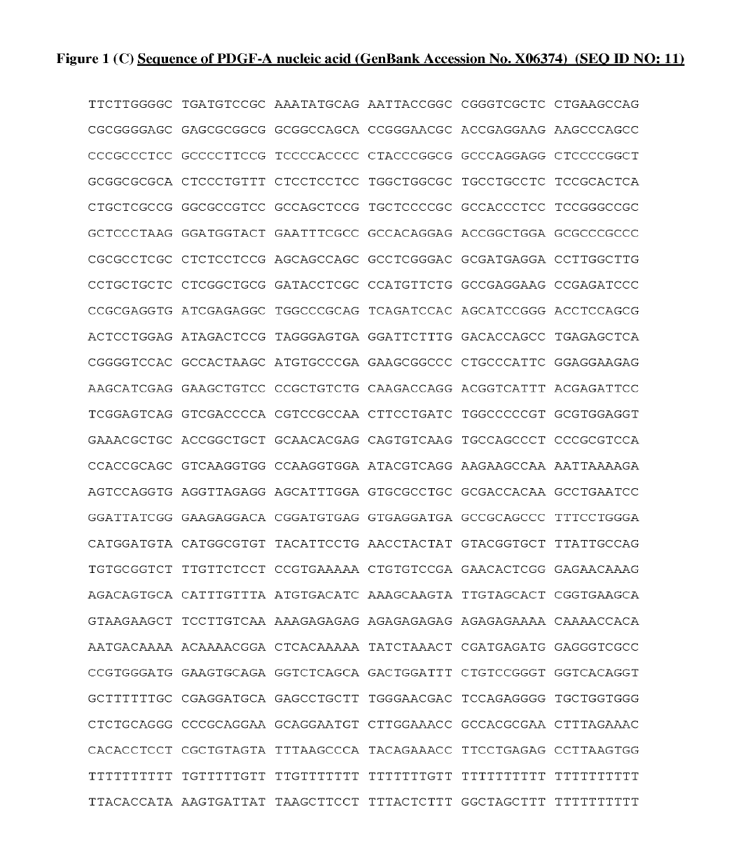

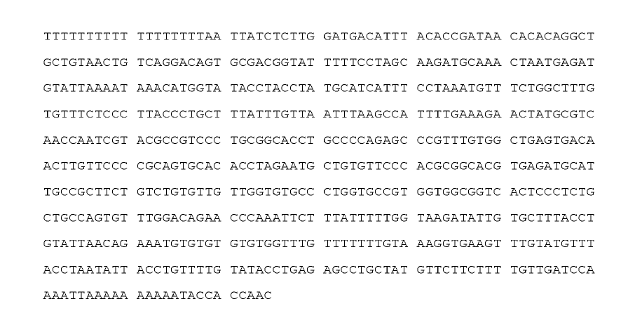

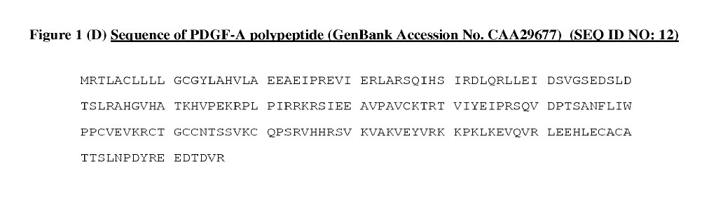

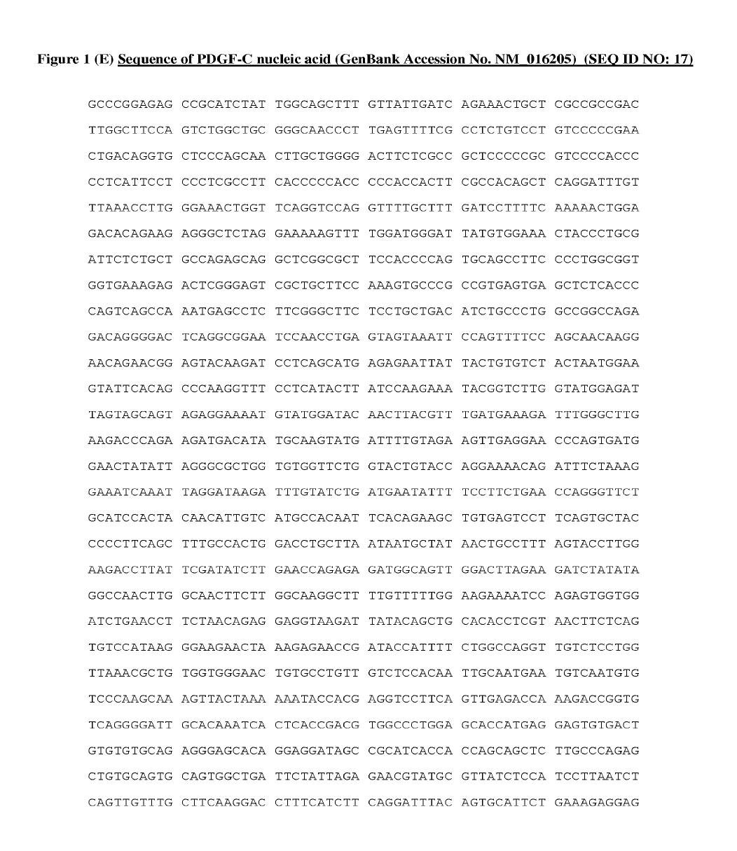

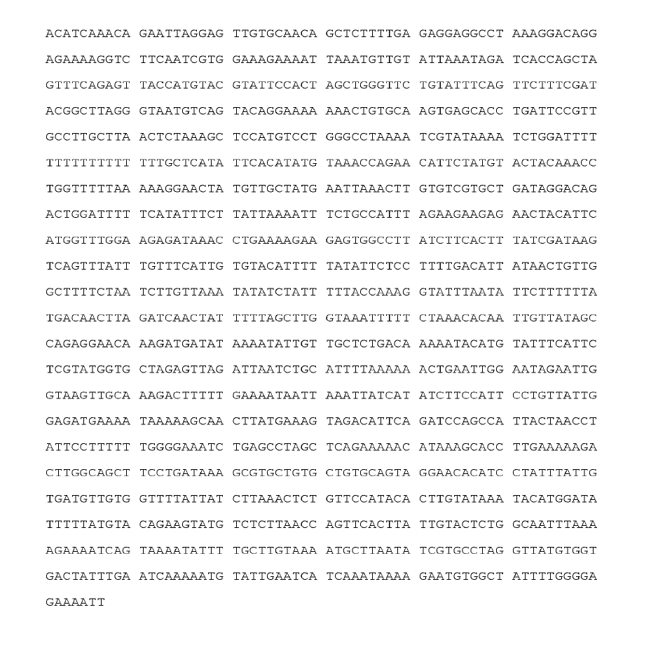

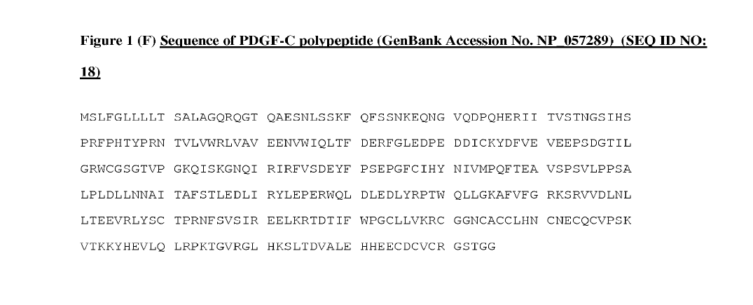

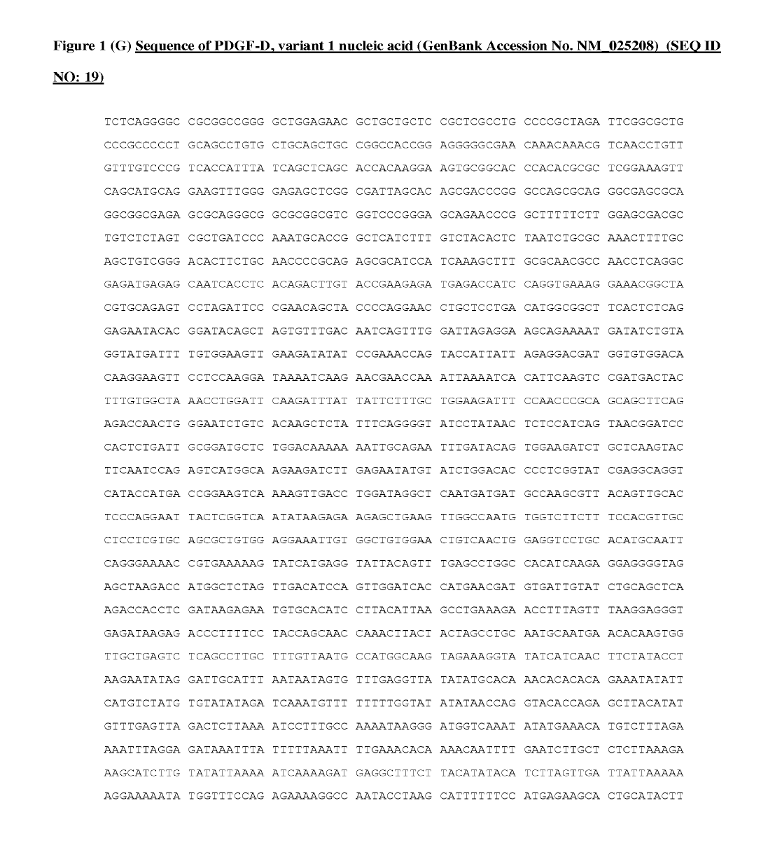

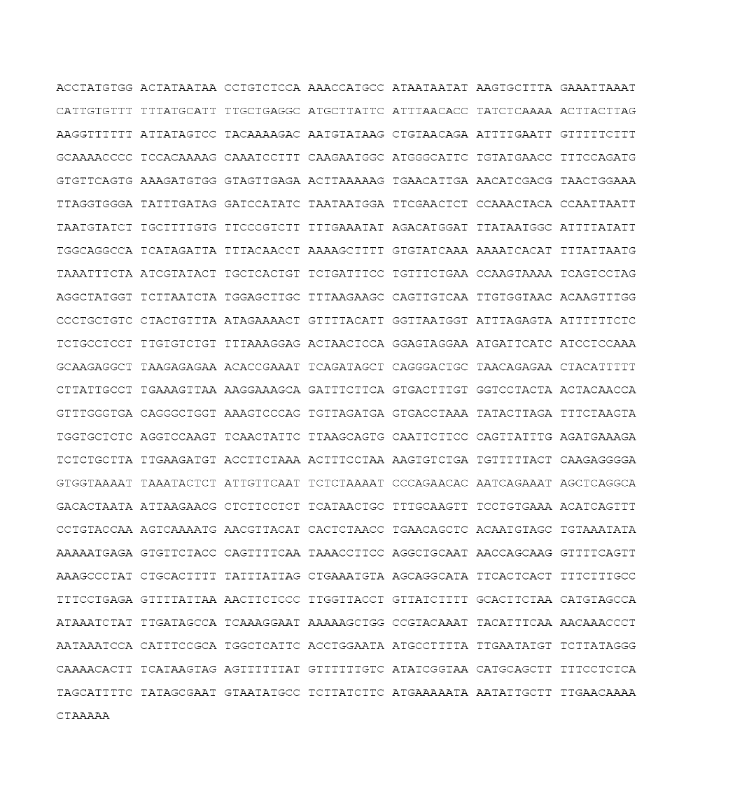

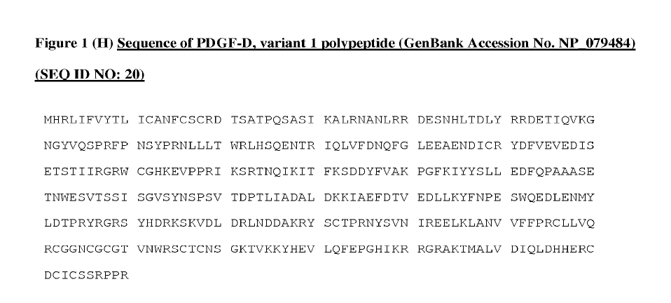

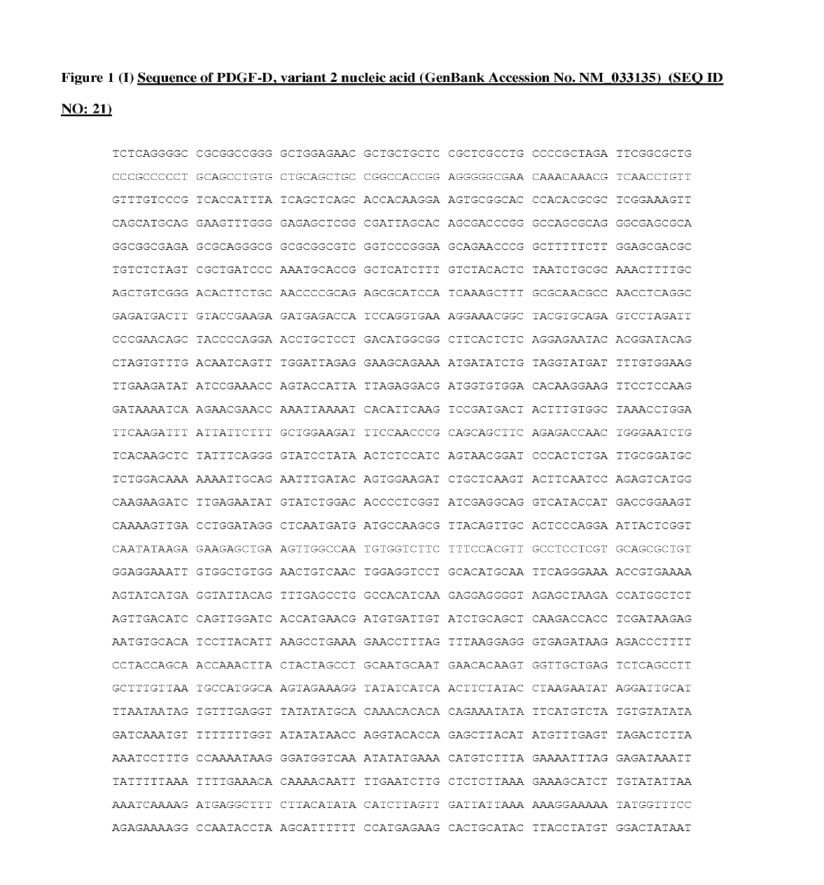

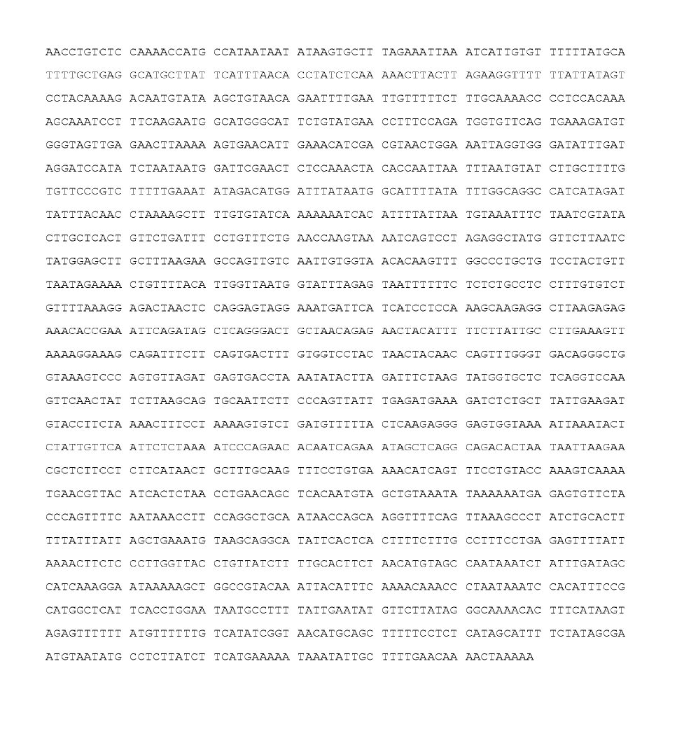

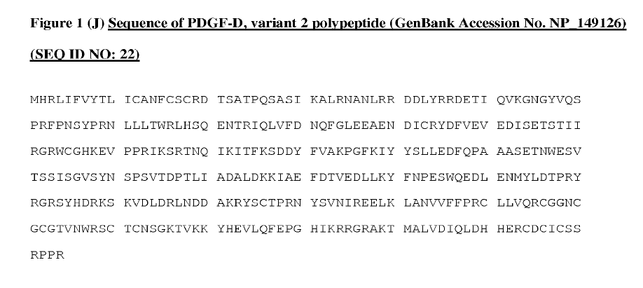

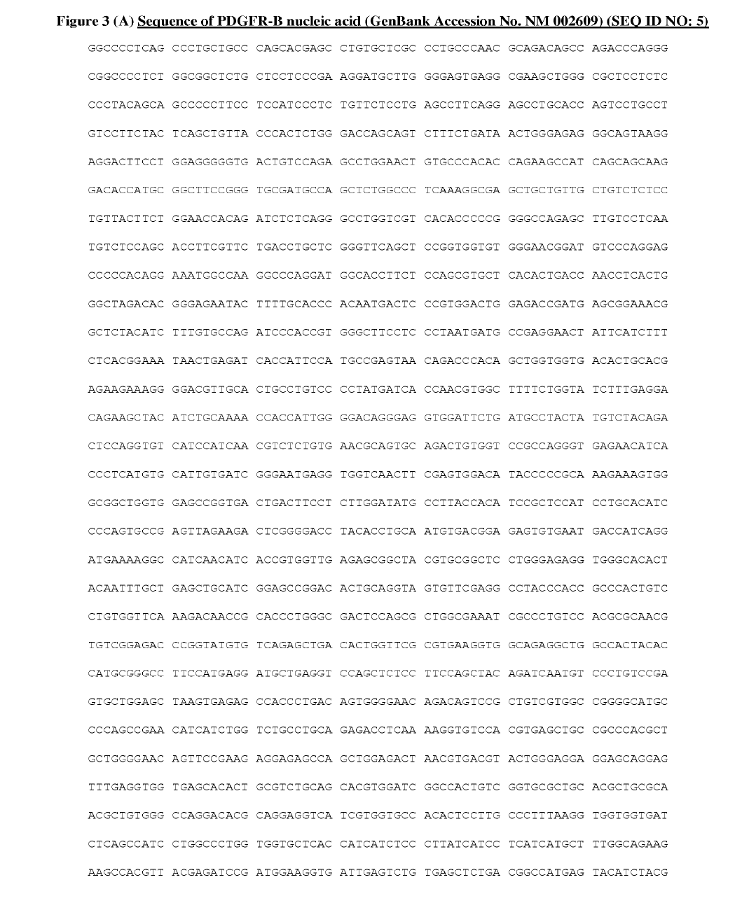

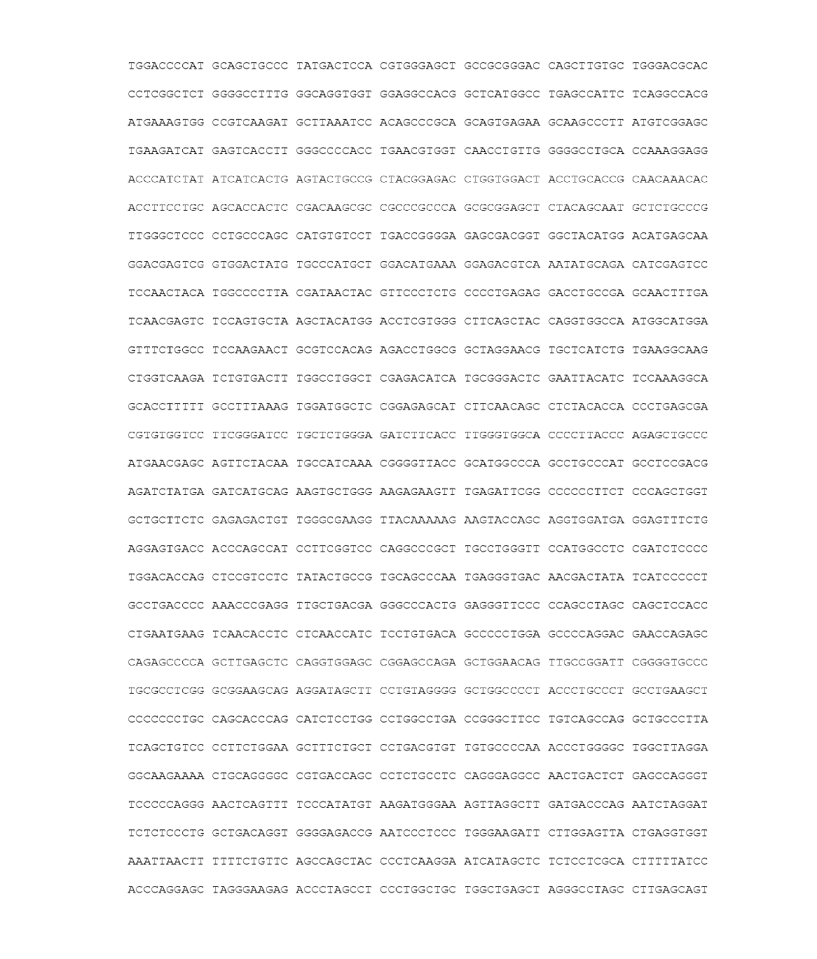

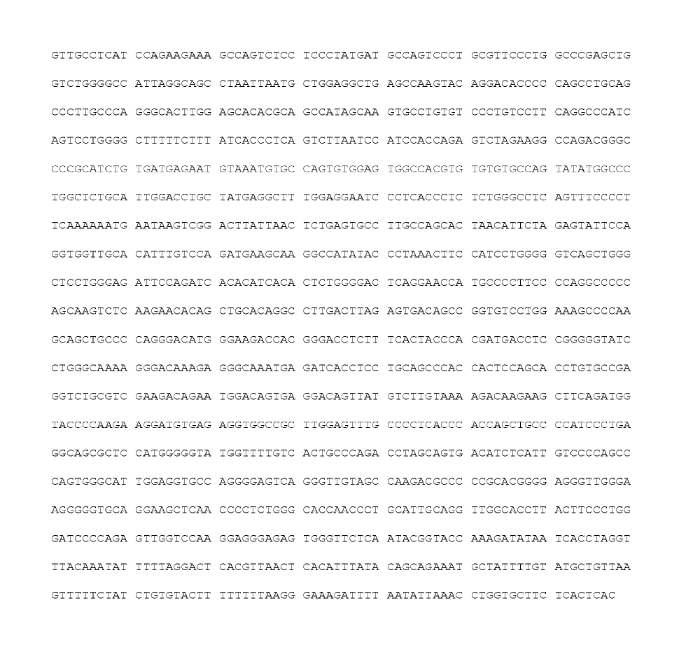

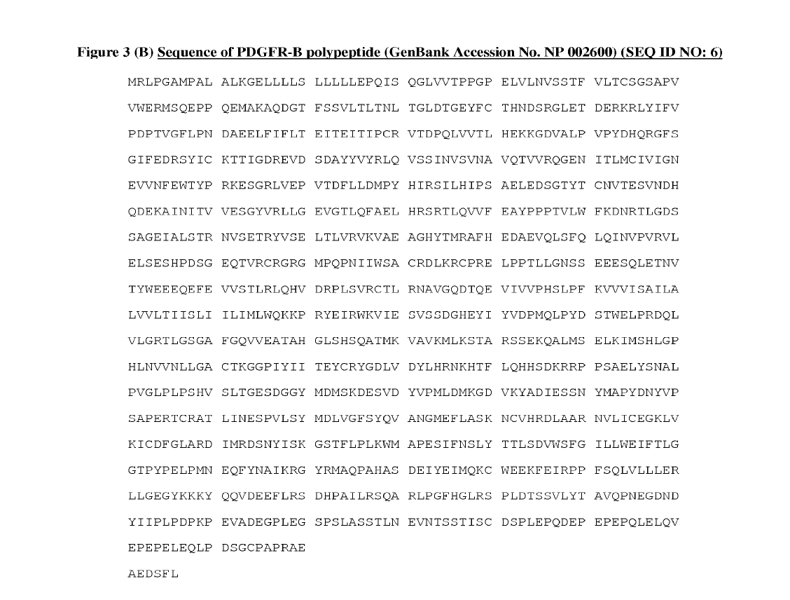

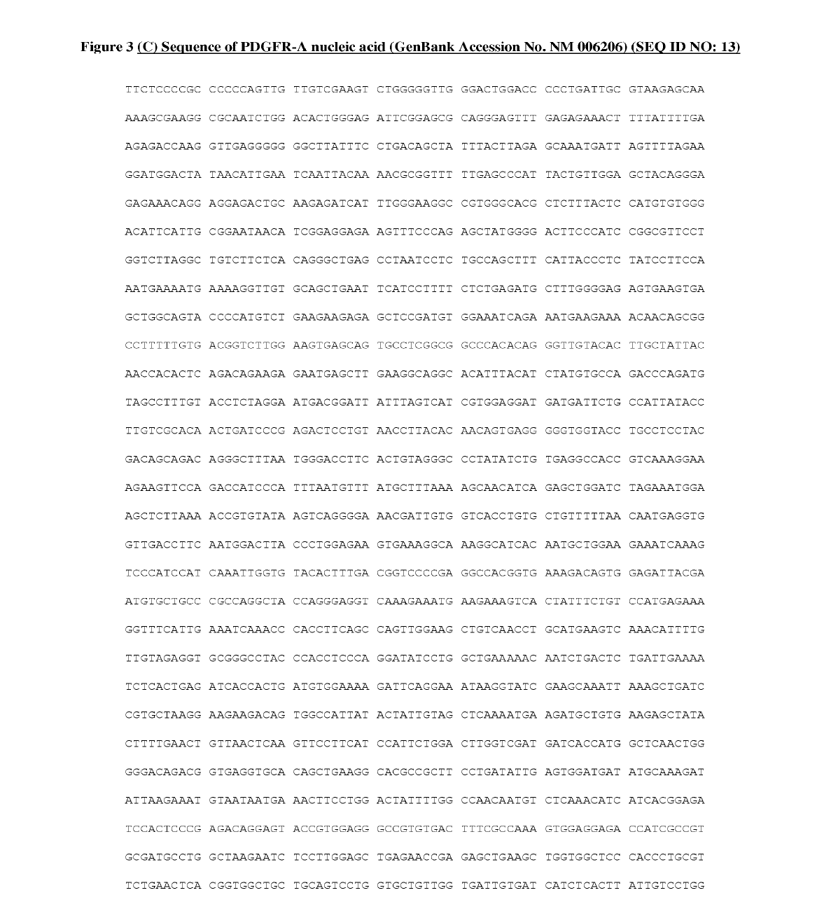

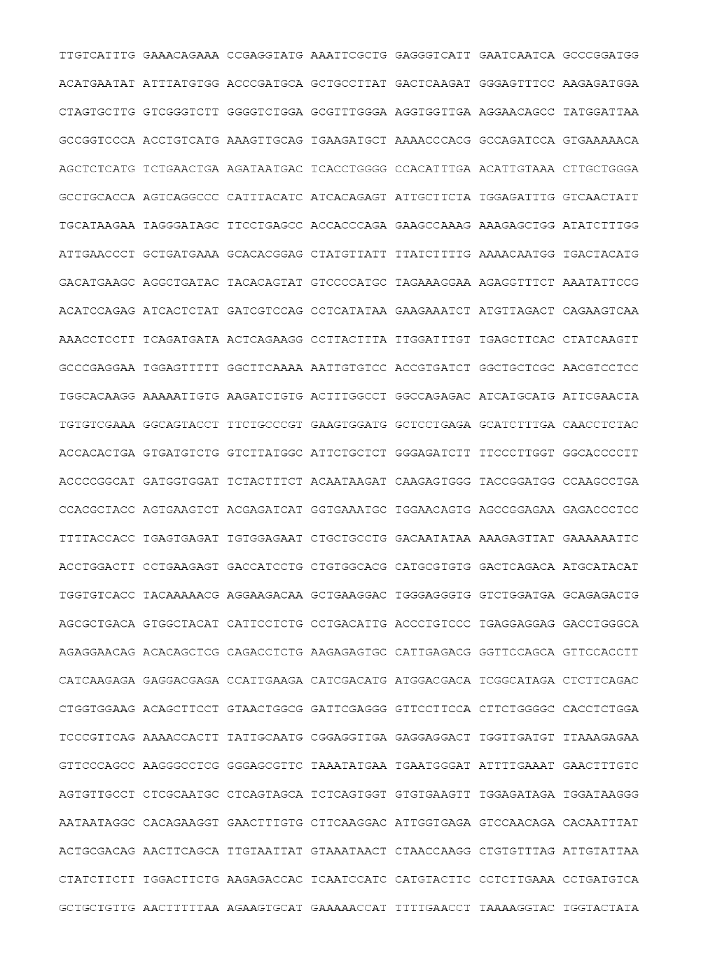

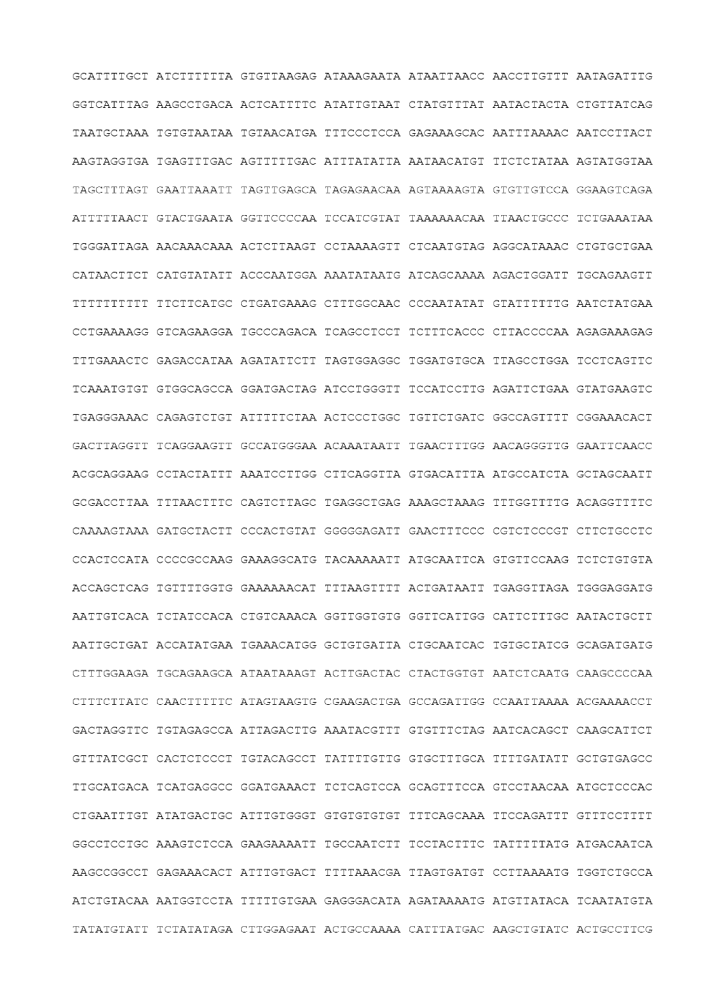

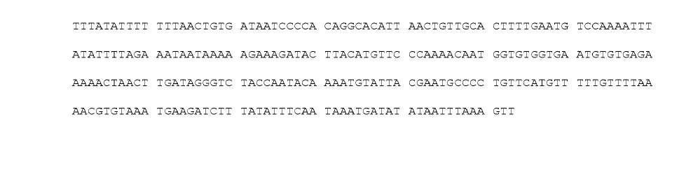

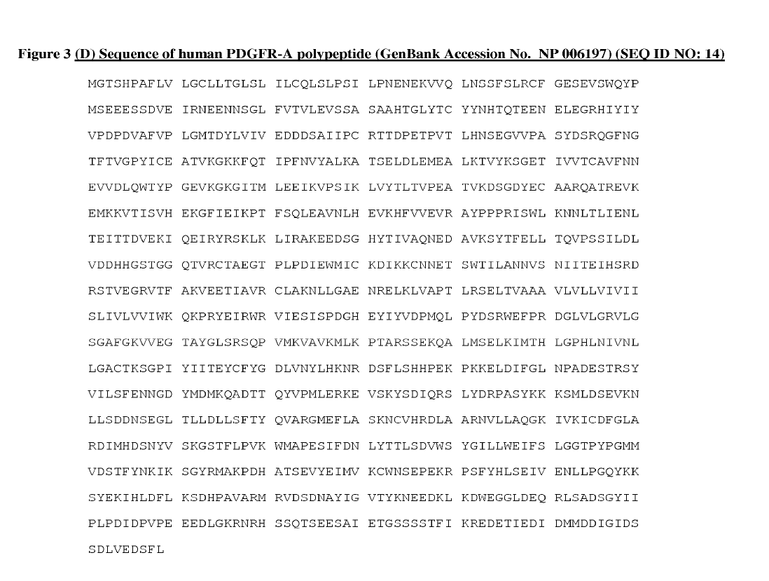

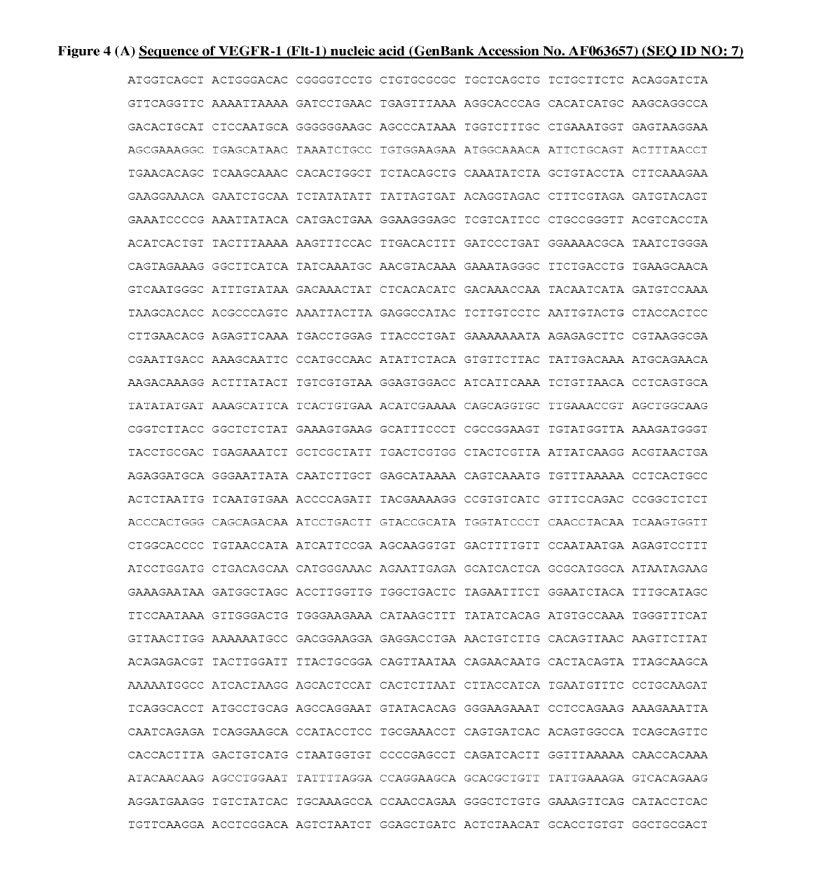

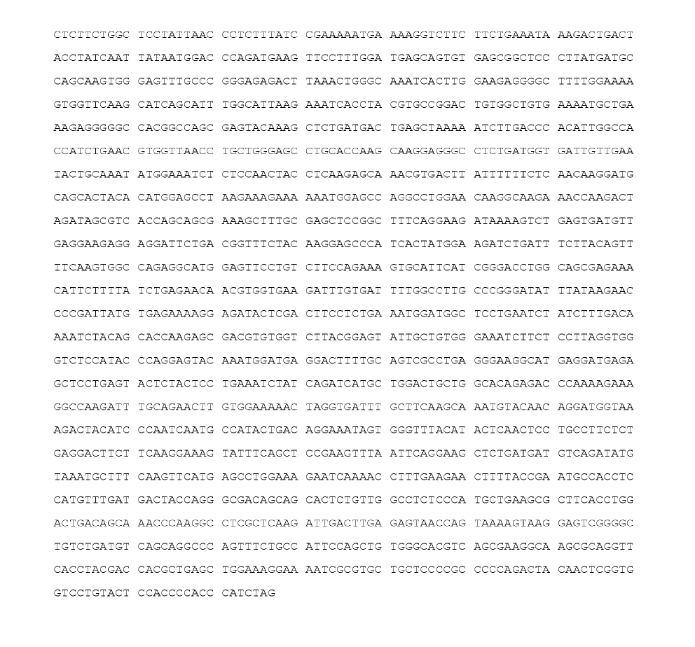

The term “PDGF” refers to a platelet-derived growth factor that regulates cell growth or division. As used herein, the term “PDGF” includes the various subtypes of PDGF including PDGF-B (see FIGS. 1(A) and (B)), PDGF-A (see FIGS. 1(C) and (D)), PDGF-C (see FIGS. 1(E) and (F)), PDGF-D, variants 1 and 2 (see FIGS. 1(G), (H), (I) and (J)), and dimerized forms thereof, including PDGF-AA, PDGF-AB, PDGF-BB, PDGF-CC, and PDGF-DD. Platelet derived growth factors includes homo- or heterodimers of A-chain (PDGF-A) and B-chain (PDGF-B) that exert their action via binding to and dimerization of two related receptor tyrosine kinase platelet-derived growth factor cell surface receptors (i.e., PDGFRs), PDGFR-α (see FIGS. 3 (C) and (D)) and PDGFR-β (see FIGS. 3 (A) and (B)). In addition, PDGF-C and PDGF-D, two additional protease-activated ligands for the PDGFR complexes, have been identified (Li et al., (2000) Nat. Cell. Biol. 2: 302-9; Bergsten et al., (2001) Nat. Cell. Biol. 3: 512-6; and Uutele et al., (2001) Circulation 103: 2242-47). Due to the different ligand binding specificities of the PDGFRs, it is known that PDGFR-α/α binds PDGF-AA, PDGF-BB, PDGF-AB, and PDGF-CC; PDGFR-β/β binds PDGF-BB and PDGF-DD; whereas PDGFR-α/β binds PDGF-AB, PDGF-BB, PDGF-CC, and PDGF-DD (Betsholtz et al., (2001) BioEssays 23: 494-507). As used herein, the term “PDGF” also refers to those members of the class of growth factors that induce DNA synthesis and mitogenesis through the binding and activation of a PDGFR on a responsive cell type. PDGFs can effect, for example: directed cell migration (chemotaxis) and cell activation; phospholipase activation; increased phosphatidylinositol turnover and prostaglandin metabolism; stimulation of both collagen and collagenase synthesis by responsive cells; alteration of cellular metabolic activities, including matrix synthesis, cytokine production, and lipoprotein uptake; induction, indirectly, of a proliferative response in cells lacking PDGF receptors; and potent vasoconstrictor activity. The term “PDGF” can be used to refer to a “PDGF” polypeptide, a “PDGF” encoding gene or nucleic acid, or a dimerized form thereof.

The term “PDGF-A” refers to an A chain polypeptide of PDGF or its corresponding encoding gene or nucleic acid.

The term “PDGF-B” refers to a B chain polypeptide of PDGF or its corresponding encoding gene or nucleic acid.

The term “PDGF-C” refers to a C chain polypeptide of PDGF or its corresponding encoding gene or nucleic acid.

The term “PDGF-D” refers to a D chain polypeptide of PDGF or its corresponding encoding gene or nucleic acid, including variants 1 and 2 of the D chain polypeptide of PDGF.

The term “PDGF-AA” refers to a dimer having two PDGF-A chain polypeptides.

The term “PDGF-AB” refers to a dimer having one PDGF-A chain polypeptide and one PDGF-B chain polypeptide.

The term “PDGF-BB” refers to a dimer having two PDGF-B chain polypeptides.

The term “PDGF-CC” refers to a dimer having two PDGF-C chain polypeptides.

The term “PDGF-DD” refers to a dimer having two PDGF-D chain polypeptides.

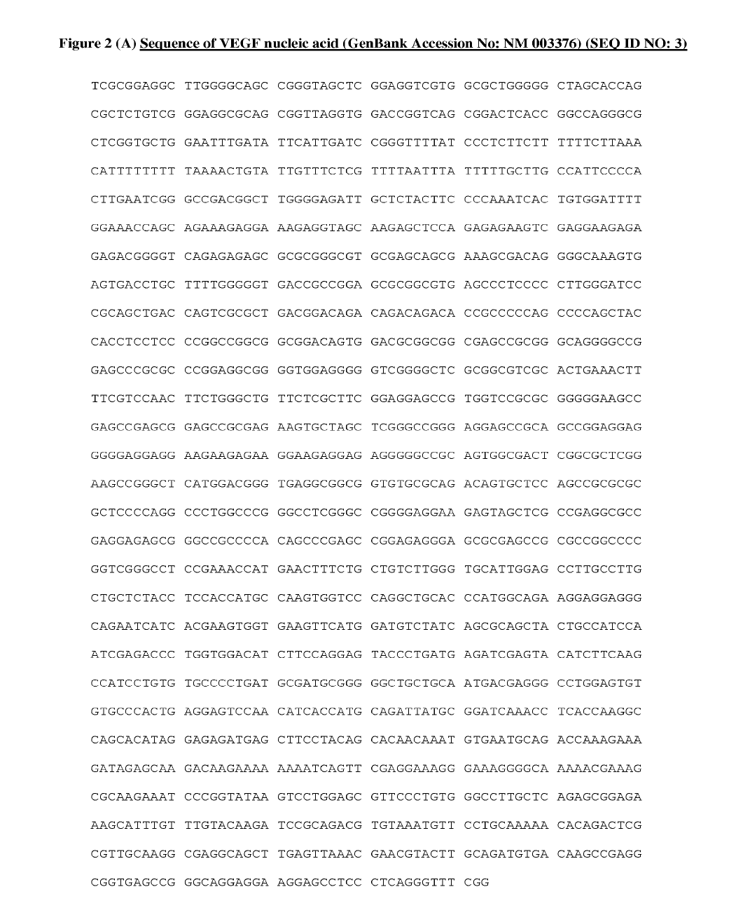

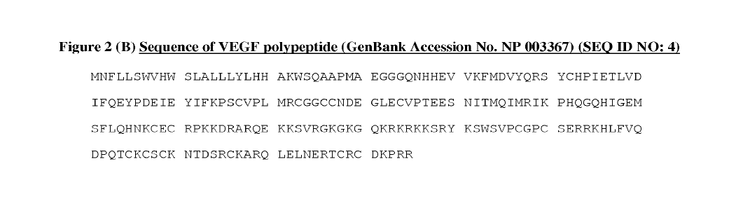

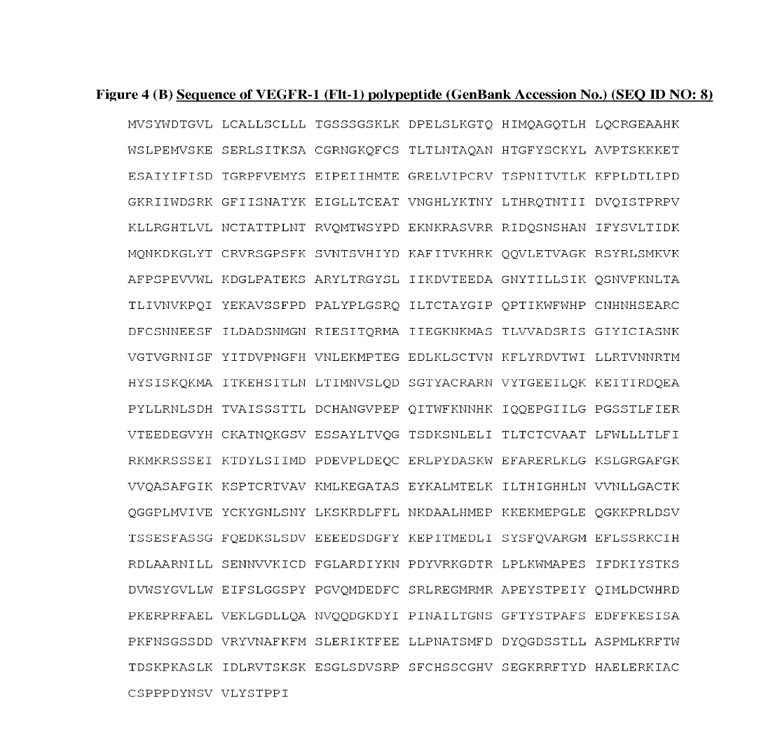

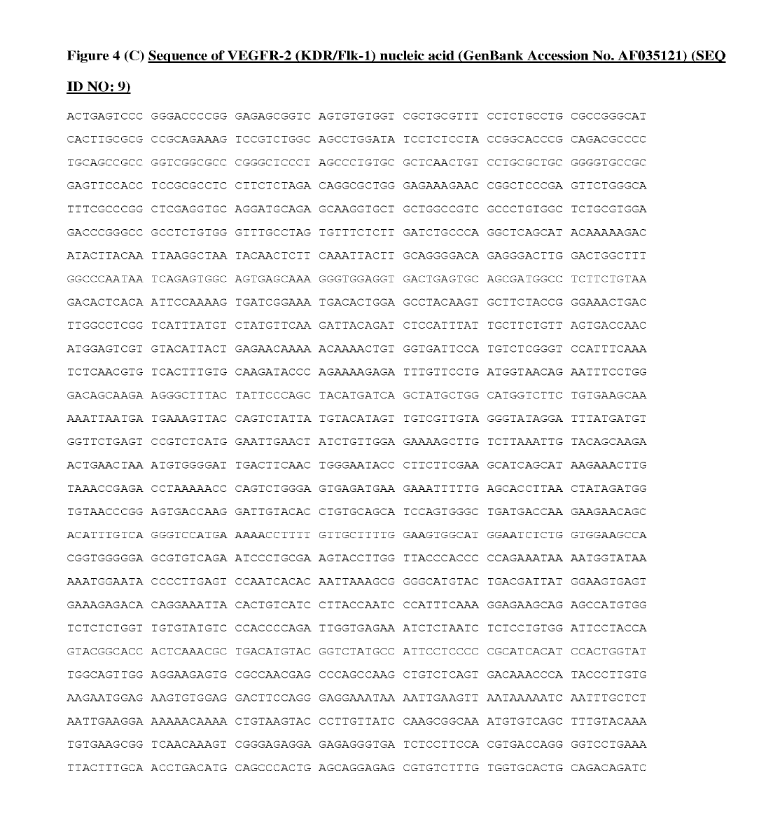

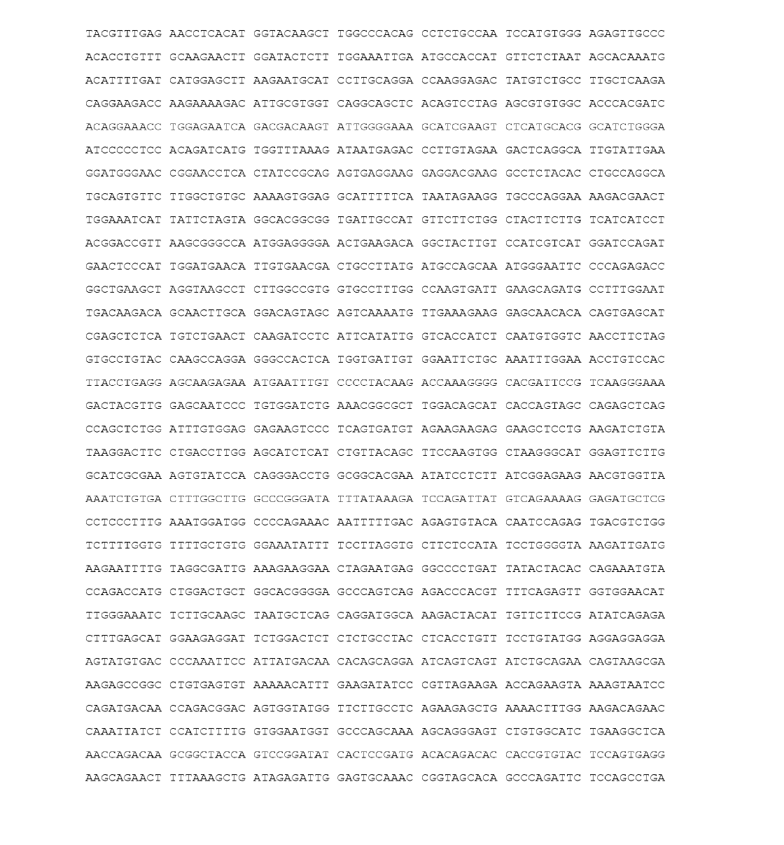

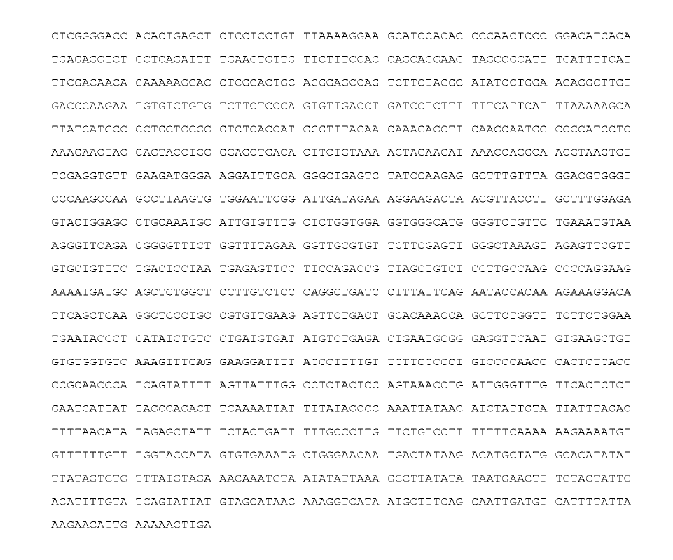

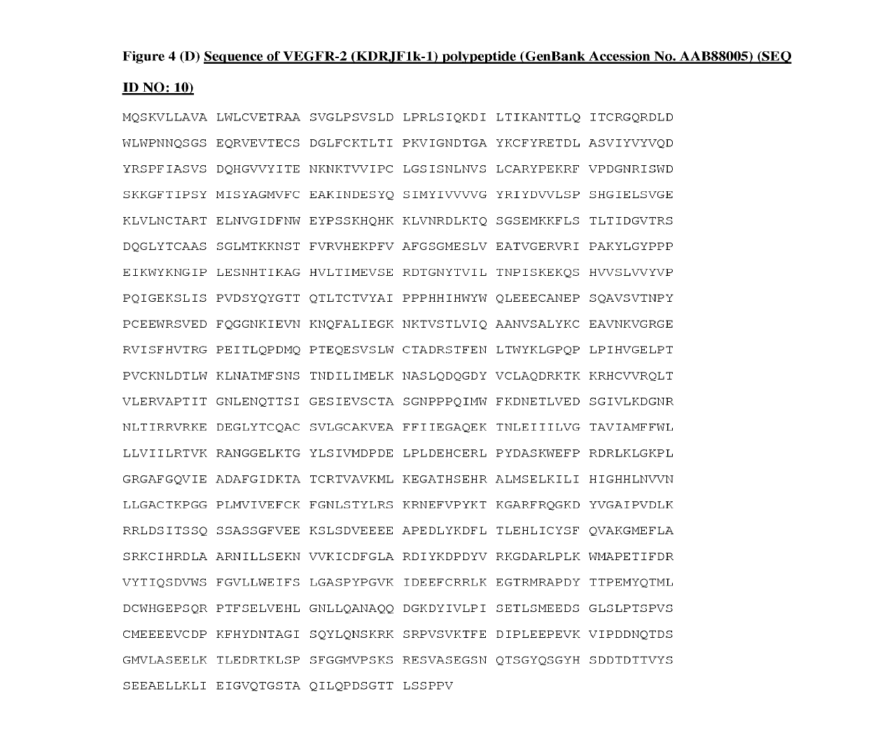

The term “VEGF” refers to a vascular endothelial growth factor that induces angiogenesis or an angiogenic process. As used herein, the term “VEGF” includes the various subtypes of VEGF (also known as vascular permeability factor (VPF) and VEGF-A) (see FIGS. 2(A) and (B)) that arise by, e.g., alternative splicing of the VEGF-A/VPF gene including VEGF₁₂₁, VEGF₁₆₅ and VEGF₁₈₉. Further, as used herein, the term “VEGF” includes VEGF-related angiogenic factors such as PlGF (placenta growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1) (see FIGS. 4(A) and (B)), VEGFR-2 (KDR/Flk-1) (see FIGS. 4(C) and (D)), or VEGFR-3 (FLT-4). The term “VEGF” can be used to refer to a “VEGF” polypeptide or a “VEGF” encoding gene or nucleic acid.

The term “PDGF antagonist” refers to an agent that reduces, or inhibits, either partially or fully, the activity or production of a PDGF. A PDGF antagonist can directly or indirectly reduce or inhibit the activity or production of a specific PDGF such as PDGF-B. Furthermore, “PDGF antagonists” consistent with the above definition of “antagonist,” include agents that act on a PDGF ligand or its cognate receptor so as to reduce or inhibit a PDGF-associated receptor signal. Examples of “PDGF antagonists” include antisense molecules, ribozymes or RNAi that target a PDGF nucleic acid; anti-PDGF aptamers, anti-PDGF antibodies to PDGF itself or its receptor, or soluble PDGF receptor decoys that prevent binding of a PDGF to its cognate receptor; antisense molecules, ribozymes or RNAi that target a cognate PDGF receptor (PDGFR) nucleic acid; anti-PDGFR aptamers or anti-PDGFR antibodies that bind to a cognate PDGFR receptor; and PDGFR tyrosine kinase inhibitors.

The term “VEGF antagonist” refers to an agent that reduces, or inhibits, either partially or fully, the activity or production of a VEGF. A VEGF antagonist can directly or indirectly reduce or inhibit the activity or production of a specific VEGF such as VEGF₁₆₅. Furthermore, “VEGF antagonists” consistent with the above definition of “antagonist,” include agents that act on either a VEGF ligand or its cognate receptor so as to reduce or inhibit a VEGF-associated receptor signal. Examples of “VEGF antagonists” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

The term “effective amount,” when used in connection with an ophthalmological disease, refers to an amount of a PDGF antagonist of Table 1 or Table (below) and a VEGF antagonist of Table 1 or Table 2 that is useful to treat or prevent an ophthalmological disease. The “effective amount” can vary depending upon the mode of administration, specific locus of the ophthalmological disease, the age, body weight, and general health of the mammal. The administration of the PDGF antagonist of Table 1 or Table 2 can occur prior to, subsequent to or concurrently with administration of the VEGF antagonist of Table 1 or Table 2. In one embodiment, the PDGF antagonist of Table 1 or Table 2 and VEGF antagonist of Table 1 or Table 2 are administered as components of the same composition. The effective amount is the total amount of the PDGF antagonist and the VEGF antagonist that is useful for treating or preventing an ophthalmological disease, even if the amount of the PDGF antagonist without the VEGF antagonist, or the VEGF antagonist without the PDGF antagonist, is ineffective to treat or prevent the ophthalmological disease.

A “variant” of polypeptide X refers to a polypeptide having the amino acid sequence of polypeptide X in which is altered in one or more amino acid residues. The variant can have “conservative” changes, wherein a substituted amino acid has similar structural or chemical properties (e.g., replacement of leucine with isoleucine). More rarely, a variant can have “nonconservative” changes (e.g., replacement of glycine with tryptophan). Analogous minor variations may also include amino acid deletions or insertions, or both. Guidance in determining which amino acid residues may be substituted, inserted, or deleted without eliminating biological or immunological activity can be determined using computer programs well known in the art, for example, LASERGENE software (DNASTAR).

The term “variant,” when used in the context of a polynucleotide sequence, can encompass a polynucleotide sequence related to that of gene or the coding sequence thereof. This definition also includes, for example, “allelic,” “splice,” “species,” or “polymorphic” variants. A splice variant can have significant identity to a reference molecule, but will generally have a greater or lesser number of polynucleotides due to alternative splicing of exons during mRNA processing. The corresponding polypeptide can possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

### 7.2 Methods for Treating or Preventing an Ophthalmological Disease

Accordingly, the invention provides methods and compositions useful for treating or preventing an ophthalmological disease. In several embodiments of the present invention, the methods for treating or preventing an ophthalmological disease comprise administration of an effective amount of (a) ARC-127, Antagonist A, Antagonist B, Antagonist C, Antagonist D, 1B3 antibody, CDP860, IMC-3G3, imatinib, 162.62 antibody, 163.31 antibody, 169.14 antibody, 169.31 antibody, αR1 antibody, 2A1E2 antibody, M4TS.11 antibody, M4TS.22 antibody, A10, brefeldin A, sunitinib, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, 5-methyl-7-diethylamino-s-triazolo (1,5-a) pyrimidine, interferon, protamine, PDGFR-B1 monoclonal antibody, PDGFR-B2 monoclonal antibody, 6D11 monoclonal antibody, S is 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, 4-(2-(N-(-2 carboxamidoindole)aminoethyl)-benzenesulfonamide, 4-(2-(N-(-2-carboxamidoindole)aminoethyl)-sulfonylurea, CGP 53716, human antibody g162, pyrazolo[3,4-g]quinoxaline, 6-[2-(methylcarbamoyl)phenylsulphanyl]-3-E-[2-(pyridine-2-yl)ethenyl]-indazole, 1-{2-[5-(2-methoxy-ethoxy)-benzoimidazole-1-yl]-quinoline-8-yl}-piperidine-4-ylamine, 4-[4-[N-(4-nitrophenyl)carbamoyl]-1-piperazinyl]-6,7-dimethoxyquinazoline 4-amino-5-fluoro-3-(6-(4-methyl-piperazine-1-yl)-1H-benzimidazole-2-yl)-1H-quinoline-2-one, (4-tert-butylphenyl) {4-[(6,7-dimethoxy-4-quinolyl)oxy]phenyl}methaneone, 5-methyl-N-[4-(trifluoromethyl)phenyl]-4-isoxazolecarboxamide, trans-4-[(6,7-dimethoxyquinoxaline-2-yl)amino]cyclohexanol, (Z)-3-[(2,4-dimethyl-5-(2-oxo-1,2-dihydroindole-3-ylidenemethyl)-1H-pyrrole-3-yl)-propionic acid, 5-(5-fluoro-2-oxo-1,2-dihydroindole-3-ylidenemethyl)-2,4-dimethyl-1H-pyrrole-3-carboxylic acid, 1-(4-chloroanilino)-4-(4-pyridylmethyl)phthalazine, N-[4-(3-amino-1H-indazole-4-yl)phenyl-N′-(2-fluoro-5-methylphenyl)urea, 1,2-dimethyl-7-(2-thiophene)imidazolo[5,4-g]quinoxaline, 1,2-dimethyl-6-phenyl imidazolo[5,4-g]quinoxaline, 1,2-dimethyl-6-(2-thiophene)imidazolo[5,4-g]quinoxaline, AG1295, AG1296, 3-arylquinoline, 4-pyridyl-2-arylpyrimidine, sorafenib, MLN518, PKC412, AMN107, suramin, or neomycin, or a pharmaceutically acceptable salt thereof; and (b) ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, or G6-31 antibody, or a pharmaceutically acceptable salt thereof (see Table 1). ARC-127, Antagonist A, Antagonist B, Antagonist C, Antagonist D, 1B3 antibody, CDP860, IMC-3G3, imatinib, 162.62 antibody, 163.31 antibody, 169.14 antibody, 169.31 antibody, αR1 antibody, 2A1E2 antibody, M4TS.11 antibody, M4TS.22 antibody, A10, brefeldin A, sunitinib, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-D goat IgG antibody, C3.1 antibody, 5-methyl-7-diethylamino-s-triazolo (1,5-a) pyrimidine, interferon, protamine, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, 4-(2-(N-(-2-carboxamidoindole)aminoethyl)-benzenesulfonamide, 4-(2-(N-(-2-carboxamidoindole)aminoethyl)-sulfonylurea, CGP 53716, human antibody g162, pyrazolo[3,4-g]quinoxaline, 6-[2-(methylcarbamoyl)phenylsulphanyl]-3-E-[2-(pyridine-2-yl)ethenyl]-indazole, 1-{2-[5-(2-methoxy-ethoxy)-benzoimidazole-1-yl]-quinoline-8-yl}-piperidine-4-ylamine, 4-[4-[N-(4-nitrophenyl)carbamoyl]-1-piperazinyl]-6,7-dimethoxyquinazoline-, 4-amino-5-fluoro-3-(6-(4-methyl-piperazine-1-yl)-1H-benzimidazole-2-yl)-1H-quinoline-2-one, (4-tert-butylphenyl) {4-[(6,7-dimethoxy-4-quinolyl)oxy]phenyl}methaneone, 5-methyl-N-[4-(trifluoromethyl)phenyl]-4-isoxazolecarboxamide, trans-4-[(6,7-dimethoxyquinoxaline-2-yl)amino]cyclohexanol, (Z)-3-[(2,4-dimethyl-5-(2-oxo-1,2-dihydroindole-3-ylidenemethyl)-1H-pyrrole-3-yl)-propionic acid, 5-(5-fluoro-2-oxo-1,2-dihydroindole-3-ylidenemethyl)-2,4-dimethyl-1H-pyrrole-3-carboxylic acid, 1-(4-chloroanilino)-4-(4-pyridylmethyl)phthalazine, N-[4-(3-amino-1H-indazole-4-yl)phenyl-N′-(2-fluoro-5-methylphenyl)urea, 1,2-dimethyl-7-(2-thiophene)imidazolo[5,4-g]quinoxaline, 1,2-dimethyl-6-phenyl imidazolo[5,4-g]quinoxaline, 1,2-dimethyl-6 (2-thiophene)imidazolo[5,4-g]quinoxaline, AG1295, AG1296, 3-arylquinoline, 4-pyridyl-2-arylpyrimidine, sorafenib, MLN518, PKC412, AMN107, suramin, and neomycin, and their pharmaceutically acceptable salts are agents that inhibit platelet-derived growth factor (PDGF). Ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG01, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, and G6-31 antibody, and their pharmaceutically acceptable salts are agents that inhibit vascular endothelial growth factor (VEGF). Specific PDGF antagonist-VEGF antagonist pairs useful in the present methods or compositions are set forth in Table 2 (pairs A-EID). The PDGF antagonist or VEGF antagonist of Tables 1 and 2 can be in the form of a pharmaceutically acceptable salt. In the present methods, the PDGF antagonist of any of pairs A-EID can be administered prior to, subsequently to or concurrently with administration of the VEGF antagonist of any of pairs A-EID. In a particular embodiment, the PDGF antagonist is Antagonist A or a pharmaceutically acceptable salt thereof. In another particular embodiment, the PDGF antagonist is Antagonist B or a pharmaceutically acceptable salt thereof. In another particular embodiment, the PDGF antagonist is Antagonist C or a pharmaceutically acceptable salt thereof. In another particular embodiment, the PDGF antagonist is Antagonist D or a pharmaceutically acceptable salt thereof. In another embodiment, the VEGF antagonist is ranibizumab, bevacizumab or aflibercept, or a pharmaceutically acceptable salt thereof. In further embodiments, the methods can further comprise administering another agent that is useful for treating or preventing an ophthalmological disease, such as volociximab.

TABLE 1
List of (a) PDGF antagonists and (b) VEGF antagonists

| (a) PDGF Antagonists | (b) VEGF Antagonists |
|---|---|
| ARC-127 | ranibizumab |
| A compound of Formula A | bevacizumab |
| Antagonist A | aflibercept |
| A compound of Formula B | KH902 VEGF receptor-Fc fusion protein |
| Antagonist B | 2C3 antibody |
| A compound of Formula C | ORA102 |
| Antagonist C | pegaptanib |
| Antagonist D | bevasiranib |
| A compound of Formula E | SIRNA-027 |
| 1B3 antibody | decursin |
| CDP860 | decursinol |
| IMC-3G3 | picropodophyllin |
| Imatinib | guggulsterone |
| 162.62 antibody | PLG101 |
| 163.31 antibody | eicosanoid LXA4 |
| 169.14 antibody | PTK787 |
| 169.31 antibody | pazopanib |
| αR1 antibody | axitinib |
| 2A1E2 antibody | CDDO-Me |
| M4TS.11 antibody | CDDO-Imm |
| M4TS.22 antibody | shikonin |
| A10 | beta-hydroxyisovalerylshikonin |
| brefeldin A | ganglioside GM3 |
| Sunitinib | DC101 antibody |
| Hyb 120.1.2.1.2 antibody | Mab25 antibody |
| Hyb 121.6.1.1.1 antibody | Mab73 antibody |
| Hyb 127.5.7.3.1 antibody | 4A5 antibody |
| Hyb 127.8.2.2.2 antibody | 4E10 antibody |
| Hyb 1.6.1 antibody | 5F12 antibody |
| Hyb 1.11.1 antibody | VA01 antibody |
| Hyb 1.17.1 antibody | BL2 antibody |
| Hyb 1.18.1 antibody | VEGF-related protein |
| Hyb 1.19.1 antibody | sFLT01 |
| Hyb 1.23.1 antibody | sFLT02 |
| Hyb 1.24 antibody | Peptide B3 |
| Hyb 1.25 antibody | TG100801 |
| Hyb 1.29 antibody | sorafenib |
| Hyb 1.33 antibody | G6-31 antibody |
| Hyb 1.38 antibody | A fusion antibody substance that specifically |
| | binds to one or more of a human vascular |
| | endothelial growth factor-A (VEGF-A), human |
| | vascular endothelial growth factor-B (VEGF- |
| | B), human vascular endothelial growth factor- |
| | C (VEGF-C), human vascular endothelial |
| | growth factor-D (VEGF-D), or human vascular |
| | endothelial growth factor-E (VEGF-E) |
| Hyb 1.39 antibody | An antibody that binds to an epitope of VEGF |
| Hyb 1.40 antibody | |
| Hyb 1.45 antibody | |
| Hyb 1.46 antibody | |
| Hyb 1.48 antibody | |
| Hyb 1.49 antibody | |
| Hyb 1.51 antibody | |
| Hyb 6.4.1 antibody | |
| F3 antibody | |
| Humanized F3 antibody | |
| C1 antibody | |
| Humanized C1 antibody | |
| 6.4 antibody | |
| anti-mPDGF-C goat IgG antibody | |
| C3.1 antibody | |
| 5-methyl-7-diethylamino-s-triazolo (1,5-a) | |
| pyrimidine | |
| Interferon | |
| Protamine | |
| PDGFR-B1 monoclonal antibody | |
| PDGFR-B2 monoclonal antibody | |
| 6D11 monoclonal antibody | |
| Sis 1 monoclonal antibody | |
| PR7212 monoclonal antibody | |
| PR292 monoclonal antibody | |
| HYB 9610 monoclonal antibody | |
| HYB 9611 monoclonal antibody | |
| HYB 9612 monoclonal antibody | |
| HYB 9613 monoclonal antibody | |
| 4-(2-(N-(-2 carboxamidoindole) aminoethyl)- | |
| benzenesulfonamide | |
| 4-(2-(N-(-2 carboxamidoindole)aminoethyl)- | |
| sulfonylurea | |
| CGP 53716 small molecule | |
| human antibody g162 | |
| pyrazolo[3,4-g]quinoxaline | |
| 6-[2-(methylcarbamoyl)phenylsulphanyl]-3-E- | |
| [2-(pyridine-2-yl)ethenyl]-indazole 1-{2-[5-(2- | |
| methoxy-ethoxy)-benzoimidazole-1-yl]- | |
| quinoline-8-yl}-piperidine-4-ylamine | |
| 4-[4-[N-(4-nitrophenyl)carbamoyl]-1- | |
| piperazinyl]-6,7-dimethoxyquinazoline | |
| 4-amino-5-fluoro-3-(6-(4-methyl-piperazine- | |
| 1-yl)-1H-benzimidazole-2-yl)-1H-quinoline-2- | |
| one | |
| (4-tert-butylphenyl){4-[(6,7-dimethoxy-4- | |
| quinolyl)oxy]phenyl}methaneone | |
| 5-methyl-N-[4-(trifluoromethyl)phenyl]-4- | |
| isoxazolecarboxamide | |
| trans-4-[(6,7-dimethoxyquinoxaline-2- | |
| yl)amino]cyclohexanol | |
| (Z)-3-[(2,4-dimethyl-5-(2-oxo-1,2- | |
| dihydroindole-3-ylidenemethyl)-1H-pyrrole-3- | |
| yl)-propionic acid | |
| 5-(5-fluoro-2-oxo-1,2-dihydroindole-3- | |
| ylidenemethyl)-2,4-dimethyl-1H-pyrrole-3- | |
| carboxylic acid | |
| 1-(4-chloroanilino)-4-(4- | |
| pyridylmethyl)phthalazine | |
| N-[4-(3-amino-1H-indazole-4-yl)phenyl-N′- | |
| (2-fluoro-5-methylphenyl)urea | |
| 1,2-dimethyl-7-(2-thiophene) imidazolo [5,4- | |
| g] quinoxaline | |
| 1,2-dimethyl-6-phenyl imidazolo [5,4-g] | |
| quinoxaline | |
| 1,2-dimethyl-6-(2-thiophene) imidazolo [5,4- | |
| g] quinoxaline | |
| AG1295 | |
| AG1296 | |
| 3-arylquinoline | |
| 4-pyridyl-2-arylpyrimidine | |
| Sorafenib | |
| MLN518 | |
| PKC412 | |
| AMN107 | |
| Suramin | |
| Neomycin | |
| A fusion antibody substance that specifically | |
| binds to one or more of a human platelet- | |
| derived growth factor-A (PDGF-A), human | |
| platelet-derived growth factor-B (PDGF-B), | |
| human platelet-derived growth factor-C | |
| (PDGF-C), or human platelet-derived growth | |
| factor-D (PDGF-D) | |
| An antibody that binds to an epitope of PDGF | |

TABLE 2
List of specific PDGF antagonist-VEGF antagonist pairs

| Pair | (a) PDGF Antagonist | (b) VEGF Antagonist |
|---|---|---|
| A | ARC-127 | ranibizumab |
| B | ARC-127 | bevacizumab |
| C | ARC-127 | aflibercept |
| D | ARC-127 | KH902 VEGF receptor-Fc fusion protein |
| E | ARC-127 | 2C3 antibody |
| F | ARC-127 | ORA102 |
| G | ARC-127 | pegaptanib |
| H | ARC-127 | bevasiranib |
| I | ARC-127 | SIRNA-027 |
| J | ARC-127 | decursin |
| K | ARC-127 | decursinol |
| L | ARC-127 | picropodophyllin |
| M | ARC-127 | guggulsterone |
| N | ARC-127 | PLG1O1 |
| 0 | ARC-127 | eicosanoid LXA4 |
| P | ARC-127 | PTK787 |
| Q | ARC-127 | pazopanib |
| R | ARC-127 | axitinib |
| S | ARC-127 | CDDO-Me |
| T | ARC-127 | CDDO-Imm |
| U | ARC-127 | shikonin |
| V | ARC-127 | beta-hydroxyisovalerylshikonin |
| W | ARC-127 | ganglioside GM3 |
| X | ARC-127 | DC101 antibody |
| Y | ARC-127 | Mab25 antibody |
| Z | ARC-127 | Mab73 antibody |
| AA | ARC-127 | 4A5 antibody |
| AB | ARC-127 | 4E10 antibody |
| AC | ARC-127 | 5F12 antibody |
| AD | ARC-127 | VA01 antibody |
| AE | ARC-127 | BL2 antibody |
| AF | ARC-127 | VEGF-related protein |
| AG | ARC-127 | sFLT01 |
| AH | ARC-127 | sFLT02 |
| AI | ARC-127 | Peptide B3 |
| AJ | ARC-127 | TG100801 |
| AK | ARC-127 | sorafenib |
| AL | ARC-127 | 06-31 antibody |
| AM | A compound of Formula A | ranibizumab |
| AN | A compound of Formula A | bevacizumab |
| AO | A compound of Formula A | aflibercept |
| AP | A compound of Formula A | KH902 VEGF receptor-Fc fusion protein |
| AQ | A compound of Formula A | 2C3 antibody |
| AR | A compound of Formula A | ORA102 |
| AS | A compound of Formula A | pegaptanib |
| AT | A compound of Formula A | bevasiranib |
| AU | A compound of Formula A | SIRNA-027 |
| AV | A compound of Formula A | decursin |
| AW | A compound of Formula A | decursinol |
| AX | A compound of Formula A | picropodophyllin |
| AY | A compound of Formula A | guggulsterone |
| AZ | A compound of Formula A | PLG1O1 |
| BA | A compound of Formula A | eicosanoid LXA4 |
| BB | A compound of Formula A | PTK787 |
| BC | A compound of Formula A | pazopanib |
| BD | A compound of Formula A | axitinib |
| BE | A compound of Formula A | CDDO-Me |
| BF | A compound of Formula A | CDDO-Imm |
| BG | A compound of Formula A | shikonin |
| BH | A compound of Formula A | beta-hydroxyisovalerylshikonin |
| BI | A compound of Formula A | ganglioside GM3 |
| BJ | A compound of Formula A | DC1O1 antibody |
| BK | A compound of Formula A | Mab25 antibody |
| BL | A compound of Formula A | Mab73 antibody |
| BM | A compound of Formula A | 4A5 antibody |
| BN | A compound of Formula A | 4E10 antibody |
| BO | A compound of Formula A | 5F12 antibody |
| BP | A compound of Formula A | VA01 antibody |
| BQ | A compound of Formula A | BL2 antibody |
| BR | A compound of Formula A | VEGF-related protein |
| BS | A compound of Formula A | sFLT01 |
| BT | A compound of Formula A | sFLT02 |
| BU | A compound of Formula A | Peptide B3 |
| BV | A compound of Formula A | TG100801 |
| BW | A compound of Formula A | sorafenib |
| BX | A compound of Formula A | G6-31 antibody |
| BY | Antagonist A | ranibizumab |
| BZ | Antagonist A | bevacizumab |
| CA | Antagonist A | aflibercept |
| CB | Antagonist A | KH902 VEGF receptor-Fc fusion protein |
| CC | Antagonist A | 2C3 antibody |
| CD | Antagonist A | ORA102 |
| CE | Antagonist A | Pegaptanib |
| CF | Antagonist A | Bevasiranib |
| CO | Antagonist A | SIRNA-027 |
| CH | Antagonist A | Decursin |
| CI | Antagonist A | Decursinol |
| CJ | Antagonist A | picropodophyllin |
| CK | Antagonist A | guggulsterone |
| CL | Antagonist A | PLG101 |
| CM | Antagonist A | eicosanoid LXA4 |
| CN | Antagonist A | PTK787 |
| CO | Antagonist A | pazopanib |
| CP | Antagonist A | Axitinib |
| CQ | Antagonist A | CDDO-Me |
| CR | Antagonist A | CDDO-Imm |
| CS | Antagonist A | Shikonin |
| CT | Antagonist A | beta-hydroxyisovalerylshikonin |
| CU | Antagonist A | ganglioside OM3 |
| CV | Antagonist A | DC101 antibody |
| CW | Antagonist A | Mab25 antibody |
| EX | Antagonist A | Mab73 antibody |
| CY | Antagonist A | 4A5 antibody |
| CZ | Antagonist A | 4E10 antibody |
| DA | Antagonist A | 5F12 antibody |
| DB | Antagonist A | VA01 antibody |
| DC | Antagonist A | BL2 antibody |
| DD | Antagonist A | VEGF-related protein |
| DE | Antagonist A | sFLT01 |
| DF | Antagonist A | sFLT02 |
| DO | Antagonist A | Peptide B3 |
| DH | Antagonist A | TG100801 |
| DI | Antagonist A | sorafenib |
| DJ | Antagonist A | G6-31 antibody |
| DK | A compound of Formula B | ranibizumab |
| DL | A compound of Formula B | bevacizumab |
| DM | A compound of Formula B | aflibercept |
| DN | A compound of Formula B | KH902 VEGF receptor-Fc fusion protein |
| DO | A compound of Formula B | 2C3 antibody |
| DP | A compound of Formula B | ORA102 |
| DQ | A compound of Formula B | pegaptanib |
| DR | A compound of Formula B | bevasiranib |
| DS | A compound of Formula B | SIRNA-027 |
| DT | A compound of Formula B | decursin |
| DU | A compound of Formula B | decursinol |
| DV | A compound of Formula B | picropodophyllin |
| DW | A compound of Formula B | guggulsterone |
| DX | A compound of Formula B | PLG101 |
| DY | A compound of Formula B | eicosanoid LXA4 |
| DZ | A compound of Formula B | PTK787 |
| EA | A compound of Formula B | pazopanib |
| EB | A compound of Formula B | axitinib |
| EC | A compound of Formula B | CDDO-Me |
| ED | A compound of Formula B | CDDO-Imm |
| EE | A compound of Formula B | shikonin |
| EF | A compound of Formula B | beta-hydroxyisovalerylshikonin |
| EG | A compound of Formula B | ganglioside GM3 |
| EH | A compound of Formula B | DC101 antibody |
| EI | A compound of Formula B | Mab25 antibody |
| EJ | A compound of Formula B | Mab73 antibody |
| EK | A compound of Formula B | 4A5 antibody |
| EL | A compound of Formula B | 4E10 antibody |
| EM | A compound of Formula B | 5F12 antibody |
| EN | A compound of Formula B | VA01 antibody |
| EO | A compound of Formula B | BL2 antibody |
| EP | A compound of Formula B | VEGF-related protein |
| EQ | A compound of Formula B | sFLT01 |
| ER | A compound of Formula B | sFLT02 |
| ES | A compound of Formula B | Peptide B3 |
| ET | A compound of Formula B | TG100801 |
| EU | A compound of Formula B | sorafenib |
| EV | A compound of Formula B | G6-31 antibody |
| EW | Antagonist B | ranibizumab |
| EX | Antagonist B | bevacizumab |
| EY | Antagonist B | aflibercept |
| EZ | Antagonist B | KH902 VEGF receptor-Fc fusion protein |
| FA | Antagonist B | 2C3 antibody |
| FB | Antagonist B | ORA102 |
| FC | Antagonist B | pegaptanib |
| FD | Antagonist B | bevasiranib |
| FE | Antagonist B | SIRNA-027 |
| FF | Antagonist B | decursin |
| FG | Antagonist B | decursinol |
| FH | Antagonist B | picropodophyllin |
| FI | Antagonist B | guggulsterone |
| FJ | Antagonist B | PLG101 |
| FK | Antagonist B | eicosanoid LXA4 |
| FL | Antagonist B | PTK787 |
| FM | Antagonist B | pazopanib |
| FN | Antagonist B | axitinib |
| FO | Antagonist B | CDDO-Me |
| FP | Antagonist B | CDDO-Imm |
| FQ | Antagonist B | shikonin |
| FR | Antagonist B | beta-hydroxyisovalerylshikonin |
| FS | Antagonist B | ganglioside GM3 |
| FT | Antagonist B | DC101 antibody |
| FU | Antagonist B | Mab25 antibody |
| FV | Antagonist B | Mab73 antibody |
| FW | Antagonist B | 4A5 antibody |
| FX | Antagonist B | 4E10 antibody |
| FY | Antagonist B | 5F12 antibody |
| FZ | Antagonist B | VA01 antibody |
| GA | Antagonist B | BL2 antibody |
| GB | Antagonist B | VEGF-related protein |
| GC | Antagonist B | sFLT01 |
| GD | Antagonist B | sFLT02 |
| GE | Antagonist B | Peptide B3 |
| GF | Antagonist B | TG100801 |
| GG | Antagonist B | sorafenib |
| GH | Antagonist B | G6-31 antibody |
| GI | A compound of Formula C | ranibizumab |
| GJ | A compound of Formula C | bevacizumab |
| GK | A compound of Formula C | aflibercept |
| GL | A compound of Formula C | KH902 VEGF receptor-Fc fusion protein |
| GM | A compound of Formula C | 2C3 antibody |
| GN | A compound of Formula C | ORA102 |
| GO | A compound of Formula C | pegaptanib |
| GP | A compound of Formula C | bevasiranib |
| GQ | A compound of Formula C | SIRNA-027 |
| GR | A compound of Formula C | decursin |
| GS | A compound of Formula C | decursinol |
| GT | A compound of Formula C | picropodophyllin |
| GU | A compound of Formula C | guggulsterone |
| GV | A compound of Formula C | PLG101 |
| GW | A compound of Formula C | eicosanoid LXA4 |
| GX | A compound of Formula C | PTK787 |
| GY | A compound of Formula C | pazopanib |
| GZ | A compound of Formula C | axitinib |
| HA | A compound of Formula C | CDDO-Me |
| HB | A compound of Formula C | CDDO-Imm |
| HC | A compound of Formula C | shikonin |
| HD | A compound of Formula C | beta-hydroxyisovalerylshikonin |
| HE | A compound of Formula C | ganglioside GM3 |
| HF | A compound of Formula C | DC101 antibody |
| HG | A compound of Formula C | Mab25 antibody |
| HH | A compound of Formula C | Mab73 antibody |
| HI | A compound of Formula C | 4A5 antibody |
| HJ | A compound of Formula C | 4E10 antibody |
| HK | A compound of Formula C | 5F12 antibody |
| HL | A compound of Formula C | VA01 antibody |
| HM | A compound of Formula C | BL2 antibody |
| HN | A compound of Formula C | VEGF-related protein |
| HO | A compound of Formula C | sFLT01 |
| HP | A compound of Formula C | sFLT02 |
| HQ | A compound of Formula C | Peptide B3 |
| HR | A compound of Formula C | TG100801 |
| HS | A compound of Formula C | sorafenib |
| HT | A compound of Formula C | 06-31 antibody |
| HU | Antagonist C | ranibizumab |
| HV | Antagonist C | bevacizumab |
| HW | Antagonist C | aflibercept |
| HX | Antagonist C | KH902 VEGF receptor-Fc fusion protein |
| HY | Antagonist C | 2C3 antibody |
| HZ | Antagonist C | ORA102 |
| IA | Antagonist C | pegaptanib |
| IB | Antagonist C | bevasiranib |
| IC | Antagonist C | SIRNA-027 |
| ID | Antagonist C | Decursin |
| IE [zzz] | Antagonist C | decursinol |
| IF | Antagonist C | picropodophyllin |
| IG | Antagonist C | guggulsterone |
| IH | Antagonist C | PLG101 |
| IK | Antagonist C | eicosanoid LXA4 |
| IL | Antagonist C | PTK787 |
| IM | Antagonist C | pazopanib |
| IN | Antagonist C | axitinib |
| IO | Antagonist C | CDDO-Me |
| IP | Antagonist C | CDDO-Imm |
| IQ | Antagonist C | shikonin |
| IR | Antagonist C | beta-hydroxyisovalerylshikonin |
| IIS | Antagonist C | ganglioside GM3 |
| IT | Antagonist C | DC101 antibody |
| IU | Antagonist C | Mab25 antibody |
| IV | Antagonist C | Mab73 antibody |
| IW | Antagonist C | 4A5 antibody |
| IX | Antagonist C | 4E10 antibody |
| IY | Antagonist C | 5F12 antibody |
| IZ | Antagonist C | VA01 antibody |
| JA | Antagonist C | BL2 antibody |
| JB | Antagonist C | VEGF-related protein |
| JC | Antagonist C | sFLT01 |
| JD | Antagonist C | sFLT02 |
| JE | Antagonist C | Peptide B3 |
| JF | Antagonist C | TG100801 |
| JG | Antagonist C | sorafenib |
| JH | Antagonist C | G6-31 antibody |
| JI | Antagonist D | ranibizumab |
| JK | Antagonist D | bevacizumab |
| JL | Antagonist D | aflibercept |
| JM | Antagonist D | KH902 VEGF receptor-Fc fusion protein |
| JN | Antagonist D | 2C3 antibody |
| JO | Antagonist D | ORA102 |
| JP | Antagonist D | pegaptanib |
| JQ | Antagonist D | bevasiranib |
| JR | Antagonist D | SIRNA-027 |
| JS | Antagonist D | decursin |
| JT | Antagonist D | decursinol |
| JU | Antagonist D | picropodophyllin |
| JV | Antagonist D | guggulsterone |
| JW | Antagonist D | PLG101 |
| JX | Antagonist D | eicosanoid LXA4 |
| JY | Antagonist D | PTK787 |
| JZ | Antagonist D | pazopanib |
| KA | Antagonist D | axitinib |
| KB | Antagonist D | CDDO-Me |
| KC | Antagonist D | CDDO-Imm |
| KD | Antagonist D | shikonin |
| KE | Antagonist D | beta-hydroxyisovalerylshikonin |
| KF | Antagonist D | ganglioside GM3 |
| KG | Antagonist D | DC101 antibody |
| KH | Antagonist D | Mab25 antibody |
| KI | Antagonist D | Mab73 antibody |
| KJ | Antagonist D | 4A5 antibody |
| KK | Antagonist D | 4E10 antibody |
| KL | Antagonist D | 5F12 antibody |
| KM | Antagonist D | VA01 antibody |
| KN | Antagonist D | BL2 antibody |
| KO | Antagonist D | VEGF-related protein |
| KP | Antagonist D | sFLT01 |
| KQ | Antagonist D | sFLT02 |
| KR | Antagonist D | Peptide B3 |
| KS | Antagonist D | TG100801 |
| KT | Antagonist D | sorafenib |
| KU | Antagonist D | G6-31 antibody |
| KV | A compound of Formula E | ranibizumab |
| KW | A compound of Formula E | bevacizumab |
| KX | A compound of Formula E | aflibercept |
| KY | A compound of Formula E | KH902 VEGF receptor-Fc fusion protein |
| KZ | A compound of Formula E | 2C3 antibody |
| LA | A compound of Formula E | ORA102 |
| LB | A compound of Formula E | pegaptanib |
| LC | A compound of Formula E | bevasiranib |
| LD | A compound of Formula E | SIRNA-027 |
| LE | A compound of Formula E | decursin |
| LF | A compound of Formula E | decursinol |
| LG | A compound of Formula E | picropodophyllin |
| LH | A compound of Formula E | guggulsterone |
| LI | A compound of Formula E | PLG101 |
| LJ | A compound of Formula E | eicosanoid LXA4 |
| LK | A compound of Formula E | PTK787 |
| LL | A compound of Formula E | pazopanib |
| LM | A compound of Formula E | axitinib |
| LN | A compound of Formula E | CDDO-Me |
| LO | A compound of Formula E | CDDO-Imm |
| LP | A compound of Formula E | shikonin |
| LQ | A compound of Formula E | beta-hydroxyisovalerylshikonin |
| LR | A compound of Formula E | ganglioside GM3 |
| LS | A compound of Formula E | DC101 antibody |
| LT | A compound of Formula E | Mab25 antibody |
| LU | A compound of Formula E | Mab73 antibody |
| LV | A compound of Formula E | 4A5 antibody |
| LW | A compound of Formula E | 4E10 antibody |
| LX | A compound of Formula E | 5F12 antibody |
| LY | A compound of Formula E | VA01 antibody |
| LZ | A compound of Formula E | BL2 antibody |
| MA | A compound of Formula E | VEGF-related protein |
| MB | A compound of Formula E | sFLT01 |
| MC | A compound of Formula E | sFLT02 |
| MD | A compound of Formula E | Peptide B3 |
| ME | A compound of Formula E | TG100801 |
| MF | A compound of Formula E | sorafenib |
| MG | A compound of Formula E | G6-31 antibody |
| MH | 1B3 antibody | ranibizumab |
| MI | 1B3 antibody | bevacizumab |
| MJ | 1B3 antibody | aflibercept |
| MK | 1B3 antibody | KH902 VEGF receptor-Fc fusion protein |
| ML | 1B3 antibody | 2C3 antibody |
| MM | 1B3 antibody | ORA102 |
| MN | 1B3 antibody | pegaptanib |
| MO | 1B3 antibody | bevasiranib |
| MP | 1B3 antibody | SIRNA-027 |
| MQ | 1B3 antibody | decursin |
| MR | 1B3 antibody | decursinol |
| MS | 1B3 antibody | picropodophyllin |
| MT | 1B3 antibody | guggulsterone |
| MU | 1B3 antibody | PLG101 |
| MV | 1B3 antibody | eicosanoid LXA4 |
| MW | 1B3 antibody | PTK787 |
| MX | 1B3 antibody | pazopanib |
| MY | 1B3 antibody | axitinib |
| MZ | 1B3 antibody | CDDO-Me |
| NA | 1B3 antibody | CDDO-Imm |
| NB | 1B3 antibody | shikonin |
| NC | 1B3 antibody | beta-hydroxyisovalerylshikonin |
| ND | 1B3 antibody | ganglioside GM3 |
| NE | 1B3 antibody | DC101 antibody |
| NF | 1B3 antibody | Mab25 antibody |
| NG | 1B3 antibody | Mab73 antibody |
| NH | 1B3 antibody | 4A5 antibody |
| NI | 1B3 antibody | 4E10 antibody |
| NJ | 1B3 antibody | 5F12 antibody |
| NK | 1B3 antibody | VA01 antibody |
| NL | 1B3 antibody | BL2 antibody |
| NM | 1B3 antibody | VEGF-related protein |
| NN | 1B3 antibody | sFLT01 |
| NO | 1B3 antibody | sFLT02 |
| NP | 1B3 antibody | Peptide B3 |
| NQ | 1B3 antibody | TG100801 |
| NR | 1B3 antibody | sorafenib |
| NS | 1B3 antibody | G6-31 antibody |
| NT | CDP860 | ranibizumab |
| NY | CDP860 | bevacizumab |
| NV | CDP860 | aflibercept |
| NW | CDP860 | KH902 VEGF receptor-Fc fusion protein |
| NX | CDP860 | 2C3 antibody |
| NY | CDP860 | ORA102 |
| NZ | CDP860 | pegaptanib |
| OA | CDP860 | bevasiranib |
| OB | CDP860 | SIRNA-027 |
| OC | CDP860 | decursin |
| OD | CDP860 | decursinol |
| OE | CDP860 | picropodophyllin |
| OF | CDP860 | guggulsterone |
| OG | CDP860 | PLG101 |
| OH | CDP860 | eicosanoid LXA4 |
| OI | CDP860 | PTK787 |
| OJ | CDP860 | pazopanib |
| OK | CDP860 | axitinib |
| OL | CDP860 | CDDO-Me |
| OM | CDP860 | CDDO-Imm |
| ON | CDP860 | shikonin |
| OO | CDP860 | beta-hydroxyisovalerylshikonin |
| OP | CDP860 | ganglioside GM3 |
| OQ | CDP860 | DC101 antibody |
| OR | CDP860 | Mab25 antibody |
| OS | CDP860 | Mab73 antibody |
| OT | CDP860 | 4A5 antibody |
| OY | CDP860 | 4E10 antibody |
| OV | CDP860 | 5F12 antibody |
| OW | CDP860 | VA01 antibody |
| OX | CDP860 | BL2 antibody |
| OY | CDP860 | VEGF-related protein |
| OZ | CDP860 | sFLT01 |
| PA | CDP860 | sFLT02 |
| PB | CDP860 | Peptide B3 |
| PC | CDP860 | TG100801 |
| PD | CDP860 | sorafenib |
| PE | CDP860 | G6-31 antibody |
| PF | IMC-3G3 | ranibizumab |
| PG | IMC-3G3 | bevacizumab |
| PH | IMC-3G3 | aflibercept |
| PI | IMC-3G3 | KH902 VEGF receptor-Fc fusion protein |
| PJ | IMC-3G3 | 2C3 antibody |
| PK | IMC-3G3 | ORA102 |
| PL | IMC-3G3 | pegaptanib |
| PM | IMC-3G3 | bevasiranib |
| PN | IMC-3G3 | SIRNA-027 |
| PO | IMC-3G3 | decursin |
| PP | IMC-3G3 | decursinol |
| PQ | IMC-3G3 | picropodophyllin |
| PR | IMC-3G3 | guggulsterone |
| PS | IMC-3G3 | PLG101 |
| PT | IMC-3G3 | eicosanoid LXA4 |
| PY | IMC-3G3 | PTK787 |
| PV | IMC-3G3 | pazopanib |
| PW | IMC-3G3 | axitinib |
| PX | IMC-3G3 | CDDO-Me |
| PY | IMC-3G3 | CDDO-Imm |
| PZ | IMC-3G3 | shikonin |
| QA | IMC-3G3 | beta-hydroxyisovalerylshikonin |
| QB | IMC-3G3 | ganglioside GM3 |
| QC | IMC-3G3 | DC101 antibody |
| QD | IMC-3G3 | Mab25 antibody |
| QE | IMC-3G3 | Mab73 antibody |
| QF | IMC-3G3 | 4A5 antibody |
| QG | IMC-3G3 | 4E10 antibody |
| QH | IMC-3G3 | 5F12 antibody |
| QI | IMC-3G3 | VA01 antibody |
| QJ | IMC-3G3 | BL2 antibody |
| QK | IMC-3G3 | VEGF-related protein |
| QL | IMC-3G3 | sFLT01 |
| QM | IMC-3G3 | sFLT02 |
| QN | IMC-3G3 | Peptide B3 |
| QO | IMC-3G3 | TG100801 |
| QP | IMC-3G3 | sorafenib |
| QQ | IMC-3G3 | G6-31 antibody |
| QR | Imatinib | ranibizumab |
| QS | Imatinib | bevacizumab |
| QT | Imatinib | aflibercept |
| QY | Imatinib | KH902 VEGF receptor-Fc fusion protein |
| QV | Imatinib | 2C3 antibody |
| QW | Imatinib | ORA102 |
| QX | Imatinib | pegaptanib |
| QY | Imatinib | bevasiranib |
| QZ | Imatinib | SIRNA-027 |
| RA | Imatinib | decursin |
| RB | Imatinib | decursinol |
| RC | Imatinib | picropodophyllin |
| RD | Imatinib | guggulsterone |
| RE | Imatinib | PLG101 |
| RF | Imatinib | eicosanoid LXA4 |
| RG | Imatinib | PTK787 |
| RH | Imatinib | pazopanib |
| RI | Imatinib | axitinib |
| RJ | Imatinib | CDDO-Me |
| RK | Imatinib | CDDO-Imm |
| RL | Imatinib | shikonin |
| RM | Imatinib | beta-hydroxyisovalerylshikonin |
| RN | Imatinib | ganglioside GM3 |
| RO | Imatinib | DC101 antibody |
| RP | Imatinib | Mab25 antibody |
| RQ | Imatinib | Mab73 antibody |
| RR | Imatinib | 4A5 antibody |
| RS | Imatinib | 4E10 antibody |
| RT | Imatinib | 5F12 antibody |
| RY | Imatinib | VA01 antibody |
| RV | Imatinib | BL2 antibody |
| RW | Imatinib | VEGF-related protein |
| RX | Imatinib | sFLT01 |
| RY | Imatinib | sFLT02 |
| RZ | Imatinib | Peptide B3 |
| SA | Imatinib | TG100801 |
| SB | Imatinib | sorafenib |
| SC | Imatinib | G6-31 antibody |
| SD | 162.62 antibody | ranibizumab |
| SE | 162.62 antibody | bevacizumab |
| SF | 162.62 antibody | aflibercept |
| SG | 162.62 antibody | KH902 VEGF receptor-Fc fusion protein |
| SH | 162.62 antibody | 2C3 antibody |
| SI | 162.62 antibody | ORA102 |
| SJ | 162.62 antibody | pegaptanib |
| SK | 162.62 antibody | bevasiranib |
| SL | 162.62 antibody | SIRNA-027 |
| SM | 162.62 antibody | decursin |
| SN | 162.62 antibody | decursinol |
| SO | 162.62 antibody | picropodophyllin |
| SP | 162.62 antibody | guggulsterone |
| SQ | 162.62 antibody | PLG101 |
| SR | 162.62 antibody | eicosanoid LXA4 |
| SS | 162.62 antibody | PTK787 |
| ST | 162.62 antibody | pazopanib |
| SY | 162.62 antibody | axitinib |
| SV | 162.62 antibody | CDDO-Me |
| SW | 162.62 antibody | CDDO-Imm |
| SX | 162.62 antibody | shikonin |
| SY | 162.62 antibody | beta-hydroxyisovalerylshikonin |
| SZ | 162.62 antibody | ganglioside GM3 |
| TA | 162.62 antibody | DC101 antibody |
| TB | 162.62 antibody | Mab25 antibody |
| TC | 162.62 antibody | Mab73 antibody |
| TD | 162.62 antibody | 4A5 antibody |
| TE | 162.62 antibody | 4E10 antibody |
| TF | 162.62 antibody | 5F12 antibody |
| TG | 162.62 antibody | VA01 antibody |
| TH | 162.62 antibody | BL2 antibody |
| TI | 162.62 antibody | VEGF-related protein |
| TJ | 162.62 antibody | sFLT01 |
| TK | 162.62 antibody | sFLT02 |
| TL | 162.62 antibody | Peptide B3 |
| TM | 162.62 antibody | TG100801 |
| TN | 162.62 antibody | sorafenib |
| TO | 162.62 antibody | G6-31 antibody |
| TP | 163.31 antibody | ranibizumab |
| TQ | 163.31 antibody | bevacizumab |
| TR | 163.31 antibody | aflibercept |
| TS | 163.31 antibody | KH902 VEGF receptor-Fc fusion protein |
| TT | 163.31 antibody | 2C3 antibody |
| TY | 163.31 antibody | ORA102 |
| TV | 163.31 antibody | pegaptanib |
| TW | 163.31 antibody | bevasiranib |
| TX | 163.31 antibody | SIRNA-027 |
| TY | 163.31 antibody | decursin |
| TZ | 163.31 antibody | decursinol |
| UA | 163.31 antibody | picropodophyllin |
| UB | 163.31 antibody | guggulsterone |
| UC | 163.31 antibody | PLG101 |
| UD | 163.31 antibody | eicosanoid LXA4 |
| UE | 163.31 antibody | PTK787 |
| UF | 163.31 antibody | pazopanib |
| UG | 163.31 antibody | axitinib |
| UH | 163.31 antibody | CDDO-Me |
| UI | 163.31 antibody | CDDO-Imm |
| UJ | 163.31 antibody | shikonin |
| UK | 163.31 antibody | beta-hydroxyisovalerylshikonin |
| UL | 163.31 antibody | ganglioside GM3 |
| UM | 163.31 antibody | DC101 antibody |
| UN | 163.31 antibody | Mab25 antibody |
| UO | 163.31 antibody | Mab73 antibody |
| UP | 163.31 antibody | 4A5 antibody |
| UQ | 163.31 antibody | 4E10 antibody |
| UR | 163.31 antibody | 5F12 antibody |
| US | 163.31 antibody | VA01 antibody |
| UT | 163.31 antibody | BL2 antibody |
| UY | 163.31 antibody | VEGF-related protein |
| UV | 163.31 antibody | sFLT01 |
| UW | 163.31 antibody | sFLT02 |
| UX | 163.31 antibody | Peptide B3 |
| UY | 163.31 antibody | TG100801 |
| UZ | 163.31 antibody | sorafenib |
| VA | 163.31 antibody | G6-31 antibody |
| VB | 169.14 antibody | ranibizumab |
| VC | 169.14 antibody | bevacizumab |
| VD | 169.14 antibody | aflibercept |
| VE | 169.14 antibody | KH902 VEGF receptor-Fc fusion protein |
| VF | 169.14 antibody | 2C3 antibody |
| VG | 169.14 antibody | ORA102 |
| VH | 169.14 antibody | pegaptanib |
| VI | 169.14 antibody | bevasiranib |
| VJ | 169.14 antibody | SIRNA-027 |
| VK | 169.14 antibody | decursin |
| VL | 169.14 antibody | decursinol |
| VM | 169.14 antibody | picropodophyllin |
| VN | 169.14 antibody | guggulsterone |
| VO | 169.14 antibody | PLG101 |
| VP | 169.14 antibody | eicosanoid LXA4 |
| VQ | 169.14 antibody | PTK787 |
| VR | 169.14 antibody | pazopanib |
| VS | 169.14 antibody | axitinib |
| VT | 169.14 antibody | CDDO-Me |
| VU | 169.14 antibody | CDDO-Imm |
| VV | 169.14 antibody | shikonin |
| VW | 169.14 antibody | beta-hydroxyisovalerylshikonin |
| VX | 169.14 antibody | ganglioside GM3 |
| VY | 169.14 antibody | DC101 antibody |
| VZ | 169.14 antibody | Mab25 antibody |
| WA | 169.14 antibody | Mab73 antibody |
| WB | 169.14 antibody | 4A5 antibody |
| WC | 169.14 antibody | 4E10 antibody |
| WD | 169.14 antibody | 5F12 antibody |
| WE | 169.14 antibody | VA01 antibody |
| WF | 169.14 antibody | BL2 antibody |
| WG | 169.14 antibody | VEGF-related protein |
| WH | 169.14 antibody | sFLT01 |
| WI | 169.14 antibody | sFLT02 |
| WJ | 169.14 antibody | Peptide B3 |
| WK | 169.14 antibody | TG100801 |
| WL | 169.14 antibody | Sorafenib |
| WM | 169.14 antibody | G6-31 antibody |
| WN | 169.31 antibody | ranibizumab |
| WO | 169.31 antibody | bevacizumab |
| WP | 169.31 antibody | aflibercept |
| WQ | 169.31 antibody | KH902 VEGF receptor-Fc fusion protein |
| WR | 169.31 antibody | 2C3 antibody |
| WS | 169.31 antibody | ORA102 |
| WT | 169.31 antibody | pegaptanib |
| WU | 169.31 antibody | bevasiranib |
| WV | 169.31 antibody | SIRNA-027 |
| WW | 169.31 antibody | decursin |
| WX | 169.31 antibody | decursinol |
| WY | 169.31 antibody | picropodophyllin |
| WZ | 169.31 antibody | guggulsterone |
| XA | 169.31 antibody | PLG101 |
| XB | 169.31 antibody | eicosanoid LXA4 |
| XC | 169.31 antibody | PTK787 |
| XD | 169.31 antibody | pazopanib |
| XE | 169.31 antibody | axitinib |
| XF | 169.31 antibody | CDDO-Me |
| XG | 169.31 antibody | CDDO-Imm |
| XH | 169.31 antibody | shikonin |
| XI | 169.31 antibody | beta-hydroxyisovalerylshikonin |
| XJ | 169.31 antibody | ganglioside GM3 |
| XK | 169.31 antibody | DC101 antibody |
| XL | 169.31 antibody | Mab25 antibody |
| XM | 169.31 antibody | Mab73 antibody |
| XN | 169.31 antibody | 4A5 antibody |
| XO | 169.31 antibody | 4E10 antibody |
| XP | 169.31 antibody | 5F12 antibody |
| XQ | 169.31 antibody | VA01 antibody |
| XR | 169.31 antibody | BL2 antibody |
| XS | 169.31 antibody | VEGF-related protein |
| XT | 169.31 antibody | sFLT01 |
| XU | 169.31 antibody | sFLT02 |
| XV | 169.31 antibody | Peptide B3 |
| XW | 169.31 antibody | TG100801 |
| XX | 169.31 antibody | sorafenib |
| XY | 169.31 antibody | G6-31 antibody |
| XZ | αR1 antibody | ranibizumab |
| YA | αR1 antibody | Bevacizumab |
| YB | αR1 antibody | aflibercept |
| YC | αR1 antibody | KI-1902 VEGF receptor-Fc fusion protein |
| YD | αR1 antibody | 2C3 antibody |
| YE | αR1 antibody | ORA102 |
| YF | αR1 antibody | pegaptanib |
| YG | αR1 antibody | bevasiranib |
| YH | αR1 antibody | SIRNA-027 |
| YI | αR1 antibody | decursin |
| YJ | αR1 antibody | decursinol |
| YK | αR1 antibody | picropodophyllin |
| YL | αR1 antibody | guggulsterone |
| YM | αR1 antibody | PLG101 |
| YN | αR1 antibody | eicosanoid LXA4 |
| YO | αR1 antibody | PTK787 |
| YP | αR1 antibody | pazopanib |
| YQ | αR1 antibody | axitinib |
| YR | αR1 antibody | CDDO-Me |
| YS | αR1 antibody | CDDO-Imm |
| YT | αR1 antibody | shikonin |
| YU | αR1 antibody | beta-hydroxyisovalerylshikonin |
| YV | αR1 antibody | ganglioside GM3 |
| YW | αR1 antibody | DC101 antibody |
| YX | αR1 antibody | Mab25 antibody |
| YY | αR1 antibody | Mab73 antibody |
| YZ | αR1 antibody | 4A5 antibody |
| ZA | αR1 antibody | 4E10 antibody |
| ZB | αR1 antibody | 5F12 antibody |
| ZC | αR1 antibody | VA01 antibody |
| ZD | αR1 antibody | BL2 antibody |
| ZE | αR1 antibody | VEGF-related protein |
| ZF | αR1 antibody | sFLT01 |
| ZG | αR1 antibody | sFLT02 |
| ZH | αR1 antibody | Peptide B3 |
| ZI | αR1 antibody | TG100801 |
| ZJ | αR1 antibody | sorafenib |
| ZK | αR1 antibody | G6-31 antibody |
| ZL | 2A1E2 antibody | ranibizumab |
| ZM | 2A1E2 antibody | bevacizumab |
| ZN | 2A1E2 antibody | Aflibercept |
| ZO | 2A1E2 antibody | KH902 VEGF receptor-Fc fusion protein |
| ZP | 2A1E2 antibody | 2C3 antibody |
| ZQ | 2A1E2 antibody | ORA102 |
| ZR | 2A1E2 antibody | pegaptanib |
| ZS | 2A1E2 antibody | bevasiranib |
| ZT | 2A1E2 antibody | SIRNA-027 |
| ZU | 2A1E2 antibody | decursin |
| ZV | 2A1E2 antibody | decursinol |
| ZW | 2A1E2 antibody | picropodophyllin |
| ZX | 2A1E2 antibody | guggulsterone |
| ZY | 2A1E2 antibody | PLG101 |
| ZZ | 2A1E2 antibody | eicosanoid LXA4 |
| AAA | 2A1E2 antibody | PTK787 |
| AAB | 2A1E2 antibody | pazopanib |
| AAC | 2A1E2 antibody | axitinib |
| AAD | 2A1E2 antibody | CDDO-Me |
| AAE | 2A1E2 antibody | CDDO-Imm |
| AAF | 2A1E2 antibody | shikonin |
| AAG | 2A1E2 antibody | beta-hydroxyisovalerylshikonin |
| AAH | 2A1E2 antibody | ganglioside GM3 |
| AAI | 2A1E2 antibody | DC101 antibody |
| AAJ | 2A1E2 antibody | Mab25 antibody |
| AAK | 2A1E2 antibody | Mab73 antibody |
| AAL | 2A1E2 antibody | 4A5 antibody |
| AAM | 2A1E2 antibody | 4E10 antibody |
| AAN | 2A1E2 antibody | 5F12 antibody |
| AAO | 2A1E2 antibody | VA01 antibody |
| AAP | 2A1E2 antibody | BL2 antibody |
| AAQ | 2A1E2 antibody | VEGF-related protein |
| AAR | 2A1E2 antibody | sFLT01 |
| AAS | 2A1E2 antibody | sFLT02 |
| AAT | 2A1E2 antibody | Peptide B3 |
| AAU | 2A1E2 antibody | TG100801 |
| AAV | 2A1E2 antibody | sorafenib |
| AAW | 2A1E2 antibody | G6-31 antibody |
| AAX | M4TS.11 antibody | ranibizumab |
| AAY | M4TS.11 antibody | bevacizumab |
| AAZ | M4TS.11 antibody | aflibercept |
| ABA | M4TS.11 antibody | KH902 VEGF receptor-Fc fusion protein |
| ABB | M4TS.11 antibody | 2C3 antibody |
| ABC | M4TS.11 antibody | ORA102 |
| ABD | M4TS.11 antibody | pegaptanib |
| ABE | M4TS.11 antibody | bevasiranib |
| ABF | M4TS.11 antibody | SIRNA-027 |
| ABG | M4TS.11 antibody | decursin |
| ABH | M4TS.11 antibody | decursinol |
| ABI | M4TS.11 antibody | picropodophyllin |
| ABJ | M4TS.11 antibody | guggulsterone |
| ABK | M4TS.11 antibody | PLG101 |
| ABL | M4TS.11 antibody | eicosanoid LXA4 |
| ABM | M4TS.11 antibody | PTK787 |
| ABN | M4TS.11 antibody | pazopanib |
| ABO | M4TS.11 antibody | axitinib |
| ABP | M4TS.11 antibody | CDDO-Me |
| ABQ | M4TS.11 antibody | CDDO-Imm |
| ABR | M4TS.11 antibody | shikonin |
| ABS | M4TS.11 antibody | beta-hydroxyisovalerylshikonin |
| ABT | M4TS.11 antibody | ganglioside GM3 |
| ABU | M4TS.11 antibody | DC101 antibody |
| ABV | M4TS.11 antibody | Mab25 antibody |
| ABW | M4TS.11 antibody | Mab73 antibody |
| ABX | M4TS.11 antibody | 4A5 antibody |
| ABY | M4TS.11 antibody | 4E10 antibody |
| ABZ | M4TS.11 antibody | 5F12 antibody |
| ACA | M4TS.11 antibody | VA01 antibody |
| ACB | M4TS.11 antibody | BL2 antibody |
| ACC | M4TS.11 antibody | VEGF-related protein |
| ACD | M4TS.11 antibody | sFLT01 |
| ACE | M4TS.11 antibody | sFLT02 |
| ACF | M4TS.11 antibody | Peptide B3 |
| ACG | M4TS.11 antibody | TG100801 |
| ACH | M4TS.11 antibody | sorafenib |
| ACI | M4TS.11 antibody | G6-31 antibody |
| ACJ | M4TS.22 antibody | ranibizumab |
| ACK | M4TS.22 antibody | bevacizumab |
| ACL | M4TS.22 antibody | aflibercept |
| ACM | M4TS.22 antibody | KH902 VEGF receptor-Fc fusion protein |
| ACN | M4TS.22 antibody | 2C3 antibody |
| ACO | M4TS.22 antibody | ORA102 |
| ACP | M4TS.22 antibody | Pegaptanib |
| ACQ | M4TS.22 antibody | bevasiranib |
| ACR | M4TS.22 antibody | SIRNA-027 |
| ACS | M4TS.22 antibody | decursin |
| ACT | M4TS.22 antibody | decursinol |
| ACU | M4TS.22 antibody | picropodophyllin |
| ACV | M4TS.22 antibody | guggulsterone |
| ACW | M4TS.22 antibody | PLG101 |
| ACX | M4TS.22 antibody | eicosanoid LXA4 |
| ACY | M4TS.22 antibody | PTK787 |
| ACZ | M4TS.22 antibody | pazopanib |
| ADA | M4TS.22 antibody | axitinib |
| ADB | M4TS.22 antibody | CDDO-Me |
| ADC | M4TS.22 antibody | CDDO-Imm |
| ADD | M4TS.22 antibody | shikonin |
| ADE | M4TS.22 antibody | beta-hydroxyisovalerylshikonin |
| ADF | M4TS.22 antibody | ganglioside GM3 |
| ADG | M4TS.22 antibody | DC101 antibody |
| ADH | M4TS.22 antibody | Mab25 antibody |
| ADI | M4TS.22 antibody | Mab73 antibody |
| ADJ | M4TS.22 antibody | 4A5 antibody |
| ADK | M4TS.22 antibody | 4E10 antibody |
| ADL | M4TS.22 antibody | 5F12 antibody |
| ADM | M4TS.22 antibody | VA01 antibody |
| AND | M4TS.22 antibody | BL2 antibody |
| ADO | M4TS.22 antibody | VEGF-related protein |
| ADP | M4TS.22 antibody | sFLT01 |
| ADQ | M4TS.22 antibody | sFLT02 |
| ADR | M4TS.22 antibody | Peptide B3 |
| ADS | M4TS.22 antibody | TG100801 |
| ADT | M4TS.22 antibody | sorafenib |
| ADU | M4TS.22 antibody | G6-31 antibody |
| ADV | A10 | ranibizumab |
| ADW | A10 | bevacizumab |
| ADX | A10 | aflibercept |
| ADY | A10 | KH902 VEGF receptor-Fc fusion protein |
| ADZ | A10 | 2C3 antibody |
| AEA | A10 | ORA102 |
| AEB | A10 | pegaptanib |
| AEC | A10 | bevasiranib |
| AED | A10 | SIRNA-027 |
| AEE | A10 | decursin |
| AEF | A10 | decursinol |
| AEG | A10 | picropodophyllin |
| AEH | A10 | guggulsterone |
| AEI | A10 | PLG101 |
| AEJ | A10 | eicosanoid LXA4 |
| AEK | A10 | PTK787 |
| AEL | A10 | pazopanib |
| AEM | A10 | axitinib |
| AEN | A10 | CDDO-Me |
| AEO | A10 | CDDO-Imm |
| AEP | A10 | shikonin |
| AEQ | A10 | beta-hydroxyisovalerylshikonin |
| AER | A10 | ganglioside GM3 |
| AES | A10 | DC101 antibody |
| AET | A10 | Mab25 antibody |
| AEU | A10 | Mab73 antibody |
| AEV | A10 | 4A5 antibody |
| AEW | A10 | 4E10 antibody |
| AEX | A10 | 5F12 antibody |
| AEY | A10 | VA01 antibody |
| AEZ | A10 | BL2 antibody |
| AFA | A10 | VEGF-related protein |
| AFB | A10 | sFLT01 |
| AFC | A10 | sFLT02 |
| AFD | A10 | Peptide B3 |
| AFE | A10 | TG100801 |
| AFF | A10 | sorafenib |
| AFG | A10 | G6-31 antibody |
| AFH | brefeldin A | ranibizumab |
| AFI | brefeldin A | bevacizumab |
| AFJ | brefeldin A | aflibercept |
| AFK | brefeldin A | KH902 VEGF receptor-Fc fusion protein |
| AFL | brefeldin A | 2C3 antibody |
| AFM | brefeldin A | ORA102 |
| AFN | brefeldin A | pegaptanib |
| AFO | brefeldin A | bevasiranib |
| AFP | brefeldin A | SIRNA-027 |
| AFQ | brefeldin A | decursin |
| AFR | brefeldin A | Decursinol |
| AFS | brefeldin A | picropodophyllin |
| AFT | brefeldin A | Guggulsterone |
| AFU | brefeldin A | PLG101 |
| AFV | brefeldin A | eicosanoid LXA4 |
| AFW | brefeldin A | PTK787 |
| AFX | brefeldin A | pazopanib |
| AFY | brefeldin A | axitinib |
| AFZ | brefeldin A | CDDO-Me |
| AGA | brefeldin A | CDDO-Imm |
| AGB | brefeldin A | shikonin |
| AGC | brefeldin A | beta-hydroxyisovalerylshikonin |
| AGD | brefeldin A | ganglioside GM3 |
| AGE | brefeldin A | DC101 antibody |
| AGF | brefeldin A | Mab25 antibody |
| AGG | brefeldin A | Mab73 antibody |
| AGH | brefeldin A | 4A5 antibody |
| AGI | brefeldin A | 4E10 antibody |
| AGJ | brefeldin A | 5F12 antibody |
| AGK | brefeldin A | VA01 antibody |
| AGL | brefeldin A | BL2 antibody |
| AGM | brefeldin A | VEGF-related protein |
| AGN | brefeldin A | sFLT01 |
| AGO | brefeldin A | sFLT02 |
| AGP | brefeldin A | Peptide B3 |
| AGQ | brefeldin A | TG100801 |
| AGR | brefeldin A | sorafenib |
| AGS | brefeldin A | G6-31 antibody |
| AGT | sunitinib | ranibizumab |
| AGU | sunitinib | bevacizumab |
| AGV | sunitinib | aflibercept |
| AGW | sunitinib | KH902 VEGF receptor-Fc fusion protein |
| AGX | sunitinib | 2C3 antibody |
| AGY | sunitinib | ORA102 |
| AGZ | sunitinib | pegaptanib |
| AHA | sunitinib | bevasiranib |
| AHB | sunitinib | SIRNA-027 |
| AHC | sunitinib | decursin |
| AHD | sunitinib | decursinol |
| AHE | sunitinib | picropodophyllin |
| AHF | sunitinib | guggulsterone |
| AHG | sunitinib | PLG101 |
| AHH | sunitinib | eicosanoid LXA4 |
| AHI | sunitinib | PTK787 |
| AHJ | sunitinib | pazopanib |
| AHK | sunitinib | axitinib |
| AHL | sunitinib | CDDO-Me |
| AHM | sunitinib | CDDO-Imm |
| AHN | sunitinib | shikonin |
| AHO | sunitinib | beta-hydroxyisovalerylshikonin |
| AHP | sunitinib | ganglioside GM3 |
| AHQ | sunitinib | DC101 antibody |
| AHR | sunitinib | Mab25 antibody |
| AHS | sunitinib | Mab73 antibody |
| AHT | sunitinib | 4A5 antibody |
| AHU | sunitinib | 4E10 antibody |
| AHV | sunitinib | 5F12 antibody |
| AHW | sunitinib | VA01 antibody |
| AHX | sunitinib | BL2 antibody |
| AHY | sunitinib | VEGF-related protein |
| AHZ | sunitinib | sFLT01 |
| AIA | sunitinib | sFLT02 |
| AIB | sunitinib | Peptide B3 |
| AIC | sunitinib | TG100801 |
| AID | sunitinib | sorafenib |
| AIE | Sunitinib | G6-31 antibody |
| AIF | Hyb 120.1.2.1.2 antibody | ranibizumab |
| AIG | Hyb 120.1.2.1.2 antibody | bevacizumab |
| AIH | Hyb 120.1.2.1.2 antibody | aflibercept |
| AII | Hyb 120.1.2.1.2 antibody | KH902 VEGF receptor-Fc fusion protein |
| AIJ | Hyb 120.1.2.1.2 antibody | 2C3 antibody |
| AIK | Hyb 120.1.2.1.2 antibody | ORA102 |
| AIL | Hyb 120.1.2.1.2 antibody | pegaptanib |
| AIM | Hyb 120.1.2.1.2 antibody | bevasiranib |
| AIN | Hyb 120.1.2.1.2 antibody | SIRNA-027 |
| AIO | Hyb 120.1.2.1.2 antibody | decursin |
| AIP | Hyb 120.1.2.1.2 antibody | decursinol |
| AIQ | Hyb 120.1.2.1.2 antibody | picropodophyllin |
| AIR | Hyb 120.1.2.1.2 antibody | guggulsterone |
| AIS | Hyb 120.1.2.1.2 antibody | PLG101 |
| AIT | Hyb 120.1.2.1.2 antibody | eicosanoid LXA4 |
| AIU | Hyb 120.1.2.1.2 antibody | PTK787 |
| AIV | Hyb 120.1.2.1.2 antibody | pazopanib |
| AIW | Hyb 120.1.2.1.2 antibody | axitinib |
| AIX | Hyb 120.1.2.1.2 antibody | CDDO-Me |
| AIY | Hyb 120.1.2.1.2 antibody | CDDO-Imm |
| AIZ | Hyb 120.1.2.1.2 antibody | shikonin |
| AJA | Hyb 120.1.2.1.2 antibody | beta-hydroxyisovalerylshikonin |
| AJB | Hyb 120.1.2.1.2 antibody | ganglioside GM3 |
| AJC | Hyb 120.1.2.1.2 antibody | DC101 antibody |
| AJD | Hyb 120.1.2.1.2 antibody | Mab25 antibody |
| AJE | Hyb 120.1.2.1.2 antibody | Mab73 antibody |
| AJF | Hyb 120.1.2.1.2 antibody | 4A5 antibody |
| AJG | Hyb 120.1.2.1.2 antibody | 4E10 antibody |
| AJH | Hyb 120.1.2.1.2 antibody | 5F12 antibody |
| AJI | Hyb 120.1.2.1.2 antibody | VA01 antibody |
| AJJ | Hyb 120.1.2.1.2 antibody | BL2 antibody |
| AJK | Hyb 120.1.2.1.2 antibody | VEGF-related protein |
| AJL | Hyb 120.1.2.1.2 antibody | sFLT01 |
| AJM | Hyb 120.1.2.1.2 antibody | sFLT02 |
| AJN | Hyb 120.1.2.1.2 antibody | Peptide B3 |
| AJO | Hyb 120.1.2.1.2 antibody | TG100801 |
| AJP | Hyb 120.1.2.1.2 antibody | sorafenib |
| AJQ | Hyb 120.1.2.1.2 antibody | G6-31 antibody |
| AJR | Hyb 121.6.1.1.1 antibody | ranibizumab |
| AJS | Hyb 121.6.1.1.1 antibody | bevacizumab |
| AJT | Hyb 121.6.1.1.1 antibody | aflibercept |
| AJU | Hyb 121.6.1.1.1 antibody | KH902 VEGF receptor-Fc fusion protein |
| AJV | Hyb 121.6.1.1.1 antibody | 2C3 antibody |
| AJW | Hyb 121.6.1.1.1 antibody | ORA102 |
| AJX | Hyb 121.6.1.1.1 antibody | pegaptanib |
| AJY | Hyb 121.6.1.1.1 antibody | bevasiranib |
| AJZ | Hyb 121.6.1.1.1 antibody | SIRNA-027 |
| AKA | Hyb 121.6.1.1.1 antibody | decursin |
| AKB | Hyb 121.6.1.1.1 antibody | decursinol |
| AKC | Hyb 121.6.1.1.1 antibody | picropodophyllin |
| AKD | Hyb 121.6.1.1.1 antibody | guggulsterone |
| AKE | Hyb 121.6.1.1.1 antibody | PLG101 |
| AKF | Hyb 121.6.1.1.1 antibody | eicosanoid LXA4 |
| AKG | Hyb 121.6.1.1.1 antibody | PTK787 |
| AKH | Hyb 121.6.1.1.1 antibody | pazopanib |
| AKI | Hyb 121.6.1.1.1 antibody | axitinib |
| AKJ | Hyb 121.6.1.1.1 antibody | CDDO-Me |
| AKK | Hyb 121.6.1.1.1 antibody | CDDO-Imm |
| AKL | Hyb 121.6.1.1.1 antibody | shikonin |
| AKM | Hyb 121.6.1.1.1 antibody | beta-hydroxyisovalerylshikonin |
| AKN | Hyb 121.6.1.1.1 antibody | ganglioside GM3 |
| AKO | Hyb 121.6.1.1.1 antibody | DC101 antibody |
| AKP | Hyb 121.6.1.1.1 antibody | Mab25 antibody |
| AKQ | Hyb 121.6.1.1.1 antibody | Mab73 antibody |
| AKR | Hyb 121.6.1.1.1 antibody | 4A5 antibody |
| AKS | Hyb 121.6.1.1.1 antibody | 4E10 antibody |
| AKT | Hyb 121.6.1.1.1 antibody | 5F12 antibody |
| AKU | Hyb 121.6.1.1.1 antibody | VA01 antibody |
| AKV | Hyb 121.6.1.1.1 antibody | BL2 antibody |
| AKW | Hyb 121.6.1.1.1 antibody | VEGF-related protein |
| AKX | Hyb 121.6.1.1.1 antibody | sFLT01 |
| AKY | Hyb 121.6.1.1.1 antibody | sFLT02 |
| AKZ | Hyb 121.6.1.1.1 antibody | Peptide B3 |
| ALA | Hyb 121.6.1.1.1 antibody | TG100801 |
| ALB | Hyb 121.6.1.1.1 antibody | sorafenib |
| ALC | Hyb 121.6.1.1.1 antibody | G6-31 antibody |
| ALD | Hyb 127.5.7.3.1 antibody | ranibizumab |
| ALE | Hyb 127.5.7.3.1 antibody | bevacizumab |
| ALF | Hyb 127.5.7.3.1 antibody | aflibercept |
| ALG | Hyb 127.5.7.3.1 antibody | KH902 VEGF receptor-Fc fusion protein |
| ALH | Hyb 127.5.7.3.1 antibody | 2C3 antibody |
| ALI | Hyb 127.5.7.3.1 antibody | ORA102 |
| ALJ | Hyb 127.5.7.3.1 antibody | pegaptanib |
| ALK | Hyb 127.5.7.3.1 antibody | bevasiranib |
| ALL | Hyb 127.5.7.3.1 antibody | SIRNA-027 |
| ALM | Hyb 127.5.7.3.1 antibody | decursin |
| ALN | Hyb 127.5.7.3.1 antibody | decursinol |
| ALO | Hyb 127.5.7.3.1 antibody | picropodophyllin |
| ALP | Hyb 127.5.7.3.1 antibody | guggulsterone |
| ALQ | Hyb 127.5.7.3.1 antibody | PLG101 |
| ALR | Hyb 127.5.7.3.1 antibody | eicosanoid LXA4 |
| ALS | Hyb 127.5.7.3.1 antibody | PTK787 |
| ALT | Hyb 127.5.7.3.1 antibody | pazopanib |
| ALU | Hyb 127.5.7.3.1 antibody | axitinib |
| ALV | Hyb 127.5.7.3.1 antibody | CDDO-Me |
| ALW | Hyb 127.5.7.3.1 antibody | CDDO-Imm |
| ALX | Hyb 127.5.7.3.1 antibody | shikonin |
| ALY | Hyb 127.5.7.3.1 antibody | beta-hydroxyisovalerylshikonin |
| ALZ | Hyb 127.5.7.3.1 antibody | ganglioside GM3 |
| AMA | Hyb 127.5.7.3.1 antibody | DC101 antibody |
| AMB | Hyb 127.5.7.3.1 antibody | Mab25 antibody |
| AMC | Hyb 127.5.7.3.1 antibody | Mab73 antibody |
| AMD | Hyb 127.5.7.3.1 antibody | 4A5 antibody |
| AME | Hyb 127.5.7.3.1 antibody | 4E10 antibody |
| AMF | Hyb 127.5.7.3.1 antibody | 5F12 antibody |
| AMG | Hyb 127.5.7.3.1 antibody | VA01 antibody |
| AMH | Hyb 127.5.7.3.1 antibody | BL2 antibody |
| AMI | Hyb 127.5.7.3.1 antibody | VEGF-related protein |
| AMJ | Hyb 127.5.7.3.1 antibody | sFLT01 |
| AMK | Hyb 127.5.7.3.1 antibody | sFLT02 |
| AML | Hyb 127.5.7.3.1 antibody | Peptide B3 |
| AMM | Hyb 127.5.7.3.1 antibody | TG100801 |
| AMN | Hyb 127.5.7.3.1 antibody | sorafenib |
| AMO | Hyb 127.5.7.3.1 antibody | G6-31 antibody |
| AMP | Hyb 127.8.2.2.2 antibody | ranibizumab |
| AMQ | Hyb 127.8.2.2.2 antibody | bevacizumab |
| AMR | Hyb 127.8.2.2.2 antibody | aflibercept |
| AMS | Hyb 127.8.2.2.2 antibody | KH902 VEGF receptor-Fc fusion protein |
| AMT | Hyb 127.8.2.2.2 antibody | 2C3 antibody |
| AMU | Hyb 127.8.2.2.2 antibody | ORA102 |
| AMV | Hyb 127.8.2.2.2 antibody | pegaptanib |
| AMW | Hyb 127.8.2.2.2 antibody | bevasiranib |
| AMX | Hyb 127.8.2.2.2 antibody | SIRNA-027 |
| AMY | Hyb 127.8.2.2.2 antibody | decursin |
| AMZ | Hyb 127.8.2.2.2 antibody | decursinol |
| ANA | Hyb 127.8.2.2.2 antibody | picropodophyllin |
| ANB | Hyb 127.8.2.2.2 antibody | guggulsterone |
| ANC | Hyb 127.8.2.2.2 antibody | PLG101 |
| AND | Hyb 127.8.2.2.2 antibody | eicosanoid LXA4 |
| ANE | Hyb 127.8.2.2.2 antibody | PTK787 |
| ANF | Hyb 127.8.2.2.2 antibody | pazopanib |
| ANG | Hyb 127.8.2.2.2 antibody | axitinib |
| ANH | Hyb 127.8.2.2.2 antibody | CDDO-Me |
| ANI | Hyb 127.8.2.2.2 antibody | CDDO-Imm |
| ANJ | Hyb 127.8.2.2.2 antibody | shikonin |
| ANK | Hyb 127.8.2.2.2 antibody | beta-hydroxyisovalerylshikonin |
| ANL | Hyb 127.8.2.2.2 antibody | ganglioside GM3 |
| ANM | Hyb 127.8.2.2.2 antibody | DC101 antibody |
| ANN | Hyb 127.8.2.2.2 antibody | Mab25 antibody |
| ANO | Hyb 127.8.2.2.2 antibody | Mab73 antibody |
| ANP | Hyb 127.8.2.2.2 antibody | 4A5 antibody |
| ANQ | Hyb 127.8.2.2.2 antibody | 4E10 antibody |
| ANR | Hyb 127.8.2.2.2 antibody | 5F12 antibody |
| ANS | Hyb 127.8.2.2.2 antibody | VA01 antibody |
| ANT | Hyb 127.8.2.2.2 antibody | BL2 antibody |
| ANU | Hyb 127.8.2.2.2 antibody | VEGF-related protein |
| ANV | Hyb 127.8.2.2.2 antibody | sFLT01 |
| ANW | Hyb 127.8.2.2.2 antibody | sFLT02 |
| ANX | Hyb 127.8.2.2.2 antibody | Peptide B3 |
| ANY | Hyb 127.8.2.2.2 antibody | TG100801 |
| ANZ | Hyb 127.8.2.2.2 antibody | sorafenib |
| AOA | Hyb 127.8.2.2.2 antibody | G6-31 antibody |
| AOB | Hyb 1.6.1 antibody | ranibizumab |
| AOC | Hyb 1.6.1 antibody | bevacizumab |
| AOD | Hyb 1.6.1 antibody | aflibercept |
| AOE | Hyb 1.6.1 antibody | KH902 VEGF receptor-Fc fusion protein |
| AOF | Hyb 1.6.1 antibody | 2C3 antibody |
| AOG | Hyb 1.6.1 antibody | ORA102 |
| AOH | Hyb 1.6.1 antibody | pegaptanib |
| AOI | Hyb 1.6.1 antibody | bevasiranib |
| AOJ | Hyb 1.6.1 antibody | SIRNA-027 |
| AOK | Hyb 1.6.1 antibody | decursin |
| AOL | Hyb 1.6.1 antibody | decursinol |
| AOM | Hyb 1.6.1 antibody | picropodophyllin |
| AON | Hyb 1.6.1 antibody | guggulsterone |
| AOO | Hyb 1.6.1 antibody | PLG101 |
| AOP | Hyb 1.6.1 antibody | eicosanoid LXA4 |
| AOQ | Hyb 1.6.1 antibody | PTK787 |
| AOR | Hyb 1.6.1 antibody | pazopanib |
| AOS | Hyb 1.6.1 antibody | axitinib |
| AOT | Hyb 1.6.1 antibody | CDDO-Me |
| AOU | Hyb 1.6.1 antibody | CDDO-Imm |
| AOV | Hyb 1.6.1 antibody | shikonin |
| AOW | Hyb 1.6.1 antibody | beta-hydroxyisovalerylshikonin |
| AOX | Hyb 1.6.1 antibody | ganglioside GM3 |
| AOY | Hyb 1.6.1 antibody | DC101 antibody |
| AOZ | Hyb 1.6.1 antibody | Mab25 antibody |
| APA | Hyb 1.6.1 antibody | Mab73 antibody |
| APB | Hyb 1.6.1 antibody | 4A5 antibody |
| APC | Hyb 1.6.1 antibody | 4E10 antibody |
| APD | Hyb 1.6.1 antibody | 5F12 antibody |
| APE | Hyb 1.6.1 antibody | VA01 antibody |
| APF | Hyb 1.6.1 antibody | BL2 antibody |
| APG | Hyb 1.6.1 antibody | VEGF-related protein |
| APH | Hyb 1.6.1 antibody | sFLT01 |
| API | Hyb 1.6.1 antibody | sFLT02 |
| APJ | Hyb 1.6.1 antibody | Peptide B3 |
| APK | Hyb 1.6.1 antibody | TG100801 |
| APL | Hyb 1.6.1 antibody | sorafenib |
| APM | Hyb 1.6.1 antibody | G6-31 antibody |
| APN | Hyb 1.11.1 antibody | ranibizumab |
| APO | Hyb 1.11.1 antibody | bevacizumab |
| APP | Hyb 1.11.1 antibody | aflibercept |
| APQ | Hyb 1.11.1 antibody | KH902 VEGF receptor-Fc fusion protein |
| APR | Hyb 1.11.1 antibody | 2C3 antibody |
| APS | Hyb 1.11.1 antibody | ORA102 |
| APT | Hyb 1.11.1 antibody | pegaptanib |
| APU | Hyb 1.11.1 antibody | bevasiranib |
| APV | Hyb 1.11.1 antibody | SIRNA-027 |
| APW | Hyb 1.11.1 antibody | decursin |
| APX | Hyb 1.11.1 antibody | decursinol |
| APY | Hyb 1.11.1 antibody | picropodophyllin |
| APZ | Hyb 1.11.1 antibody | guggulsterone |
| AQA | Hyb 1.11.1 antibody | PLG101 |
| AQB | Hyb 1.11.1 antibody | eicosanoid LXA4 |
| AQC | Hyb 1.11.1 antibody | PTK787 |
| AQD | Hyb 1.11.1 antibody | pazopanib |
| AQE | Hyb 1.11.1 antibody | axitinib |
| AQF | Hyb 1.11.1 antibody | CDDO-Me |
| AQG | Hyb 1.11.1 antibody | CDDO-Imm |
| AQH | Hyb 1.11.1 antibody | shikonin |
| AQI | Hyb 1.11.1 antibody | beta-hydroxyisovalerylshikonin |
| AQJ | Hyb 1.11.1 antibody | ganglioside GM3 |
| AQK | Hyb 1.11.1 antibody | DC101 antibody |
| AQL | Hyb 1.11.1 antibody | Mab25 antibody |
| AQM | Hyb 1.11.1 antibody | Mab73 antibody |
| AQN | Hyb 1.11.1 antibody | 4A5 antibody |
| AQO | Hyb 1.11.1 antibody | 4E10 antibody |
| AQP | Hyb 1.11.1 antibody | 5F12 antibody |
| AQQ | Hyb 1.11.1 antibody | VA01 antibody |
| AQR | Hyb 1.11.1 antibody | BL2 antibody |
| AQS | Hyb 1.11.1 antibody | VEGF-related protein |
| AQT | Hyb 1.11.1 antibody | sFLT01 |
| AQU | Hyb 1.11.1 antibody | sFLT02 |
| AQV | Hyb 1.11.1 antibody | Peptide B3 |
| AQW | Hyb 1.11.1 antibody | TG100801 |
| AQX | Hyb 1.11.1 antibody | sorafenib |
| AQY | Hyb 1.11.1 antibody | G6-31 antibody |
| AQZ | Hyb 1.17.1 antibody | ranibizumab |
| ARA | Hyb 1.17.1 antibody | bevacizumab |
| ARB | Hyb 1.17.1 antibody | aflibercept |
| ARC | Hyb 1.17.1 antibody | KH902 VEGF receptor-Fc fusion protein |
| ARD | Hyb 1.17.1 antibody | 2C3 antibody |
| ARE | Hyb 1.17.1 antibody | ORA102 |
| ARF | Hyb 1.17.1 antibody | pegaptanib |
| ARG | Hyb 1.17.1 antibody | bevasiranib |
| ARH | Hyb 1.17.1 antibody | SIRNA-027 |
| ARI | Hyb 1.17.1 antibody | decursin |
| ARJ | Hyb 1.17.1 antibody | decursinol |
| ARK | Hyb 1.17.1 antibody | picropodophyllin |
| ARL | Hyb 1.17.1 antibody | guggulsterone |
| ARM | Hyb 1.17.1 antibody | PLG101 |
| ARN | Hyb 1.17.1 antibody | eicosanoid LXA4 |
| ARO | Hyb 1.17.1 antibody | PTK787 |
| ARP | Hyb 1.17.1 antibody | pazopanib |
| ARQ | Hyb 1.17.1 antibody | axitinib |
| ARR | Hyb 1.17.1 antibody | CDDO-Me |
| ARS | Hyb 1.17.1 antibody | CDDO-Imm |
| ART | Hyb 1.17.1 antibody | shikonin |
| ARU | Hyb 1.17.1 antibody | beta-hydroxyisovalerylshikonin |
| ARV | Hyb 1.17.1 antibody | ganglioside GM3 |
| ARW | Hyb 1.17.1 antibody | DC101 antibody |
| ARX | Hyb 1.17.1 antibody | Mab25 antibody |
| ARY | Hyb 1.17.1 antibody | Mab73 antibody |
| ARZ | Hyb 1.17.1 antibody | 4A5 antibody |
| ASA | Hyb 1.17.1 antibody | 4E10 antibody |
| ASB | Hyb 1.17.1 antibody | 5F12 antibody |
| ASC | Hyb 1.17.1 antibody | VA01 antibody |
| ASD | Hyb 1.17.1 antibody | BL2 antibody |
| ASE | Hyb 1.17.1 antibody | VEGF-related protein |
| ASF | Hyb 1.17.1 antibody | sFLT01 |
| ASG | Hyb 1.17.1 antibody | sFLT02 |
| ASH | Hyb 1.17.1 antibody | Peptide B3 |
| ASI | Hyb 1.17.1 antibody | TG100801 |
| ASJ | Hyb 1.17.1 antibody | Sorafenib |
| ASK | Hyb 1.17.1 antibody | G6-31 antibody |
| ASL | Hyb 1.18.1 antibody | ranibizumab |
| ASM | Hyb 1.18.1 antibody | bevacizumab |
| ASN | Hyb 1.18.1 antibody | aflibercept |
| ASO | Hyb 1.18.1 antibody | KH902 VEGF receptor-Fc fusion protein |
| ASP | Hyb 1.18.1 antibody | 2C3 antibody |
| ASQ | Hyb 1.18.1 antibody | ORA102 |
| ASR | Hyb 1.18.1 antibody | pegaptanib |
| ASS | Hyb 1.18.1 antibody | bevasiranib |
| AST | Hyb 1.18.1 antibody | SIRNA-027 |
| ASU | Hyb 1.18.1 antibody | decursin |
| ASV | Hyb 1.18.1 antibody | decursinol |
| ASW | Hyb 1.18.1 antibody | picropodophyllin |
| ASX | Hyb 1.18.1 antibody | guggulsterone |
| ASY | Hyb 1.18.1 antibody | PLG101 |
| ASZ | Hyb 1.18.1 antibody | eicosanoid LXA4 |
| ATA | Hyb 1.18.1 antibody | PTK787 |
| ATB | Hyb 1.18.1 antibody | pazopanib |
| ATC | Hyb 1.18.1 antibody | axitinib |
| ATD | Hyb 1.18.1 antibody | CDDO-Me |
| ATE | Hyb 1.18.1 antibody | CDDO-Imm |
| ATF | Hyb 1.18.1 antibody | shikonin |
| ATG | Hyb 1.18.1 antibody | beta-hydroxyisovalerylshikonin |
| ATH | Hyb 1.18.1 antibody | ganglioside GM3 |
| ATI | Hyb 1.18.1 antibody | DC101 antibody |
| ATJ | Hyb 1.18.1 antibody | Mab25 antibody |
| ATK | Hyb 1.18.1 antibody | Mab73 antibody |
| ATL | Hyb 1.18.1 antibody | 4A5 antibody |
| ATM | Hyb 1.18.1 antibody | 4E10 antibody |
| ATN | Hyb 1.18.1 antibody | 5F12 antibody |
| ATO | Hyb 1.18.1 antibody | VA01 antibody |
| ATP | Hyb 1.18.1 antibody | BL2 antibody |
| ATQ | Hyb 1.18.1 antibody | VEGF-related protein |
| ATR | Hyb 1.18.1 antibody | sFLT01 |
| ATS | Hyb 1.18.1 antibody | sFLT02 |
| ATT | Hyb 1.18.1 antibody | Peptide B3 |
| ATU | Hyb 1.18.1 antibody | TG100801 |
| ATV | Hyb 1.18.1 antibody | sorafenib |
| ATW | Hyb 1.18.1 antibody | G6-31 antibody |
| ATX | Hyb 1.19.1 antibody | ranibizumab |
| ATY | Hyb 1.19.1 antibody | Bevacizumab |
| ATZ | Hyb 1.19.1 antibody | aflibercept |
| AUA | Hyb 1.19.1 antibody | KH902 VEGF receptor-Fc fusion protein |
| AUB | Hyb 1.19.1 antibody | 2C3 antibody |
| AUC | Hyb 1.19.1 antibody | ORA102 |
| AUD | Hyb 1.19.1 antibody | pegaptanib |
| AUE | Hyb 1.19.1 antibody | bevasiranib |
| AUF | Hyb 1.19.1 antibody | SIRNA-027 |
| AUG | Hyb 1.19.1 antibody | decursin |
| AUH | Hyb 1.19.1 antibody | decursinol |
| AUI | Hyb 1.19.1 antibody | picropodophyllin |
| AUJ | Hyb 1.19.1 antibody | guggulsterone |
| AUK | Hyb 1.19.1 antibody | PLG101 |
| AUL | Hyb 1.19.1 antibody | eicosanoid LXA4 |
| AUM | Hyb 1.19.1 antibody | PTK787 |
| AUN | Hyb 1.19.1 antibody | pazopanib |
| AUG | Hyb 1.19.1 antibody | axitinib |
| AUP | Hyb 1.19.1 antibody | CDDO-Me |
| AUQ | Hyb 1.19.1 antibody | CDDO-Imm |
| AUR | Hyb 1.19.1 antibody | shikonin |
| AUS | Hyb 1.19.1 antibody | beta-hydroxyisovalerylshikonin |
| AUT | Hyb 1.19.1 antibody | ganglioside GM3 |
| AUU | Hyb 1.19.1 antibody | DC101 antibody |
| AUV | Hyb 1.19.1 antibody | Mab25 antibody |
| AUX | Hyb 1.19.1 antibody | Mab73 antibody |
| AUY | Hyb 1.19.1 antibody | 4A5 antibody |
| AUZ | Hyb 1.19.1 antibody | 4E10 antibody |
| AVA | Hyb 1.19.1 antibody | 5F12 antibody |
| AVB | Hyb 1.19.1 antibody | VA01 antibody |
| AVC | Hyb 1.19.1 antibody | BL2 antibody |
| AVD | Hyb 1.19.1 antibody | VEGF-related protein |
| AVE | Hyb 1.19.1 antibody | sFLT01 |
| AVF | Hyb 1.19.1 antibody | sFLT02 |
| AVG | Hyb 1.19.1 antibody | Peptide B3 |
| AVH | Hyb 1.19.1 antibody | TG100801 |
| AVI | Hyb 1.19.1 antibody | sorafenib |
| AVJ | Hyb 1.19.1 antibody | G6-31 antibody |
| AVK | Hyb 1.23.1 antibody | ranibizumab |
| AVL | Hyb 1.23.1 antibody | bevacizumab |
| AVM | Hyb 1.23.1 antibody | aflibercept |
| AVN | Hyb 1.23.1 antibody | KH902 VEGF receptor-Fc fusion protein |
| AVO | Hyb 1.23.1 antibody | 2C3 antibody |
| AVP | Hyb 1.23.1 antibody | ORA102 |
| AVQ | Hyb 1.23.1 antibody | pegaptanib |
| AVR | Hyb 1.23.1 antibody | bevasiranib |
| AVS | Hyb 1.23.1 antibody | SIRNA-027 |
| AVT | Hyb 1.23.1 antibody | decursin |
| AVU | Hyb 1.23.1 antibody | decursinol |
| AVV | Hyb 1.23.1 antibody | picropodophyllin |
| AVW | Hyb 1.23.1 antibody | guggulsterone |
| AVX | Hyb 1.23.1 antibody | PLG101 |
| AVY | Hyb 1.23.1 antibody | eicosanoid LXA4 |
| AVZ | Hyb 1.23.1 antibody | PTK787 |
| AWA | Hyb 1.23.1 antibody | pazopanib |
| AWB | Hyb 1.23.1 antibody | axitinib |
| AWC | Hyb 1.23.1 antibody | CDDO-Me |
| AWD | Hyb 1.23.1 antibody | CDDO-Imm |
| AWE | Hyb 1.23.1 antibody | shikonin |
| AWF | Hyb 1.23.1 antibody | beta-hydroxyisovalerylshikonin |
| AWG | Hyb 1.23.1 antibody | ganglioside GM3 |
| AWH | Hyb 1.23.1 antibody | DC101 antibody |
| AWI | Hyb 1.23.1 antibody | Mab25 antibody |
| AWJ | Hyb 1.23.1 antibody | Mab73 antibody |
| AWK | Hyb 1.23.1 antibody | 4A5 antibody |
| AWL | Hyb 1.23.1 antibody | 4E10 antibody |
| AWM | Hyb 1.23.1 antibody | 5F12 antibody |
| AWN | Hyb 1.23.1 antibody | VA01 antibody |
| AWO | Hyb 1.23.1 antibody | BL2 antibody |
| AWP | Hyb 1.23.1 antibody | VEGF-related protein |
| AWQ | Hyb 1.23.1 antibody | sFLT01 |
| AWR | Hyb 1.23.1 antibody | sFLT02 |
| AWS | Hyb 1.23.1 antibody | Peptide B3 |
| AWT | Hyb 1.23.1 antibody | TG100801 |
| AWU | Hyb 1.23.1 antibody | sorafenib |
| AWV | Hyb 1.23.1 antibody | G6-31 antibody |
| AWW | Hyb 1.24 antibody | ranibizumab |
| AWX | Hyb 1.24 antibody | bevacizumab |
| AWY | Hyb 1.24 antibody | aflibercept |
| AWZ | Hyb 1.24 antibody | KH902 VEGF receptor-Fc fusion protein |
| AXA | Hyb 1.24 antibody | 2C3 antibody |
| AXB | Hyb 1.24 antibody | ORA102 |
| AXC | Hyb 1.24 antibody | pegaptanib |
| AXD | Hyb 1.24 antibody | Bevasiranib |
| AXE | Hyb 1.24 antibody | SIRNA-027 |
| AXF | Hyb 1.24 antibody | decursin |
| AXG | Hyb 1.24 antibody | decursinol |
| AXH | Hyb 1.24 antibody | picropodophyllin |
| AXI | Hyb 1.24 antibody | guggulsterone |
| AXJ | Hyb 1.24 antibody | PLG101 |
| AXK | Hyb 1.24 antibody | eicosanoid LXA4 |
| AXL | Hyb 1.24 antibody | PTK787 |
| AXM | Hyb 1.24 antibody | pazopanib |
| AXN | Hyb 1.24 antibody | axitinib |
| AXO | Hyb 1.24 antibody | CDDO-Me |
| AXP | Hyb 1.24 antibody | CDDO-Imm |
| AXQ | Hyb 1.24 antibody | shikonin |
| AXR | Hyb 1.24 antibody | beta-hydroxyisovalerylshikonin |
| AXS | Hyb 1.24 antibody | ganglioside GM3 |
| AXT | Hyb 1.24 antibody | DC101 antibody |
| AXU | Hyb 1.24 antibody | Mab25 antibody |
| AXV | Hyb 1.24 antibody | Mab73 antibody |
| AXW | Hyb 1.24 antibody | 4A5 antibody |
| AXX | Hyb 1.24 antibody | 4E10 antibody |
| AXY | Hyb 1.24 antibody | 5F12 antibody |
| AXZ | Hyb 1.24 antibody | VA01 antibody |
| AYA | Hyb 1.24 antibody | BL2 antibody |
| AYB | Hyb 1.24 antibody | VEGF-related protein |
| AYC | Hyb 1.24 antibody | sFLT01 |
| AYD | Hyb 1.24 antibody | sFLT02 |
| AYE | Hyb 1.24 antibody | Peptide B3 |
| AYF | Hyb 1.24 antibody | TG100801 |
| AYG | Hyb 1.24 antibody | sorafenib |
| AYH | Hyb 1.24 antibody | G6-31 antibody |
| AYI | Hyb 1.25 antibody | ranibizumab |
| AYJ | Hyb 1.25 antibody | bevacizumab |
| AYK | Hyb 1.25 antibody | aflibercept |
| AYL | Hyb 1.25 antibody | KH902 VEGF receptor-Fc fusion protein |
| AYM | Hyb 1.25 antibody | 2C3 antibody |
| AYN | Hyb 1.25 antibody | ORA102 |
| AYO | Hyb 1.25 antibody | pegaptanib |
| AYP | Hyb 1.25 antibody | bevasiranib |
| AYQ | Hyb 1.25 antibody | SIRNA-027 |
| AYR | Hyb 1.25 antibody | decursin |
| AYS | Hyb 1.25 antibody | Decursinol |
| AYT | Hyb 1.25 antibody | picropodophyllin |
| AYU | Hyb 1.25 antibody | guggulsterone |
| AYV | Hyb 1.25 antibody | PLG101 |
| AYW | Hyb 1.25 antibody | eicosanoid LXA4 |
| AYX | Hyb 1.25 antibody | PTK787 |
| AYY | Hyb 1.25 antibody | pazopanib |
| AYZ | Hyb 1.25 antibody | axitinib |
| AZA | Hyb 1.25 antibody | CDDO-Me |
| AZB | Hyb 1.25 antibody | CDDO-Imm |
| AZC | Hyb 1.25 antibody | shikonin |
| AZD | Hyb 1.25 antibody | beta-hydroxyisovalerylshikonin |
| AZE | Hyb 1.25 antibody | ganglioside GM3 |
| AZF | Hyb 1.25 antibody | DC101 antibody |
| AZG | Hyb 1.25 antibody | Mab25 antibody |
| AZH | Hyb 1.25 antibody | Mab73 antibody |
| AZI | Hyb 1.25 antibody | 4A5 antibody |
| AZT | Hyb 1.25 antibody | 4E10 antibody |
| AZK | Hyb 1.25 antibody | 5F12 antibody |
| AZL | Hyb 1.25 antibody | VA01 antibody |
| AZM | Hyb 1.25 antibody | BL2 antibody |
| AZN | Hyb 1.25 antibody | VEGF-related protein |
| AZO | Hyb 1.25 antibody | sFLT01 |
| AZP | Hyb 1.25 antibody | sFLT02 |
| AZQ | Hyb 1.25 antibody | Peptide B3 |
| AZR | Hyb 1.25 antibody | TG100801 |
| AZS | Hyb 1.25 antibody | sorafenib |
| AZT | Hyb 1.25 antibody | G6-31 antibody |
| AZU | Hyb 1.29 antibody | ranibizumab |
| AZV | Hyb 1.29 antibody | bevacizumab |
| AZW | Hyb 1.29 antibody | aflibercept |
| AZX | Hyb 1.29 antibody | KH902 VEGF receptor-Fc fusion protein |
| AZY | Hyb 1.29 antibody | 2C3 antibody |
| AZZ | Hyb 1.29 antibody | ORA102 |
| BAA | Hyb 1.29 antibody | pegaptanib |
| BAB | Hyb 1.29 antibody | bevasiranib |
| BAC | Hyb 1.29 antibody | SIRNA-027 |
| BAD | Hyb 1.29 antibody | decursin |
| BAE | Hyb 1.29 antibody | decursinol |
| BAF | Hyb 1.29 antibody | picropodophyllin |
| BAG | Hyb 1.29 antibody | guggulsterone |
| BAH | Hyb 1.29 antibody | PLG101 |
| BAI | Hyb 1.29 antibody | eicosanoid LXA4 |
| BAJ | Hyb 1.29 antibody | PTK787 |
| BAK | Hyb 1.29 antibody | pazopanib |
| BAL | Hyb 1.29 antibody | axitinib |
| BAM | Hyb 1.29 antibody | CDDO-Me |
| BAN | Hyb 1.29 antibody | CDDO-Imm |
| BAO | Hyb 1.29 antibody | shikonin |
| BAP | Hyb 1.29 antibody | beta-hydroxyisovalerylshikonin |
| BAQ | Hyb 1.29 antibody | ganglioside GM3 |
| BAR | Hyb 1.29 antibody | DC101 antibody |
| ABS | Hyb 1.29 antibody | Mab25 antibody |
| BAT | Hyb 1.29 antibody | Mab73 antibody |
| BAU | Hyb 1.29 antibody | 4A5 antibody |
| BAV | Hyb 1.29 antibody | 4E10 antibody |
| BAW | Hyb 1.29 antibody | 5F12 antibody |
| BAX | Hyb 1.29 antibody | VA01 antibody |
| BAY | Hyb 1.29 antibody | BL2 antibody |
| BAZ | Hyb 1.29 antibody | VEGF-related protein |
| BBA | Hyb 1.29 antibody | sFLT01 |
| BBB | Hyb 1.29 antibody | sFLT02 |
| BBC | Hyb 1.29 antibody | Peptide B3 |
| BBD | Hyb 1.29 antibody | TG100801 |
| BBE | Hyb 1.29 antibody | sorafenib |
| BBF | Hyb 1.29 antibody | G6-31 antibody |
| BBG | Hyb 1.33 antibody | ranibizumab |
| BBH | Hyb 1.33 antibody | bevacizumab |
| BBI | Hyb 1.33 antibody | aflibercept |
| BBJ | Hyb 1.33 antibody | KH902 VEGF receptor-Fc fusion protein |
| BBK | Hyb 1.33 antibody | 2C3 antibody |
| BBL | Hyb 1.33 antibody | ORA102 |
| BBM | Hyb 1.33 antibody | pegaptanib |
| BBN | Hyb 1.33 antibody | bevasiranib |
| BBO | Hyb 1.33 antibody | SIRNA-027 |
| BBP | Hyb 1.33 antibody | decursin |
| BBQ | Hyb 1.33 antibody | decursinol |
| BBR | Hyb 1.33 antibody | picropodophyllin |
| BBS | Hyb 1.33 antibody | guggulsterone |
| BBT | Hyb 1.33 antibody | PLG101 |
| BBU | Hyb 1.33 antibody | eicosanoid LXA4 |
| BBV | Hyb 1.33 antibody | PTK787 |
| BBW | Hyb 1.33 antibody | Pazopanib |
| BBX | Hyb 1.33 antibody | axitinib |
| BBY | Hyb 1.33 antibody | CDDO-Me |
| BBZ | Hyb 1.33 antibody | CDDO-Imm |
| BCA | Hyb 1.33 antibody | shikonin |
| BCB | Hyb 1.33 antibody | beta-hydroxyisovalerylshikonin |
| BCC | Hyb 1.33 antibody | ganglioside GM3 |
| BCD | Hyb 1.33 antibody | DC101 antibody |
| BCE | Hyb 1.33 antibody | Mab25 antibody |
| BCF | Hyb 1.33 antibody | Mab73 antibody |
| BCG | Hyb 1.33 antibody | 4A5 antibody |
| BCH | Hyb 1.33 antibody | 4E10 antibody |
| BCI | Hyb 1.33 antibody | 5F12 antibody |
| BCJ | Hyb 1.33 antibody | VA01 antibody |
| BCK | Hyb 1.33 antibody | BL2 antibody |
| BCL | Hyb 1.33 antibody | VEGF-related protein |
| BCM | Hyb 1.33 antibody | sFLT01 |
| BCN | Hyb 1.33 antibody | sFLT02 |
| BCO | Hyb 1.33 antibody | Peptide B3 |
| BCP | Hyb 1.33 antibody | TG100801 |
| BCQ | Hyb 1.33 antibody | sorafenib |
| BCR | Hyb 1.33 antibody | G6-31 antibody |
| BCS | Hyb 1.38 antibody | ranibizumab |
| BCT | Hyb 1.38 antibody | bevacizumab |
| BCU | Hyb 1.38 antibody | aflibercept |
| BCV | Hyb 1.38 antibody | KH902 VEGF receptor-Fc fusion protein |
| BCW | Hyb 1.38 antibody | 2C3 antibody |
| BCX | Hyb 1.38 antibody | ORA102 |
| BCY | Hyb 1.38 antibody | pegaptanib |
| BCZ | Hyb 1.38 antibody | bevasiranib |
| BDA | Hyb 1.38 antibody | SIRNA-027 |
| BDB | Hyb 1.38 antibody | decursin |
| BDC | Hyb 1.38 antibody | decursinol |
| BDD | Hyb 1.38 antibody | picropodophyllin |
| BDE | Hyb 1.38 antibody | guggulsterone |
| BDF | Hyb 1.38 antibody | PLG101 |
| BDG | Hyb 1.38 antibody | eicosanoid LXA4 |
| BDH | Hyb 1.38 antibody | PTK787 |
| BDI | Hyb 1.38 antibody | pazopanib |
| BDJ | Hyb 1.38 antibody | axitinib |
| BDK | Hyb 1.38 antibody | CDDO-Me |
| BDL | Hyb 1.38 antibody | CDDO-Imm |
| BDM | Hyb 1.38 antibody | shikonin |
| BDN | Hyb 1.38 antibody | beta-hydroxyisovalerylshikonin |
| BDO | Hyb 1.38 antibody | ganglioside GM3 |
| BDP | Hyb 1.38 antibody | DC101 antibody |
| BDQ | Hyb 1.38 antibody | Mab25 antibody |
| BDR | Hyb 1.38 antibody | Mab73 antibody |
| BDS | Hyb 1.38 antibody | 4A5 antibody |
| BDT | Hyb 1.38 antibody | 4E10 antibody |
| BDU | Hyb 1.38 antibody | 5F12 antibody |
| BDV | Hyb 1.38 antibody | VA01 antibody |
| BDW | Hyb 1.38 antibody | BL2 antibody |
| BDX | Hyb 1.38 antibody | VEGF-related protein |
| BDY | Hyb 1.38 antibody | sFLT01 |
| BDZ | Hyb 1.38 antibody | sFLT02 |
| BEA | Hyb 1.38 antibody | Peptide B3 |
| BEB | Hyb 1.38 antibody | TG100801 |
| BEC | Hyb 1.38 antibody | sorafenib |
| BED | Hyb 1.38 antibody | G6-31 antibody |
| BEF | Hyb 1.39 antibody | ranibizumab |
| BEG | Hyb 1.39 antibody | bevacizumab |
| BEH | Hyb 1.39 antibody | aflibercept |
| BEI | Hyb 1.39 antibody | KH902 VEGF receptor-Fc fusion protein |
| BEJ | Hyb 1.39 antibody | 2C3 antibody |
| BEK | Hyb 1.39 antibody | ORA102 |
| BEL | Hyb 1.39 antibody | pegaptanib |
| BEM | Hyb 1.39 antibody | bevasiranib |
| BEN | Hyb 1.39 antibody | SIRNA-027 |
| BEO | Hyb 1.39 antibody | decursin |
| BEP | Hyb 1.39 antibody | decursinol |
| BEQ | Hyb 1.39 antibody | picropodophyllin |
| BER | Hyb 1.39 antibody | guggulsterone |
| BES | Hyb 1.39 antibody | PLG101 |
| BET | Hyb 1.39 antibody | eicosanoid LXA4 |
| BEU | Hyb 1.39 antibody | PTK787 |
| BEV | Hyb 1.39 antibody | pazopanib |
| BEW | Hyb 1.39 antibody | axitinib |
| BEX | Hyb 1.39 antibody | CDDO-Me |
| BEY | Hyb 1.39 antibody | CDDO-Imm |
| BEZ | Hyb 1.39 antibody | shikonin |
| BFA | Hyb 1.39 antibody | beta-hydroxyisovalerylshikonin |
| BFB | Hyb 1.39 antibody | ganglioside GM3 |
| BFC | Hyb 1.39 antibody | DC101 antibody |
| BFD | Hyb 1.39 antibody | Mab25 antibody |
| BFE | Hyb 1.39 antibody | Mab73 antibody |
| BFF | Hyb 1.39 antibody | 4A5 antibody |
| BFG | Hyb 1.39 antibody | 4E10 antibody |
| BFH | Hyb 1.39 antibody | 5F12 antibody |
| BFI | Hyb 1.39 antibody | VA01 antibody |
| BFJ | Hyb 1.39 antibody | BL2 antibody |
| BFK | Hyb 1.39 antibody | VEGF-related protein |
| BFL | Hyb 1.39 antibody | sFLT01 |
| BFM | Hyb 1.39 antibody | sFLT02 |
| BFN | Hyb 1.39 antibody | Peptide B3 |
| BFO | Hyb 1.39 antibody | TG100801 |
| BFP | Hyb 1.39 antibody | sorafenib |
| BFQ | Hyb 1.39 antibody | G6-31 antibody |
| BFR | Hyb 1.40 antibody | ranibizumab |
| BFS | Hyb 1.40 antibody | bevacizumab |
| BFT | Hyb 1.40 antibody | aflibercept |
| BFU | Hyb 1.40 antibody | KH902 VEGF receptor-Fc fusion protein |
| BFV | Hyb 1.40 antibody | 2C3 antibody |
| BFW | Hyb 1.40 antibody | ORA102 |
| BFX | Hyb 1.40 antibody | pegaptanib |
| BFY | Hyb 1.40 antibody | bevasiranib |
| BFZ | Hyb 1.40 antibody | SIRNA-027 |
| BGA | Hyb 1.40 antibody | decursin |
| BGB | Hyb 1.40 antibody | decursinol |
| BGC | Hyb 1.40 antibody | picropodophyllin |
| BGD | Hyb 1.40 antibody | guggulsterone |
| BGE | Hyb 1.40 antibody | PLG101 |
| BGF | Hyb 1.40 antibody | eicosanoid LXA4 |
| BGG | Hyb 1.40 antibody | PTK787 |
| BGH | Hyb 1.40 antibody | pazopanib |
| BGI | Hyb 1.40 antibody | axitinib |
| BGJ | Hyb 1.40 antibody | CDDO-Me |
| BGK | Hyb 1.40 antibody | CDDO-Imm |
| BGL | Hyb 1.40 antibody | shikonin |
| BGM | Hyb 1.40 antibody | beta-hydroxyisovalerylshikonin |
| BGN | Hyb 1.40 antibody | ganglioside GM3 |
| BOO | Hyb 1.40 antibody | DC101 antibody |
| BGP | Hyb 1.40 antibody | Mab25 antibody |
| BGBGQ | Hyb 1.40 antibody | Mab73 antibody |
| BGR | Hyb 1.40 antibody | 4A5 antibody |
| BGS | Hyb 1.40 antibody | 4E10 antibody |
| BGT | Hyb 1.40 antibody | 5F12 antibody |
| BGU | Hyb 1.40 antibody | VA01 antibody |
| BGV | Hyb 1.40 antibody | BL2 antibody |
| BGW | Hyb 1.40 antibody | VEGF-related protein |
| BGX | Hyb 1.40 antibody | sFLT01 |
| BGY | Hyb 1.40 antibody | sFLT02 |
| BGZ | Hyb 1.40 antibody | Peptide B3 |
| BHA | Hyb 1.40 antibody | TG100801 |
| BHB | Hyb 1.40 antibody | sorafenib |
| BHC | Hyb 1.40 antibody | G6-31 antibody |
| BHD | Hyb 1.45 antibody | ranibizumab |
| BHE | Hyb 1.45 antibody | bevacizumab |
| BHF | Hyb 1.45 antibody | aflibercept |
| BHG | Hyb 1.45 antibody | KH902 VEGF receptor-Fc fusion protein |
| BHH | Hyb 1.45 antibody | 2C3 antibody |
| BHI | Hyb 1.45 antibody | ORA102 |
| BHJ | Hyb 1.45 antibody | pegaptanib |
| BHK | Hyb 1.45 antibody | bevasiranib |
| BHL | Hyb 1.45 antibody | SIRNA-027 |
| BHM | Hyb 1.45 antibody | decursin |
| BHN | Hyb 1.45 antibody | decursinol |
| BHO | Hyb 1.45 antibody | picropodophyllin |
| BHP | Hyb 1.45 antibody | guggulsterone |
| BHQ | Hyb 1.45 antibody | PLG101 |
| BHR | Hyb 1.45 antibody | eicosanoid LXA4 |
| BHS | Hyb 1.45 antibody | PTK787 |
| BHT | Hyb 1.45 antibody | pazopanib |
| BHU | Hyb 1.45 antibody | axitinib |
| BHV | Hyb 1.45 antibody | CDDO-Me |
| BHW | Hyb 1.45 antibody | CDDO-Imm |
| BHX | Hyb 1.45 antibody | shikonin |
| BHY | Hyb 1.45 antibody | beta-hydroxyisovalerylshikonin |
| BHZ | Hyb 1.45 antibody | ganglioside GM3 |
| BIA | Hyb 1.45 antibody | DC101 antibody |
| BIB | Hyb 1.45 antibody | Mab25 antibody |
| BIC | Hyb 1.45 antibody | Mab73 antibody |
| BID | Hyb 1.45 antibody | 4A5 antibody |
| BIE | Hyb 1.45 antibody | 4E10 antibody |
| BIF | Hyb 1.45 antibody | 5F12 antibody |
| BIG | Hyb 1.45 antibody | VA01 antibody |
| BIH | Hyb 1.45 antibody | BL2 antibody |
| BIJ | Hyb 1.45 antibody | VEGF-related protein |
| BIK | Hyb 1.45 antibody | sFLT01 |
| BIL | Hyb 1.45 antibody | sFLT02 |
| BIM | Hyb 1.45 antibody | Peptide B3 |
| BIN | Hyb 1.45 antibody | TG100801 |
| BIO | Hyb 1.45 antibody | sorafenib |
| BIP | Hyb 1.45 antibody | G6-31 antibody |
| BIQ | Hyb 1.46 antibody | ranibizumab |
| BIR | Hyb 1.46 antibody | bevacizumab |
| BIS | Hyb 1.46 antibody | aflibercept |
| BIT | Hyb 1.46 antibody | KH902 VEGF receptor-Fc fusion protein |
| BIU | Hyb 1.46 antibody | 2C3 antibody |
| BIV | Hyb 1.46 antibody | ORA102 |
| BIW | Hyb 1.46 antibody | pegaptanib |
| BIX | Hyb 1.46 antibody | bevasiranib |
| BIY | Hyb 1.46 antibody | SIRNA-027 |
| BIZ | Hyb 1.46 antibody | decursin |
| BJA | Hyb 1.46 antibody | decursinol |
| BJB | Hyb 1.46 antibody | picropodophyllin |
| BJC | Hyb 1.46 antibody | guggulsterone |
| BJD | Hyb 1.46 antibody | PLG101 |
| BJE | Hyb 1.46 antibody | eicosanoid LXA4 |
| BJF | Hyb 1.46 antibody | PTK787 |
| BJG | Hyb 1.46 antibody | pazopanib |
| BJH | Hyb 1.46 antibody | axitinib |
| BJI | Hyb 1.46 antibody | CDDO-Me |
| BJJ | Hyb 1.46 antibody | CDDO-Imm |
| BJK | Hyb 1.46 antibody | shikonin |
| BJL | Hyb 1.46 antibody | beta-hydroxyisovalerylshikonin |
| BJM | Hyb 1.46 antibody | ganglioside GM3 |
| BJN | Hyb 1.46 antibody | DC101 antibody |
| BJO | Hyb 1.46 antibody | Mab25 antibody |
| BJP | Hyb 1.46 antibody | Mab73 antibody |
| BJQ | Hyb 1.46 antibody | 4A5 antibody |
| BJR | Hyb 1.46 antibody | 4E10 antibody |
| BJS | Hyb 1.46 antibody | 5F12 antibody |
| BJT | Hyb 1.46 antibody | VA01 antibody |
| BJU | Hyb 1.46 antibody | BL2 antibody |
| BJV | Hyb 1.46 antibody | VEGF-related protein |
| BJW | Hyb 1.46 antibody | sFLT01 |
| BJX | Hyb 1.46 antibody | sFLT02 |
| BJY | Hyb 1.46 antibody | Peptide B3 |
| BJZ | Hyb 1.46 antibody | TG100801 |
| BKA | Hyb 1.46 antibody | sorafenib |
| BKB | Hyb 1.46 antibody | G6-31 antibody |
| BKC | Hyb 1.48 antibody | ranibizumab |
| BKD | Hyb 1.48 antibody | bevacizumab |
| BKE | Hyb 1.48 antibody | aflibercept |
| BKF | Hyb 1.48 antibody | KH902 VEGF receptor-Fc fusion protein |
| BKG | Hyb 1.48 antibody | 2C3 antibody |
| BKH | Hyb 1.48 antibody | ORA102 |
| BKI | Hyb 1.48 antibody | pegaptanib |
| BKJ | Hyb 1.48 antibody | bevasiranib |
| BKK | Hyb 1.48 antibody | SIRNA-027 |
| BKL | Hyb 1.48 antibody | decursin |
| BKM | Hyb 1.48 antibody | decursinol |
| BKN | Hyb 1.48 antibody | picropodophyllin |
| BKO | Hyb 1.48 antibody | guggulsterone |
| BKP | Hyb 1.48 antibody | PLG101 |
| BKQ | Hyb 1.48 antibody | eicosanoid LXA4 |
| BKR | Hyb 1.48 antibody | PTK787 |
| BKS | Hyb 1.48 antibody | pazopanib |
| BKT | Hyb 1.48 antibody | axitinib |
| BKU | Hyb 1.48 antibody | CDDO-Me |
| BKV | Hyb 1.48 antibody | CDDO-Imm |
| BKW | Hyb 1.48 antibody | shikonin |
| BKX | Hyb 1.48 antibody | beta-hydroxyisovalerylshikonin |
| BKY | Hyb 1.48 antibody | ganglioside GM3 |
| BKZ | Hyb 1.48 antibody | DC101 antibody |
| BLA | Hyb 1.48 antibody | Mab25 antibody |
| BLB | Hyb 1.48 antibody | Mab73 antibody |
| BLC | Hyb 1.48 antibody | 4A5 antibody |
| BLD | Hyb 1.48 antibody | 4E10 antibody |
| BLE | Hyb 1.48 antibody | 5F12 antibody |
| BLF | Hyb 1.48 antibody | VA01 antibody |
| BLG | Hyb 1.48 antibody | BL2 antibody |
| BLH | Hyb 1.48 antibody | VEGF-related protein |
| BLI | Hyb 1.48 antibody | sFLT01 |
| BLJ | Hyb 1.48 antibody | sFLT02 |
| BLK | Hyb 1.48 antibody | Peptide B3 |
| BLL | Hyb 1.48 antibody | TG100801 |
| BLM | Hyb 1.48 antibody | sorafenib |
| BLN | Hyb 1.48 antibody | G6-31 antibody |
| BLO | Hyb 1.49 antibody | ranibizumab |
| BLP | Hyb 1.49 antibody | bevacizumab |
| BLQ | Hyb 1.49 antibody | aflibercept |
| BLR | Hyb 1.49 antibody | KH902 VEGF receptor-Fc fusion protein |
| BLS | Hyb 1.49 antibody | 2C3 antibody |
| BLT | Hyb 1.49 antibody | ORA102 |
| BLU | Hyb 1.49 antibody | pegaptanib |
| BLV | Hyb 1.49 antibody | bevasiranib |
| BLW | Hyb 1.49 antibody | SIRNA-027 |
| BLX | Hyb 1.49 antibody | decursin |
| BLY | Hyb 1.49 antibody | decursinol |
| BLZ | Hyb 1.49 antibody | picropodophyllin |
| BMA | Hyb 1.49 antibody | guggulsterone |
| BMB | Hyb 1.49 antibody | PLG101 |
| BMC | Hyb 1.49 antibody | eicosanoid LXA4 |
| BMD | Hyb 1.49 antibody | PTK787 |
| BME | Hyb 1.49 antibody | pazopanib |
| BMF | Hyb 1.49 antibody | axitinib |
| BMG | Hyb 1.49 antibody | CDDO-Me |
| BMH | Hyb 1.49 antibody | CDDO-Imm |
| BMI | Hyb 1.49 antibody | shikonin |
| BMJ | Hyb 1.49 antibody | beta-hydroxyisovalerylshikonin |
| BMK | Hyb 1.49 antibody | ganglioside GM3 |
| BML | Hyb 1.49 antibody | DC101 antibody |
| BMM | Hyb 1.49 antibody | Mab25 antibody |
| BMN | Hyb 1.49 antibody | Mab73 antibody |
| BMO | Hyb 1.49 antibody | 4A5 antibody |
| BMP | Hyb 1.49 antibody | 4E10 antibody |
| BMQ | Hyb 1.49 antibody | 5F12 antibody |
| BMR | Hyb 1.49 antibody | VA01 antibody |
| BMS | Hyb 1.49 antibody | BL2 antibody |
| BMT | Hyb 1.49 antibody | VEGF-related protein |
| BMU | Hyb 1.49 antibody | sFLT01 |
| BMV | Hyb 1.49 antibody | sFLT02 |
| BMW | Hyb 1.49 antibody | Peptide B3 |
| BMX | Hyb 1.49 antibody | TG100801 |
| BMY | Hyb 1.49 antibody | sorafenib |
| BMZ | Hyb 1.49 antibody | G6-31 antibody |
| BNA | Hyb 1.51 antibody | ranibizumab |
| BNB | Hyb 1.51 antibody | bevacizumab |
| BNC | Hyb 1.51 antibody | aflibercept |
| BND | Hyb 1.51 antibody | KH902 VEGF receptor-Fc fusion protein |
| BNE | Hyb 1.51 antibody | 2C3 antibody |
| BNF | Hyb 1.51 antibody | ORA102 |
| BNG | Hyb 1.51 antibody | pegaptanib |
| BNH | Hyb 1.51 antibody | bevasiranib |
| BNI | Hyb 1.51 antibody | SIRNA-027 |
| BNJ | Hyb 1.51 antibody | decursin |
| BNK | Hyb 1.51 antibody | decursinol |
| BNL | Hyb 1.51 antibody | picropodophyllin |
| BNM | Hyb 1.51 antibody | guggulsterone |
| BNN | Hyb 1.51 antibody | PLG101 |
| BNO | Hyb 1.51 antibody | eicosanoid LXA4 |
| BNP | Hyb 1.51 antibody | PTK787 |
| BNQ | Hyb 1.51 antibody | pazopanib |
| BNR | Hyb 1.51 antibody | axitinib |
| BNS | Hyb 1.51 antibody | CDDO-Me |
| BNT | Hyb 1.51 antibody | CDDO-Imm |
| BNU | Hyb 1.51 antibody | shikonin |
| BNV | Hyb 1.51 antibody | beta-hydroxyisovalerylshikonin |
| BNW | Hyb 1.51 antibody | ganglioside GM3 |
| BNX | Hyb 1.51 antibody | DC101 antibody |
| BNY | Hyb 1.51 antibody | Mab25 antibody |
| BNZ | Hyb 1.51 antibody | Mab73 antibody |
| BOA | Hyb 1.51 antibody | 4A5 antibody |
| BOB | Hyb 1.51 antibody | 4E10 antibody |
| BOC | Hyb 1.51 antibody | 5F12 antibody |
| BOD | Hyb 1.51 antibody | VA01 antibody |
| BOE | Hyb 1.51 antibody | BL2 antibody |
| BOF | Hyb 1.51 antibody | VEGF-related protein |
| BOG | Hyb 1.51 antibody | sFLT01 |
| BOH | Hyb 1.51 antibody | sFLT02 |
| BOI | Hyb 1.51 antibody | Peptide B3 |
| BOJ | Hyb 1.51 antibody | TG100801 |
| BOK | Hyb 1.51 antibody | sorafenib |
| BOL | Hyb 1.51 antibody | G6-31 antibody |
| BOM | Hyb 6.4.1 antibody | ranibizumab |
| BON | Hyb 6.4.1 antibody | bevacizumab |
| BOP | Hyb 6.4.1 antibody | Aflibercept |
| BOQ | Hyb 6.4.1 antibody | KH902 VEGF receptor-Fc fusion protein |
| BOR | Hyb 6.4.1 antibody | 2C3 antibody |
| BOS | Hyb 6.4.1 antibody | ORA102 |
| BOT | Hyb 6.4.1 antibody | pegaptanib |
| BOU | Hyb 6.4.1 antibody | bevasiranib |
| BOV | Hyb 6.4.1 antibody | SIRNA-027 |
| BOW | Hyb 6.4.1 antibody | decursin |
| BOX | Hyb 6.4.1 antibody | decursinol |
| BOY | Hyb 6.4.1 antibody | picropodophyllin |
| BOZ | Hyb 6.4.1 antibody | guggulsterone |
| BPA | Hyb 6.4.1 antibody | PLG101 |
| BPB | Hyb 6.4.1 antibody | eicosanoid LXA4 |
| BPC | Hyb 6.4.1 antibody | PTK787 |
| BPD | Hyb 6.4.1 antibody | pazopanib |
| BPE | Hyb 6.4.1 antibody | axitinib |
| BPF | Hyb 6.4.1 antibody | CDDO-Me |
| BPG | Hyb 6.4.1 antibody | CDDO-Imm |
| BPH | Hyb 6.4.1 antibody | shikonin |
| BPI | Hyb 6.4.1 antibody | beta-hydroxyisovalerylshikonin |
| BPJ | Hyb 6.4.1 antibody | ganglioside GM3 |
| BPK | Hyb 6.4.1 antibody | DC101 antibody |
| BPL | Hyb 6.4.1 antibody | Mab25 antibody |
| BPM | Hyb 6.4.1 antibody | Mab73 antibody |
| BPN | Hyb 6.4.1 antibody | 4A5 antibody |
| BPO | Hyb 6.4.1 antibody | 4E10 antibody |
| BPP | Hyb 6.4.1 antibody | 5F12 antibody |
| BPQ | Hyb 6.4.1 antibody | VA01 antibody |
| BPR | Hyb 6.4.1 antibody | BL2 antibody |
| BPS | Hyb 6.4.1 antibody | VEGF-related protein |
| BPT | Hyb 6.4.1 antibody | sFLT01 |
| BPU | Hyb 6.4.1 antibody | sFLT02 |
| BPV | Hyb 6.4.1 antibody | Peptide B3 |
| BPW | Hyb 6.4.1 antibody | TG100801 |
| BPX | Hyb 6.4.1 antibody | sorafenib |
| BPY | Hyb 6.4.1 antibody | G6-31 antibody |
| BPZ | F3 antibody | ranibizumab |
| BQA | F3 antibody | bevacizumab |
| BQB | F3 antibody | aflibercept |
| BQC | F3 antibody | KH902 VEGF receptor-Fc fusion protein |
| BQD | F3 antibody | 2C3 antibody |
| BQE | F3 antibody | ORA102 |
| BQF | F3 antibody | pegaptanib |
| BQG | F3 antibody | bevasiranib |
| BQH | F3 antibody | SIRNA-027 |
| BQI | F3 antibody | decursin |
| BQJ | F3 antibody | decursinol |
| BQK | F3 antibody | picropodophyllin |
| BQL | F3 antibody | guggulsterone |
| BQM | F3 antibody | PLG101 |
| BQN | F3 antibody | eicosanoid LXA4 |
| BQO | F3 antibody | PTK787 |
| BQP | F3 antibody | pazopanib |
| BQQ | F3 antibody | axitinib |
| BQR | F3 antibody | CDDO-Me |
| BQS | F3 antibody | CDDO-Imm |
| BQT | F3 antibody | shikonin |
| BQU | F3 antibody | beta-hydroxyisovalerylshikonin |
| BQV | F3 antibody | ganglioside GM3 |
| BQW | F3 antibody | DC101 antibody |
| BQX | F3 antibody | Mab25 antibody |
| BQY | F3 antibody | Mab73 antibody |
| BQZ | F3 antibody | 4A5 antibody |
| BRA | F3 antibody | 4E10 antibody |
| BRB | F3 antibody | 5F12 antibody |
| BRC | F3 antibody | VA01 antibody |
| BRD | F3 antibody | BL2 antibody |
| BRE | F3 antibody | VEGF-related protein |
| BRF | F3 antibody | sFLT01 |
| BRG | F3 antibody | sFLT02 |
| BRH | F3 antibody | Peptide B3 |
| BRI | F3 antibody | TG100801 |
| BRJ | F3 antibody | sorafenib |
| BRK | F3 antibody | G6-31 antibody |
| BRL | Humanized F3 antibody | ranibizumab |
| BRM | Humanized F3 antibody | bevacizumab |
| BRN | Humanized F3 antibody | aflibercept |
| BRO | Humanized F3 antibody | KH902 VEGF receptor-Fc fusion protein |
| BRP | Humanized F3 antibody | 2C3 antibody |
| BRQ | Humanized F3 antibody | ORA102 |
| BRR | Humanized F3 antibody | pegaptanib |
| BRS | Humanized F3 antibody | bevasiranib |
| BRT | Humanized F3 antibody | SIRNA-027 |
| BRU | Humanized F3 antibody | decursin |
| BRV | Humanized F3 antibody | decursinol |
| BRW | Humanized F3 antibody | picropodophyllin |
| BRX | Humanized F3 antibody | guggulsterone |
| BRY | Humanized F3 antibody | PLG101 |
| BRZ | Humanized F3 antibody | eicosanoid LXA4 |
| BSA | Humanized F3 antibody | PTK787 |
| BSB | Humanized F3 antibody | pazopanib |
| BSC | Humanized F3 antibody | axitinib |
| BSD | Humanized F3 antibody | CDDO-Me |
| BSE | Humanized F3 antibody | CDDO-Imm |
| BSF | Humanized F3 antibody | shikonin |
| BSG | Humanized F3 antibody | beta-hydroxyisovalerylshikonin |
| BSH | Humanized F3 antibody | ganglioside GM3 |
| BSI | Humanized F3 antibody | DC101 antibody |
| BSJ | Humanized F3 antibody | Mab25 antibody |
| BSK | Humanized F3 antibody | Mab73 antibody |
| BSL | Humanized F3 antibody | 4A5 antibody |
| BSM | Humanized F3 antibody | 4E10 antibody |
| BSN | Humanized F3 antibody | 5F12 antibody |
| BSO | Humanized F3 antibody | VA01 antibody |
| BSP | Humanized F3 antibody | BL2 antibody |
| BSQ | Humanized F3 antibody | VEGF-related protein |
| BSR | Humanized F3 antibody | sFLT01 |
| BSS | Humanized F3 antibody | sFLT02 |
| BST | Humanized F3 antibody | Peptide B3 |
| BSU | Humanized F3 antibody | TG100801 |
| BSV | Humanized F3 antibody | sorafenib |
| BSW | Humanized F3 antibody | G6-31 antibody |
| BSX | C1 antibody | ranibizumab |
| BSY | C1 antibody | bevacizumab |
| BSZ | C1 antibody | aflibercept |
| BTA | C1 antibody | KH902 VEGF receptor-Fc fusion protein |
| BTB | C1 antibody | 2C3 antibody |
| BTC | C1 antibody | ORA102 |
| BTD | C1 antibody | pegaptanib |
| BTE | C1 antibody | bevasiranib |
| BTF | C1 antibody | SIRNA-027 |
| BTG | C1 antibody | decursin |
| BTH | C1 antibody | decursinol |
| BTI | C1 antibody | Picropodophyllin |
| BTJ | C1 antibody | guggulsterone |
| BTK | C1 antibody | PLG101 |
| BTL | C1 antibody | eicosanoid LXA4 |
| BTM | C1 antibody | PTK787 |
| BTN | C1 antibody | pazopanib |
| BTO | C1 antibody | axitinib |
| BTP | C1 antibody | CDDO-Me |
| BTQ | C1 antibody | CDDO-Imm |
| BTR | C1 antibody | shikonin |
| BTS | C1 antibody | beta-hydroxyisovalerylshikonin |
| BTT | C1 antibody | ganglioside GM3 |
| BTU | C1 antibody | DC101 antibody |
| BTV | C1 antibody | Mab25 antibody |
| BTW | C1 antibody | Mab73 antibody |
| BTX | C1 antibody | 4A5 antibody |
| BTY | C1 antibody | 4E10 antibody |
| BTZ | C1 antibody | 5F12 antibody |
| BUA | C1 antibody | VA01 antibody |
| BUB | C1 antibody | BL2 antibody |
| BUC | C1 antibody | VEGF-related protein |
| BUD | C1 antibody | sFLT01 |
| BUE | C1 antibody | sFLT02 |
| BUF | C1 antibody | Peptide B3 |
| BUG | C1 antibody | TG100801 |
| BUH | C1 antibody | sorafenib |
| BUI | C1 antibody | G6-31 antibody |
| BUJ | Humanized C1 antibody | ranibizumab |
| BUK | Humanized C1 antibody | bevacizumab |
| BUL | Humanized C1 antibody | aflibercept |
| BUM | Humanized C1 antibody | KH902 VEGF receptor-Fc fusion protein |
| BUN | Humanized C1 antibody | 2C3 antibody |
| BUO | Humanized C1 antibody | ORA102 |
| BUP | Humanized C1 antibody | pegaptanib |
| BUQ | Humanized C1 antibody | bevasiranib |
| BUR | Humanized C1 antibody | SIRNA-027 |
| BUS | Humanized C1 antibody | decursin |
| BUT | Humanized C1 antibody | decursinol |
| BUU | Humanized C1 antibody | picropodophyllin |
| BUV | Humanized C1 antibody | guggulsterone |
| BUW | Humanized C1 antibody | PLG101 |
| BUX | Humanized C1 antibody | eicosanoid LXA4 |
| BUY | Humanized C1 antibody | PTK787 |
| BUZ | Humanized C1 antibody | pazopanib |
| BVA | Humanized C1 antibody | axitinib |
| BVB | Humanized C1 antibody | CDDO-Me |
| BVC | Humanized C1 antibody | CDDO-Imm |
| BVD | Humanized C1 antibody | shikonin |
| BVE | Humanized C1 antibody | beta-hydroxyisovalerylshikonin |
| BVF | Humanized C1 antibody | ganglioside GM3 |
| BVG | Humanized C1 antibody | DC101 antibody |
| BVH | Humanized C1 antibody | Mab25 antibody |
| BVI | Humanized C1 antibody | Mab73 antibody |
| BVJ | Humanized C1 antibody | 4A5 antibody |
| BVK | Humanized C1 antibody | 4E10 antibody |
| BVL | Humanized C1 antibody | 5F12 antibody |
| BVM | Humanized C1 antibody | VA01 antibody |
| BVN | Humanized C1 antibody | BL2 antibody |
| BVO | Humanized C1 antibody | VEGF-related protein |
| BVP | Humanized C1 antibody | sFLT01 |
| BVQ | Humanized C1 antibody | sFLT02 |
| BVR | Humanized C1 antibody | Peptide B3 |
| BVS | Humanized C1 antibody | TG100801 |
| BVT | Humanized C1 antibody | sorafenib |
| BVU | Humanized C1 antibody | G6-31 antibody |
| BVV | 6.4 antibody | ranibizumab |
| BVW | 6.4 antibody | bevacizumab |
| BVX | 6.4 antibody | aflibercept |
| BVY | 6.4 antibody | KH902 VEGF receptor-Fc fusion protein |
| BVZ | 6.4 antibody | 2C3 antibody |
| BWA | 6.4 antibody | ORA102 |
| BWB | 6.4 antibody | pegaptanib |
| BWC | 6.4 antibody | bevasiranib |
| BWD | 6.4 antibody | SIRNA-027 |
| BWE | 6.4 antibody | decursin |
| BWF | 6.4 antibody | decursinol |
| BWG | 6.4 antibody | picropodophyllin |
| BWH | 6.4 antibody | guggulsterone |
| BWI | 6.4 antibody | PLG101 |
| BWJ | 6.4 antibody | eicosanoid LXA4 |
| BWK | 6.4 antibody | PTK787 |
| BWL | 6.4 antibody | pazopanib |
| BWM | 6.4 antibody | Axitinib |
| BWN | 6.4 antibody | CDDO-Me |
| BWO | 6.4 antibody | CDDO-Imm |
| BWP | 6.4 antibody | shikonin |
| BWQ | 6.4 antibody | beta-hydroxyisovalerylshikonin |
| BWR | 6.4 antibody | ganglioside GM3 |
| BWS | 6.4 antibody | DC101 antibody |
| BWT | 6.4 antibody | Mab25 antibody |
| BWU | 6.4 antibody | Mab73 antibody |
| BWV | 6.4 antibody | 4A5 antibody |
| BWW | 6.4 antibody | 4E10 antibody |
| BWX | 6.4 antibody | 5F12 antibody |
| BWY | 6.4 antibody | VA01 antibody |
| BWZ | 6.4 antibody | BL2 antibody |
| BXA | 6.4 antibody | VEGF-related protein |
| BXB | 6.4 antibody | sFLT01 |
| BXC | 6.4 antibody | sFLT02 |
| BXD | 6.4 antibody | Peptide B3 |
| BXE | 6.4 antibody | TG100801 |
| BXF | 6.4 antibody | sorafenib |
| BXG | 6.4 antibody | G6-31 antibody |
| BXH | anti-mPDGF-C goat IgG antibody | ranibizumab |
| BXI | anti-mPDGF-C goat IgG antibody | bevacizumab |
| BXJ | anti-mPDGF-C goat IgG antibody | aflibercept |
| BXK | anti-mPDGF-C goat IgG antibody | KH902 VEGF receptor-Fc fusion protein |
| BXL | anti-mPDGF-C goat IgG antibody | 2C3 antibody |
| BXM | anti-mPDGF-C goat IgG antibody | ORA102 |
| BXN | anti-mPDGF-C goat IgG antibody | pegaptanib |
| BXO | anti-mPDGF-C goat IgG antibody | bevasiranib |
| BXP | anti-mPDGF-C goat IgG antibody | SIRNA-027 |
| BXQ | anti-mPDGF-C goat IgG antibody | decursin |
| BXR | anti-mPDGF-C goat IgG antibody | decursinol |
| BXS | anti-mPDGF-C goat IgG antibody | picropodophyllin |
| BXT | anti-mPDGF-C goat IgG antibody | guggulsterone |
| BXU | anti-mPDGF-C goat IgG antibody | PLG101 |
| BXV | anti-mPDGF-C goat IgG antibody | eicosanoid LXA4 |
| BXW | anti-mPDGF-C goat IgG antibody | PTK787 |
| BXX | anti-mPDGF-C goat IgG antibody | pazopanib |
| BXY | anti-mPDGF-C goat IgG antibody | axitinib |
| BXZ | anti-mPDGF-C goat IgG antibody | CDDO-Me |
| BYA | anti-mPDGF-C goat IgG antibody | CDDO-Imm |
| BYB | anti-mPDGF-C goat IgG antibody | Shikonin |
| BYC | anti-mPDGF-C goat IgG antibody | beta-hydroxyisovalerylshikonin |
| BYD | anti-mPDGF-C goat IgG antibody | ganglioside GM3 |
| BYE | anti-mPDGF-C goat IgG antibody | DC101 antibody |
| BYF | anti-mPDGF-C goat IgG antibody | Mab25 antibody |
| BYG | anti-mPDGF-C goat IgG antibody | Mab73 antibody |
| BYH | anti-mPDGF-C goat IgG antibody | 4A5 antibody |
| BYI | anti-mPDGF-C goat IgG antibody | 4E10 antibody |
| BYJ | anti-mPDGF-C goat IgG antibody | 5F12 antibody |
| BYK | anti-mPDGF-C goat IgG antibody | VA01 antibody |
| BYL | anti-mPDGF-C goat IgG antibody | BL2 antibody |
| BYM | anti-mPDGF-C goat IgG antibody | VEGF-related protein |
| BYN | anti-mPDGF-C goat IgG antibody | sFLT01 |
| BYO | anti-mPDGF-C goat IgG antibody | sFLT02 |
| BYP | anti-mPDGF-C goat IgG antibody | Peptide B3 |
| BYQ | anti-mPDGF-C goat IgG antibody | TG100801 |
| BYR | anti-mPDGF-C goat IgG antibody | sorafenib |
| BYS | anti-mPDGF-C goat IgG antibody | G6-31 antibody |
| BYT | C3.1 antibody | ranibizumab |
| BYU | C3.1 antibody | bevacizumab |
| BYV | C3.1 antibody | aflibercept |
| BYW | C3.1 antibody | KH902 VEGF receptor-Fc fusion protein |
| BYX | C3.1 antibody | 2C3 antibody |
| BYY | C3.1 antibody | ORA102 |
| BYZ | C3.1 antibody | pegaptanib |
| BZA | C3.1 antibody | bevasiranib |
| BZB | C3.1 antibody | SIRNA-027 |
| BZC | C3.1 antibody | decursin |
| BZD | C3.1 antibody | decursinol |
| BZE | C3.1 antibody | picropodophyllin |
| BZF | C3.1 antibody | guggulsterone |
| BZG | C3.1 antibody | PLG101 |
| BZH | C3.1 antibody | eicosanoid LXA4 |
| BZI | C3.1 antibody | PTK787 |
| BZJ | C3.1 antibody | pazopanib |
| BZK | C3.1 antibody | axitinib |
| BZL | C3.1 antibody | CDDO-Me |
| BZM | C3.1 antibody | CDDO-Imm |
| BZN | C3.1 antibody | shikonin |
| BZO | C3.1 antibody | beta-hydroxyisovalerylshikonin |
| BZP | C3.1 antibody | ganglioside GM3 |
| BZQ | C3.1 antibody | DC101 antibody |
| BZR | C3.1 antibody | Mab25 antibody |
| BZS | C3.1 antibody | Mab73 antibody |
| BZT | C3.1 antibody | 4A5 antibody |
| BZU | C3.1 antibody | 4E10 antibody |
| BZV | C3.1 antibody | 5F12 antibody |
| BZW | C3.1 antibody | VA01 antibody |
| BZX | C3.1 antibody | BL2 antibody |
| BZY | C3.1 antibody | VEGF-related protein |
| BZZ | C3.1 antibody | sFLT01 |
| CAA | C3.1 antibody | sFLT02 |
| CAB | C3.1 antibody | Peptide B3 |
| CAC | C3.1 antibody | TG100801 |
| CAD | C3.1 antibody | sorafenib |
| CAE | C3.1 antibody | G6-31 antibody |
| CAF | 5-methyl-7-diethylamino-s-triazolo | ranibizumab |
| | (1,5-a) pyrimidine | |
| CAG | 5-methyl-7-diethylamino-s-triazolo | bevacizumab |
| | (1,5-a) pyrimidine | |
| CAH | 5-methyl-7-diethylamino-s-triazolo | aflibercept |
| | (1,5-a) pyrimidine | |
| CAI | 5-methyl-7-diethylamino-s-triazolo | KI-1902 VEGF receptor-Fc fusion protein |
| | (1,5-a) pyrimidine | |
| CAJ | 5-methyl-7-diethylamino-s-triazolo | 2C3 antibody |
| | (1,5-a) pyrimidine | |
| CAK | 5-methyl-7-diethylamino-s-triazolo | ORA102 |
| | (1,5-a) pyrimidine | |
| CAL | 5-methyl-7-diethylamino-s-triazolo | pegaptanib |
| | (1,5-a) pyrimidine | |
| CAM | 5-methyl-7-diethylamino-s-triazolo | bevasiranib |
| | (1,5-a) pyrimidine | |
| CAN | 5-methyl-7-diethylamino-s-triazolo | SIRNA-027 |
| | (1,5-a) pyrimidine | |
| CAO | 5-methyl-7-diethylamino-s-triazolo | decursin |
| | (1,5-a) pyrimidine | |
| CAP | 5-methyl-7-diethylamino-s-triazolo | decursinol |
| | (1,5-a) pyrimidine | |
| CAQ | 5-methyl-7-diethylamino-s-triazolo | picropodophyllin |
| | (1,5-a) pyrimidine | |
| CAR | 5-methyl-7-diethylamino-s-triazolo | guggulsterone |
| | (1,5-a) pyrimidine | |
| CAS | 5-methyl-7-diethylamino-s-triazolo | PLG101 |
| | (1,5-a) pyrimidine | |
| CAT | 5-methyl-7-diethylamino-s-triazolo | eicosanoid LXA4 |
| | (1,5-a) pyrimidine | |
| CAU | 5-methyl-7-diethylamino-s-triazolo | PTK787 |
| | (1,5-a) pyrimidine | |
| CAV | 5-methyl-7-diethylamino-s-triazolo | pazopanib |
| | (1,5-a) pyrimidine | |
| CAW | 5-methyl-7-diethylamino-s-triazolo | axitinib |
| | (1,5-a) pyrimidine | |
| CAX | 5-methyl-7-diethylamino-s-triazolo | CDDO-Me |
| | (1,5-a) pyrimidine | |
| CAY | 5-methyl-7-diethylamino-s-triazolo | CDDO-Imm |
| | (1,5-a) pyrimidine | |
| CAZ | 5-methyl-7-diethylamino-s-triazolo | shikonin |
| | (1,5-a) pyrimidine | |
| CBA | 5-methyl-7-diethylamino-s-triazolo | beta-hydroxyisovalerylshikonin |
| | (1,5-a) pyrimidine | |
| CBB | 5-methyl-7-diethylamino-s-triazolo | ganglioside GM3 |
| | (1,5-a) pyrimidine | |
| CBC | 5-methyl-7-diethylamino-s-triazolo | DC101 antibody |
| | (1,5-a) pyrimidine | |
| CBD | 5-methyl-7-diethylamino-s-triazolo | Mab25 antibody |
| | (1,5-a) pyrimidine | |
| CBE | 5-methyl-7-diethylamino-s-triazolo | Mab73 antibody |
| | (1,5-a) pyrimidine | |
| CBF | 5-methyl-7-diethylamino-s-triazolo | 4A5 antibody |
| | (1,5-a) pyrimidine | |
| CBG | 5-methyl-7-diethylamino-s-triazolo | 4E10 antibody |
| | (1,5-a) pyrimidine | |
| CBH | 5-methyl-7-diethylamino-s-triazolo | 5F12 antibody |
| | (1,5-a) pyrimidine | |
| CBI | 5-methyl-7-diethylamino-s-triazolo | VA01 antibody |
| | (1,5-a) pyrimidine | |
| CBJ | 5-methyl-7-diethylamino-s-triazolo | BL2 antibody |
| | (1,5-a) pyrimidine | |
| CBK | 5-methyl-7-diethylamino-s-triazolo | VEGF-related protein |
| | (1,5-a) pyrimidine | |
| CBL | 5-methyl-7-diethylamino-s-triazolo | sFLT01 |
| | (1,5-a) pyrimidine | |
| CBM | 5-methyl-7-diethylamino-s-triazolo | sFLT02 |
| | (1,5-a) pyrimidine | |
| CBN | 5-methyl-7-diethylamino-s-triazolo | Peptide B3 |
| | (1,5-a) pyrimidine | |
| CBO | 5-methyl-7-diethylamino-s-triazolo | TG100801 |
| | (1,5-a) pyrimidine | |
| CBP | 5-methyl-7-diethylamino-s-triazolo | sorafenib |
| | (1,5-a) pyrimidine | |
| CBQ | 5-methyl-7-diethylamino-s-triazolo | G6-31 antibody |
| | (1,5-a) pyrimidine | |
| CBR | Interferon | ranibizumab |
| CBS | Interferon | bevacizumab |
| CBT | Interferon | aflibercept |
| CBU | Interferon | KH902 VEGF receptor-Fc fusion protein |
| CBV | Interferon | 2C3 antibody |
| CBW | Interferon | ORA102 |
| CBX | Interferon | pegaptanib |
| CBY | Interferon | bevasiranib |
| CBZ | Interferon | SIRNA-027 |
| CCA | Interferon | decursin |
| CCB | Interferon | decursinol |
| CCC | Interferon | picropodophyllin |
| CCD | Interferon | guggulsterone |
| CCE | Interferon | PLG101 |
| CCF | Interferon | eicosanoid LXA4 |
| CCG | Interferon | PTK787 |
| CCH | Interferon | pazopanib |
| CCI | Interferon | axitinib |
| CCJ | Interferon | CDDO-Me |
| CCK | Interferon | CDDO-Imm |
| CCL | Interferon | shikonin |
| CCM | Interferon | beta-hydroxyisovalerylshikonin |
| CCN | Interferon | ganglioside GM3 |
| CCO | Interferon | DC101 antibody |
| CCP | Interferon | Mab25 antibody |
| CCQ | Interferon | Mab73 antibody |
| CCR | Interferon | 4A5 antibody |
| CCS | Interferon | 4E10 antibody |
| CCT | Interferon | 5F12 antibody |
| CCU | Interferon | VA01 antibody |
| CCV | Interferon | BL2 antibody |
| CCW | Interferon | VEGF-related protein |
| CCX | Interferon | sFLT01 |
| CCY | Interferon | sFLT02 |
| CCZ | Interferon | Peptide B3 |
| CDA | Interferon | TG100801 |
| CDB | Interferon | sorafenib |
| CDC | Interferon | G6-31 antibody |
| CDD | Protamine | ranibizumab |
| CDE | Protamine | bevacizumab |
| CDF | Protamine | aflibercept |
| CDG | Protamine | KH902 VEGF receptor-Fc fusion protein |
| CDH | Protamine | 2C3 antibody |
| CDI | Protamine | ORA102 |
| CDJ | Protamine | pegaptanib |
| CDK | Protamine | bevasiranib |
| CDL | Protamine | SIRNA-027 |
| CDM | Protamine | decursin |
| CDN | Protamine | decursinol |
| CDO | Protamine | picropodophyllin |
| CDP | Protamine | guggulsterone |
| CDQ | Protamine | PLG101 |
| CDR | Protamine | eicosanoid LXA4 |
| CDS | Protamine | PTK787 |
| CDT | Protamine | pazopanib |
| CDU | Protamine | axitinib |
| CDV | Protamine | CDDO-Me |
| CDW | Protamine | CDDO-Imm |
| CDX | Protamine | shikonin |
| CDY | Protamine | beta-hydroxyisovalerylshikonin |
| CDZ | Protamine | ganglioside GM3 |
| CEA | Protamine | DC101 antibody |
| CEB | Protamine | Mab25 antibody |
| CEC | Protamine | Mab73 antibody |
| CED | Protamine | 4A5 antibody |
| CEE | Protamine | 4E10 antibody |
| CEF | Protamine | 5F12 antibody |
| CEG | Protamine | VA01 antibody |
| CEH | Protamine | BL2 antibody |
| CEI | Protamine | VEGF-related protein |
| CEJ | Protamine | sFLT01 |
| CEK | Protamine | sFLT02 |
| CEL | Protamine | Peptide B3 |
| CEM | Protamine | TG100801 |
| CEN | Protamine | sorafenib |
| CEO | Protamine | G6-31 antibody |
| CEP | PDGFR-B1 monoclonal antibody | ranibizumab |
| CEQ | PDGFR-B1 monoclonal antibody | bevacizumab |
| CER | PDGFR-B1 monoclonal antibody | aflibercept |
| CES | PDGFR-B1 monoclonal antibody | KH902 VEGF receptor-Fc fusion protein |
| CET | PDGFR-B1 monoclonal antibody | 2C3 antibody |
| CEU | PDGFR-B1 monoclonal antibody | ORA102 |
| CEV | PDGFR-B1 monoclonal antibody | pegaptanib |
| CEW | PDGFR-B1 monoclonal antibody | bevasiranib |
| CEX | PDGFR-B1 monoclonal antibody | SIRNA-027 |
| CEY | PDGFR-B1 monoclonal antibody | decursin |
| CEZ | PDGFR-B1 monoclonal antibody | decursinol |
| CFA | PDGFR-B1 monoclonal antibody | picropodophyllin |
| CFB | PDGFR-B1 monoclonal antibody | guggulsterone |
| CFC | PDGFR-B1 monoclonal antibody | PLG101 |
| CFD | PDGFR-B1 monoclonal antibody | eicosanoid LXA4 |
| CFE | PDGFR-B1 monoclonal antibody | PTK787 |
| CFF | PDGFR-B1 monoclonal antibody | pazopanib |
| CFG | PDGFR-B1 monoclonal antibody | axitinib |
| CFH | PDGFR-B1 monoclonal antibody | CDDO-Me |
| CFI | PDGFR-B1 monoclonal antibody | CDDO-Imm |
| CFJ | PDGFR-B1 monoclonal antibody | shikonin |
| CFK | PDGFR-B1 monoclonal antibody | beta-hydroxyisovalerylshikonin |
| CFL | PDGFR-B1 monoclonal antibody | ganglioside GM3 |
| CFM | PDGFR-B1 monoclonal antibody | DC101 antibody |
| CFN | PDGFR-B1 monoclonal antibody | Mab25 antibody |
| CFO | PDGFR-B1 monoclonal antibody | Mab73 antibody |
| CFP | PDGFR-B1 monoclonal antibody | 4A5 antibody |
| CFQ | PDGFR-B1 monoclonal antibody | 4E10 antibody |
| CFR | PDGFR-B1 monoclonal antibody | 5F12 antibody |
| CFS | PDGFR-B1 monoclonal antibody | VA01 antibody |
| CFT | PDGFR-B1 monoclonal antibody | BL2 antibody |
| CFU | PDGFR-B1 monoclonal antibody | VEGF-related protein |
| CFV | PDGFR-B1 monoclonal antibody | sFLT01 |
| CFW | PDGFR-B1 monoclonal antibody | sFLT02 |
| CFX | PDGFR-B1 monoclonal antibody | Peptide B3 |
| CFY | PDGFR-B1 monoclonal antibody | TG100801 |
| CFZ | PDGFR-B1 monoclonal antibody | sorafenib |
| CGA | PDGFR-B1 monoclonal antibody | G6-31 antibody |
| CGB | PDGFR-B2 monoclonal antibody | ranibizumab |
| CGC | PDGFR-B2 monoclonal antibody | bevacizumab |
| CGD | PDGFR-B2 monoclonal antibody | Aflibercept |
| CGE | PDGFR-B2 monoclonal antibody | KH902 VEGF receptor-Fc fusion protein |
| CGF | PDGFR-B2 monoclonal antibody | 2C3 antibody |
| CGG | PDGFR-B2 monoclonal antibody | ORA102 |
| CGH | PDGFR-B2 monoclonal antibody | pegaptanib |
| CGI | PDGFR-B2 monoclonal antibody | bevasiranib |
| CGJ | PDGFR-B2 monoclonal antibody | SIRNA-027 |
| CGK | PDGFR-B2 monoclonal antibody | decursin |
| CGL | PDGFR-B2 monoclonal antibody | decursinol |
| CGM | PDGFR-B2 monoclonal antibody | picropodophyllin |
| CGN | PDGFR-B2 monoclonal antibody | guggulsterone |
| CGO | PDGFR-B2 monoclonal antibody | PLG101 |
| CGP | PDGFR-B2 monoclonal antibody | eicosanoid LXA4 |
| CGQ | PDGFR-B2 monoclonal antibody | PTK787 |
| CGR | PDGFR-B2 monoclonal antibody | pazopanib |
| CGS | PDGFR-B2 monoclonal antibody | axitinib |
| CGT | PDGFR-B2 monoclonal antibody | CDDO-Me |
| CGU | PDGFR-B2 monoclonal antibody | CDDO-Imm |
| CGV | PDGFR-B2 monoclonal antibody | shikonin |
| CGW | PDGFR-B2 monoclonal antibody | beta-hydroxyisovalerylshikonin |
| CGX | PDGFR-B2 monoclonal antibody | ganglioside GM3 |
| CGY | PDGFR-B2 monoclonal antibody | DC101 antibody |
| CGZ | PDGFR-B2 monoclonal antibody | Mab25 antibody |
| CHA | PDGFR-B2 monoclonal antibody | Mab73 antibody |
| CHB | PDGFR-B2 monoclonal antibody | 4A5 antibody |
| CHC | PDGFR-B2 monoclonal antibody | 4E10 antibody |
| CHD | PDGFR-B2 monoclonal antibody | 5F12 antibody |
| CHE | PDGFR-B2 monoclonal antibody | VA01 antibody |
| CHF | PDGFR-B2 monoclonal antibody | BL2 antibody |
| CHG | PDGFR-B2 monoclonal antibody | VEGF-related protein |
| CHH | PDGFR-B2 monoclonal antibody | sFLT01 |
| CHI | PDGFR-B2 monoclonal antibody | sFLT02 |
| CHJ | PDGFR-B2 monoclonal antibody | Peptide B3 |
| CHK | PDGFR-B2 monoclonal antibody | TG100801 |
| CHL | PDGFR-B2 monoclonal antibody | sorafenib |
| CHM | PDGFR-B2 monoclonal antibody | G6-31 antibody |
| CHN | 6D11 monoclonal antibody | ranibizumab |
| CHO | 6D11 monoclonal antibody | bevacizumab |
| CHP | 6D11 monoclonal antibody | aflibercept |
| CHQ | 6D11 monoclonal antibody | KH902 VEGF receptor-Fc fusion protein |
| CHR | 6D11 monoclonal antibody | 2C3 antibody |
| CHS | 6D11 monoclonal antibody | ORA102 |
| CHT | 6D11 monoclonal antibody | pegaptanib |
| CHU | 6D11 monoclonal antibody | bevasiranib |
| CHV | 6D11 monoclonal antibody | SIRNA-027 |
| CHW | 6D11 monoclonal antibody | decursin |
| CHX | 6D11 monoclonal antibody | decursinol |
| CHY | 6D11 monoclonal antibody | picropodophyllin |
| CHZ | 6D11 monoclonal antibody | guggulsterone |
| CIA | 6D11 monoclonal antibody | PLG101 |
| CIB | 6D11 monoclonal antibody | eicosanoid LXA4 |
| CIC | 6D11 monoclonal antibody | PTK787 |
| CID | 6D11 monoclonal antibody | pazopanib |
| CIE | 6D11 monoclonal antibody | axitinib |
| CIF | 6D11 monoclonal antibody | CDDO-Me |
| CIG | 6D11 monoclonal antibody | CDDO-Imm |
| CIH | 6D11 monoclonal antibody | shikonin |
| CII | 6D11 monoclonal antibody | beta-hydroxyisovalerylshikonin |
| CIJ | 6D11 monoclonal antibody | ganglioside GM3 |
| CIK | 6D11 monoclonal antibody | DC101 antibody |
| CIL | 6D11 monoclonal antibody | Mab25 antibody |
| CIM | 6D11 monoclonal antibody | Mab73 antibody |
| CIN | 6D11 monoclonal antibody | 4A5 antibody |
| CIO | 6D11 monoclonal antibody | 4E10 antibody |
| CIP | 6D11 monoclonal antibody | 5F12 antibody |
| CIQ | 6D11 monoclonal antibody | VA01 antibody |
| CIR | 6D11 monoclonal antibody | BL2 antibody |
| CIS | 6D11 monoclonal antibody | VEGF-related protein |
| CIT | 6D11 monoclonal antibody | sFLT01 |
| CIU | 6D11 monoclonal antibody | sFLT02 |
| CIV | 6D11 monoclonal antibody | Peptide B3 |
| CIW | 6D11 monoclonal antibody | TG100801 |
| CIX | 6D11 monoclonal antibody | sorafenib |
| CIY | 6D11 monoclonal antibody | G6-31 antibody |
| CIZ | Sis 1 monoclonal antibody | ranibizumab |
| CJA | Sis 1 monoclonal antibody | bevacizumab |
| CJB | Sis 1 monoclonal antibody | aflibercept |
| CJC | Sis 1 monoclonal antibody | KH902 VEGF receptor-Fc fusion protein |
| CJD | Sis 1 monoclonal antibody | 2C3 antibody |
| CJE | Sis 1 monoclonal antibody | ORA102 |
| CJF | Sis 1 monoclonal antibody | pegaptanib |
| CJG | Sis 1 monoclonal antibody | bevasiranib |
| CJH | Sis 1 monoclonal antibody | SIRNA-027 |
| CJI | Sis 1 monoclonal antibody | decursin |
| CJJ | Sis 1 monoclonal antibody | decursinol |
| CJK | Sis 1 monoclonal antibody | picropodophyllin |
| CJL | Sis 1 monoclonal antibody | guggulsterone |
| CJM | Sis 1 monoclonal antibody | PLG101 |
| CJN | Sis 1 monoclonal antibody | eicosanoid LXA4 |
| CJO | Sis 1 monoclonal antibody | PTK787 |
| CJP | Sis 1 monoclonal antibody | pazopanib |
| CJQ | Sis 1 monoclonal antibody | axitinib |
| CJR | Sis 1 monoclonal antibody | CDDO-Me |
| CJS | Sis 1 monoclonal antibody | CDDO-Imm |
| CJT | Sis 1 monoclonal antibody | shikonin |
| CJU | Sis 1 monoclonal antibody | beta-hydroxyisovalerylshikonin |
| CJV | Sis 1 monoclonal antibody | ganglioside GM3 |
| CJW | Sis 1 monoclonal antibody | DC101 antibody |
| CJX | Sis 1 monoclonal antibody | Mab25 antibody |
| CJY | Sis 1 monoclonal antibody | Mab73 antibody |
| CJZ | Sis 1 monoclonal antibody | 4A5 antibody |
| CKA | Sis 1 monoclonal antibody | 4E10 antibody |
| CKB | Sis 1 monoclonal antibody | 5F12 antibody |
| CKC | Sis 1 monoclonal antibody | VA01 antibody |
| CKD | Sis 1 monoclonal antibody | BL2 antibody |
| CKE | Sis 1 monoclonal antibody | VEGF-related protein |
| CKF | Sis 1 monoclonal antibody | sFLT01 |
| CKG | Sis 1 monoclonal antibody | sFLT02 |
| CKH | Sis 1 monoclonal antibody | Peptide B3 |
| CKI | Sis 1 monoclonal antibody | TG100801 |
| CKJ | Sis 1 monoclonal antibody | sorafenib |
| CKK | Sis 1 monoclonal antibody | G6-31 antibody |
| CKL | PR7212 monoclonal antibody | ranibizumab |
| CKM | PR7212 monoclonal antibody | bevacizumab |
| CKN | PR7212 monoclonal antibody | aflibercept |
| CKO | PR7212 monoclonal antibody | KH902 VEGF receptor-Fc fusion protein |
| CKP | PR7212 monoclonal antibody | 2C3 antibody |
| CKQ | PR7212 monoclonal antibody | ORA102 |
| CKR | PR7212 monoclonal antibody | pegaptanib |
| CKS | PR7212 monoclonal antibody | bevasiranib |
| CKT | PR7212 monoclonal antibody | SIRNA-027 |
| CKU | PR7212 monoclonal antibody | decursin |
| CKV | PR7212 monoclonal antibody | decursinol |
| CKW | PR7212 monoclonal antibody | Picropodophyllin |
| CKX | PR7212 monoclonal antibody | guggulsterone |
| CKY | PR7212 monoclonal antibody | PLG101 |
| CKZ | PR7212 monoclonal antibody | eicosanoid LXA4 |
| CLA | PR7212 monoclonal antibody | PTK787 |
| CLB | PR7212 monoclonal antibody | pazopanib |
| CLC | PR7212 monoclonal antibody | axitinib |
| CLD | PR7212 monoclonal antibody | CDDO-Me |
| CLE | PR7212 monoclonal antibody | CDDO-Imm |
| CLF | PR7212 monoclonal antibody | shikonin |
| CLG | PR7212 monoclonal antibody | beta-hydroxyisovalerylshikonin |
| CLH | PR7212 monoclonal antibody | ganglioside GM3 |
| CLI | PR7212 monoclonal antibody | DC101 antibody |
| CLJ | PR7212 monoclonal antibody | Mab25 antibody |
| CLK | PR7212 monoclonal antibody | Mab73 antibody |
| CLL | PR7212 monoclonal antibody | 4A5 antibody |
| CLM | PR7212 monoclonal antibody | 4E10 antibody |
| CLN | PR7212 monoclonal antibody | 5F12 antibody |
| CLO | PR7212 monoclonal antibody | VA01 antibody |
| CLP | PR7212 monoclonal antibody | BL2 antibody |
| CLQ | PR7212 monoclonal antibody | VEGF-related protein |
| CLR | PR7212 monoclonal antibody | sFLT01 |
| CLS | PR7212 monoclonal antibody | sFLT02 |
| CLT | PR7212 monoclonal antibody | Peptide B3 |
| CLU | PR7212 monoclonal antibody | TG100801 |
| CLV | PR7212 monoclonal antibody | sorafenib |
| CLW | PR7212 monoclonal antibody | G6-31 antibody |
| CLX | PR292 monoclonal antibody | ranibizumab |
| CLY | PR292 monoclonal antibody | bevacizumab |
| CLZ | PR292 monoclonal antibody | aflibercept |
| CMA | PR292 monoclonal antibody | KH902 VEGF receptor-Fc fusion protein |
| CMB | PR292 monoclonal antibody | 2C3 antibody |
| CMC | PR292 monoclonal antibody | ORA102 |
| CMD | PR292 monoclonal antibody | pegaptanib |
| CME | PR292 monoclonal antibody | bevasiranib |
| CME | PR292 monoclonal antibody | SIRNA-027 |
| CMG | PR292 monoclonal antibody | decursin |
| CMH | PR292 monoclonal antibody | decursinol |
| CMI | PR292 monoclonal antibody | picropodophyllin |
| CMJ | PR292 monoclonal antibody | guggulsterone |
| CMK | PR292 monoclonal antibody | PLG101 |
| CML | PR292 monoclonal antibody | eicosanoid LXA4 |
| CMM | PR292 monoclonal antibody | PTK787 |
| CMN | PR292 monoclonal antibody | pazopanib |
| CMO | PR292 monoclonal antibody | axitinib |
| CMP | PR292 monoclonal antibody | CDDO-Me |
| CMQ | PR292 monoclonal antibody | CDDO-Imm |
| CMR | PR292 monoclonal antibody | shikonin |
| CMS | PR292 monoclonal antibody | beta-hydroxyisovalerylshikonin |
| CMT | PR292 monoclonal antibody | ganglioside GM3 |
| CMU | PR292 monoclonal antibody | DC101 antibody |
| CMV | PR292 monoclonal antibody | Mab25 antibody |
| CMW | PR292 monoclonal antibody | Mab73 antibody |
| CMX | PR292 monoclonal antibody | 4A5 antibody |
| CMY | PR292 monoclonal antibody | 4E10 antibody |
| CMZ | PR292 monoclonal antibody | 5F12 antibody |
| CNA | PR292 monoclonal antibody | VA01 antibody |
| CNB | PR292 monoclonal antibody | BL2 antibody |
| CNC | PR292 monoclonal antibody | VEGF-related protein |
| CND | PR292 monoclonal antibody | sFLT01 |
| CNE | PR292 monoclonal antibody | sFLT02 |
| CNF | PR292 monoclonal antibody | Peptide B3 |
| CNG | PR292 monoclonal antibody | TG100801 |
| CNH | PR292 monoclonal antibody | sorafenib |
| CNI | PR292 monoclonal antibody | G6-31 antibody |
| CNJ | HYB 9610 monoclonal antibody | ranibizumab |
| CNK | HYB 9610 monoclonal antibody | bevacizumab |
| CNL | HYB 9610 monoclonal antibody | aflibercept |
| CNM | HYB 9610 monoclonal antibody | KH902 VEGF receptor-Fc fusion protein |
| CNN | HYB 9610 monoclonal antibody | 2C3 antibody |
| CNO | HYB 9610 monoclonal antibody | ORA102 |
| CNP | HYB 9610 monoclonal antibody | pegaptanib |
| CNQ | HYB 9610 monoclonal antibody | bevasiranib |
| CNR | HYB 9610 monoclonal antibody | SIRNA-027 |
| CNS | HYB 9610 monoclonal antibody | decursin |
| CNT | HYB 9610 monoclonal antibody | decursinol |
| CNU | HYB 9610 monoclonal antibody | picropodophyllin |
| CNV | HYB 9610 monoclonal antibody | guggulsterone |
| CNW | HYB 9610 monoclonal antibody | PLG101 |
| CNX | HYB 9610 monoclonal antibody | eicosanoid LXA4 |
| CNY | HYB 9610 monoclonal antibody | PTK787 |
| CNZ | HYB 9610 monoclonal antibody | pazopanib |
| COA | HYB 9610 monoclonal antibody | Axitinib |
| COB | HYB 9610 monoclonal antibody | CDDO-Me |
| COC | HYB 9610 monoclonal antibody | CDDO-Imm |
| COD | HYB 9610 monoclonal antibody | shikonin |
| COE | HYB 9610 monoclonal antibody | beta-hydroxyisovalerylshikonin |
| COF | HYB 9610 monoclonal antibody | ganglioside GM3 |
| COG | HYB 9610 monoclonal antibody | DC101 antibody |
| COH | HYB 9610 monoclonal antibody | Mab25 antibody |
| COI | HYB 9610 monoclonal antibody | Mab73 antibody |
| COJ | HYB 9610 monoclonal antibody | 4A5 antibody |
| COK | HYB 9610 monoclonal antibody | 4E10 antibody |
| COL | HYB 9610 monoclonal antibody | 5F12 antibody |
| COM | HYB 9610 monoclonal antibody | VA01 antibody |
| CON | HYB 9610 monoclonal antibody | BL2 antibody |
| COO | HYB 9610 monoclonal antibody | VEGF-related protein |
| COP | HYB 9610 monoclonal antibody | sFLT01 |
| COQ | HYB 9610 monoclonal antibody | sFLT02 |
| COR | HYB 9610 monoclonal antibody | Peptide B3 |
| COS | HYB 9610 monoclonal antibody | TG100801 |
| COT | HYB 9610 monoclonal antibody | sorafenib |
| COU | HYB 9610 monoclonal antibody | G6-31 antibody |
| COV | HYB 9611 monoclonal antibody | ranibizumab |
| COW | HYB 9611 monoclonal antibody | bevacizumab |
| COX | HYB 9611 monoclonal antibody | aflibercept |
| COY | HYB 9611 monoclonal antibody | KH902 VEGF receptor-Fc fusion protein |
| COZ | HYB 9611 monoclonal antibody | 2C3 antibody |
| CPA | HYB 9611 monoclonal antibody | ORA102 |
| CPB | HYB 9611 monoclonal antibody | pegaptanib |
| CPC | HYB 9611 monoclonal antibody | bevasiranib |
| CPD | HYB 9611 monoclonal antibody | SIRNA-027 |
| CPE | HYB 9611 monoclonal antibody | decursin |
| CPF | HYB 9611 monoclonal antibody | decursinol |
| CPG | HYB 9611 monoclonal antibody | picropodophyllin |
| CPH | HYB 9611 monoclonal antibody | guggulsterone |
| CPI | HYB 9611 monoclonal antibody | PLG101 |
| CPJ | HYB 9611 monoclonal antibody | eicosanoid LXA4 |
| CPK | HYB 9611 monoclonal antibody | PTK787 |
| CPL | HYB 9611 monoclonal antibody | pazopanib |
| CPM | HYB 9611 monoclonal antibody | axitinib |
| CPN | HYB 9611 monoclonal antibody | CDDO-Me |
| CPO | HYB 9611 monoclonal antibody | CDDO-Imm |
| CPP | HYB 9611 monoclonal antibody | Shikonin |
| CPQ | HYB 9611 monoclonal antibody | beta-hydroxyisovalerylshikonin |
| CPR | HYB 9611 monoclonal antibody | ganglioside GM3 |
| CPS | HYB 9611 monoclonal antibody | DC101 antibody |
| CPT | HYB 9611 monoclonal antibody | Mab25 antibody |
| CPU | HYB 9611 monoclonal antibody | Mab73 antibody |
| CPV | HYB 9611 monoclonal antibody | 4A5 antibody |
| CPW | HYB 9611 monoclonal antibody | 4E10 antibody |
| CPX | HYB 9611 monoclonal antibody | 5F12 antibody |
| CPY | HYB 9611 monoclonal antibody | VA01 antibody |
| CPZ | HYB 9611 monoclonal antibody | BL2 antibody |
| CQA | HYB 9611 monoclonal antibody | VEGF-related protein |
| CQB | HYB 9611 monoclonal antibody | sFLT01 |
| CQC | HYB 9611 monoclonal antibody | sFLT02 |
| CQD | HYB 9611 monoclonal antibody | Peptide B3 |
| CQE | HYB 9611 monoclonal antibody | TG100801 |
| CQF | HYB 9611 monoclonal antibody | sorafenib |
| CQG | HYB 9611 monoclonal antibody | G6-31 antibody |
| CQH | HYB 9612 monoclonal antibody | ranibizumab |
| CQI | HYB 9612 monoclonal antibody | bevacizumab |
| CQJ | HYB 9612 monoclonal antibody | aflibercept |
| CQK | HYB 9612 monoclonal antibody | KH902 VEGF receptor-Fc fusion protein |
| CQL | HYB 9612 monoclonal antibody | 2C3 antibody |
| CQM | HYB 9612 monoclonal antibody | ORA102 |
| CQN | HYB 9612 monoclonal antibody | pegaptanib |
| CQO | HYB 9612 monoclonal antibody | bevasiranib |
| CQP | HYB 9612 monoclonal antibody | SIRNA-027 |
| CQQ | HYB 9612 monoclonal antibody | decursin |
| CQR | HYB 9612 monoclonal antibody | decursinol |
| CQS | HYB 9612 monoclonal antibody | picropodophyllin |
| CQT | HYB 9612 monoclonal antibody | guggulsterone |
| CQU | HYB 9612 monoclonal antibody | PLG101 |
| CQV | HYB 9612 monoclonal antibody | eicosanoid LXA4 |
| CQW | HYB 9612 monoclonal antibody | PTK787 |
| CQX | HYB 9612 monoclonal antibody | pazopanib |
| CQY | HYB 9612 monoclonal antibody | axitinib |
| CQZ | HYB 9612 monoclonal antibody | CDDO-Me |
| CRA | HYB 9612 monoclonal antibody | CDDO-Imm |
| CRB | HYB 9612 monoclonal antibody | shikonin |
| CRC | HYB 9612 monoclonal antibody | beta-hydroxyisovalerylshikonin |
| CRD | HYB 9612 monoclonal antibody | ganglioside GM3 |
| CRE | HYB 9612 monoclonal antibody | DC101 antibody |
| CRF | HYB 9612 monoclonal antibody | Mab25 antibody |
| CRG | HYB 9612 monoclonal antibody | Mab73 antibody |
| CRH | HYB 9612 monoclonal antibody | 4A5 antibody |
| CRI | HYB 9612 monoclonal antibody | 4E10 antibody |
| CRJ | HYB 9612 monoclonal antibody | 5F12 antibody |
| CRK | HYB 9612 monoclonal antibody | VA01 antibody |
| CRL | HYB 9612 monoclonal antibody | BL2 antibody |
| CRM | HYB 9612 monoclonal antibody | VEGF-related protein |
| CRN | HYB 9612 monoclonal antibody | sFLT01 |
| CRO | HYB 9612 monoclonal antibody | sFLT02 |
| CRP | HYB 9612 monoclonal antibody | Peptide B3 |
| CRQ | HYB 9612 monoclonal antibody | TG100801 |
| CRR | HYB 9612 monoclonal antibody | sorafenib |
| CRS | HYB 9612 monoclonal antibody | G6-31 antibody |
| CRT | HYB 9613 monoclonal antibody | ranibizumab |
| CRU | HYB 9613 monoclonal antibody | bevacizumab |
| CRV | HYB 9613 monoclonal antibody | aflibercept |
| CRW | HYB 9613 monoclonal antibody | KH902 VEGF receptor-Fc fusion protein |
| CRX | HYB 9613 monoclonal antibody | 2C3 antibody |
| CRY | HYB 9613 monoclonal antibody | ORA102 |
| CRZ | HYB 9613 monoclonal antibody | pegaptanib |
| CSA | HYB 9613 monoclonal antibody | bevasiranib |
| CSB | HYB 9613 monoclonal antibody | SIRNA-027 |
| CSC | HYB 9613 monoclonal antibody | decursin |
| CSD | HYB 9613 monoclonal antibody | decursinol |
| CSE | HYB 9613 monoclonal antibody | picropodophyllin |
| CSF | HYB 9613 monoclonal antibody | guggulsterone |
| CSG | HYB 9613 monoclonal antibody | PLG101 |
| CSH | HYB 9613 monoclonal antibody | eicosanoid LXA4 |
| CSI | HYB 9613 monoclonal antibody | PTK787 |
| CSJ | HYB 9613 monoclonal antibody | pazopanib |
| CSK | HYB 9613 monoclonal antibody | axitinib |
| CSL | HYB 9613 monoclonal antibody | CDDO-Me |
| CSM | HYB 9613 monoclonal antibody | CDDO-Imm |
| CSN | HYB 9613 monoclonal antibody | shikonin |
| CSO | HYB 9613 monoclonal antibody | beta-hydroxyisovalerylshikonin |
| CSP | HYB 9613 monoclonal antibody | ganglioside GM3 |
| CSQ | HYB 9613 monoclonal antibody | DC101 antibody |
| CSR | HYB 9613 monoclonal antibody | Mab25 antibody |
| CSS | HYB 9613 monoclonal antibody | Mab73 antibody |
| CST | HYB 9613 monoclonal antibody | 4A5 antibody |
| CSU | HYB 9613 monoclonal antibody | 4E10 antibody |
| CSV | HYB 9613 monoclonal antibody | 5F12 antibody |
| CSW | HYB 9613 monoclonal antibody | VA01 antibody |
| CSX | HYB 9613 monoclonal antibody | BL2 antibody |
| CSY | HYB 9613 monoclonal antibody | VEGF-related protein |
| CSZ | HYB 9613 monoclonal antibody | sFLT01 |
| CTA | HYB 9613 monoclonal antibody | sFLT02 |
| CTB | HYB 9613 monoclonal antibody | Peptide B3 |
| CTC | HYB 9613 monoclonal antibody | TG100801 |
| CTD | HYB 9613 monoclonal antibody | sorafenib |
| CTE | HYB 9613 monoclonal antibody | G6-31 antibody |
| CTF | 4-(2-(N-(-2 carboxamidoindole) | ranibizumab |
| | aminoethyl)-benzenesulfonamide | |
| CTG | 4-(2-(N-(-2 carboxamidoindole) | bevacizumab |
| | aminoethyl)-benzenesulfonamide | |
| CTH | 4-(2-(N-(-2 carboxamidoindole) | aflibercept |
| | aminoethyl)-benzenesulfonamide | |
| CTI | 4-(2-(N-(-2 carboxamidoindole) | KH902 VEGF receptor-Fc fusion protein |
| | aminoethyl)-benzenesulfonamide | |
| CTJ | 4-(2-(N-(-2 carboxamidoindole) | 2C3 antibody |
| | aminoethyl)-benzenesulfonamide | |
| CTK | 4-(2-(N-(-2 carboxamidoindole) | ORA102 |
| | aminoethyl)-benzenesulfonamide | |
| CTL | 4-(2-(N-(-2 carboxamidoindole) | pegaptanib |
| | aminoethyl)-benzenesulfonamide | |
| CTM | 4-(2-(N-(-2 carboxamidoindole) | bevasiranib |
| | aminoethyl)-benzenesulfonamide | |
| CTN | 4-(2-(N-(-2 carboxamidoindole) | SIRNA-027 |
| | aminoethyl)-benzenesulfonamide | |
| CTO | 4-(2-(N-(-2 carboxamidoindole) | decursin |
| | aminoethyl)-benzenesulfonamide | |
| CTP | 4-(2-(N-(-2 carboxamidoindole) | decursinol |
| | aminoethyl)-benzenesulfonamide | |
| CTQ | 4-(2-(N-(-2 carboxamidoindole) | picropodophyllin |
| | aminoethyl)-benzenesulfonamide | |
| CTR | 4-(2-(N-(-2 carboxamidoindole) | guggulsterone |
| | aminoethyl)-benzenesulfonamide | |
| CTS | 4-(2-(N-(-2 carboxamidoindole) | PLG101 |
| | aminoethyl)-benzenesulfonamide | |
| CTT | 4-(2-(N-(-2 carboxamidoindole) | eicosanoid LXA4 |
| | aminoethyl)-benzenesulfonamide | |
| CTU | 4-(2-(N-(-2 carboxamidoindole) | PTK787 |
| | aminoethyl)-benzenesulfonamide | |
| CTV | 4-(2-(N-(-2 carboxamidoindole) | pazopanib |
| | aminoethyl)-benzenesulfonamide | |
| CTW | 4-(2-(N-(-2 carboxamidoindole) | axitinib |
| | aminoethyl)-benzenesulfonamide | |
| CTX | 4-(2-(N-(-2 carboxamidoindole) | CDDO-Me |
| | aminoethyl)-benzenesulfonamide | |
| CTY | 4-(2-(N-(-2 carboxamidoindole) | CDDO-Imm |
| | aminoethyl)-benzenesulfonamide | |
| CTZ | 4-(2-(N-(-2 carboxamidoindole) | shikonin |
| | aminoethyl)-benzenesulfonamide | |
| CUA | 4-(2-(N-(-2 carboxamidoindole) | beta-hydroxyisovalerylshikonin |
| | aminoethyl)-benzenesulfonamide | |
| CUB | 4-(2-(N-(-2 carboxamidoindole) | ganglioside GM3 |
| | aminoethyl)-benzenesulfonamide | |
| CUC | 4-(2-(N-(-2 carboxamidoindole) | DC101 antibody |
| | aminoethyl)-benzenesulfonamide | |
| CUD | 4-(2-(N-(-2 carboxamidoindole) | Mab25 antibody |
| | aminoethyl)-benzenesulfonamide | |
| CUE | 4-(2-(N-(-2 carboxamidoindole) | Mab73 antibody |
| | aminoethyl)-benzenesulfonamide | |
| CUF | 4-(2-(N-(-2 carboxamidoindole) | 4A5 antibody |
| | aminoethyl)-benzenesulfonamide | |
| CUG | 4-(2-(N-(-2 carboxamidoindole) | 4E10 antibody |
| | aminoethyl)-benzenesulfonamide | |
| CUH | 4-(2-(N-(-2 carboxamidoindole) | 5F12 antibody |
| | aminoethyl)-benzenesulfonamide | |
| CUI | 4-(2-(N-(-2 carboxamidoindole) | VA01 antibody |
| | aminoethyl)-benzenesulfonamide | |
| CUJ | 4-(2-(N-(-2 carboxamidoindole) | BL2 antibody |
| | aminoethyl)-benzenesulfonamide | |
| CUK | 4-(2-(N-(-2 carboxamidoindole) | VEGF-related protein |
| | aminoethyl)-benzenesulfonamide | |
| CUL | 4-(2-(N-(-2 carboxamidoindole) | sFLT01 |
| | aminoethyl)-benzenesulfonamide | |
| CUM | 4-(2-(N-(-2 carboxamidoindole) | sFLT02 |
| | aminoethyl)-benzenesulfonamide | |
| CUN | 4-(2-(N-(-2 carboxamidoindole) | Peptide B3 |
| | aminoethyl)-benzenesulfonamide | |
| CUO | 4-(2-(N-(-2 carboxamidoindole) | TG100801 |
| | aminoethyl)-benzenesulfonamide | |
| CUP | 4-(2-(N-(-2 carboxamidoindole) | sorafenib |
| | aminoethyl)-benzenesulfonamide | |
| CUQ | 4-(2-(N-(-2 carboxamidoindole) | G6-31 antibody |
| | aminoethyl)-benzenesulfonamide | |
| CUR | 4-(2-(N-(-2 | ranibizumab |
| | carboxamidoindole)aminoethyl)- | |
| | sulfonylurea | |
| CUS | 4-(2-(N-(-2 | bevacizumab |
| | carboxamidoindole)aminoethyl)- | |
| | sulfonylurea | |
| CUT | 4-(2-(N-(-2 | aflibercept |
| | carboxamidoindole)aminoethyl)- | |
| | sulfonylurea | |
| CUU | 4-(2-(N-(-2 | 1(11902 VEGF receptor-Fc fusion protein |
| | carboxamidoindole)aminoethyl)- | |
| | sulfonylurea | |
| CUV | 4-(2-(N-(-2 | 2C3 antibody |
| | carboxamidoindole)aminoethyl)- | |
| | sulfonylurea | |
| CUW | 4-(2-(N-(-2 | ORA102 |
| | carboxamidoindole)aminoethyl)- | |
| | sulfonylurea | |
| CUX | 4-(2-(N-(-2 | pegaptanib |
| | carboxamidoindole)aminoethyl)- | |
| | sulfonylurea | |
| CUY | 4-(2-(N-(-2 | bevasiranib |
| | carboxamidoindole)aminoethyl)- | |
| | sulfonylurea | |
| CUZ | 4-(2-(N-(-2 | SIRNA-027 |
| | carboxamidoindole)aminoethyl)- | |
| | sulfonylurea | |
| CVA | 4-(2-(N-(-2 | decursin |
| | carboxamidoindole)aminoethyl)- | |
| | sulfonylurea | |
| CVB | 4-(2-(N-(-2 | decursinol |
| | carboxamidoindole)aminoethyl)- | |
| | sulfonylurea | |
| CVC | 4-(2-(N-(-2 | picropodophyllin |
| | carboxamidoindole)aminoethyl)- | |
| | sulfonylurea | |
| CVD | 4-(2-(N-(-2 | Guggulsterone |
| | carboxamidoindole)aminoethyl)- | |
| | sulfonylurea | |
| CVE | 4-(2-(N-(-2 | PLG101 |
| | carboxamidoindole)aminoethyl)- | |
| | sulfonylurea | |
| CVF | 4-(2-(N-(-2 | eicosanoid LXA4 |
| | carboxamidoindole)aminoethyl)- | |
| | sulfonylurea | |
| CVG | 4-(2-(N-(-2 | PTK787 |
| | carboxamidoindole)aminoethyl)- | |
| | sulfonylurea | |
| CVH | 4-(2-(N-(-2 | pazopanib |
| | carboxamidoindole)aminoethyl)- | |
| | sulfonylurea | |
| CVI | 4-(2-(N-(-2 | axitinib |
| | carboxamidoindole)aminoethyl)- | |
| | sulfonylurea | |
| CVJ | 4-(2-(N-(-2 | CDDO-Me |
| | carboxamidoindole)aminoethyl)- | |
| | sulfonylurea | |
| CVK | 4-(2-(N-(-2 | CDDO-Imm |
| | carboxamidoindole)aminoethyl)- | |
| | sulfonylurea | |
| CVL | 4-(2-(N-(-2 | shikonin |
| | carboxamidoindole)aminoethyl)- | |
| | sulfonylurea | |
| CVM | 4-(2-(N-(-2 | beta-hydroxyisovalerylshikonin |
| | carboxamidoindole)aminoethyl)- | |
| | sulfonylurea | |
| CVN | 4-(2-(N-(-2 | ganglioside GM3 |
| | carboxamidoindole)aminoethyl)- | |
| | sulfonylurea | |
| CVO | 4-(2-(N-(-2 | DC101 antibody |
| | carboxamidoindole)aminoethyl)- | |
| | sulfonylurea | |
| CVP | 4-(2-(N-(-2 | Mab25 antibody |
| | carboxamidoindole)aminoethyl)- | |
| | sulfonylurea | |
| CVQ | 4-(2-(N-(-2 | Mab73 antibody |
| | carboxamidoindole)aminoethyl)- | |
| | sulfonylurea | |
| CVR | 4-(2-(N-(-2 | 4A5 antibody |
| | carboxamidoindole)aminoethyl)- | |
| | sulfonylurea | |
| CVS | 4-(2-(N-(-2 | 4E10 antibody |
| | carboxamidoindole)aminoethyl)- | |
| | sulfonylurea | |
| CVT | 4-(2-(N-(-2 | 5F12 antibody |
| | carboxamidoindole)aminoethyl)- | |
| | sulfonylurea | |
| CVU | 4-(2-(N-(-2 | VA01 antibody |
| | carboxamidoindole)aminoethyl)- | |
| | sulfonylurea | |
| CVV | 4-(2-(N-(-2 | BL2 antibody |
| | carboxamidoindole)aminoethyl)- | |
| | sulfonylurea | |
| CVW | 4-(2-(N-(-2 | VEGF-related protein |
| | carboxamidoindole)aminoethyl)- | |
| | sulfonylurea | |
| CVX | 4-(2-(N-(-2 | sFLT01 |
| | carboxamidoindole)aminoethyl)- | |
| | sulfonylurea | |
| CVY | 4-(2-(N-(-2 | sFLT02 |
| | carboxamidoindole)aminoethyl)- | |
| | sulfonylurea | |
| CVZ | 4-(2-(N-(-2 | Peptide B3 |
| | carboxamidoindole)aminoethyl)- | |
| | sulfonylurea | |
| CWA | 4-(2-(N-(-2 | TG100801 |
| | carboxamidoindole)aminoethyl)- | |
| | sulfonylurea | |
| CWB | 4-(2-(N-(-2 | sorafenib |
| | carboxamidoindole)aminoethyl)- | |
| | sulfonylurea | |
| CWC | 4-(2-(N-(-2 | G6-31 antibody |
| | carboxamidoindole)aminoethyl)- | |
| | sulfonylurea | |
| CWD | CGP 53716 | ranibizumab |
| CWE | CGP 53716 | bevacizumab |
| CWF | CGP 53716 | aflibercept |
| CWG | CGP 53716 | KH902 VEGF receptor-Fc fusion protein |
| CWH | CGP 53716 | 2C3 antibody |
| CWI | CGP 53716 | ORA102 |
| CWJ | CGP 53716 | pegaptanib |
| CWK | CGP 53716 | bevasiranib |
| CWL | CGP 53716 | SIRNA-027 |
| CWM | CGP 53716 | decursin |
| CWN | CGP 53716 | decursinol |
| CWO | CGP 53716 | picropodophyllin |
| CWP | CGP 53716 | guggulsterone |
| CWQ | CGP 53716 | PLG101 |
| CWR | CGP 53716 | eicosanoid LXA4 |
| CWS | CGP 53716 | PTK787 |
| CWT | CGP 53716 | pazopanib |
| CWU | CGP 53716 | axitinib |
| CWV | CGP 53716 | CDDO-Me |
| CWW | CGP 53716 | CDDO-Imm |
| CWX | CGP 53716 | shikonin |
| CWY | CGP 53716 | beta-hydroxyisovalerylshikonin |
| CWZ | CGP 53716 | ganglioside GM3 |
| CXA | CGP 53716 | DC101 antibody |
| CXB | CGP 53716 | Mab25 antibody |
| CXC | CGP 53716 | Mab73 antibody |
| CXD | CGP 53716 | 4A5 antibody |
| CXE | CGP 53716 | 4E10 antibody |
| CXF | CGP 53716 | 5F12 antibody |
| CXG | CGP 53716 | VA01 antibody |
| CXH | CGP 53716 | BL2 antibody |
| CXI | CGP 53716 | VEGF-related protein |
| CXJ | CGP 53716 | sFLT01 |
| CXK | CGP 53716 | sFLT02 |
| CXL | CGP 53716 | Peptide B3 |
| CXM | CGP 53716 | TG100801 |
| CXN | CGP 53716 | sorafenib |
| CXO | CGP 53716 | G6-31 antibody |
| CXP | G162 antibody | ranibizumab |
| CXQ | G162 antibody | bevacizumab |
| CXR | G162 antibody | aflibercept |
| CXS | G162 antibody | KH902 VEGF receptor-Fc fusion protein |
| CXT | G162 antibody | 2C3 antibody |
| CXU | G162 antibody | ORA102 |
| CXV | G162 antibody | pegaptanib |
| CXW | G162 antibody | bevasiranib |
| CXX | G162 antibody | SIRNA-027 |
| CXY | G162 antibody | decursin |
| CXZ | G162 antibody | decursinol |
| CYA | G162 antibody | Picropodophyllin |
| CYB | G162 antibody | guggulsterone |
| CYC | G162 antibody | PLG101 |
| CYD | G162 antibody | eicosanoid LXA4 |
| CYE | G162 antibody | PTK787 |
| CYF | G162 antibody | pazopanib |
| CYG | G162 antibody | axitinib |
| CYH | G162 antibody | CDDO-Me |
| CYI | G162 antibody | CDDO-Imm |
| CYJ | G162 antibody | shikonin |
| CYK | G162 antibody | beta-hydroxyisovalerylshikonin |
| CYL | G162 antibody | ganglioside GM3 |
| CYM | G162 antibody | DC101 antibody |
| CYN | G162 antibody | Mab25 antibody |
| CYO | G162 antibody | Mab73 antibody |
| CYP | G162 antibody | 4A5 antibody |
| CYQ | G162 antibody | 4E10 antibody |
| CYR | G162 antibody | 5F12 antibody |
| CYS | G162 antibody | VA01 antibody |
| CYT | G162 antibody | BL2 antibody |
| CYU | G162 antibody | VEGF-related protein |
| CYV | G162 antibody | sFLT01 |
| CYW | G162 antibody | sFLT02 |
| CYX | G162 antibody | Peptide B3 |
| CYY | G162 antibody | TG100801 |
| CYZ | G162 antibody | sorafenib |
| CZA | G162 antibody | G6-31 antibody |
| CZB | pyrazolo[3,4-g]quinoxaline | ranibizumab |
| CZC | pyrazolo[3,4-g]quinoxaline | bevacizumab |
| CZD | pyrazolo[3,4-g]quinoxaline | aflibercept |
| CZE | pyrazolo[3,4-g]quinoxaline | KH902 VEGF receptor-Fc fusion protein |
| CZF | pyrazolo[3,4-g]quinoxaline | 2C3 antibody |
| CZG | pyrazolo[3,4-g]quinoxaline | ORA102 |
| CZH | pyrazolo[3,4-g]quinoxaline | pegaptanib |
| CZI | pyrazolo[3,4-g]quinoxaline | bevasiranib |
| CZJ | pyrazolo[3,4-g]quinoxaline | SIRNA-027 |
| CZK | pyrazolo[3,4-g]quinoxaline | decursin |
| CZL | pyrazolo[3,4-g]quinoxaline | decursinol |
| CZM | pyrazolo[3,4-g]quinoxaline | picropodophyllin |
| CZN | pyrazolo[3,4-g]quinoxaline | guggulsterone |
| CZO | pyrazolo[3,4-g]quinoxaline | PLG101 |
| CZP | pyrazolo[3,4-g]quinoxaline | eicosanoid LXA4 |
| CZQ | pyrazolo[3,4-g]quinoxaline | PTK787 |
| CZR | pyrazolo[3,4-g]quinoxaline | pazopanib |
| CZS | pyrazolo[3,4-g]quinoxaline | axitinib |
| CZT | pyrazolo[3,4-g]quinoxaline | CDDO-Me |
| CZU | pyrazolo[3,4-g]quinoxaline | CDDO-Imm |
| CZV | pyrazolo[3,4-g]quinoxaline | shikonin |
| CZW | pyrazolo[3,4-g]quinoxaline | beta-hydroxyisovalerylshikonin |
| CZX | pyrazolo[3,4-g]quinoxaline | ganglioside GM3 |
| CZY | pyrazolo[3,4-g]quinoxaline | DC101 antibody |
| CZZ | pyrazolo[3,4-g]quinoxaline | Mab25 antibody |
| DAA | pyrazolo[3,4-g]quinoxaline | Mab73 antibody |
| DAB | pyrazolo[3,4-g]quinoxaline | 4A5 antibody |
| DAC | pyrazolo[3,4-g]quinoxaline | 4E10 antibody |
| DAD | pyrazolo[3,4-g]quinoxaline | 5F12 antibody |
| DAE | pyrazolo[3,4-g]quinoxaline | VA01 antibody |
| DAF | pyrazolo[3,4-g]quinoxaline | BL2 antibody |
| DAG | pyrazolo[3,4-g]quinoxaline | VEGF-related protein |
| DAH | pyrazolo[3,4-g]quinoxaline | sFLT01 |
| DAI | pyrazolo[3,4-g]quinoxaline | sFLT02 |
| DAJ | pyrazolo[3,4-g]quinoxaline | Peptide B3 |
| DAK | pyrazolo[3,4-g]quinoxaline | TG100801 |
| DAL | pyrazolo[3,4-g]quinoxaline | sorafenib |
| DAM | pyrazolo[3,4-g]quinoxaline | G6-31 antibody |
| DAN | 6-[2- | ranibizumab |
| | (methylcarbamoyl)phenylsulphanyl]- | |
| | 3-E[2-(pyridine-2-yl)ethenyl]- | |
| | indazole | |
| DAO | 6-[2- | bevacizumab |
| | (methylcarbamoyl)phenylsulphanyl]- | |
| | 3-E[2-(pyridine-2-yl)ethenyl]- | |
| | indazole | |
| DAP | 6-[2- | Aflibercept |
| | (methylcarbamoyl)phenylsulphanyl]- | |
| | 3-E[2-(pyridine-2-yl)ethenyl]- | |
| | indazole | |
| DAQ | 6-[2- | KH902 VEGF receptor-Fc fusion protein |
| | (methylcarbamoyl)phenylsulphanyl]- | |
| | 3-E[2-(pyridine-2-yl)ethenyl]- | |
| | indazole | |
| DAR | 6-[2- | 2C3 antibody |
| | (methylcarbamoyl)phenylsulphanyl]- | |
| | 3-E[2-(pyridine-2-yl)ethenyl]- | |
| | indazole | |
| DAS | 6-[2- | ORA102 |
| | (methylcarbamoyl)phenylsulphanyl]- | |
| | 3-E[2-(pyridine-2-yl)ethenyl]- | |
| | indazole | |
| DAT | 6-[2- | pegaptanib |
| | (methylcarbamoyl)phenylsulphanyl]- | |
| | 3-E[2-(pyridine-2-yl)ethenyl]- | |
| | indazole | |
| DAU | 6-[2- | bevasiranib |
| | (methylcarbamoyl)phenylsulphanyl]- | |
| | 3-E[2-(pyridine-2-yl)ethenyl]- | |
| | indazole | |
| DAV | 6-[2- | SIRNA-027 |
| | (methylcarbamoyl)phenylsulphanyl]- | |
| | 3-E[2-(pyridine-2-yl)ethenyl]- | |
| | indazole | |
| DAW | 6-[2- | decursin |
| | (methylcarbamoyl)phenylsulphanyl]- | |
| | 3-E[2-(pyridine-2-yl)ethenyl]- | |
| | indazole | |
| DAX | 6-[2- | decursinol |
| | (methylcarbamoyl)phenylsulphanyl]- | |
| | 3-E[2-(pyridine-2-yl)ethenyl]- | |
| | indazole | |
| DAY | 6-[2- | picropodophyllin |
| | (methylcarbamoyl)phenylsulphanyl]- | |
| | 3-E[2-(pyridine-2-yl)ethenyl]- | |
| | indazole | |
| DAZ | 6-[2- | guggulsterone |
| | (methylcarbamoyl)phenylsulphanyl]- | |
| | 3-E[2-(pyridine-2-yl)ethenyl]- | |
| | indazole | |
| DBA | 6-[2- | PLG101 |
| | (methylcarbamoyl)phenylsulphanyl]- | |
| | 3-E[2-(pyridine-2-yl)ethenyl]- | |
| | indazole | |
| DBB | 6-[2- | eicosanoid LXA4 |
| | (methylcarbamoyl)phenylsulphanyl]- | |
| | 3-E[2-(pyridine-2-yl)ethenyl]- | |
| | indazole | |
| DBC | 6-[2- | PTK787 |
| | (methylcarbamoyl)phenylsulphanyl]- | |
| | 3-E[2-(pyridine-2-yl)ethenyl]- | |
| | indazole | |
| DBD | 6-[2- | pazopanib |
| | (methylcarbamoyl)phenylsulphanyl]- | |
| | 3-E[2-(pyridine-2-yl)ethenyl]- | |
| | indazole | |
| DBE | 6-[2- | axitinib |
| | (methylcarbamoyl)phenylsulphanyl]- | |
| | 3-E[2-(pyridine-2-yl)ethenyl]- | |
| | indazole | |
| DBF | 6-[2- | CDDO-Me |
| | (methylcarbamoyl)phenylsulphanyl]- | |
| | 3-E[2-(pyridine-2-yl)ethenyl]- | |
| | indazole | |
| DBG | 6-[2- | CDDO-Imm |
| | (methylcarbamoyl)phenylsulphanyl]- | |
| | 3-E[2-(pyridine-2-yl)ethenyl]- | |
| | indazole | |
| DBH | 6-[2- | shikonin |
| | (methylcarbamoyl)phenylsulphanyl]- | |
| | 3-E[2-(pyridine-2-yl)ethenyl]- | |
| | indazole | |
| DBI | 6-[2- | beta-hydroxyisovalerylshikonin |
| | (methylcarbamoyl)phenylsulphanyl]- | |
| | 3-E[2-(pyridine-2-yl)ethenyl]- | |
| | indazole | |
| DBJ | 6-[2- | ganglioside GM3 |
| | (methylcarbamoyl)phenylsulphanyl]- | |
| | 3-E[2-(pyridine-2-yl)ethenyl]- | |
| | indazole | |
| DBK | 6-[2- | DC101 antibody |
| | (methylcarbamoyl)phenylsulphanyl]- | |
| | 3-E[2-(pyridine-2-yl)ethenyl]- | |
| | indazole | |
| DBL | 6-[2- | Mab25 antibody |
| | (methylcarbamoyl)phenylsulphanyl]- | |
| | 3-E[2-(pyridine-2-yl)ethenyl]-indazole | |
| DBM | 6-[2- | Mab73 antibody |
| | (methylcarbamoyl)phenylsulphanyl]- | |
| | 3-E[2-(pyridine-2-yl)ethenyl]-indazole | |
| DBN | 6-[2- | 4A5 antibody |
| | (methylcarbamoyl)phenylsulphanyl]- | |
| | 3-E[2-(pyridine-2-yl)ethenyl]-indazole | |
| DBO | 6-[2- | 4E10 antibody |
| | (methylcarbamoyl)phenylsulphanyl]- | |
| | 3-E[2-(pyridine-2-yl)ethenyl]-indazole | |
| DBP | 6-[2- | 5F12 antibody |
| | (methylcarbamoyl)phenylsulphanyl]- | |
| | 3-E[2-(pyridine-2-yl)ethenyl]-indazole | |
| DB Q | 6-[2- | VA01 antibody |
| | (methylcarbamoyl)phenylsulphanyl]- | |
| | 3-E[2-(pyridine-2-yl)ethenyl]-indazole | |
| DBR | 6-[2- | BL2 antibody |
| | (methylcarbamoyl)phenylsulphanyl]- | |
| | 3-E[2-(pyridine-2-yl)ethenyl]-indazole | |
| DB S | 6-[2- | VEGF-related protein |
| | (methylcarbamoyl)phenylsulphanyl]- | |
| | 3-E[2-(pyridine-2-yl)ethenyl]-indazole | |
| DBT | 6-[2- | sFLT01 |
| | (methylcarbamoyl)phenylsulphanyl]- | |
| | 3-E[2-(pyridine-2-yl)ethenyl]-indazole | |
| DBU | 6-[2- | sFLT02 |
| | (methylcarbamoyl)phenylsulphanyl]- | |
| | 3-E[2-(pyridine-2-yl)ethenyl]-indazole | |
| DBV | 6-[2- | Peptide B3 |
| | (methylcarbamoyl)phenylsulphanyl]- | |
| | 3-E[2-(pyridine-2-yl)ethenyl]-indazole | |
| DBW | 6-[2- | TG100801 |
| | (methylcarbamoyl)phenylsulphanyl]- | |
| | 3-E[2-(pyridine-2-yl)ethenyl]-indazole | |
| DBX | 6-[2- | Sorafenib |
| | (methylcarbamoyl)phenylsulphanyl]- | |
| | 3-E[2-(pyridine-2-yl)ethenyl]-indazole | |
| DBY | 6-[2- | G6-31 antibody |
| | (methylcarbamoyl)phenylsulphanyl]- | |
| | 3-E[2-(pyridine-2-yl)ethenyl]-indazole | |
| DBZ | 1-{2-[5-(2-methoxy-ethoxy)- | ranibizumab |
| | benzoimidazole-1-yl]-quinoline-8- | |
| | yl}-piperidine-4-ylamine | |
| DCA | 1-{2-[5-(2-methoxy-ethoxy)- | bevacizumab |
| | benzoimidazole-1-yl]-quinoline-8-yl}piperidine- | |
| | 4-ylamine | |
| DCB | 1-{2-[5-(2-methoxy-ethoxy)- | aflibercept |
| | benzoimidazole-1-yl]-quinoline-8-yl}piperidine- | |
| | 4-ylamine | |
| DCC | 1-{2-[5-(2-methoxy-ethoxy)- | KI-1902 VEGF receptor-Fc fusion protein |
| | benzoimidazole-1-yl]-quinoline-8-yl}piperidine- | |
| | 4-ylamine | |
| DCD | 1-{2-[5-(2-methoxy-ethoxy)- | 2C3 antibody |
| | benzoimidazole-1-yl]-quinoline-8-yl}piperidine- | |
| | 4-ylamine | |
| DCE | 1-{2-[5-(2-methoxy-ethoxy)- | ORA102 |
| | benzoimidazole-1-yl]-quinoline-8-yl}piperidine- | |
| | 4-ylamine | |
| DCF | 1-{2-[5-(2-methoxy-ethoxy)- | pegaptanib |
| | benzoimidazole-1-yl]-quinoline-8-yl}piperidine- | |
| | 4-ylamine | |
| DCG | 1-{2-[5-(2-methoxy-ethoxy)- | bevasiranib |
| | benzoimidazole-1-yl]-quinoline-8-yl}piperidine- | |
| | 4-ylamine | |
| DCH | 1-{2-[5-(2-methoxy-ethoxy)- | SIRNA-027 |
| | benzoimidazole-1-yl]-quinoline-8-yl}piperidine- | |
| | 4-ylamine | |
| DCI | 1-{2-[5-(2-methoxy-ethoxy)- | decursin |
| | benzoimidazole-1-yl]-quinoline-8-yl}piperidine- | |
| | 4-ylamine | |
| DCJ | 1-{2-[5-(2-methoxy-ethoxy)- | decursinol |
| | benzoimidazole-1-yl]-quinoline-8-yl}piperidine- | |
| | 4-ylamine | |
| DCK | 1-{2-[5-(2-methoxy-ethoxy)- | Picropodophyllin |
| | benzoimidazole-1-yl]-quinoline-8-yl}piperidine- | |
| | 4-ylamine | |
| DCL | 1-{2-[5-(2-methoxy-ethoxy)- | guggulsterone |
| | benzoimidazole-1-yl]-quinoline-8-yl}piperidine- | |
| | 4-ylamine | |
| DCM | 1-{2-[5-(2-methoxy-ethoxy)- | PLG101 |
| | benzoimidazole-1-yl]-quinoline-8-yl}piperidine- | |
| | 4-ylamine | |
| DCN | 1-{2-[5-(2-methoxy-ethoxy)- | eicosanoid LXA4 |
| | benzoimidazole-1-yl]-quinoline-8-yl}piperidine- | |
| | 4-ylamine | |
| DCO | 1-{2-[5-(2-methoxy-ethoxy)- | PTK787 |
| | benzoimidazole-1-yl]-quinoline-8-yl}piperidine- | |
| | 4-ylamine | |
| DCP | 1-{2-[5-(2-methoxy-ethoxy)- | pazopanib |
| | benzoimidazole-1-yl]-quinoline-8-yl}piperidine- | |
| | 4-ylamine | |
| DCQ | 1-{2-[5-(2-methoxy-ethoxy)- | axitinib |
| | benzoimidazole-1-yl]-quinoline-8-yl}piperidine- | |
| | 4-ylamine | |
| DCR | 1-{2-[5-(2-methoxy-ethoxy)- | CDDO-Me |
| | benzoimidazole-1-yl]-quinoline-8-yl}piperidine- | |
| | 4-ylamine | |
| DCS | 1-{2-[5-(2-methoxy-ethoxy)- | CDDO-Imm |
| | benzoimidazole-1-yl]-quinoline-8-yl}piperidine- | |
| | 4-ylamine | |
| DCT | 1-{2-[5-(2-methoxy-ethoxy)- | shikonin |
| | benzoimidazole-1-yl]-quinoline-8-yl}piperidine- | |
| | 4-ylamine | |
| DCU | 1-{2-[5-(2-methoxy-ethoxy)- | beta-hydroxyisovalerylshikonin |
| | benzoimidazole-1-yl]-quinoline-8-yl}piperidine- | |
| | 4-ylamine | |
| DCV | 1-{2-[5-(2-methoxy-ethoxy)- | ganglioside GM3 |
| | benzoimidazole-1-yl]-quinoline-8-yl}piperidine- | |
| | 4-ylamine | |
| DCW | 1-{2-[5-(2-methoxy-ethoxy)- | DC101 antibody |
| | benzoimidazole-1-yl]-quinoline-8-yl}piperidine- | |
| | 4-ylamine | |
| DCX | 1-{2-[5-(2-methoxy-ethoxy)- | Mab25 antibody |
| | benzoimidazole-1-yl]-quinoline-8-yl}piperidine- | |
| | 4-ylamine | |
| DCY | 1-{2-[5-(2-methoxy-ethoxy)- | Mab73 antibody |
| | benzoimidazole-1-yl]-quinoline-8-yl}piperidine- | |
| | 4-ylamine | |
| DCZ | 1-{2-[5-(2-methoxy-ethoxy)- | 4A5 antibody |
| | benzoimidazole-1-yl]-quinoline-8-yl}piperidine- | |
| | 4-ylamine | |
| DDA | 1-{2-[5-(2-methoxy-ethoxy)- | 4E10 antibody |
| | benzoimidazole-1-yl]-quinoline-8-yl}piperidine- | |
| | 4-ylamine | |
| DDB | 1-{2-[5-(2-methoxy-ethoxy)- | 5F12 antibody |
| | benzoimidazole-1-yl]-quinoline-8-yl}piperidine- | |
| | 4-ylamine | |
| DDC | 1-{2-[5-(2-methoxy-ethoxy)- | VA01 antibody |
| | benzoimidazole-1-yl]-quinoline-8-yl}piperidine- | |
| | 4-ylamine | |
| DDD | 1-{2-[5-(2-methoxy-ethoxy)- | BL2 antibody |
| | benzoimidazole-1-yl]-quinoline-8-yl}piperidine- | |
| | 4-ylamine | |
| DDE | 1-{2-[5-(2-methoxy-ethoxy)- | VEGF-related protein |
| | benzoimidazole-1-yl]-quinoline-8-yl}piperidine- | |
| | 4-ylamine | |
| DDF | 1-{2-[5-(2-methoxy-ethoxy)- | sFLT01 |
| | benzoimidazole-1-yl]-quinoline-8-yl}piperidine- | |
| | 4-ylamine | |
| DDG | 1-{2-[5-(2-methoxy-ethoxy)- | sFLT02 |
| | benzoimidazole-1-yl]-quinoline-8-yl}piperidine- | |
| | 4-ylamine | |
| DDH | 1-{2-[5-(2-methoxy-ethoxy)- | Peptide B3 |
| | benzoimidazole-1-yl]-quinoline-8-yl}piperidine- | |
| | 4-ylamine | |
| DDI | 1-{2-[5-(2-methoxy-ethoxy)- | TG100801 |
| | benzoimidazole-1-yl]-quinoline-8-yl}piperidine- | |
| | 4-ylamine | |
| DDJ | 1-{2-[5-(2-methoxy-ethoxy)- | sorafenib |
| | benzoimidazole-1-yl]-quinoline-8-yl}piperidine- | |
| | 4-ylamine | |
| DDK | 1-{2-[5-(2-methoxy-ethoxy)- | G6-31 antibody |
| | benzoimidazole-1-yl]-quinoline-8-yl}piperidine- | |
| | 4-ylamine | |
| DDL | 4-[4-[N-(4-nitrophenyl)carbamoyl]- | ranibizumab |
| | 1-piperazinyl]-6,7- | |
| | dimethoxyquinazoline | |
| DDM | 4-[4-[N-(4-nitrophenyl)carbamoyl]- | bevacizumab |
| | 1-piperazinyl]-6,7- | |
| | dimethoxyquinazoline | |
| DDN | 4-[4-[N-(4-nitrophenyl)carbamoyl]- | aflibercept |
| | 1-piperazinyl]-6,7- | |
| | dimethoxyquinazoline | |
| DDO | 4-[4-[N-(4-nitrophenyl)carbamoyl]- | KI-1902 VEGF receptor-Fc fusion protein |
| | 1-piperazinyl]-6,7- | |
| | dimethoxyquinazoline | |
| DDP | 4-[4-[N-(4-nitrophenyl)carbamoyl]- | 2C3 antibody |
| | 1-piperazinyl]-6,7- | |
| | dimethoxyquinazoline | |
| DDQ | 4-[4-[N-(4-nitrophenyl)carbamoyl]- | ORA102 |
| | 1-piperazinyl]-6,7- | |
| | dimethoxyquinazoline | |
| DDR | 4-[4-[N-(4-nitrophenyl)carbamoyl]- | pegaptanib |
| | 1-piperazinyl]-6,7- | |
| | dimethoxyquinazoline | |
| DDS | 4-[4-[N-(4-nitrophenyl)carbamoyl]- | bevasiranib |
| | 1-piperazinyl]-6,7- | |
| | dimethoxyquinazoline | |
| DDT | 4-[4-[N-(4-nitrophenyl)carbamoyl]- | SIRNA-027 |
| | 1-piperazinyl]-6,7- | |
| | dimethoxyquinazoline | |
| DDU | 4-[4-[N-(4-nitrophenyl)carbamoyl]- | decursin |
| | 1-piperazinyl]-6,7- | |
| | dimethoxyquinazoline | |
| DDV | 4-[4-[N-(4-nitrophenyl)carbamoyl]- | decursinol |
| | 1-piperazinyl]-6,7- | |
| | dimethoxyquinazoline | |
| DDW | 4-[4-[N-(4-nitrophenyl)carbamoyl]- | picropodophyllin |
| | 1-piperazinyl]-6,7- | |
| | dimethoxyquinazoline | |
| DDX | 4-[4-[N-(4-nitrophenyl)carbamoyl]- | guggulsterone |
| | 1-piperazinyl]-6,7- | |
| | dimethoxyquinazoline | |
| DDY | 4-[4-[N-(4-nitrophenyl)carbamoyl]- | PLG101 |
| | 1-piperazinyl]-6,7- | |
| | dimethoxyquinazoline | |
| DDZ | 4-[4-[N-(4-nitrophenyl)carbamoyl]- | eicosanoid LXA4 |
| | 1-piperazinyl]-6,7- | |
| | dimethoxyquinazoline | |
| DEA | 4-[4-[N-(4-nitrophenyl)carbamoyl]- | PTK787 |
| | 1-piperazinyl]-6,7- | |
| | dimethoxyquinazoline | |
| DEB | 4-[4-[N-(4-nitrophenyl)carbamoyl]- | pazopanib |
| | 1-piperazinyl]-6,7- | |
| | dimethoxyquinazoline | |
| DEC | 4-[4-[N-(4-nitrophenyl)carbamoyl]- | axitinib |
| | 1-piperazinyl]-6,7- | |
| | dimethoxyquinazoline | |
| DED | 4-[4-[N-(4-nitrophenyl)carbamoyl]- | CDDO-Me |
| | 1-piperazinyl]-6,7- | |
| | dimethoxyquinazoline | |
| DEE | 4-[4-[N-(4-nitrophenyl)carbamoyl]- | CDDO-Imm |
| | 1-piperazinyl]-6,7- | |
| | dimethoxyquinazoline | |
| DEF | 4-[4-[N-(4-nitrophenyl)carbamoyl]- | shikonin |
| | 1-piperazinyl]-6,7- | |
| | dimethoxyquinazoline | |
| DEG | 4-[4-[N-(4-nitrophenyl)carbamoyl]- | beta-hydroxyisovalerylshikonin |
| | 1-piperazinyl]-6,7- | |
| | dimethoxyquinazoline | |
| DEH | 4-[4-[N-(4-nitrophenyl)carbamoyl]- | ganglioside GM3 |
| | 1-piperazinyl]-6,7- | |
| | dimethoxyquinazoline | |
| DEI | 4-[4-[N-(4-nitrophenyl)carbamoyl]- | DC101 antibody |
| | 1-piperazinyl]-6,7- | |
| | dimethoxyquinazoline | |
| DEJ | 4-[4-[N-(4-nitrophenyl)carbamoyl]- | Mab25 antibody |
| | 1-piperazinyl]-6,7- | |
| | dimethoxyquinazoline | |
| DEK | 4-[4-[N-(4-nitrophenyl)carbamoyl]- | Mab73 antibody |
| | 1-piperazinyl]-6,7- | |
| | dimethoxyquinazoline | |
| DEL | 4-[4-[N-(4-nitrophenyl)carbamoyl]- | 4A5 antibody |
| | 1-piperazinyl]-6,7- | |
| | dimethoxyquinazoline | |
| DEM | 4-[4-[N-(4-nitrophenyl)carbamoyl]- | 4E10 antibody |
| | 1-piperazinyl]-6,7- | |
| | dimethoxyquinazoline | |
| DEN | 4-[4-[N-(4-nitrophenyl)carbamoyl]- | 5F12 antibody |
| | 1-piperazinyl]-6,7- | |
| | dimethoxyquinazoline | |
| DEO | 4-[4-[N-(4-nitrophenyl)carbamoyl]- | VA01 antibody |
| | 1-piperazinyl]-6,7- | |
| | dimethoxyquinazoline | |
| DEP | 4-[4-[N-(4-nitrophenyl)carbamoyl]- | BL2 antibody |
| | 1-piperazinyl]-6,7- | |
| | dimethoxyquinazoline | |
| DEQ | 4-[4-[N-(4-nitrophenyl)carbamoyl]- | VEGF-related protein |
| | 1-piperazinyl]-6,7- | |
| | dimethoxyquinazoline | |
| DER | 4-[4-[N-(4-nitrophenyl)carbamoyl]- | sFLT01 |
| | 1-piperazinyl]-6,7- | |
| | dimethoxyquinazoline | |
| DES | 4-[4-[N-(4-nitrophenyl)carbamoyl]- | sFLT02 |
| | 1-piperazinyl]-6,7- | |
| | dimethoxyquinazoline | |
| DET | 4-[4-[N-(4-nitrophenyl)carbamoyl]- | Peptide B3 |
| | 1-piperazinyl]-6,7- | |
| | dimethoxyquinazoline | |
| DEU | 4-[4-[N-(4-nitrophenyl)carbamoyl]- | TG100801 |
| | 1-piperazinyl]-6,7- | |
| | dimethoxyquinazoline | |
| DEV | 4-[4-[N-(4-nitrophenyl)carbamoyl]- | sorafenib |
| | 1-piperazinyl]-6,7- | |
| | dimethoxyquinazoline | |
| DEW | 4-[4-[N-(4-nitrophenyl)carbamoyl]- | G6-31 antibody |
| | 1-piperazinyl]-6,7- | |
| | dimethoxyquinazoline | |
| DEX | 4-amino-5-fluoro-3-(6-(4-methyl- | ranibizumab |
| | piperazine-1-yl)-1H-benzimidazole- | |
| | 2-yl)-1H-quinoline-2-one | |
| DEY | 4-amino-5-fluoro-3-(6-(4-methyl- | bevacizumab |
| | piperazine-1-yl)-1H-benzimidazole- | |
| | 2-yl)-1H-quinoline-2-one | |
| DEZ | 4-amino-5-fluoro-3-(6-(4-methyl- | aflibercept |
| | piperazine-1-yl)-1H-benzimidazole- | |
| | 2-yl)-1H-quinoline-2-one | |
| DFA | 4-amino-5-fluoro-3-(6-(4-methyl- | KI-1902 VEGF receptor-Fc fusion protein |
| | piperazine-1-yl)-1H-benzimidazole- | |
| | 2-yl)-1H-quinoline-2-one | |
| DFB | 4-amino-5-fluoro-3-(6-(4-methyl- | 2C3 antibody |
| | piperazine-1-yl)-1H-benzimidazole- | |
| | 2-yl)-1H-quinoline-2-one | |
| DFC | 4-amino-5-fluoro-3-(6-(4-methyl- | ORA102 |
| | piperazine-1-yl)-1H-benzimidazole- | |
| | 2-yl)-1H-quinoline-2-one | |
| DFD | 4-amino-5-fluoro-3-(6-(4-methyl- | pegaptanib |
| | piperazine-1-yl)-1H-benzimidazole- | |
| | 2-yl)-1H-quinoline-2-one | |
| DFE | 4-amino-5-fluoro-3-(6-(4-methyl- | bevasiranib |
| | piperazine-1-yl)-1H-benzimidazole- | |
| | 2-yl)-1H-quinoline-2-one | |
| DFF | 4-[4-[N-(4-nitrophenyl)carbamoyl]- | SIRNA-027 |
| | 1-piperazinyl]-6,7- | |
| | dimethoxyquinazoline | |
| DFG | 4-amino-5-fluoro-3-(6-(4-methyl- | decursin |
| | piperazine-1-yl)-1H-benzimidazole- | |
| | 2-yl)-1H-quinoline-2-one | |
| DFH | 4-amino-5-fluoro-3-(6-(4-methyl- | decursinol |
| | piperazine-1-yl)-1H-benzimidazole- | |
| | 2-yl)-1H-quinoline-2-one | |
| DFI | 4-amino-5-fluoro-3-(6-(4-methyl- | picropodophyllin |
| | piperazine-1-yl)-1H-benzimidazole- | |
| | 2-yl)-1H-quinoline-2-one | |
| DFJ | 4-amino-5-fluoro-3-(6-(4-methyl- | guggulsterone |
| | piperazine-1-yl)-1H-benzimidazole- | |
| | 2-yl)-1H-quinoline-2-one | |
| DFK | 4-amino-5-fluoro-3-(6-(4-methyl- | PLG101 |
| | piperazine-1-yl)-1H-benzimidazole- | |
| | 2-yl)-1H-quinoline-2-one | |
| DFL | 4-amino-5-fluoro-3-(6-(4-methyl- | eicosanoid LXA4 |
| | piperazine-1-yl)-1H-benzimidazole- | |
| | 2-yl)-1H-quinoline-2-one | |
| DFM | 4-amino-5-fluoro-3-(6-(4-methyl- | PTK787 |
| | piperazine-1-yl)-1H-benzimidazole- | |
| | 2-yl)-1H-quinoline-2-one | |
| DFN | 4-amino-5-fluoro-3-(6-(4-methyl- | pazopanib |
| | piperazine-1-yl)-1H-benzimidazole- | |
| | 2-yl)-1H-quinoline-2-one | |
| DFO | 4-[4-[N-(4-nitrophenyl)carbamoyl]- | Axitinib |
| | 1-piperazinyl]-6,7- | |
| | dimethoxyquinazoline | |
| DFP | 4-amino-5-fluoro-3-(6-(4-methyl- | CDDO-Me |
| | piperazine-1-yl)-1H-benzimidazole- | |
| | 2-yl)-1H-quinoline-2-one | |
| DFQ | 4-amino-5-fluoro-3-(6-(4-methyl- | CDDO-Imm |
| | piperazine-1-yl)-1H-benzimidazole- | |
| | 2-yl)-1H-quinoline-2-one | |
| DFR | 4-amino-5-fluoro-3-(6-(4-methyl- | shikonin |
| | piperazine-1-yl)-1H-benzimidazole- | |
| | 2-yl)-1H-quinoline-2-one | |
| DFS | 4-amino-5-fluoro-3-(6-(4-methyl- | beta-hydroxyisovalerylshikonin |
| | piperazine-1-yl)-1H-benzimidazole- | |
| | 2-yl)-1H-quinoline-2-one | |
| DFT | 4-amino-5-fluoro-3-(6-(4-methyl- | ganglioside GM3 |
| | piperazine-1-yl)-1H-benzimidazole- | |
| | 2-yl)-1H-quinoline-2-one | |
| DFU | 4-amino-5-fluoro-3-(6-(4-methyl- | DC101 antibody |
| | piperazine-1-yl)-1H-benzimidazole- | |
| | 2-yl)-1H-quinoline-2-one | |
| DFV | 4-amino-5-fluoro-3-(6-(4-methyl- | Mab25 antibody |
| | piperazine-1-yl)-1H-benzimidazole- | |
| | 2-yl)-1H-quinoline-2-one | |
| DFW | 4-amino-5-fluoro-3-(6-(4-methyl- | Mab73 antibody |
| | piperazine-1-yl)-1H-benzimidazole- | |
| | 2-yl)-1H-quinoline-2-one | |
| DFX | 4-amino-5-fluoro-3-(6-(4-methyl- | 4A5 antibody |
| | piperazine-1-yl)-1H-benzimidazole- | |
| | 2-yl)-1H-quinoline-2-one | |
| DFY | 4-amino-5-fluoro-3-(6-(4-methyl- | 4E10 antibody |
| | piperazine-1-yl)-1H-benzimidazole- | |
| | 2-yl)-1H-quinoline-2-one | |
| DFZ | 4-amino-5-fluoro-3-(6-(4-methyl- | 5F12 antibody |
| | piperazine-1-yl)-1H-benzimidazole- | |
| | 2-yl)-1H-quinoline-2-one | |
| DGA | 4-amino-5-fluoro-3-(6-(4-methyl- | VA01 antibody |
| | piperazine-1-yl)-1H-benzimidazole- | |
| | 2-yl)-1H-quinoline-2-one | |
| DGB | 4-amino-5-fluoro-3-(6-(4-methyl- | BL2 antibody |
| | piperazine-1-yl)-1H-benzimidazole- | |
| | 2-yl)-1H-quinoline-2-one | |
| DGC | 4-amino-5-fluoro-3-(6-(4-methyl- | VEGF-related protein |
| | piperazine-1-yl)-1H-benzimidazole- | |
| | 2-yl)-1H-quinoline-2-one | |
| DGD | 4-amino-5-fluoro-3-(6-(4-methyl- | sFLT01 |
| | piperazine-1-yl)-1H-benzimidazole- | |
| | 2-yl)-1H-quinoline-2-one | |
| DGE | 4-amino-5-fluoro-3-(6-(4-methyl- | sFLT02 |
| | piperazine-1-yl)-1H-benzimidazole- | |
| | 2-yl)-1H-quinoline-2-one | |
| DGF | 4-amino-5-fluoro-3-(6-(4-methyl- | Peptide B3 |
| | piperazine-1-yl)-1H-benzimidazole- | |
| | 2-yl)-1H-quinoline-2-one | |
| DGG | 4-amino-5-fluoro-3-(6-(4-methyl- | TG100801 |
| | piperazine-1-yl)-1H-benzimidazole- | |
| | 2-yl)-1H-quinoline-2-one | |
| DGH | 4-amino-5-fluoro-3-(6-(4-methyl- | sorafenib |
| | piperazine-1-yl)-1H-benzimidazole- | |
| | 2-yl)-1H-quinoline-2-one | |
| DGI | 4-amino-5-fluoro-3-(6-(4-methyl- | G6-31 antibody |
| | piperazine-1-yl)-1H-benzimidazole- | |
| | 2-yl)-1H-quinoline-2-one | |
| DGJ | (4-tert-butylphenyl) {4-[(6,7- | ranibizumab |
| | dimethoxy-4- | |
| | quinolyl)oxy]phenyl}methaneone | |
| DGK | (4-tert-butylphenyl) {4-[(6,7- | bevacizumab |
| | dimethoxy-4- | |
| | quinolyl)oxy]phenyl}methaneone | |
| DGL | (4-tert-butylphenyl) {4-[(6,7- | aflibercept |
| | dimethoxy-4- | |
| | quinolyl)oxy]phenyl}methaneone | |
| DGM | (4-tert-butylphenyl) {4-[(6,7- | KH902 VEGF receptor-Fc fusion protein |
| | dimethoxy-4- | |
| | quinolyl)oxy]phenyl}methaneone | |
| DGN | (4-tert-butylphenyl) {4-[(6,7- | 2C3 antibody |
| | dimethoxy-4- | |
| | quinolyl)oxy]phenyl}methaneone | |
| DGO | (4-tert-butylphenyl) {4-[(6,7- | ORA102 |
| | dimethoxy-4- | |
| | quinolyl)oxy]phenyl}methaneone | |
| DGP | (4-tert-butylphenyl) {4-[(6,7- | pegaptanib |
| | dimethoxy-4- | |
| | quinolyl)oxy]phenyl}methaneone | |
| DGQ | (4-tert-butylphenyl) {4-[(6,7- | bevasiranib |
| | dimethoxy-4- | |
| | quinolyl)oxy]phenyl}methaneone | |
| DGR | (4-tert-butylphenyl) {4-[(6,7- | SIRNA-027 |
| | dimethoxy-4- | |
| | quinolyl)oxy]phenyl}methaneone | |
| DGS | (4-tert-butylphenyl) {4-[(6,7- | decursin |
| | dimethoxy-4- | |
| | quinolyl)oxy]phenyl}methaneone | |
| DGT | (4-tert-butylphenyl) {4-[(6,7- | decursinol |
| | dimethoxy-4- | |
| | quinolyl)oxy]phenyl}methaneone | |
| DGU | (4-tert-butylphenyl) {4-[(6,7- | picropodophyllin |
| | dimethoxy-4- | |
| | quinolyl)oxy]phenyl}methaneone | |
| DGV | (4-tert-butylphenyl) {4-[(6,7- | guggulsterone |
| | dimethoxy-4- | |
| | quinolyl)oxy]phenyl}methaneone | |
| DGW | (4-tert-butylphenyl) {4-[(6,7- | PLG101 |
| | dimethoxy-4- | |
| | quinolyl)oxy]phenyl}methaneone | |
| DGX | (4-tert-butylphenyl) {4-[(6,7- | eicosanoid LXA4 |
| | dimethoxy-4- | |
| | quinolyl)oxy]phenyl}methaneone | |
| DGY | (4-tert-butylphenyl) {4-[(6,7- | PTK787 |
| | dimethoxy-4- | |
| | quinolyl)oxy]phenyl}methaneone | |
| DGZ | (4-tert-butylphenyl) {4-[(6,7- | pazopanib |
| | dimethoxy-4- | |
| | quinolyl)oxy]phenyl}methaneone | |
| DHA | (4-tert-butylphenyl) {4-[(6,7- | axitinib |
| | dimethoxy-4- | |
| | quinolyl)oxy]phenyl}methaneone | |
| DHB | (4-tert-butylphenyl) {4-[(6,7- | CDDO-Me |
| | dimethoxy-4- | |
| | quinolyl)oxy]phenyl}methaneone | |
| DHC | (4-tert-butylphenyl) {4-[(6,7- | CDDO-Imm |
| | dimethoxy-4- | |
| | quinolyl)oxy]phenyl}methaneone | |
| DHD | (4-tert-butylphenyl) {4-[(6,7- | shikonin |
| | dimethoxy-4- | |
| | quinolyl)oxy]phenyl}methaneone | |
| DHE | (4-tert-butylphenyl) {4-[(6,7- | beta-hydroxyisovalerylshikonin |
| | dimethoxy-4- | |
| | quinolyl)oxy]phenyl}methaneone | |
| DHF | (4-tert-butylphenyl) {4-[(6,7- | ganglioside GM3 |
| | dimethoxy-4- | |
| | quinolyl)oxy]phenyl}methaneone | |
| DHG | (4-tert-butylphenyl) {4-[(6,7- | DC101 antibody |
| | dimethoxy-4- | |
| | quinolyl)oxy]phenyl}methaneone | |
| DHH | (4-tert-butylphenyl) {4-[(6,7- | Mab25 antibody |
| | dimethoxy-4- | |
| | quinolyl)oxy]phenyl}methaneone | |
| DHI | (4-tert-butylphenyl) {4-[(6,7- | Mab73 antibody |
| | dimethoxy-4- | |
| | quinolyl)oxy]phenyl}methaneone | |
| DHJ | (4-tert-butylphenyl) {4-[(6,7- | 4A5 antibody |
| | dimethoxy-4- | |
| | quinolyl)oxy]phenyl}methaneone | |
| DHK | (4-tert-butylphenyl) {4-[(6,7- | 4E10 antibody |
| | dimethoxy-4- | |
| | quinolyl)oxy]phenyl}methaneone | |
| DHL | (4-tert-butylphenyl) {4-[(6,7- | 5F12 antibody |
| | dimethoxy-4- | |
| | quinolyl)oxy]phenyl}methaneone | |
| DHM | (4-tert-butylphenyl) {4-[(6,7- | VA01 antibody |
| | dimethoxy-4- | |
| | quinolyl)oxy]phenyl}methaneone | |
| DHN | (4-tert-butylphenyl) {4-[(6,7- | BL2 antibody |
| | dimethoxy-4- | |
| | quinolyl)oxy]phenyl}methaneone | |
| DHO | (4-tert-butylphenyl) {4-[(6,7- | VEGF-related protein |
| | dimethoxy-4- | |
| | quinolyl)oxy]phenyl}methaneone | |
| DHP | (4-tert-butylphenyl) {4-[(6,7- | sFLT01 |
| | dimethoxy-4- | |
| | quinolyl)oxy]phenyl}methaneone | |
| DHQ | (4-tert-butylphenyl) {4-[(6,7- | sFLT02 |
| | dimethoxy-4- | |
| | quinolyl)oxy]phenyl}methaneone | |
| DHR | (4-tert-butylphenyl) {4-[(6,7- | Peptide B3 |
| | dimethoxy-4- | |
| | quinolyl)oxy]phenyl}methaneone | |
| DHS | (4-tert-butylphenyl) {4-[(6,7- | TG100801 |
| | dimethoxy-4- | |
| | quinolyl)oxy]phenyl}methaneone | |
| DHT | (4-tert-butylphenyl) {4-[(6,7- | sorafenib |
| | dimethoxy-4- | |
| | quinolyl)oxy]phenyl}methaneone | |
| DHU | (4-tert-butylphenyl) {4-[(6,7- | G6-31 antibody |
| | dimethoxy-4- | |
| | quinolyl)oxy]phenyl}methaneone | |
| DHV | 5-methyl-N-[4- | ranibizumab |
| | (trifluoromethyl)phenyl]-4- | |
| | isoxazolecarboxamide | |
| DHW | 5-methyl-N-[4- | bevacizumab |
| | (trifluoromethyl)phenyl]-4- | |
| | isoxazolecarboxamide | |
| DHX | 5-methyl-N-[4- | aflibercept |
| | (trifluoromethyl)phenyl]-4- | |
| | isoxazolecarboxamide | |
| DHY | 5-methyl-N-[4- | KH902 VEGF receptor-Fc fusion protein |
| | (trifluoromethyl)phenyl]-4- | |
| | isoxazolecarboxamide | |
| DHZ | 5-methyl-N-[4- | 2C3 antibody |
| | (trifluoromethyl)phenyl]-4- | |
| | isoxazolecarboxamide | |
| DIA | 5-methyl-N-[4- | ORA102 |
| | (trifluoromethyl)phenyl]-4- | |
| | isoxazolecarboxamide | |
| DIB | 5-methyl-N-[4- | pegaptanib |
| | (trifluoromethyl)phenyl]-4- | |
| | isoxazolecarboxamide | |
| DIC | 5-methyl-N-[4- | bevasiranib |
| | (trifluoromethyl)phenyl]-4- | |
| | isoxazolecarboxamide | |
| DID | 5-methyl-N-[4- | SIRNA-027 |
| | (trifluoromethyl)phenyl]-4- | |
| | isoxazolecarboxamide | |
| DIE | 5-methyl-N-[4- | decursin |
| | (trifluoromethyl)phenyl]-4- | |
| | isoxazolecarboxamide | |
| DIF | 5-methyl-N-[4- | decursinol |
| | (trifluoromethyl)phenyl]-4- | |
| | isoxazolecarboxamide | |
| DIG | 5-methyl-N-[4- | picropodophyllin |
| | (trifluoromethyl)phenyl]-4- | |
| | isoxazolecarboxamide | |
| DIH | 5-methyl-N-[4- | guggulsterone |
| | (trifluoromethyl)phenyl]-4- | |
| | isoxazolecarboxamide | |
| DII | 5-methyl-N-[4- | PLG101 |
| | (trifluoromethyl)phenyl]-4- | |
| | isoxazolecarboxamide | |
| DIJ | 5-methyl-N-[4- | eicosanoid LXA4 |
| | (trifluoromethyl)phenyl]-4- | |
| | isoxazolecarboxamide | |
| DIK | 5-methyl-N-[4- | PTK787 |
| | (trifluoromethyl)phenyl]-4- | |
| | isoxazolecarboxamide | |
| DIL | 5-methyl-N-[4- | pazopanib |
| | (trifluoromethyl)phenyl]-4- | |
| | isoxazolecarboxamide | |
| DIM | 5-methyl-N-[4- | axitinib |
| | (trifluoromethyl)phenyl]-4- | |
| | isoxazolecarboxamide | |
| DIN | 5-methyl-N-[4- | CDDO-Me |
| | (trifluoromethyl)phenyl]-4- | |
| | isoxazolecarboxamide | |
| DIO | 5-methyl-N-[4- | CDDO-Imm |
| | (trifluoromethyl)phenyl]-4- | |
| | isoxazolecarboxamide | |
| DIP | 5-methyl-N-[4- | shikonin |
| | (trifluoromethyl)phenyl]-4- | |
| | isoxazolecarboxamide | |
| DIQ | 5-methyl-N-[4- | beta-hydroxyisovalerylshikonin |
| | (trifluoromethyl)phenyl]-4- | |
| | isoxazolecarboxamide | |
| DIR | 5-methyl-N-[4- | ganglioside GM3 |
| | (trifluoromethyl)phenyl]-4- | |
| | isoxazolecarboxamide | |
| DIS | 5-methyl-N-[4- | DC101 antibody |
| | (trifluoromethyl)phenyl]-4- | |
| | isoxazolecarboxamide | |
| DIT | 5-methyl-N-[4- | Mab25 antibody |
| | (trifluoromethyl)phenyl]-4- | |
| | isoxazolecarboxamide | |
| DIU | 5-methyl-N-[4- | Mab73 antibody |
| | (trifluoromethyl)phenyl]-4- | |
| | isoxazolecarboxamide | |
| DIV | 5-methyl-N-[4- | 4A5 antibody |
| | (trifluoromethyl)phenyl]-4- | |
| | isoxazolecarboxamide | |
| DIW | 5-methyl-N-[4- | 4E10 antibody |
| | (trifluoromethyl)phenyl]-4- | |
| | isoxazolecarboxamide | |
| DIX | 5-methyl-N-[4- | 5F12 antibody |
| | (trifluoromethyl)phenyl]-4- | |
| | isoxazolecarboxamide | |
| DIY | 5-methyl-N-[4- | VA01 antibody |
| | (trifluoromethyl)phenyl]-4- | |
| | isoxazolecarboxamide | |
| DIZ | 5-methyl-N-[4- | BL2 antibody |
| | (trifluoromethyl)phenyl]-4- | |
| | isoxazolecarboxamide | |
| DJA | 5-methyl-N-[4- | VEGF-related protein |
| | (trifluoromethyl)phenyl]-4- | |
| | isoxazolecarboxamide | |
| DJB | 5-methyl-N-[4- | sFLT01 |
| | (trifluoromethyl)phenyl]-4- | |
| | isoxazolecarboxamide | |
| DJC | 5-methyl-N-[4- | sFLT02 |
| | (trifluoromethyl)phenyl]-4- | |
| | isoxazolecarboxamide | |
| DJD | 5-methyl-N-[4- | Peptide B3 |
| | (trifluoromethyl)phenyl]-4- | |
| | isoxazolecarboxamide | |
| DJE | 5-methyl-N-[4- | TG100801 |
| | (trifluoromethyl)phenyl]-4- | |
| | isoxazolecarboxamide | |
| DJF | 5-methyl-N-[4- | sorafenib |
| | (trifluoromethyl)phenyl]-4- | |
| | isoxazolecarboxamide | |
| DJG | 5-methyl-N-[4- | G6-31 antibody |
| | (trifluoromethyl)phenyl]-4- | |
| | isoxazolecarboxamide | |
| DJH | trans-4-[(6,7-dimethoxyquinoxaline-2- | ranibizumab |
| | yl)amino]cyclohexanol | |
| DJI | trans-4-[(6,7-dimethoxyquinoxaline-2- | bevacizumab |
| | yl)amino]cyclohexanol | |
| DJJ | trans-4-[(6,7-dimethoxyquinoxaline-2- | aflibercept |
| | yl)amino]cyclohexanol | |
| DJK | trans-4-[(6,7-dimethoxyquinoxaline-2- | KH902 VEGF receptor-Fc fusion protein |
| | yl)amino]cyclohexanol | |
| DJL | trans-4-[(6,7-dimethoxyquinoxaline-2- | 2C3 antibody |
| | yl)amino]cyclohexanol | |
| DJM | trans-4-[(6,7-dimethoxyquinoxaline-2- | ORA102 |
| | yl)amino]cyclohexanol | |
| DJN | trans-4-[(6,7-dimethoxyquinoxaline-2- | pegaptanib |
| | yl)amino]cyclohexanol | |
| DJO | trans-4-[(6,7-dimethoxyquinoxaline-2- | bevasiranib |
| | yl)amino]cyclohexanol | |
| DJP | trans-4-[(6,7-dimethoxyquinoxaline-2- | SIRNA-027 |
| | yl)amino]cyclohexanol | |
| DJQ | trans-4-[(6,7-dimethoxyquinoxaline-2- | decursin |
| | yl)amino]cyclohexanol | |
| DJR | trans-4-[(6,7-dimethoxyquinoxaline-2- | decursinol |
| | yl)amino]cyclohexanol | |
| DJS | trans-4-[(6,7-dimethoxyquinoxaline-2- | picropodophyllin |
| | yl)amino]cyclohexanol | |
| DJT | trans-4-[(6,7-dimethoxyquinoxaline-2- | guggulsterone |
| | yl)amino]cyclohexanol | |
| DJU | trans-4-[(6,7-dimethoxyquinoxaline-2- | PLG101 |
| | yl)amino]cyclohexanol | |
| DJV | trans-4-[(6,7-dimethoxyquinoxaline-2- | eicosanoid LXA4 |
| | yl)amino]cyclohexanol | |
| DJW | trans-4-[(6,7-dimethoxyquinoxaline-2- | PTK787 |
| | yl)amino]cyclohexanol | |
| DJX | trans-4-[(6,7-dimethoxyquinoxaline-2- | pazopanib |
| | yl)amino]cyclohexanol | |
| DJY | trans-4-[(6,7-dimethoxyquinoxaline-2- | axitinib |
| | yl)amino]cyclohexanol | |
| DJZ | trans-4-[(6,7-dimethoxyquinoxaline-2- | CDDO-Me |
| | yl)amino]cyclohexanol | |
| DKA | trans-4-[(6,7-dimethoxyquinoxaline-2- | CDDO-Imm |
| | yl)amino]cyclohexanol | |
| DKB | trans-4-[(6,7-dimethoxyquinoxaline-2- | shikonin |
| | yl)amino]cyclohexanol | |
| DKC | trans-4-[(6,7-dimethoxyquinoxaline-2- | beta-hydroxyisovalerylshikonin |
| | yl)amino]cyclohexanol | |
| DKD | trans-4-[(6,7-dimethoxyquinoxaline-2- | ganglioside GM3 |
| | yl)amino]cyclohexanol | |
| DKE | trans-4-[(6,7-dimethoxyquinoxaline-2- | DC101 antibody |
| | yl)amino]cyclohexanol | |
| DKF | trans-4-[(6,7-dimethoxyquinoxaline-2- | Mab25 antibody |
| | yl)amino]cyclohexanol | |
| DKG | trans-4-[(6,7-dimethoxyquinoxaline-2- | Mab73 antibody |
| | yl)amino]cyclohexanol | |
| DKH | trans-4-[(6,7-dimethoxyquinoxaline-2- | 4A5 antibody |
| | yl)amino]cyclohexanol | |
| DKI | trans-4-[(6,7-dimethoxyquinoxaline-2- | 4E10 antibody |
| | yl)amino]cyclohexanol | |
| DKJ | trans-4-[(6,7-dimethoxyquinoxaline-2- | 5F12 antibody |
| | yl)amino]cyclohexanol | |
| DKK | trans-4-[(6,7-dimethoxyquinoxaline-2- | VA01 antibody |
| | yl)amino]cyclohexanol | |
| DKL | trans-4-[(6,7-dimethoxyquinoxaline-2- | BL2 antibody |
| | yl)amino]cyclohexanol | |
| DKM | trans-4-[(6,7-dimethoxyquinoxaline-2- | VEGF-related protein |
| | yl)amino]cyclohexanol | |
| DKN | trans-4-[(6,7-dimethoxyquinoxaline-2- | sFLT01 |
| | yl)amino]cyclohexanol | |
| DKO | trans-4-[(6,7-dimethoxyquinoxaline-2- | sFLT02 |
| | yl)amino]cyclohexanol | |
| DKP | trans-4-[(6,7-dimethoxyquinoxaline-2- | Peptide B3 |
| | yl)amino]cyclohexanol | |
| DKQ | trans-4-[(6,7-dimethoxyquinoxaline-2- | TG100801 |
| | yl)amino]cyclohexanol | |
| DKR | trans-4-[(6,7-dimethoxyquinoxaline-2- | sorafenib |
| | yl)amino]cyclohexanol | |
| DKS | trans-4-[(6,7-dimethoxyquinoxaline-2- | G6-31 antibody |
| | yl)amino]cyclohexanol | |
| DKT | (Z)-3-[(2,4-dimethyl-5-(2-oxo-1,2- | ranibizumab |
| | dihydroindole-3-ylidenemethyl)-1H- | |
| | pyrrole-3-yl)-propionic acid | |
| DKU | (Z)-3-[(2,4-dimethyl-5-(2-oxo-1,2- | bevacizumab |
| | dihydroindole-3-ylidenemethyl)-1H- | |
| | pyrrole-3-yl)-propionic acid | |
| DKV | (Z)-3-[(2,4-dimethyl-5-(2-oxo-1,2- | aflibercept |
| | dihydroindole-3-ylidenemethyl)-1H- | |
| | pyrrole-3-yl)-propionic acid | |
| DKW | (Z)-3-[(2,4-dimethyl-5-(2-oxo-1,2- | KH902 VEGF receptor-Fc fusion protein |
| | dihydroindole-3-ylidenemethyl)-1H- | |
| | pyrrole-3-yl)-propionic acid | |
| DKX | (Z)-3-[(2,4-dimethyl-5-(2-oxo-1,2- | 2C3 antibody |
| | dihydroindole-3-ylidenemethyl)-1H- | |
| | pyrrole-3-yl)-propionic acid | |
| DKY | (Z)-3-[(2,4-dimethyl-5-(2-oxo-1,2- | ORA102 |
| | dihydroindole-3-ylidenemethyl)-1H- | |
| | pyrrole-3-yl)-propionic acid | |
| DKZ | (Z)-3-[(2,4-dimethyl-5-(2-oxo-1,2- | pegaptanib |
| | dihydroindole-3-ylidenemethyl)-1H- | |
| | pyrrole-3-yl)-propionic acid | |
| DLA | (Z)-3-[(2,4-dimethyl-5-(2-oxo-1,2- | bevasiranib |
| | dihydroindole-3-ylidenemethyl)-1H- | |
| | pyrrole-3-yl)-propionic acid | |
| DLB | (Z)-3-[(2,4-dimethyl-5-(2-oxo-1,2- | SIRNA-027 |
| | dihydroindole-3-ylidenemethyl)-1H- | |
| | pyrrole-3-yl)-propionic acid | |
| DLC | (Z)-3-[(2,4-dimethyl-5-(2-oxo-1,2- | decursin |
| | dihydroindole-3-ylidenemethyl)-1H- | |
| | pyrrole-3-yl)-propionic acid | |
| DLD | (Z)-3-[(2,4-dimethyl-5-(2-oxo-1,2- | decursinol |
| | dihydroindole-3-ylidenemethyl)-1H- | |
| | pyrrole-3-yl)-propionic acid | |
| DLE | (Z)-3-[(2,4-dimethyl-5-(2-oxo-1,2- | picropodophyllin |
| | dihydroindole-3-ylidenemethyl)-1H- | |
| | pyrrole-3-yl)-propionic acid | |
| DLF | (Z)-3-[(2,4-dimethyl-5-(2-oxo-1,2- | guggulsterone |
| | dihydroindole-3-ylidenemethyl)-1H- | |
| | pyrrole-3-yl)-propionic acid | |
| DLG | (Z)-3-[(2,4-dimethyl-5-(2-oxo-1,2- | PLG101 |
| | dihydroindole-3-ylidenemethyl)-1H- | |
| | pyrrole-3-yl)-propionic acid | |
| DLH | (Z)-3-[(2,4-dimethyl-5-(2-oxo-1,2- | eicosanoid LXA4 |
| | dihydroindole-3-ylidenemethyl)-1H- | |
| | pyrrole-3-yl)-propionic acid | |
| DLI | (Z)-3-[(2,4-dimethyl-5-(2-oxo-1,2- | PTK787 |
| | dihydroindole-3-ylidenemethyl)-1H- | |
| | pyrrole-3-yl)-propionic acid | |
| DLJ | (Z)-3-[(2,4-dimethyl-5-(2-oxo-1,2- | pazopanib |
| | dihydroindole-3-ylidenemethyl)-1H- | |
| | pyrrole-3-yl)-propionic acid | |
| DLK | (Z)-3-[(2,4-dimethyl-5-(2-oxo-1,2- | axitinib |
| | dihydroindole-3-ylidenemethyl)-1H- | |
| | pyrrole-3-yl)-propionic acid | |
| DLL | (Z)-3-[(2,4-dimethyl-5-(2-oxo-1,2- | CDDO-Me |
| | dihydroindole-3-ylidenemethyl)-1H- | |
| | pyrrole-3-yl)-propionic acid | |
| DLM | (Z)-3-[(2,4-dimethyl-5-(2-oxo-1,2- | CDDO-Imm |
| | dihydroindole-3-ylidenemethyl)-1H- | |
| | pyrrole-3-yl)-propionic acid | |
| DLN | (Z)-3-[(2,4-dimethyl-5-(2-oxo-1,2- | shikonin |
| | dihydroindole-3-ylidenemethyl)-1H- | |
| | pyrrole-3-yl)-propionic acid | |
| DLO | (Z)-3-[(2,4-dimethyl-5-(2-oxo-1,2- | beta-hydroxyisovalerylshikonin |
| | dihydroindole-3-ylidenemethyl)-1H- | |
| | pyrrole-3-yl)-propionic acid | |
| DLP | (Z)-3-[(2,4-dimethyl-5-(2-oxo-1,2- | ganglioside GM3 |
| | dihydroindole-3-ylidenemethyl)-1H- | |
| | pyrrole-3-yl)-propionic acid | |
| DLQ | (Z)-3-[(2,4-dimethyl-5-(2-oxo-1,2- | DC101 antibody |
| | dihydroindole-3-ylidenemethyl)-1H- | |
| | pyrrole-3-yl)-propionic acid | |
| DLR | (Z)-3-[(2,4-dimethyl-5-(2-oxo-1,2- | Mab25 antibody |
| | dihydroindole-3-ylidenemethyl)-1H- | |
| | pyrrole-3-yl)-propionic acid | |
| DLS | (Z)-3-[(2,4-dimethyl-5-(2-oxo-1,2- | Mab73 antibody |
| | dihydroindole-3-ylidenemethyl)-1H- | |
| | pyrrole-3-yl)-propionic acid | |
| DLT | (Z)-3-[(2,4-dimethyl-5-(2-oxo-1,2- | 4A5 antibody |
| | dihydroindole-3-ylidenemethyl)-1H- | |
| | pyrrole-3-yl)-propionic acid | |
| DLU | (Z)-3-[(2,4-dimethyl-5-(2-oxo-1,2- | 4E10 antibody |
| | dihydroindole-3-ylidenemethyl)-1H- | |
| | pyrrole-3-yl)-propionic acid | |
| DLV | (Z)-3-[(2,4-dimethyl-5-(2-oxo-1,2- | 5F12 antibody |
| | dihydroindole-3-ylidenemethyl)-1H- | |
| | pyrrole-3-yl)-propionic acid | |
| DLW | (Z)-3-[(2,4-dimethyl-5-(2-oxo-1,2- | VA01 antibody |
| | dihydroindole-3-ylidenemethyl)-1H- | |
| | pyrrole-3-yl)-propionic acid | |
| DLX | (Z)-3-[(2,4-dimethyl-5-(2-oxo-1,2- | BL2 antibody |
| | dihydroindole-3-ylidenemethyl)-1H- | |
| | pyrrole-3-yl)-propionic acid | |
| DLY | (Z)-3-[(2,4-dimethyl-5-(2-oxo-1,2- | VEGF-related protein |
| | dihydroindole-3-ylidenemethyl)-1H- | |
| | pyrrole-3-yl)-propionic acid | |
| DLZ | (Z)-3-[(2,4-dimethyl-5-(2-oxo-1,2- | sFLT01 |
| | dihydroindole-3-ylidenemethyl)-1H- | |
| | pyrrole-3-yl)-propionic acid | |
| DMA | (Z)-3-[(2,4-dimethyl-5-(2-oxo-1,2- | sFLT02 |
| | dihydroindole-3-ylidenemethyl)-1H- | |
| | pyrrole-3-yl)-propionic acid | |
| DMB | (Z)-3-[(2,4-dimethyl-5-(2-oxo-1,2- | Peptide B3 |
| | dihydroindole-3-ylidenemethyl)-1H- | |
| | pyrrole-3-yl)-propionic acid | |
| DMC | (Z)-3-[(2,4-dimethyl-5-(2-oxo-1,2- | TG100801 |
| | dihydroindole-3-ylidenemethyl)-1H- | |
| | pyrrole-3-yl)-propionic acid | |
| DMD | (Z)-3-[(2,4-dimethyl-5-(2-oxo-1,2- | sorafenib |
| | dihydroindole-3-ylidenemethyl)-1H- | |
| | pyrrole-3-yl)-propionic acid | |
| DME | (Z)-3-[(2,4-dimethyl-5-(2-oxo-1,2- | G6-31 antibody |
| | dihydroindole-3-ylidenemethyl)-1H- | |
| | pyrrole-3-yl)-propionic acid | |
| DMF | 5-(5-fluoro-2-oxo-1,2- | ranibizumab |
| | dihydroindole-3-ylidenemethyl)-2,4- | |
| | dimethyl-1H-pyrrole-3-carboxylic | |
| | acid | |
| DMG | 5-(5-fluoro-2-oxo-1,2- | bevacizumab |
| | dihydroindole-3-ylidenemethyl)-2,4- | |
| | dimethyl-1H-pyrrole-3-carboxylic | |
| | acid | |
| DMH | 5-(5-fluoro-2-oxo-1,2- | aflibercept |
| | dihydroindole-3-ylidenemethyl)-2,4- | |
| | dimethyl-1H-pyrrole-3-carboxylic | |
| | acid | |
| DMI | 5-(5-fluoro-2-oxo-1,2- | KH902 VEGF receptor-Fc fusion protein |
| | dihydroindole-3-ylidenemethyl)-2,4- | |
| | dimethyl-1H-pyrrole-3-carboxylic | |
| | acid | |
| DMJ | 5-(5-fluoro-2-oxo-1,2- | 2C3 antibody |
| | dihydroindole-3-ylidenemethyl)-2,4- | |
| | dimethyl-1H-pyrrole-3-carboxylic | |
| | acid | |
| DMK | 5-(5-fluoro-2-oxo-1,2- | ORA102 |
| | dihydroindole-3-ylidenemethyl)-2,4- | |
| | dimethyl-1H-pyrrole-3-carboxylic | |
| | acid | |
| DML | 5-(5-fluoro-2-oxo-1,2- | pegaptanib |
| | dihydroindole-3-ylidenemethyl)-2,4- | |
| | dimethyl-1H-pyrrole-3-carboxylic | |
| | acid | |
| DMM | 5-(5-fluoro-2-oxo-1,2- | bevasiranib |
| | dihydroindole-3-ylidenemethyl)-2,4- | |
| | dimethyl-1H-pyrrole-3-carboxylic | |
| | acid | |
| DMN | 5-(5-fluoro-2-oxo-1,2- | SIRNA-027 |
| | dihydroindole-3-ylidenemethyl)-2,4- | |
| | dimethyl-1H-pyrrole-3-carboxylic | |
| | acid | |
| DMO | 5-(5-fluoro-2-oxo-1,2- | decursin |
| | dihydroindole-3-ylidenemethyl)-2,4- | |
| | dimethyl-1H-pyrrole-3-carboxylic | |
| | acid | |
| DMP | 5-(5-fluoro-2-oxo-1,2- | decursinol |
| | dihydroindole-3-ylidenemethyl)-2,4- | |
| | dimethyl-1H-pyrrole-3-carboxylic | |
| | acid | |
| DMQ | 5-(5-fluoro-2-oxo-1,2- | picropodophyllin |
| | dihydroindole-3-ylidenemethyl)-2,4- | |
| | dimethyl-1H-pyrrole-3-carboxylic | |
| | acid | |
| DMR | 5-(5-fluoro-2-oxo-1,2- | guggulsterone |
| | dihydroindole-3-ylidenemethyl)-2,4- | |
| | dimethyl-1H-pyrrole-3-carboxylic | |
| | acid | |
| DMS | 5-(5-fluoro-2-oxo-1,2- | PLG101 |
| | dihydroindole-3-ylidenemethyl)-2,4- | |
| | dimethyl-1H-pyrrole-3-carboxylic | |
| | acid | |
| DMT | 5-(5-fluoro-2-oxo-1,2- | eicosanoid LXA4 |
| | dihydroindole-3-ylidenemethyl)-2,4- | |
| | dimethyl-1H-pyrrole-3-carboxylic | |
| | acid | |
| DMU | 5-(5-fluoro-2-oxo-1,2- | PTK787 |
| | dihydroindole-3-ylidenemethyl)-2,4- | |
| | dimethyl-1H-pyrrole-3-carboxylic | |
| | acid | |
| DMV | 5-(5-fluoro-2-oxo-1,2- | pazopanib |
| | dihydroindole-3-ylidenemethyl)-2,4- | |
| | dimethyl-1H-pyrrole-3-carboxylic | |
| | acid | |
| DMW | 5-(5-fluoro-2-oxo-1,2- | axitinib |
| | dihydroindole-3-ylidenemethyl)-2,4- | |
| | dimethyl-1H-pyrrole-3-carboxylic | |
| | acid | |
| DMX | 5-(5-fluoro-2-oxo-1,2- | CDDO-Me |
| | dihydroindole-3-ylidenemethyl)-2,4- | |
| | dimethyl-1H-pyrrole-3-carboxylic | |
| | acid | |
| DMY | 5-(5-fluoro-2-oxo-1,2- | CDDO-Imm |
| | dihydroindole-3-ylidenemethyl)-2,4- | |
| | dimethyl-1H-pyrrole-3-carboxylic | |
| | acid | |
| DMZ | 5-(5-fluoro-2-oxo-1,2- | shikonin |
| | dihydroindole-3-ylidenemethyl)-2,4- | |
| | dimethyl-1H-pyrrole-3-carboxylic | |
| | acid | |
| DNA | 5-(5-fluoro-2-oxo-1,2- | beta-hydroxyisovalerylshikonin |
| | dihydroindole-3-ylidenemethyl)-2,4- | |
| | dimethyl-1H-pyrrole-3-carboxylic | |
| | acid | |
| DNB | 5-(5-fluoro-2-oxo-1,2- | ganglioside GM3 |
| | dihydroindole-3-ylidenemethyl)-2,4- | |
| | dimethyl-1H-pyrrole-3-carboxylic | |
| | acid | |
| DNC | 5-(5-fluoro-2-oxo-1,2- | DC101 antibody |
| | dihydroindole-3-ylidenemethyl)-2,4- | |
| | dimethyl-1H-pyrrole-3-carboxylic | |
| | acid | |
| DND | 5-(5-fluoro-2-oxo-1,2- | Mab25 antibody |
| | dihydroindole-3-ylidenemethyl)-2,4- | |
| | dimethyl-1H-pyrrole-3-carboxylic | |
| | acid | |
| DNE | 5-(5-fluoro-2-oxo-1,2- | Mab73 antibody |
| | dihydroindole-3-ylidenemethyl)-2,4- | |
| | dimethyl-1H-pyrrole-3-carboxylic | |
| | acid | |
| DNF | 5-(5-fluoro-2-oxo-1,2- | 4A5 antibody |
| | dihydroindole-3-ylidenemethyl)-2,4- | |
| | dimethyl-1H-pyrrole-3-carboxylic | |
| | acid | |
| DNG | 5-(5-fluoro-2-oxo-1,2- | 4E10 antibody |
| | dihydroindole-3-ylidenemethyl)-2,4- | |
| | dimethyl-1H-pyrrole-3-carboxylic | |
| | acid | |
| DNH | 5-(5-fluoro-2-oxo-1,2- | 5F12 antibody |
| | dihydroindole-3-ylidenemethyl)-2,4- | |
| | dimethyl-1H-pyrrole-3-carboxylic | |
| | acid | |
| DNI | 5-(5-fluoro-2-oxo-1,2- | VA01 antibody |
| | dihydroindole-3-ylidenemethyl)-2,4- | |
| | dimethyl-1H-pyrrole-3-carboxylic | |
| | acid | |
| DNJ | 5-(5-fluoro-2-oxo-1,2- | BL2 antibody |
| | dihydroindole-3-ylidenemethyl)-2,4- | |
| | dimethyl-1H-pyrrole-3-carboxylic | |
| | acid | |
| DNK | 5-(5-fluoro-2-oxo-1,2- | VEGF-related protein |
| | dihydroindole-3-ylidenemethyl)-2,4- | |
| | dimethyl-1H-pyrrole-3-carboxylic | |
| | acid | |
| DNL | 5-(5-fluoro-2-oxo-1,2- | sFLT01 |
| | dihydroindole-3-ylidenemethyl)-2,4- | |
| | dimethyl-1H-pyrrole-3-carboxylic | |
| | acid | |
| DNM | 5-(5-fluoro-2-oxo-1,2- | sFLT02 |
| | dihydroindole-3-ylidenemethyl)-2,4- | |
| | dimethyl-1H-pyrrole-3-carboxylic | |
| | acid | |
| DNN | 5-(5-fluoro-2-oxo-1,2- | Peptide B3 |
| | dihydroindole-3-ylidenemethyl)-2,4- | |
| | dimethyl-1H-pyrrole-3-carboxylic | |
| | acid | |
| DNO | 5-(5-fluoro-2-oxo-1,2- | TG100801 |
| | dihydroindole-3-ylidenemethyl)-2,4- | |
| | dimethyl-1H-pyrrole-3-carboxylic | |
| | acid | |
| DNP | 5-(5-fluoro-2-oxo-1,2- | sorafenib |
| | dihydroindole-3-ylidenemethyl)-2,4- | |
| | dimethyl-1H-pyrrole-3-carboxylic | |
| | acid | |
| DNQ | 5-(5-fluoro-2-oxo-1,2- | G6-31 antibody |
| | dihydroindole-3-ylidenemethyl)-2,4- | |
| | dimethyl-1H-pyrrole-3-carboxylic | |
| | acid | |
| DNR | 1-(4-chloroanilino)-4-(4- | ranibizumab |
| | pyridylmethyl)phthalazine | |
| DNS | 1-(4-chloroanilino)-4-(4- | bevacizumab |
| | pyridylmethyl)phthalazine | |
| DNT | 1-(4-chloroanilino)-4-(4- | aflibercept |
| | pyridylmethyl)phthalazine | |
| DNU | 1-(4-chloroanilino)-4-(4- | KH902 VEGF receptor-Fc fusion protein |
| | pyridylmethyl)phthalazine | |
| DNV | 1-(4-chloroanilino)-4-(4- | 2C3 antibody |
| | pyridylmethyl)phthalazine | |
| DNW | 1-(4-chloroanilino)-4-(4- | ORA102 |
| | pyridylmethyl)phthalazine | |
| DNX | 1-(4-chloroanilino)-4-(4- | pegaptanib |
| | pyridylmethyl)phthalazine | |
| DNY | 1-(4-chloroanilino)-4-(4- | bevasiranib |
| | pyridylmethyl)phthalazine | |
| DNZ | 1-(4-chloroanilino)-4-(4- | SIRNA-027 |
| | pyridylmethyl)phthalazine | |
| DOA | 1-(4-chloroanilino)-4-(4- | decursin |
| | pyridylmethyl)phthalazine | |
| DOB | 1-(4-chloroanilino)-4-(4- | decursinol |
| | pyridylmethyl)phthalazine | |
| DOC | 1-(4-chloroanilino)-4-(4- | picropodophyllin |
| | pyridylmethyl)phthalazine | |
| DOD | 1-(4-chloroanilino)-4-(4- | guggulsterone |
| | pyridylmethyl)phthalazine | |
| DOE | 1-(4-chloroanilino)-4-(4- | PLG101 |
| | pyridylmethyl)phthalazine | |
| DOF | 1-(4-chloroanilino)-4-(4- | eicosanoid LXA4 |
| | pyridylmethyl)phthalazine | |
| DOG | 1-(4-chloroanilino)-4-(4- | PTK787 |
| | pyridylmethyl)phthalazine | |
| DOH | 1-(4-chloroanilino)-4-(4- | pazopanib |
| | pyridylmethyl)phthalazine | |
| DOI | 1-(4-chloroanilino)-4-(4- | axitinib |
| | pyridylmethyl)phthalazine | |
| DOJ | 1-(4-chloroanilino)-4-(4- | CDDO-Me |
| | pyridylmethyl)phthalazine | |
| DOK | 1-(4-chloroanilino)-4-(4- | CDDO-Imm |
| | pyridylmethyl)phthalazine | |
| DOL | 1-(4-chloroanilino)-4-(4- | shikonin |
| | pyridylmethyl)phthalazine | |
| DOM | 1-(4-chloroanilino)-4-(4- | beta-hydroxyisovalerylshikonin |
| | pyridylmethyl)phthalazine | |
| DON | 1-(4-chloroanilino)-4-(4- | ganglioside GM3 |
| | pyridylmethyl)phthalazine | |
| DOO | 1-(4-chloroanilino)-4-(4- | DC101 antibody |
| | pyridylmethyl)phthalazine | |
| DOP | 1-(4-chloroanilino)-4-(4- | Mab25 antibody |
| | pyridylmethyl)phthalazine | |
| DOQ | 1-(4-chloroanilino)-4-(4- | Mab73 antibody |
| | pyridylmethyl)phthalazine | |
| DOR | 1-(4-chloroanilino)-4-(4- | 4A5 antibody |
| | pyridylmethyl)phthalazine | |
| DOS | 1-(4-chloroanilino)-4-(4- | 4E10 antibody |
| | pyridylmethyl)phthalazine | |
| DOT | 1-(4-chloroanilino)-4-(4- | 5F12 antibody |
| | pyridylmethyl)phthalazine | |
| DOU | 1-(4-chloroanilino)-4-(4- | VA01 antibody |
| | pyridylmethyl)phthalazine | |
| DOV | 1-(4-chloroanilino)-4-(4- | BL2 antibody |
| | pyridylmethyl)phthalazine | |
| DOW | 1-(4-chloroanilino)-4-(4- | VEGF-related protein |
| | pyridylmethyl)phthalazine | |
| DOX | 1-(4-chloroanilino)-4-(4- | sFLT01 |
| | pyridylmethyl)phthalazine | |
| DOY | 1-(4-chloroanilino)-4-(4- | sFLT02 |
| | pyridylmethyl)phthalazine | |
| DOZ | 1-(4-chloroanilino)-4-(4- | Peptide B3 |
| | pyridylmethyl)phthalazine | |
| DPA | 1-(4-chloroanilino)-4-(4- | TG100801 |
| | pyridylmethyl)phthalazine | |
| DPB | 1-(4-chloroanilino)-4-(4- | sorafenib |
| | pyridylmethyl)phthalazine | |
| DPC | 1-(4-chloroanilino)-4-(4- | G6-31 antibody |
| | pyridylmethyl)phthalazine | |
| DPD | N-[4-(3-amino-1H-indazole-4- | ranibizumab |
| | yl)phenyl-N′-(2-fluoro-5- | |
| | methylphenyl)urea | |
| DPE | N-[4-(3-amino-1H-indazole-4- | bevacizumab |
| | yl)phenyl-N′-(2-fluoro-5- | |
| | methylphenyl)urea | |
| DPF | N-[4-(3-amino-1H-indazole-4- | aflibercept |
| | yl)phenyl-N′-(2-fluoro-5- | |
| | methylphenyl)urea | |
| DPG | N-[4-(3-amino-1H-indazole-4- | KH902 VEGF receptor-Fc fusion protein |
| | yl)phenyl-N′-(2-fluoro-5- | |
| | methylphenyl)urea | |
| DPH | N-[4-(3-amino-1H-indazole-4- | 2C3 antibody |
| | yl)phenyl-N′-(2-fluoro-5- | |
| | methylphenyl)urea | |
| DPI | N-[4-(3-amino-1H-indazole-4- | ORA102 |
| | yl)phenyl-N′-(2-fluoro-5- | |
| | methylphenyl)urea | |
| DPJ | N-[4-(3-amino-1H-indazole-4- | pegaptanib zzzz |
| | yl)phenyl-N′-(2-fluoro-5- | |
| | methylphenyl)urea | |
| DPK | N-[4-(3-amino-1H-indazole-4- | bevasiranib |
| | yl)phenyl-N′-(2-fluoro-5- | |
| | methylphenyl)urea | |
| DPL | N-[4-(3-amino-1H-indazole-4- | SIRNA-027 |
| | yl)phenyl-N′-(2-fluoro-5- | |
| | methylphenyl)urea | |
| DPM | N-[4-(3-amino-1H-indazole-4- | decursin |
| | yl)phenyl-N′-(2-fluoro-5- | |
| | methylphenyl)urea | |
| DPN | N-[4-(3-amino-1H-indazole-4- | decursinol |
| | yl)phenyl-N′-(2-fluoro-5- | |
| | methylphenyl)urea | |
| DPO | N-[4-(3-amino-1H-indazole-4- | picropodophyllin |
| | yl)phenyl-N′-(2-fluoro-5- | |
| | methylphenyl)urea | |
| DPP | N-[4-(3-amino-1H-indazole-4- | guggulsterone |
| | yl)phenyl-N′-(2-fluoro-5- | |
| | methylphenyl)urea | |
| DPQ | N-[4-(3-amino-1H-indazole-4- | PLG101 |
| | yl)phenyl-N′-(2-fluoro-5- | |
| | methylphenyl)urea | |
| DPR | N-[4-(3-amino-1H-indazole-4- | eicosanoid LXA4 |
| | yl)phenyl-N′-(2-fluoro-5- | |
| | methylphenyl)urea | |
| DPS | N-[4-(3-amino-1H-indazole-4- | PTK787 |
| | yl)phenyl-N′-(2-fluoro-5- | |
| | methylphenyl)urea | |
| DPT | N-[4-(3-amino-1H-indazole-4- | pazopanib |
| | yl)phenyl-N′-(2-fluoro-5- | |
| | methylphenyl)urea | |
| DPU | N-[4-(3-amino-1H-indazole-4- | axitinib |
| | yl)phenyl-N′-(2-fluoro-5- | |
| | methylphenyl)urea | |
| DPV | N-[4-(3-amino-1H-indazole-4- | CDDO-Me |
| | yl)phenyl-N′-(2-fluoro-5- | |
| | methylphenyl)urea | |
| DPW | N-[4-(3-amino-1H-indazole-4- | CDDO-Imm |
| | yl)phenyl-N′-(2-fluoro-5- | |
| | methylphenyl)urea | |
| DPX | N-[4-(3-amino-1H-indazole-4- | shikonin |
| | yl)phenyl-N′-(2-fluoro-5- | |
| | methylphenyl)urea | |
| DPY | N-[4-(3-amino-1H-indazole-4- | beta-hydroxyisovalerylshikonin |
| | yl)phenyl-N′-(2-fluoro-5- | |
| | methylphenyl)urea | |
| DPZ | N-[4-(3-amino-1H-indazole-4- | ganglioside GM3 |
| | yl)phenyl-N′-(2-fluoro-5- | |
| | methylphenyl)urea | |
| DQA | N-[4-(3-amino-1H-indazole-4- | DC101 antibody |
| | yl)phenyl-N′-(2-fluoro-5- | |
| | methylphenyl)urea | |
| DQB | N-[4-(3-amino-1H-indazole-4- | Mab25 antibody |
| | yl)phenyl-N′-(2-fluoro-5- | |
| | methylphenyl)urea | |
| DQC | N-[4-(3-amino-1H-indazole-4- | Mab73 antibody |
| | yl)phenyl-N′-(2-fluoro-5- | |
| | methylphenyl)urea | |
| DQD | N-[4-(3-amino-1H-indazole-4- | 4A5 antibody |
| | yl)phenyl-N′-(2-fluoro-5- | |
| | methylphenyl)urea | |
| DQE | N-[4-(3-amino-1H-indazole-4- | 4E10 antibody |
| | yl)phenyl-N′-(2-fluoro-5- | |
| | methylphenyl)urea | |
| DQF | N-[4-(3-amino-1H-indazole-4- | 5F12 antibody |
| | yl)phenyl-N′-(2-fluoro-5- | |
| | methylphenyl)urea | |
| DQG | N-[4-(3-amino-1H-indazole-4- | VA01 antibody |
| | yl)phenyl-N′-(2-fluoro-5- | |
| | methylphenyl)urea | |
| DQH | N-[4-(3-amino-1H-indazole-4- | BL2 antibody |
| | yl)phenyl-N′-(2-fluoro-5- | |
| | methylphenyl)urea | |
| DQI | N-[4-(3-amino-1H-indazole-4- | VEGF-related protein |
| | yl)phenyl-N′-(2-fluoro-5- | |
| | methylphenyl)urea | |
| DQJ | N-[4-(3-amino-1H-indazole-4- | sFLT01 |
| | yl)phenyl-N′-(2-fluoro-5- | |
| | methylphenyl)urea | |
| DQK | N-[4-(3-amino-1H-indazole-4- | sFLT02 |
| | yl)phenyl-N′-(2-fluoro-5- | |
| | methylphenyl)urea | |
| DQL | N-[4-(3-amino-1H-indazole-4- | Peptide B3 |
| | yl)phenyl-N′-(2-fluoro-5- | |
| | methylphenyl)urea | |
| DQM | N-[4-(3-amino-1H-indazole-4- | TG100801 |
| | yl)phenyl-N′-(2-fluoro-5- | |
| | methylphenyl)urea | |
| DQN | N-[4-(3-amino-1H-indazole-4- | sorafenib |
| | yl)phenyl-N′-(2-fluoro-5- | |
| | methylphenyl)urea | |
| DQO | N-[4-(3-amino-1H-indazole-4- | G6-31 antibody |
| | yl)phenyl-N′-(2-fluoro-5- | |
| | methylphenyl)urea | |
| DQP | 1,2-dimethyl-7-(2-thiophene) | ranibizumab |
| | imidazolo [5,4-g] quinoxaline | |
| DQQ | 1,2-dimethyl-7-(2-thiophene) | bevacizumab |
| | imidazolo [5,4-g] quinoxaline | |
| DQR | 1,2-dimethyl-7-(2-thiophene) | aflibercept |
| | imidazolo [5,4-g] quinoxaline | |
| DQS | 1,2-dimethyl-7-(2-thiophene) | KH902 VEGF receptor-Fc fusion protein |
| | imidazolo [5,4-g] quinoxaline | |
| DQT | 1,2-dimethyl-7-(2-thiophene) | 2C3 antibody |
| | imidazolo [5,4-g] quinoxaline | |
| DQU | 1,2-dimethyl-7-(2-thiophene) | ORA102 |
| | imidazolo [5,4-g] quinoxaline | |
| DQV | 1,2-dimethyl-7-(2-thiophene) | pegaptanib |
| | imidazolo [5,4-g] quinoxaline | |
| DQW | 1,2-dimethyl-7-(2-thiophene) | bevasiranib |
| | imidazolo [5,4-g] quinoxaline | |
| DQX | 1,2-dimethyl-7-(2-thiophene) | SIRNA-027 |
| | imidazolo [5,4-g] quinoxaline | |
| DQY | 1,2-dimethyl-7-(2-thiophene) | decursin |
| | imidazolo [5,4-g] quinoxaline | |
| DQZ | 1,2-dimethyl-7-(2-thiophene) | decursinol |
| | imidazolo [5,4-g] quinoxaline | |
| DRA | 1,2-dimethyl-7-(2-thiophene) | picropodophyllin |
| | imidazolo [5,4-g] quinoxaline | |
| DRB | 1,2-dimethyl-7-(2-thiophene) | guggulsterone |
| | imidazolo [5,4-g] quinoxaline | |
| DRC | 1,2-dimethyl-7-(2-thiophene) | PLG101 |
| | imidazolo [5,4-g] quinoxaline | |
| DRD | 1,2-dimethyl-7-(2-thiophene) | eicosanoid LXA4 |
| | imidazolo [5,4-g] quinoxaline | |
| DRE | 1,2-dimethyl-7-(2-thiophene) | PTK787 |
| | imidazolo [5,4-g] quinoxaline | |
| DRF | 1,2-dimethyl-7-(2-thiophene) | pazopanib |
| | imidazolo [5,4-g] quinoxaline | |
| DRG | 1,2-dimethyl-7-(2-thiophene) | axitinib |
| | imidazolo [5,4-g] quinoxaline | |
| DRH | 1,2-dimethyl-7-(2-thiophene) | CDDO-Me |
| | imidazolo [5,4-g] quinoxaline | |
| DRI | 1,2-dimethyl-7-(2-thiophene) | CDDO-Imm |
| | imidazolo [5,4-g] quinoxaline | |
| DRJ | 1,2-dimethyl-7-(2-thiophene) | shikonin |
| | imidazolo [5,4-g] quinoxaline | |
| DRK | 1,2-dimethyl-7-(2-thiophene) | beta-hydroxyisovalerylshikonin |
| | imidazolo [5,4-g] quinoxaline | |
| DRL | 1,2-dimethyl-7-(2-thiophene) | ganglioside GM3 |
| | imidazolo [5,4-g] quinoxaline | |
| DRM | 1,2-dimethyl-7-(2-thiophene) | DC101 antibody |
| | imidazolo [5,4-g] quinoxaline | |
| DRN | 1,2-dimethyl-7-(2-thiophene) | Mab25 antibody |
| | imidazolo [5,4-g] quinoxaline | |
| DRO | 1,2-dimethyl-7-(2-thiophene) | Mab73 antibody |
| | imidazolo [5,4-g] quinoxaline | |
| DRP | 1,2-dimethyl-7-(2-thiophene) | 4A5 antibody |
| | imidazolo [5,4-g] quinoxaline | |
| DRQ | 1,2-dimethyl-7-(2-thiophene) | 4E10 antibody |
| | imidazolo [5,4-g] quinoxaline | |
| DRR | 1,2-dimethyl-7-(2-thiophene) | 5F12 antibody |
| | imidazolo [5,4-g] quinoxaline | |
| DRS | 1,2-dimethyl-7-(2-thiophene) | VA01 antibody |
| | imidazolo [5,4-g] quinoxaline | |
| DRT | 1,2-dimethyl-7-(2-thiophene) | BL2 antibody |
| | imidazolo [5,4-g] quinoxaline | |
| DRU | 1,2-dimethyl-7-(2-thiophene) | VEGF-related protein |
| | imidazolo [5,4-g] quinoxaline | |
| DRV | 1,2-dimethyl-7-(2-thiophene) | sFLT01 |
| | imidazolo [5,4-g] quinoxaline | |
| DRW | 1,2-dimethyl-7-(2-thiophene) | sFLT02 |
| | imidazolo [5,4-g] quinoxaline | |
| DRX | 1,2-dimethyl-7-(2-thiophene) | Peptide B3 |
| | imidazolo [5,4-g] quinoxaline | |
| DRY | 1,2-dimethyl-7-(2-thiophene) | TG100801 |
| | imidazolo [5,4-g] quinoxaline | |
| DRZ | 1,2-dimethyl-7-(2-thiophene) | sorafenib |
| | imidazolo [5,4-g] quinoxaline | |
| DSA | 1,2-dimethyl-7-(2-thiophene) | G6-31 antibody |
| | imidazolo [5,4-g] quinoxaline | |
| DSB | 1,2-dimethyl-6-phenyl imidazolo [5, | ranibizumab |
| | 4-g] quinoxaline | |
| DSC | 1,2-dimethyl-6-phenyl imidazolo [5, | bevacizumab |
| | 4-g] quinoxaline | |
| DSD | 1,2-dimethyl-6-phenyl imidazolo [5, | aflibercept |
| | 4-g] quinoxaline | |
| DSE | 1,2-dimethyl-6-phenyl imidazolo [5, | KH902 VEGF receptor-Fc fusion protein |
| | 4-g] quinoxaline | |
| DSF | 1,2-dimethyl-6-phenyl imidazolo [5, | 2C3 antibody |
| | 4-g] quinoxaline | |
| DSG | 1,2-dimethyl-6-phenyl imidazolo [5, | ORA102 |
| | 4-g] quinoxaline | |
| DSH | 1,2-dimethyl-6-phenyl imidazolo [5, | pegaptanib |
| | 4-g] quinoxaline | |
| DSI | 1,2-dimethyl-6-phenyl imidazolo [5, | bevasiranib |
| | 4-g] quinoxaline | |
| DSJ | 1,2-dimethyl-6-phenyl imidazolo [5, | SIRNA-027 |
| | 4-g] quinoxaline | |
| DSK | 1,2-dimethyl-6-phenyl imidazolo [5, | decursin |
| | 4-g] quinoxaline | |
| DSL | 1,2-dimethyl-6-phenyl imidazolo [5, | decursinol |
| | 4-g] quinoxaline | |
| DSM | 1,2-dimethyl-6-phenyl imidazolo [5, | picropodophyllin |
| | 4-g] quinoxaline | |
| DSN | 1,2-dimethyl-6-phenyl imidazolo [5, | guggulsterone |
| | 4-g] quinoxaline | |
| DSO | 1,2-dimethyl-6-phenyl imidazolo [5, | PLG101 |
| | 4-g] quinoxaline | |
| DSP | 1,2-dimethyl-6-phenyl imidazolo [5, | eicosanoid LXA4 |
| | 4-g] quinoxaline | |
| DSQ | 1,2-dimethyl-6-phenyl imidazolo [5, | PTK787 |
| | 4-g] quinoxaline | |
| DSR | 1,2-dimethyl-6-phenyl imidazolo [5, | pazopanib |
| | 4-g] quinoxaline | |
| DSS | 1,2-dimethyl-6-phenyl imidazolo [5, | axitinib |
| | 4-g] quinoxaline | |
| DST | 1,2-dimethyl-6-phenyl imidazolo [5, | CDDO-Me |
| | 4-g] quinoxaline | |
| DSU | 1,2-dimethyl-6-phenyl imidazolo [5, | CDDO-Imm |
| | 4-g] quinoxaline | |
| DSV | 1,2-dimethyl-6-phenyl imidazolo [5, | shikonin |
| | 4-g] quinoxaline | |
| DSW | 1,2-dimethyl-6-phenyl imidazolo [5, | beta-hydroxyisovalerylshikonin |
| | 4-g] quinoxaline | |
| DSX | 1,2-dimethyl-6-phenyl imidazolo [5, | ganglioside GM3 |
| | 4-g] quinoxaline | |
| DSY | 1,2-dimethyl-6-phenyl imidazolo [5, | DC101 antibody |
| | 4-g] quinoxaline | |
| DSZ | 1,2-dimethyl-6-phenyl imidazolo [5, | Mab25 antibody |
| | 4-g] quinoxaline | |
| DTA | 1,2-dimethyl-6-phenyl imidazolo [5, | Mab73 antibody |
| | 4-g] quinoxaline | |
| DTB | 1,2-dimethyl-6-phenyl imidazolo [5, | 4A5 antibody |
| | 4-g] quinoxaline | |
| DTC | 1,2-dimethyl-6-phenyl imidazolo [5, | 4E10 antibody |
| | 4-g] quinoxaline | |
| DTD | 1,2-dimethyl-6-phenyl imidazolo [5, | 5F12 antibody |
| | 4-g] quinoxaline | |
| DTE | 1,2-dimethyl-6-phenyl imidazolo [5, | VA01 antibody |
| | 4-g] quinoxaline | |
| DTF | 1,2-dimethyl-6-phenyl imidazolo [5, | BL2 antibody |
| | 4-g] quinoxaline | |
| DTG | 1,2-dimethyl-6-phenyl imidazolo [5, | VEGF-related protein |
| | 4-g] quinoxaline | |
| DTH | 1,2-dimethyl-6-phenyl imidazolo [5, | sFLT01 |
| | 4-g] quinoxaline | |
| DTI | 1,2-dimethyl-6-phenyl imidazolo [5, | sFLT02 |
| | 4-g] quinoxaline | |
| DTJ | 1,2-dimethyl-6-phenyl imidazolo [5, | Peptide B3 |
| | 4-g] quinoxaline | |
| DTK | 1,2-dimethyl-6-phenyl imidazolo [5, | TG100801 |
| | 4-g] quinoxaline | |
| DTL | 1,2-dimethyl-6-phenyl imidazolo [5, | sorafenib |
| | 4-g] quinoxaline | |
| DTM | 1,2-dimethyl-6-phenyl imidazolo [5, | G6-31 antibody |
| | 4-g] quinoxaline | |
| DTN | 1,2-dimethyl-6-(2-thiophene) | ranibizumab |
| | imidazolo [5,4-g] quinoxaline | |
| DTO | 1,2-dimethyl-6-(2-thiophene) | bevacizumab |
| | imidazolo [5,4-g] quinoxaline | |
| DTP | 1,2-dimethyl-6-(2-thiophene) | aflibercept |
| | imidazolo [5,4-g] quinoxaline | |
| DTQ | 1,2-dimethyl-6-(2-thiophene) | KH902 VEGF receptor-Fc fusion protein |
| | imidazolo [5,4-g] quinoxaline | |
| DTR | 1,2-dimethyl-6-(2-thiophene) | 2C3 antibody |
| | imidazolo [5,4-g] quinoxaline | |
| DTS | 1,2-dimethyl-6-(2-thiophene) | ORA102 |
| | imidazolo [5,4-g] quinoxaline | |
| DTT | 1,2-dimethyl-6-(2-thiophene) | pegaptanib |
| | imidazolo [5,4-g] quinoxaline | |
| DTU | 1,2-dimethyl-6-(2-thiophene) | bevasiranib |
| | imidazolo [5,4-g] quinoxaline | |
| DTV | 1,2-dimethyl-6-(2-thiophene) | SIRNA-027 |
| | imidazolo [5,4-g] quinoxaline | |
| DTW | 1,2-dimethyl-6-(2-thiophene) | decursin |
| | imidazolo [5,4-g] quinoxaline | |
| DTX | 1,2-dimethyl-6-(2-thiophene) | decursinol |
| | imidazolo [5,4-g] quinoxaline | |
| DTY | 1,2-dimethyl-6-(2-thiophene) | picropodophyllin |
| | imidazolo [5,4-g] quinoxaline | |
| DTZ | 1,2-dimethyl-6-(2-thiophene) | guggulsterone |
| | imidazolo [5,4-g] quinoxaline | |
| DUA | 1,2-dimethyl-6-(2-thiophene) | PLG101 |
| | imidazolo [5,4-g] quinoxaline | |
| DUB | 1,2-dimethyl-6-(2-thiophene) | eicosanoid LXA4 |
| | imidazolo [5,4-g] quinoxaline | |
| DUC | 1,2-dimethyl-6-(2-thiophene) | PTK787 |
| | imidazolo [5,4-g] quinoxaline | |
| DUD | 1,2-dimethyl-6-(2-thiophene) | pazopanib |
| | imidazolo [5,4-g] quinoxaline | |
| DUE | 1,2-dimethyl-6-(2-thiophene) | axitinib |
| | imidazolo [5,4-g] quinoxaline | |
| DUF | 1,2-dimethyl-6-(2-thiophene) | CDDO-Me |
| | imidazolo [5,4-g] quinoxaline | |
| DUG | 1,2-dimethyl-6-(2-thiophene) | CDDO-Imm |
| | imidazolo [5,4-g] quinoxaline | |
| DUH | 1,2-dimethyl-6-(2-thiophene) | shikonin |
| | imidazolo [5,4-g] quinoxaline | |
| DUI | 1,2-dimethyl-6-(2-thiophene) | beta-hydroxyisovalerylshikonin |
| | imidazolo [5,4-g] quinoxaline | |
| DUJ | 1,2-dimethyl-6-(2-thiophene) | ganglioside GM3 |
| | imidazolo [5,4-g] quinoxaline | |
| DUK | 1,2-dimethyl-6-(2-thiophene) | DC101 antibody |
| | imidazolo [5,4-g] quinoxaline | |
| DUL | 1,2-dimethyl-6-(2-thiophene) | Mab25 antibody |
| | imidazolo [5,4-g] quinoxaline | |
| DUM | 1,2-dimethyl-6-(2-thiophene) | Mab73 antibody |
| | imidazolo [5,4-g] quinoxaline | |
| DUN | 1,2-dimethyl-6-(2-thiophene) | 4A5 antibody |
| | imidazolo [5,4-g] quinoxaline | |
| DUO | 1,2-dimethyl-6-(2-thiophene) | 4E10 antibody |
| | imidazolo [5,4-g] quinoxaline | |
| DUP | 1,2-dimethyl-6-(2-thiophene) | 5F12 antibody |
| | imidazolo [5,4-g] quinoxaline | |
| DUQ | 1,2-dimethyl-6-(2-thiophene) | VA01 antibody |
| | imidazolo [5,4-g] quinoxaline | |
| DUR | 1,2-dimethyl-6-(2-thiophene) | BL2 antibody |
| | imidazolo [5,4-g] quinoxaline | |
| DUS | 1,2-dimethyl-6-(2-thiophene) | VEGF-related protein |
| | imidazolo [5,4-g] quinoxaline | |
| DUT | 1,2-dimethyl-6-(2-thiophene) | sFLT01 |
| | imidazolo [5,4-g] quinoxaline | |
| DUU | 1,2-dimethyl-6-(2-thiophene) | sFLT02 |
| | imidazolo [5,4-g] quinoxaline | |
| DUV | 1,2-dimethyl-6-(2-thiophene) | Peptide B3 |
| | imidazolo [5,4-g] quinoxaline | |
| DUW | 1,2-dimethyl-6-(2-thiophene) | TG100801 |
| | imidazolo [5,4-g] quinoxaline | |
| DUX | 1,2-dimethyl-6-(2-thiophene) | sorafenib |
| | imidazolo [5,4-g] quinoxaline | |
| DUY | 1,2-dimethyl-6-(2-thiophene) | G6-31 antibody |
| | imidazolo [5,4-g] quinoxaline | |
| DUZ | AG1295 | ranibizumab |
| DVA | AG1295 | bevacizumab |
| DVB | AG1295 | aflibercept |
| DVC | AG1295 | KH902 VEGF receptor-Fc fusion protein |
| DVD | AG1295 | 2C3 antibody |
| DVE | AG1295 | ORA102 |
| DVF | AG1295 | Pegaptanib |
| DVG | AG1295 | bevasiranib |
| DVH | AG1295 | SIRNA-027 |
| DVI | AG1295 | decursin |
| DVJ | AG1295 | decursinol |
| DVK | AG1295 | picropodophyllin |
| DVL | AG1295 | guggulsterone |
| DVM | AG1295 | PLG101 |
| DVN | AG1295 | eicosanoid LXA4 |
| DVO | AG1295 | PTK787 |
| DVP | AG1295 | pazopanib |
| DVQ | AG1295 | axitinib |
| DVR | AG1295 | CDDO-Me |
| DVS | AG1295 | CDDO-Imm |
| DVT | AG1295 | shikonin |
| DVU | AG1295 | beta-hydroxyisovalerylshikonin |
| DVV | AG1295 | ganglioside GM3 |
| DVW | AG1295 | DC101 antibody |
| DVX | AG1295 | Mab25 antibody |
| DVY | AG1295 | Mab73 antibody |
| DVZ | AG1295 | 4A5 antibody |
| DWA | AG1295 | 4E10 antibody |
| DWB | AG1295 | 5F12 antibody |
| DWC | AG1295 | VA01 antibody |
| DWD | AG1295 | BL2 antibody |
| DWE | AG1295 | VEGF-related protein |
| DWF | AG1295 | sFLT01 |
| DWG | AG1295 | sFLT02 |
| DWH | AG1295 | Peptide B3 |
| DWI | AG1295 | TG100801 |
| DWJ | AG1295 | sorafenib |
| DWK | AG1295 | G6-31 antibody |
| DWL | AG1296 | ranibizumab |
| DWM | AG1296 | bevacizumab |
| DWN | AG1296 | aflibercept |
| DWO | AG1296 | KH902 VEGF receptor-Fc fusion protein |
| DWP | AG1296 | 2C3 antibody |
| DWQ | AG1296 | ORA102 |
| DWR | AG1296 | pegaptanib |
| DWS | AG1296 | bevasiranib |
| DWT | AG1296 | SIRNA-027 |
| DWU | AG1296 | Decursin |
| DWV | AG1296 | decursinol |
| DWW | AG1296 | picropodophyllin |
| DWX | AG1296 | guggulsterone |
| DWY | AG1296 | PLG101 |
| DWZ | AG1296 | eicosanoid LXA4 |
| DXA | AG1296 | PTK787 |
| DXB | AG1296 | pazopanib |
| DXC | AG1296 | axitinib |
| DXD | AG1296 | CDDO-Me |
| DXE | AG1296 | CDDO-Imm |
| DXF | AG1296 | shikonin |
| DXG | AG1296 | beta-hydroxyisovalerylshikonin |
| DXH | AG1296 | ganglioside GM3 |
| DXI | AG1296 | DC101 antibody |
| DXJ | AG1296 | Mab25 antibody |
| DXK | AG1296 | Mab73 antibody |
| DXL | AG1296 | 4A5 antibody |
| DXM | AG1296 | 4E10 antibody |
| DXN | AG1296 | 5F12 antibody |
| DXO | AG1296 | VA01 antibody |
| DXP | AG1296 | BL2 antibody |
| DXQ | AG1296 | VEGF-related protein |
| DXR | AG1296 | sFLT01 |
| DXS | AG1296 | sFLT02 |
| DXT | AG1296 | Peptide B3 |
| DXU | AG1296 | TG100801 |
| DXV | AG1296 | sorafenib |
| DXW | AG1296 | G6-31 antibody |
| DXX | 3-arylquinoline | ranibizumab |
| DXY | 3-arylquinoline | bevacizumab |
| DXZ | 3-arylquinoline | aflibercept |
| DYA | 3-arylquinoline | KH902 VEGF receptor-Fc fusion protein |
| DYB | 3-arylquinoline | 2C3 antibody |
| DYC | 3-arylquinoline | ORA102 |
| DYD | 3-arylquinoline | pegaptanib |
| DYE | 3-arylquinoline | bevasiranib |
| DYF | 3-arylquinoline | SIRNA-027 |
| DYG | 3-arylquinoline | decursin |
| DYH | 3-arylquinoline | decursinol |
| DYI | 3-arylquinoline | picropodophyllin |
| DYJ | 3-arylquinoline | Guggulsterone |
| DYK | 3-arylquinoline | PLG101 |
| DYL | 3-arylquinoline | eicosanoid LXA4 |
| DYM | 3-arylquinoline | PTK787 |
| DYN | 3-arylquinoline | pazopanib |
| DYO | 3-arylquinoline | axitinib |
| DYP | 3-arylquinoline | CDDO-Me |
| DYQ | 3-arylquinoline | CDDO-Imm |
| DYR | 3-arylquinoline | shikonin |
| DYS | 3-arylquinoline | beta-hydroxyisovalerylshikonin |
| DYT | 3-arylquinoline | ganglioside GM3 |
| DYU | 3-arylquinoline | DC101 antibody |
| DYV | 3-arylquinoline | Mab25 antibody |
| DYW | 3-arylquinoline | Mab73 antibody |
| DYX | 3-arylquinoline | 4A5 antibody |
| DYY | 3-arylquinoline | 4E10 antibody |
| DYZ | 3-arylquinoline | 5F12 antibody |
| DZA | 3-arylquinoline | VA01 antibody |
| DZB | 3-arylquinoline | BL2 antibody |
| DZC | 3-arylquinoline | VEGF-related protein |
| DZD | 3-arylquinoline | sFLT01 |
| DZE | 3-arylquinoline | sFLT02 |
| DZF | 3-arylquinoline | Peptide B3 |
| DZG | 3-arylquinoline | TG100801 |
| DZH | 3-arylquinoline | sorafenib |
| DZI | 3-arylquinoline | G6-31 antibody |
| DZJ | 4-pyridyl-2-arylpyrimidine | ranibizumab |
| DZK | 4-pyridyl-2-arylpyrimidine | bevacizumab |
| DZL | 4-pyridyl-2-arylpyrimidine | aflibercept |
| DZM | 4-pyridyl-2-arylpyrimidine | KH902 VEGF receptor-Fc fusion protein |
| DZN | 4-pyridyl-2-arylpyrimidine | 2C3 antibody |
| DZO | 4-pyridyl-2-arylpyrimidine | ORA102 |
| DZP | 4-pyridyl-2-arylpyrimidine | pegaptanib |
| DZQ | 4-pyridyl-2-arylpyrimidine | bevasiranib |
| DZR | 4-pyridyl-2-arylpyrimidine | SIRNA-027 |
| DZS | 4-pyridyl-2-arylpyrimidine | decursin |
| DZT | 4-pyridyl-2-arylpyrimidine | decursinol |
| DZU | 4-pyridyl-2-arylpyrimidine | picropodophyllin |
| DZV | 4-pyridyl-2-arylpyrimidine | guggulsterone |
| DZW | 4-pyridyl-2-arylpyrimidine | PLG101 |
| DZX | 4-pyridyl-2-arylpyrimidine | eicosanoid LXA4 |
| DZY | 4-pyridyl-2-arylpyrimidine | PTK787 |
| DZZ | 4-pyridyl-2-arylpyrimidine | pazopanib |
| EAA | 4-pyridyl-2-arylpyrimidine | axitinib |
| EAB | 4-pyridyl-2-arylpyrimidine | CDDO-Me |
| EAC | 4-pyridyl-2-arylpyrimidine | CDDO-Imm |
| EAD | 4-pyridyl-2-arylpyrimidine | shikonin |
| EAE | 4-pyridyl-2-arylpyrimidine | beta-hydroxyisovaleryishikonin |
| EAF | 4-pyridyl-2-arylpyrimidine | ganglioside GM3 |
| EAG | 4-pyridyl-2-arylpyrimidine | DC101 antibody |
| EAH | 4-pyridyl-2-arylpyrimidine | Mab25 antibody |
| EAI | 4-pyridyl-2-arylpyrimidine | Mab73 antibody |
| EAJ | 4-pyridyl-2-arylpyrimidine | 4A5 antibody |
| EAK | 4-pyridyl-2-arylpyrimidine | 4E10 antibody |
| EAL | 4-pyridyl-2-arylpyrimidine | 5F12 antibody |
| EAM | 4-pyridyl-2-arylpyrimidine | VA01 antibody |
| EAN | 4-pyridyl-2-arylpyrimidine | BL2 antibody |
| EAO | 4-pyridyl-2-arylpyrimidine | VEGF-related protein |
| EAP | 4-pyridyl-2-arylpyrimidine | sFLT01 |
| EAQ | 4-pyridyl-2-arylpyrimidine | sFLT02 |
| EAR | 4-pyridyl-2-arylpyrimidine | Peptide B3 |
| EAS | 4-pyridyl-2-arylpyrimidine | TG100801 |
| EAT | 4-pyridyl-2-arylpyrimidine | sorafenib |
| EAU | 4-pyridyl-2-arylpyrimidine | G6-31 antibody |
| EAV | MLN518 | ranibizumab |
| EAW | MLN518 | bevacizumab |
| EAX | MLN518 | aflibercept |
| EAY | MLN518 | KH902 VEGF receptor-Fc fusion protein |
| EAZ | MLN518 | 2C3 antibody |
| EBA | MLN518 | ORA102 |
| EBB | MLN518 | pegaptanib |
| EBC | MLN518 | bevasiranib |
| EBD | MLN518 | SIRNA-027 |
| EBE | MLN518 | decursin |
| EBF | MLN518 | decursinol |
| EBG | MLN518 | picropodophyllin |
| EBH | MLN518 | guggulsterone |
| EBI | MLN518 | PLG101 |
| EBJ | MLN518 | eicosanoid LXA4 |
| EBK | MLN518 | PTK787 |
| EBL | MLN518 | pazopanib |
| EBM | MLN518 | axitinib |
| EBN | MLN518 | CDDO-Me |
| EBO | MLN518 | CDDO-Imm |
| EBP | MLN518 | shikonin |
| EBQ | MLN518 | beta-hydroxyisovalerylshikonin |
| EBR | MLN518 | ganglioside GM3 |
| EBS | MLN518 | DC101 antibody |
| EBT | MLN518 | Mab25 antibody |
| EBU | MLN518 | Mab73 antibody |
| EBV | MLN518 | 4A5 antibody |
| EBW | MLN518 | 4E10 antibody |
| EBX | MLN518 | 5F12 antibody |
| EBY | MLN518 | VA01 antibody |
| EBZ | MLN518 | BL2 antibody |
| ECA | MLN518 | VEGF-related protein |
| ECB | MLN518 | sFLT01 |
| ECC | MLN518 | sFLT02 |
| ECD | MLN518 | Peptide B3 |
| ECE | MLN518 | TG100801 |
| ECF | MLN518 | sorafenib |
| ECG | MLN518 | G6-31 antibody |
| ECH | PKC412 | ranibizumab |
| ECI | PKC412 | bevacizumab |
| ECJ | PKC412 | aflibercept |
| ECK | PKC412 | KH902 VEGF receptor-Fc fusion protein |
| ECL | PKC412 | 2C3 antibody |
| ECM | PKC412 | ORA102 |
| ECN | PKC412 | pegaptanib |
| ECO | PKC412 | bevasiranib |
| ECP | PKC412 | SIRNA-027 |
| ECQ | PKC412 | decursin |
| ECR | PKC412 | decursinol |
| ECS | PKC412 | picropodophyllin |
| ECT | PKC412 | guggulsterone |
| ECU | PKC412 | PLG101 |
| ECV | PKC412 | eicosanoid LXA4 |
| ECW | PKC412 | PTK787 |
| ECX | PKC412 | pazopanib |
| ECY | PKC412 | axitinib |
| ECZ | PKC412 | CDDO-Me |
| EDA | PKC412 | CDDO-Imm |
| EDB | PKC412 | shikonin |
| EDC | PKC412 | beta-hydroxyisovalerylshikonin |
| EDD | PKC412 | ganglioside GM3 |
| EDE | PKC412 | DC101 antibody |
| EDF | PKC412 | Mab25 antibody |
| EDG | PKC412 | Mab73 antibody |
| EDH | PKC412 | 4A5 antibody |
| EDI | PKC412 | 4E10 antibody |
| EDJ | PKC412 | 5F12 antibody |
| EDK | PKC412 | VA01 antibody |
| EDL | PKC412 | BL2 antibody |
| EDM | PKC412 | VEGF-related protein |
| EDN | PKC412 | sFLT01 |
| EDO | PKC412 | sFLT02 |
| EDP | PKC412 | Peptide B3 |
| EDQ | PKC412 | TG100801 |
| EDR | PKC412 | sorafenib |
| EDS | PKC412 | G6-31 antibody |
| EDT | AMN107 | ranibizumab |
| EDU | AMN107 | bevacizumab |
| EDV | AMN107 | aflibercept |
| EDW | AMN107 | KH902 VEGF receptor-Fc fusion protein |
| EDX | AMN107 | 2C3 antibody |
| EDY | AMN107 | ORA102 |
| EDZ | AMN107 | pegaptanib |
| EEA | AMN107 | bevasiranib |
| EEB | AMN107 | SIRNA-027 |
| EEC | AMN107 | decursin |
| EED | AMN107 | decursinol |
| EEF | AMN107 | picropodophyllin |
| EEG | AMN107 | guggulsterone |
| EEH | AMN107 | PLG101 |
| EEI | AMN107 | eicosanoid LXA4 |
| EEJ | AMN107 | PTK787 |
| EEK | AMN107 | pazopanib |
| EEL | AMN107 | axitinib |
| EEM | AMN107 | CDDO-Me |
| EEN | AMN107 | CDDO-Imm |
| EEO | AMN107 | shikonin |
| EEP | AMN107 | beta-hydroxyisovalerylshikonin |
| EEQ | AMN107 | ganglioside GM3 |
| EER | AMN107 | DC101 antibody |
| EES | AMN107 | Mab25 antibody |
| EET | AMN107 | Mab73 antibody |
| EEU | AMN107 | 4A5 antibody |
| EEV | AMN107 | 4E10 antibody |
| EEW | AMN107 | 5F12 antibody |
| EEX | AMN107 | VA01 antibody |
| EEY | AMN107 | BL2 antibody |
| EEZ | AMN107 | VEGF-related protein |
| EFA | AMN107 | sFLT01 |
| EFB | AMN107 | sFLT02 |
| EFC | AMN107 | Peptide B3 |
| EFD | AMN107 | TG100801 |
| EFE | AMN107 | sorafenib |
| EFF | AMN107 | G6-31 antibody |
| EFG | Suramin | ranibizumab |
| EFH | Suramin | bevacizumab |
| EFI | Suramin | aflibercept |
| EFJ | Suramin | KH902 VEGF receptor-Fc fusion protein |
| EFK | Suramin | 2C3 antibody |
| EFL | Suramin | ORA102 |
| EFM | Suramin | pegaptanib |
| EFN | Suramin | bevasiranib |
| EFO | Suramin | SIRNA-027 |
| EFP | Suramin | decursin |
| EFQ | Suramin | decursinol |
| EFR | Suramin | picropodophyllin |
| EFS | Suramin | guggulsterone |
| EFT | Suramin | PLG101 |
| EFU | Suramin | eicosanoid LXA4 |
| EFV | Suramin | PTK787 |
| EFW | Suramin | pazopanib |
| EFX | Suramin | axitinib |
| EFY | Suramin | CDDO-Me |
| EFZ | Suramin | CDDO-Imm |
| EGA | Suramin | shikonin |
| EGB | Suramin | beta-hydroxyisovalerylshikonin |
| EGC | Suramin | ganglioside GM3 |
| EGD | Suramin | DC101 antibody |
| EGE | Suramin | Mab25 antibody |
| EGF | Suramin | Mab73 antibody |
| EGG | Suramin | 4A5 antibody |
| EGH | Suramin | 4E10 antibody |
| EGI | Suramin | 5F12 antibody |
| EGJ | Suramin | VA01 antibody |
| EGK | Suramin | BL2 antibody |
| EGL | Suramin | VEGF-related protein |
| EGM | Suramin | sFLT01 |
| EGN | Suramin | sFLT02 |
| EGO | Suramin | Peptide B3 |
| EGP | Suramin | TG100801 |
| EGQ | Suramin | sorafenib |
| EGR | Suramin | G6-31 antibody |
| EGS | Neomycin | ranibizumab |
| EGT | Neomycin | bevacizumab |
| EGU | Neomycin | aflibercept |
| EGV | Neomycin | KH902 VEGF receptor-Fc fusion protein |
| EGW | Neomycin | 2C3 antibody |
| EGX | Neomycin | ORA102 |
| EGY | Neomycin | pegaptanib |
| EGZ | Neomycin | bevasiranib |
| EHA | Neomycin | SIRNA-027 |
| EHB | Neomycin | decursin |
| EHC | Neomycin | decursinol |
| EHD | Neomycin | picropodophyllin |
| EHE | Neomycin | guggulsterone |
| EHF | Neomycin | PLG101 |
| EHG | Neomycin | eicosanoid LXA4 |
| EHH | Neomycin | PTK787 |
| EHI | Neomycin | pazopanib |
| EHJ | Neomycin | axitinib |
| EHK | Neomycin | CDDO-Me |
| EHL | Neomycin | CDDO-Imm |
| EHM | Neomycin | shikonin |
| EHN | Neomycin | beta-hydroxyisovalerylshikonin |
| EHO | Neomycin | ganglioside GM3 |
| EHP | Neomycin | DC101 antibody |
| EHQ | Neomycin | Mab25 antibody |
| HER | Neomycin | Mab73 antibody |
| EHS | Neomycin | 4A5 antibody |
| EHT | Neomycin | 4E10 antibody |
| EHU | Neomycin | 5F12 antibody |
| EHV | Neomycin | VA01 antibody |
| EHW | Neomycin | BL2 antibody |
| EHX | Neomycin | VEGF-related protein |
| EHY | Neomycin | sFLT01 |
| EHZ | Neomycin | sFLT02 |
| EIA | Neomycin | Peptide B3 |
| EIB | Neomycin | TG100801 |
| EIC | Neomycin | sorafenib |
| EID | Neomycin | G6-31 antibody |

The invention further provides compositions comprising an effective amount of a PDGF antagonist and a VEGF antagonist of Table 1. The compositions are useful for treating or preventing an ophthalmological disease. In another embodiment, the PDGF antagonist and VEGF antagonist are those, respectively, of any of pairs A-EID set forth in Table 2. In a particular embodiment, the PDGF antagonist of the present compositions is Antagonist A or a pharmaceutically acceptable salt thereof. In a particular embodiment, the PDGF antagonist of the present compositions is Antagonist B or a pharmaceutically acceptable salt thereof. In a particular embodiment, the PDGF antagonist of the present compositions is Antagonist C or a pharmaceutically acceptable salt thereof. In a particular embodiment, the PDGF antagonist of the present compositions is Antagonist D or a pharmaceutically acceptable salt thereof. In another embodiment, the VEGF antagonist is ranibizumab, bevacizumab or aflibercept, or a pharmaceutically acceptable salt thereof.

The methods or compositions according to the invention can be administered alone or in conjunction with another therapy and can be provided at home, a doctor's office, a clinic, a hospital's outpatient department, or a hospital. Treatment can begin at a hospital so that the doctor can observe the therapy's effects closely and make any adjustments that are needed. The duration of the administration can depend on the type of ophthalmological disease being treated or prevented, the age and condition of the mammal, the stage and type of the mammal's disease, and how the mammal responds to the treatment. Additionally, a person having a greater risk of developing an ophthalmological disease (e.g., a diabetic patient) can receive treatment to inhibit or delay the onset of symptoms. In one embodiment, the present methods or compositions allow for the administration of a relatively lower dose of each antagonist.

The dosage and frequency of administration of each antagonist can be controlled independently. For example, one antagonist can be administered three times per day, while the other antagonist can be administered once per day. Administration can be performed in on-and-off cycles that include rest periods so that the mammal's body has a chance to recover from a side effect, if any. The antagonists can also be present in the same composition.

### 7.3 Agents Useful for Treatment or Prevention of an Opthalmological Disease

7.3.1 PDGF Antagonists

In one embodiment, the PDGF antagonist of Table 1 or 2 is ARC-127. ARC-127 is a PEGylated, anti-PDGF aptamer having the sequence CAGGCUACGN CGTAGAGCAU CANTGATCCU GT (SEQ ID NO: 23) (see Examples 3 of US Patent Application No. 20050096257, incorporated herein by reference in its entirety) having 2′-fluoro-2′-deoxyuridine at positions 6, 20 and 30; 2′-fluoro-2′-deoxycytidine at positions 8, 21, 28, and 29; 2′-O-Methyl-2′-deoxyguanosine at positions 9, 15, 17, and 31; 2′-O-Methyl-2′-deoxyadenosine at position 22; “N” in positions 10 and 23 from a hexaethylene-glycol phosphoramidite; and an inverted orientation T (i.e., 3′-3′-linked) at position 32.

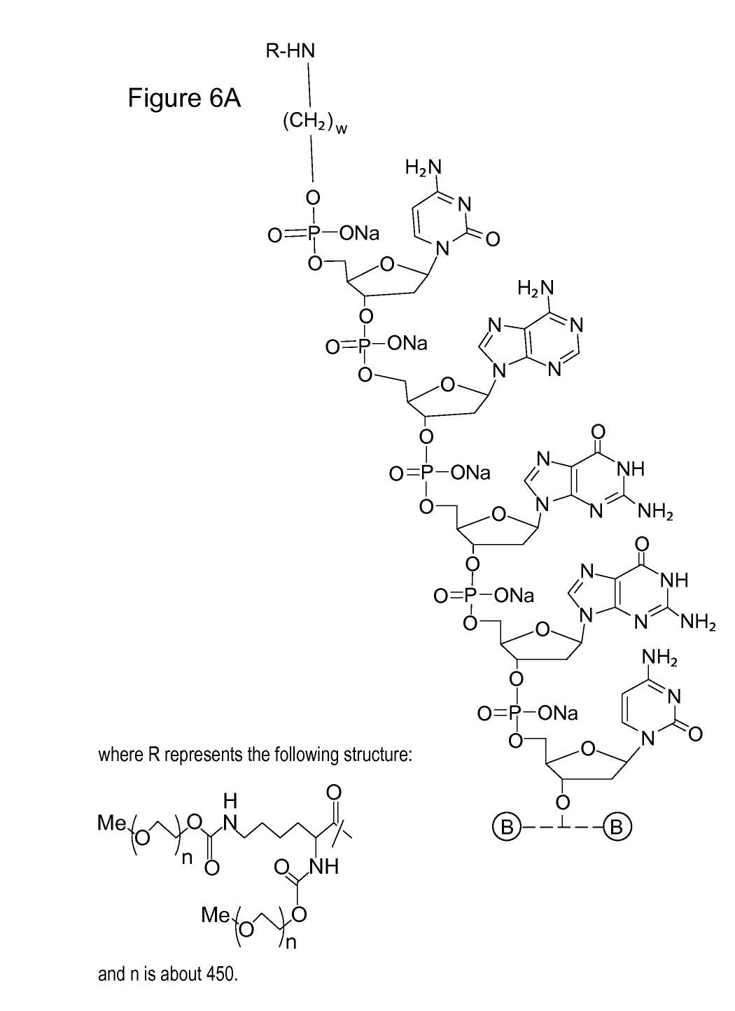

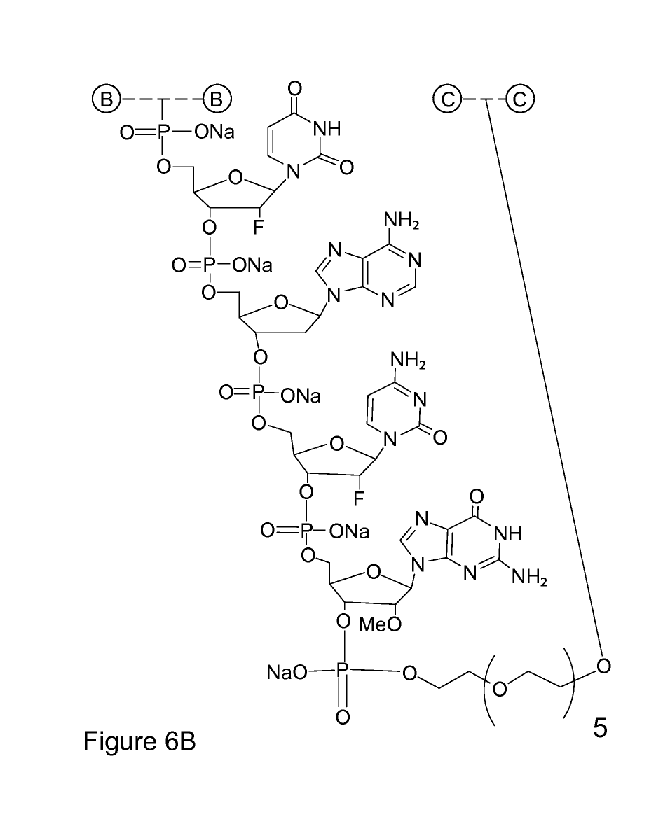

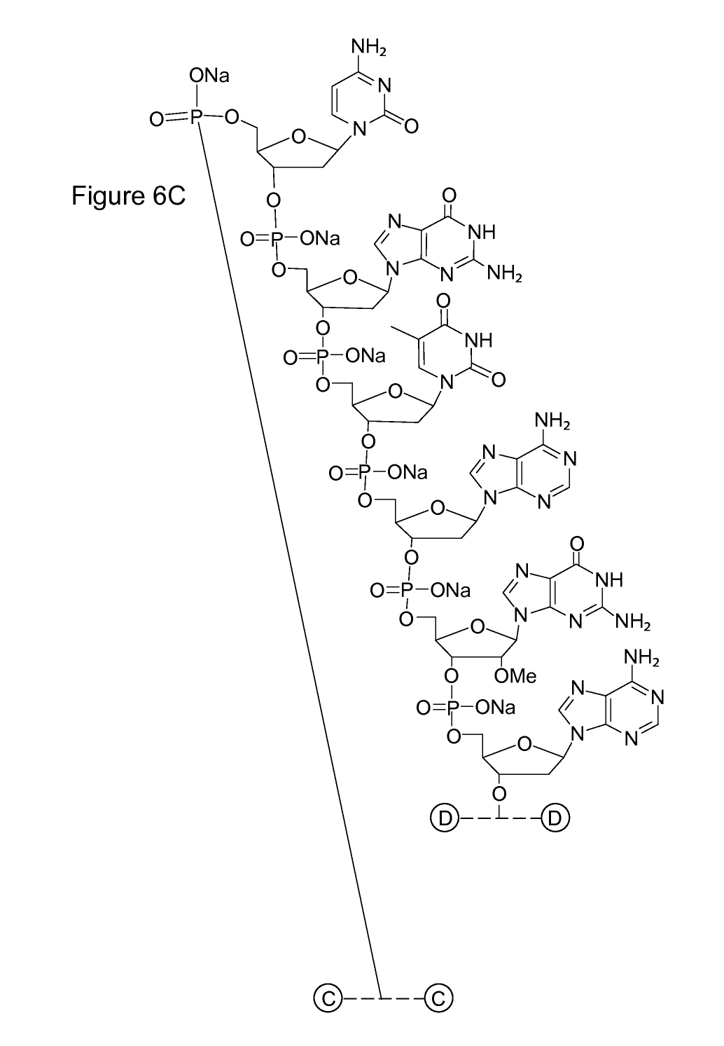

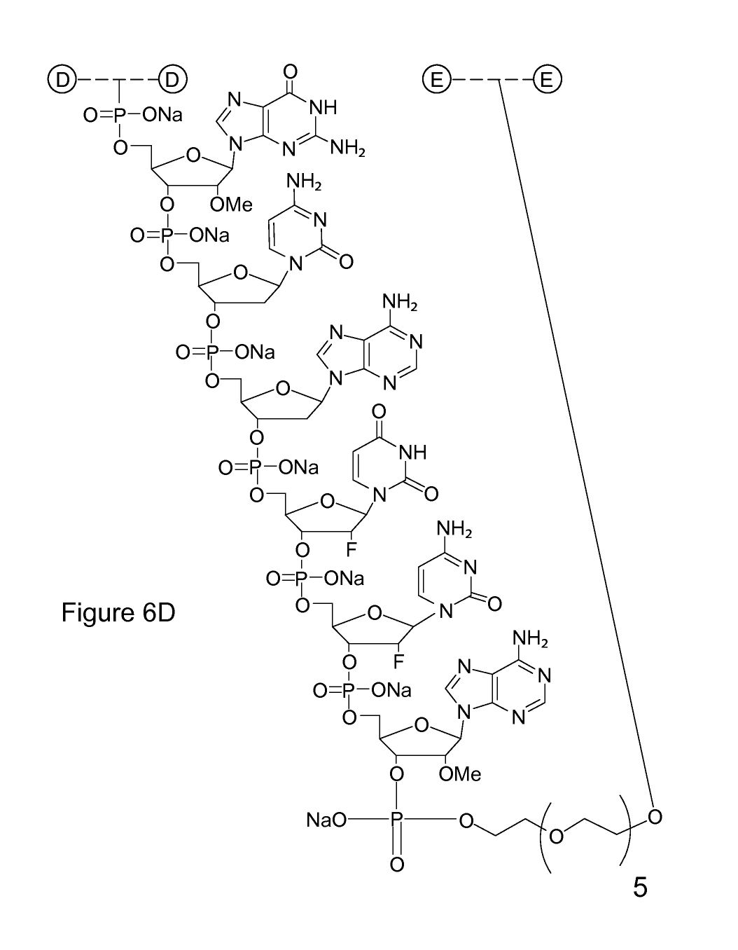

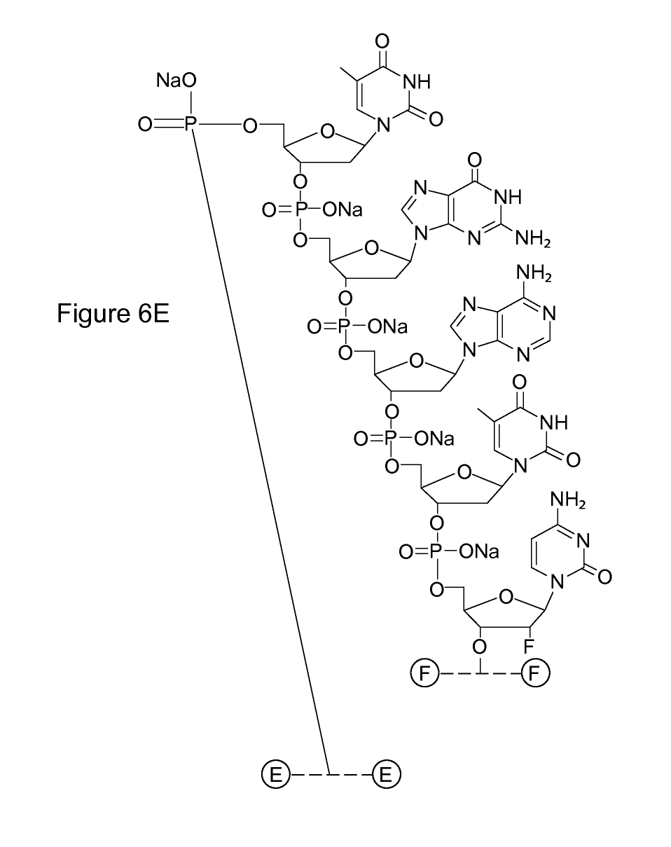

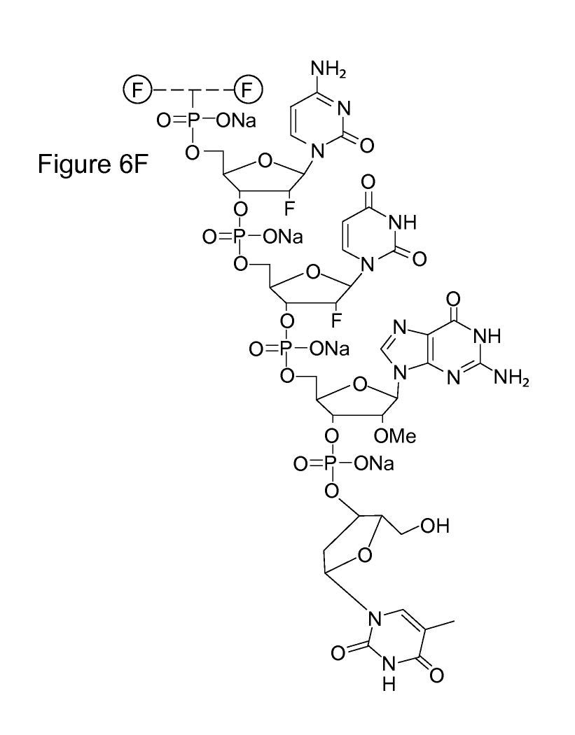

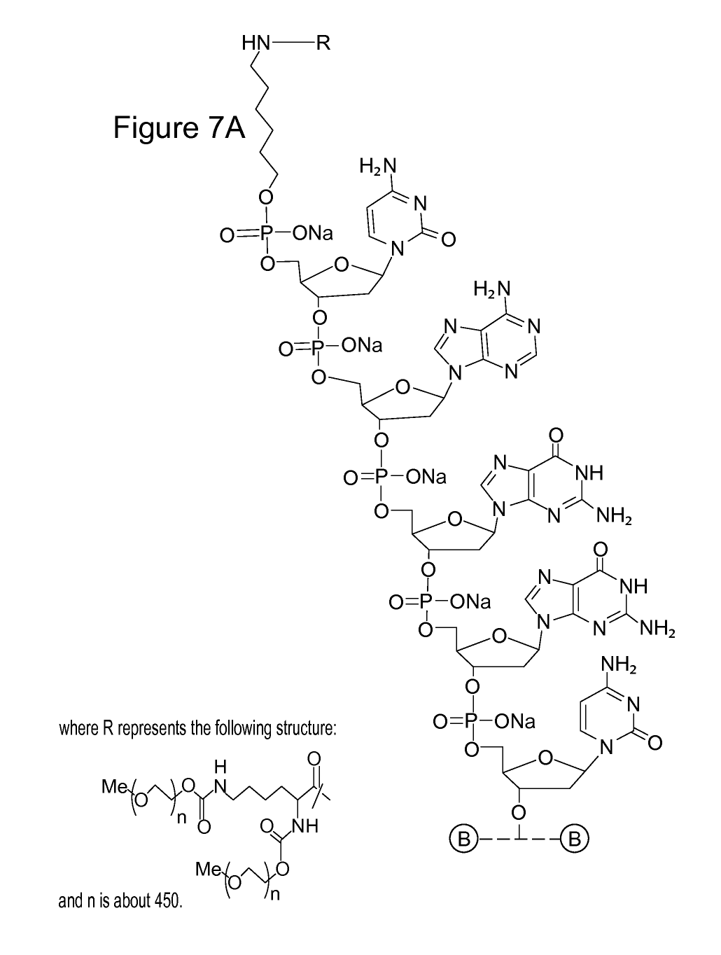

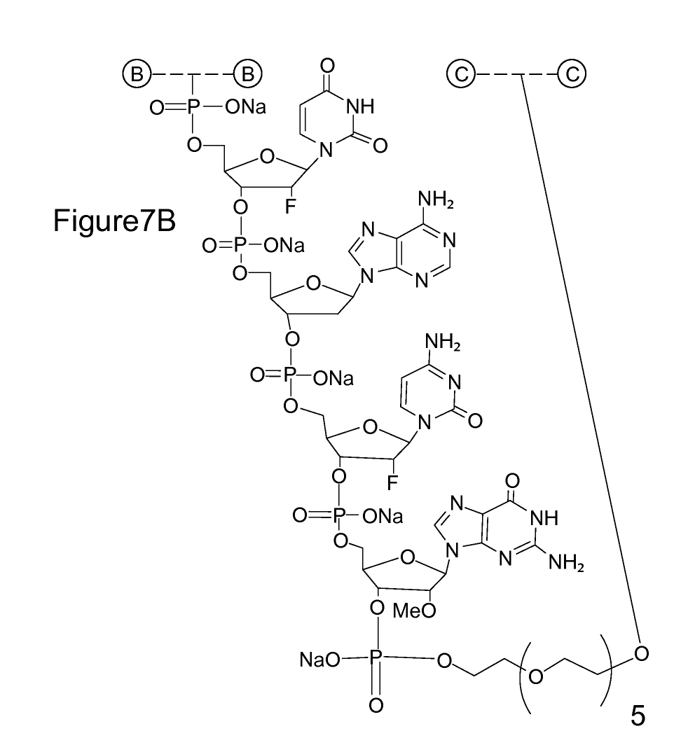

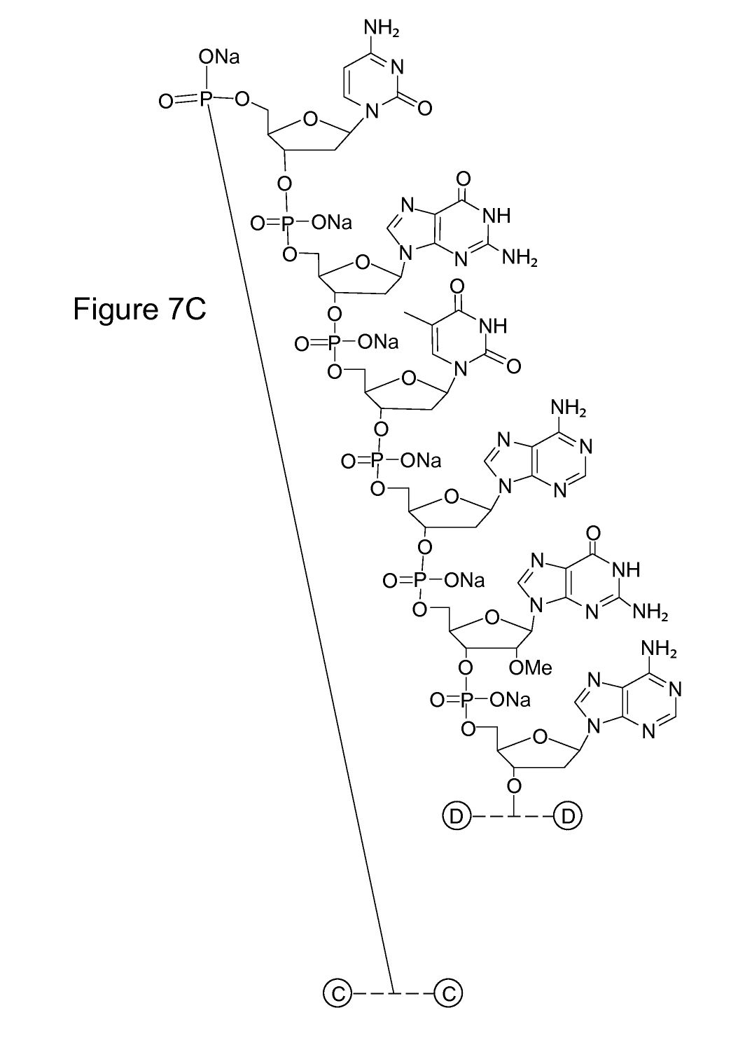

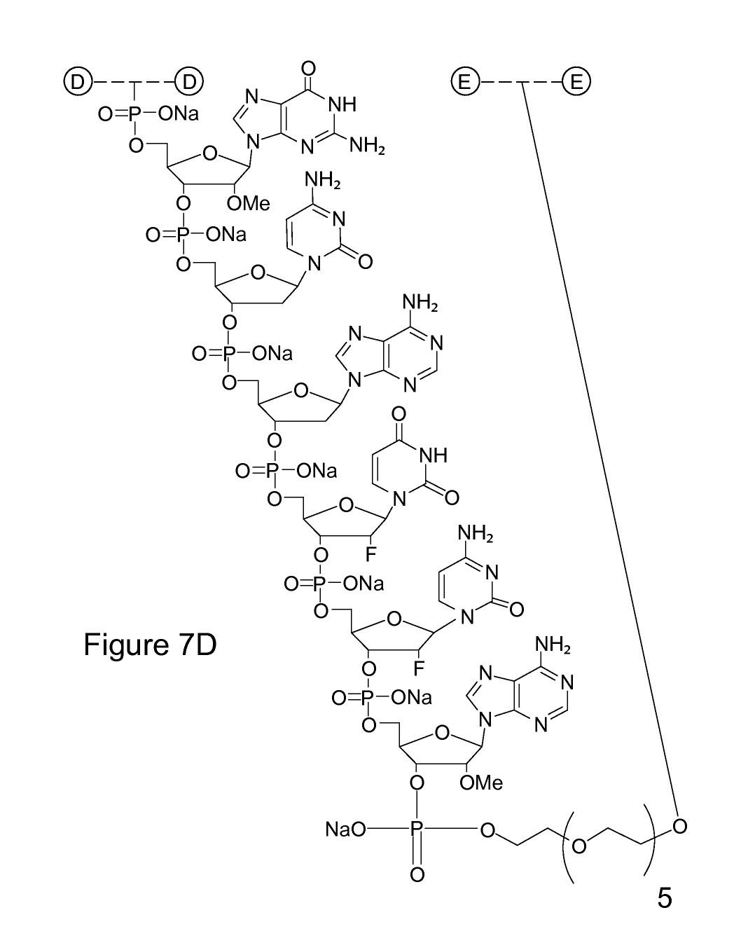

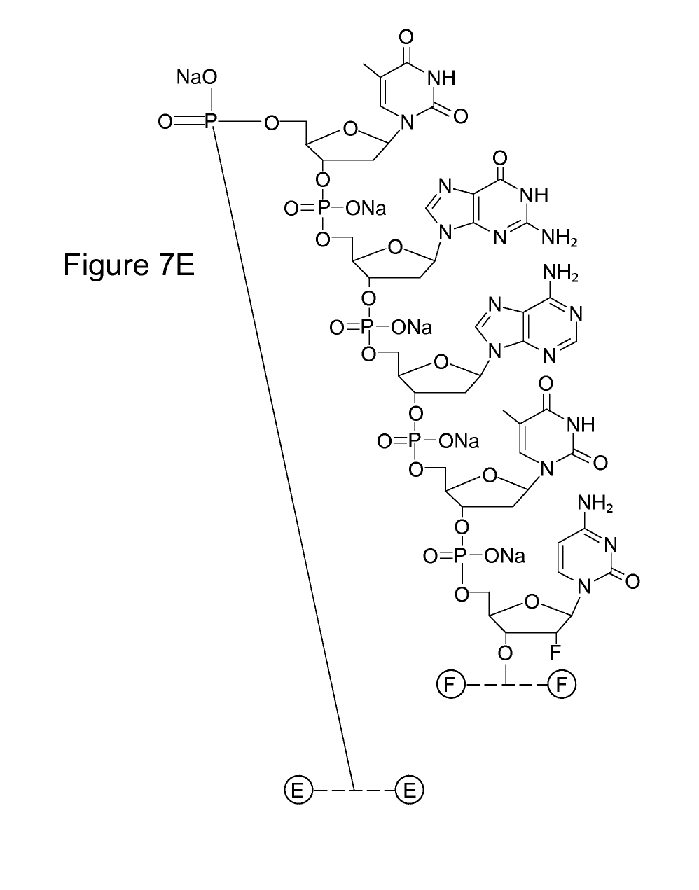

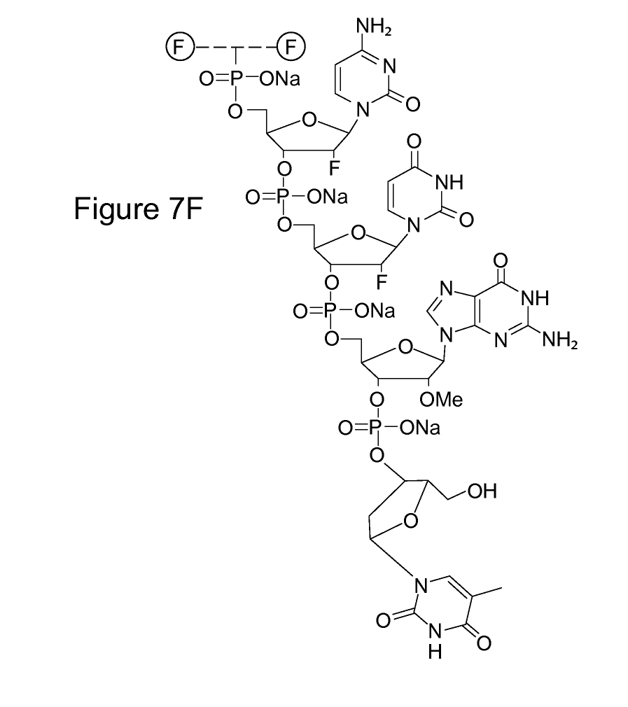

In another embodiment, the PDGF antagonist of Table 1 or 2 is a compound of Formula A (see FIG. 6), wherein w is an integer from 2 to 12. In another embodiment, the PDGF antagonist is a compound of Formula A, wherein w is an integer from 4 to 10. In another embodiment, the PDGF antagonist is a compound of Formula A, wherein w is 5, 6, 7, or 8. In one embodiment, the PDGF antagonist is a compound of Formula A, wherein w is 5. In another embodiment, the PDGF antagonist is a compound of Formula A, wherein w is 6. In another embodiment, the PDGF antagonist is a compound of Formula A, wherein w is 7. In another embodiment, the PDGF antagonist is a compound of Formula A, wherein w is 8. In one embodiment, the PDGF antagonist has the structure of FIG. 7.

In another embodiment, the PDGF antagonist of Table 1 or 2 is Antagonist A or a pharmaceutically acceptable salt thereof. The chemical name of Antagonist A is [(monomethoxy 20K polyethylene glycol carbamoyl-N2-) (monomethoxy 20K polyethylene glycol carbamoyl-N6-)]-lysine-amido-6-hexandilyl-(1-5′)-2′-deoxycytidylyl-(3′-5′)-2′-deoxyadenylyl-(3′-5′)-2′-deoxyguanylyl-(3′-5′)-2′-deoxyguanylyl-(3′-5′)-2′-deoxycytidylyl-(3′-5′)-2′-deoxy-2′-fluorouridylyl-(3′-5′)-2′-deoxyadenylyl-(3′-5′)-2′-deoxy-2′-fluorocytidylyl-(3′-5′)-2′-deoxy-2′-methoxyguanylyl-(3′-1)-PO₃-hexa(ethyloxy)-(18-5′)-2′-deoxycytidylyl-(3′-5′)-2′-deoxyguanylyl-(3′-5′)-thymidylyl-(3′-5′)-2′-deoxyadenylyl-(3′-5′)-2′-deoxy-2′-methoxyguanylyl-(3′-5′)-2′-deoxyadenylyl-(3′-5′)-2′-deoxy-2′-methoxyguanylyl-(3′-5′)-2′-deoxycytidylyl-(3′-5′)-2′-deoxyadenylyl-(3′-5′)-2′-deoxy-2′-fluorouridylyl-(3′-5′)-2′-deoxy-2′-fluorocytidylyl-(3′-5′)-2′-deoxy-2′-methoxyadenylyl-(3′-1)-PO₃-hexa(ethyloxy)-(18-5′)-thymidylyl-(3′-5′)-2′-deoxyguanylyl-(3′-5′)-2′-deoxyadenylyl-(3′-5′)-thymidylyl-(3′-5′)-2′-deoxy-2′-fluorocytidylyl-(3′-5′)-2′-deoxy-2′-fluorocytidylyl-(3′-5′)-2′-deoxy-2′-fluorouridylyl-(3′-5′)-2′-deoxy-2′-methoxyguanylyl-(3′-3′)-thymidine.

The structure of Antagonist A is shown in FIG. 7.

The sequence of Antagonist A is:

5′-[mPEG2 40 kD]-[HN—(CH₂)₆—O] CAGGCU_{f}AC_{f}Gₘ [PO₃(CH₂CH₂O)₆] CGTAGₘAGₘCAU_{f}C_{f}Aₘ [PO₃(CH₂CH₂O)₆]TGATC_{f}C_{f}U_{f}Gₘ-[3T]-3′, whose aptamer sequence is set forth in (SEQ ID NO: 23),

where:

[3T] refers to an inverted thymidine nucleotide that is attached to the 3′ end of the oligonucleotide at the 3′ position on the ribose sugar, and [mPEG2 40 kD] represents two 20 kD polyethylene glycol (PEG) polymer chains, in one embodiment two about 20 kD PEG polymer chains, that are covalently attached to the two amino groups of a lysine residue via carbamate linkages. This moiety is in turn linked with the oligonucleotide via the amino linker described below.

[HN—(CH₂)₆—O] represents a bifunctional α-hydroxy-ω-amino linker that is covalently attached to the PEG polymer via an amide bond. The linker is attached to the oligonucleotide at the 5′-end of Antagonist A by a phosphodiester linkage.

[PO₃(CH₂CH₂O)₆] represents the hexaethylene glycol (HEX) moieties that join segments of the oligonucleotide via phosphodiester linkages. Antagonist A has two HEX linkages that join together the 9^{th} and 10^{th} nucleotides and 21^{st} and 22^{nd} nucleotides via phosphodiester linkages between the linker and the respective nucleotides.

C, A, G, and T represent the single letter code for the 2′-deoxy derivatives of cytosine, adenosine, guanosine, and thymidine nucleic acids, respectively. Antagonist A has four 2′-deoxyribocytosine, six 2′-deoxyriboadenosine, four 2′-deoxyriboguanosine, and four 2′-deoxyribothymidine.

Gₘ and Aₘ represent 2′-methoxy substituted forms of guanosine and adenosine, respectively. Antagonist A has four 2′-methoxyguanosines and one 2′-methoxyadenosine. C_{f} and U_{f} represent the 2′-fluoro substituted forms of cytosine and uridine, respectively. Antagonist A has four 2′-fluorocytosines and three 2′-fluorouridines.

The phosphodiester linkages in the oligonucleotide, with the exception of the 3′-terminus, connect the 5′- and 3′-oxygens of the ribose ring with standard nucleoside phosphodiester linkages. The phosphodiester linkage between the 3′-terminal thymidine and the penultimate Gₘ links their respective 3′-oxygens, which is referred to as the 3′,3′-cap.

Antagonist A has a molecular weight from 40,000 to 60,000 Daltons, in one embodiment from about 40,000 to about 60,000 Daltons, and can be colorless to slightly yellow in solution. Antagonist A can be present in a solution of monobasic sodium phosphate monohydrate and dibasic sodium phosphate heptahydrate as buffering agents and sodium chloride as a tonicity adjuster. Antagonist A is a hydrophilic polymer. The Antagonist A sodium salt is soluble in water and in phosphate-buffered saline (PBS), as assessed by visual inspection, to at least 50 mg (based on oligonucleotide weight)/mL solution.

In one embodiment, Antagonist A is manufactured using an iterative chemical synthesis procedure to produce the oligonucleotide portion, which is then covalently bonded to a pegylation reagent, as further described in Example 4.

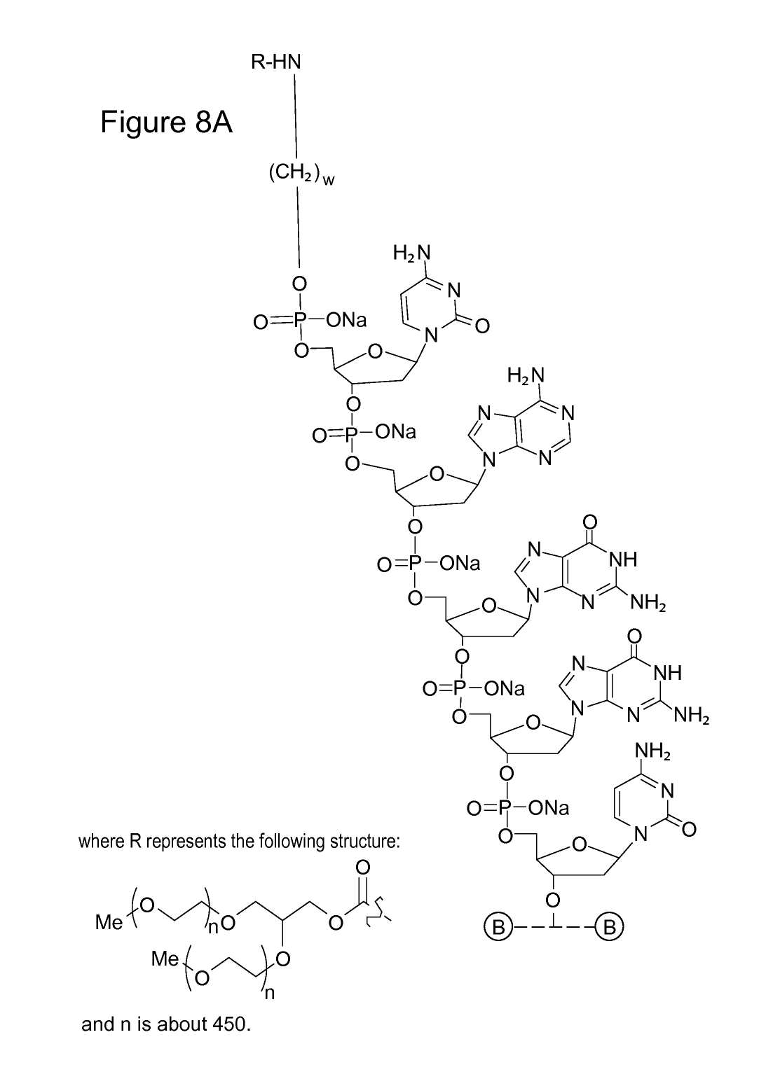

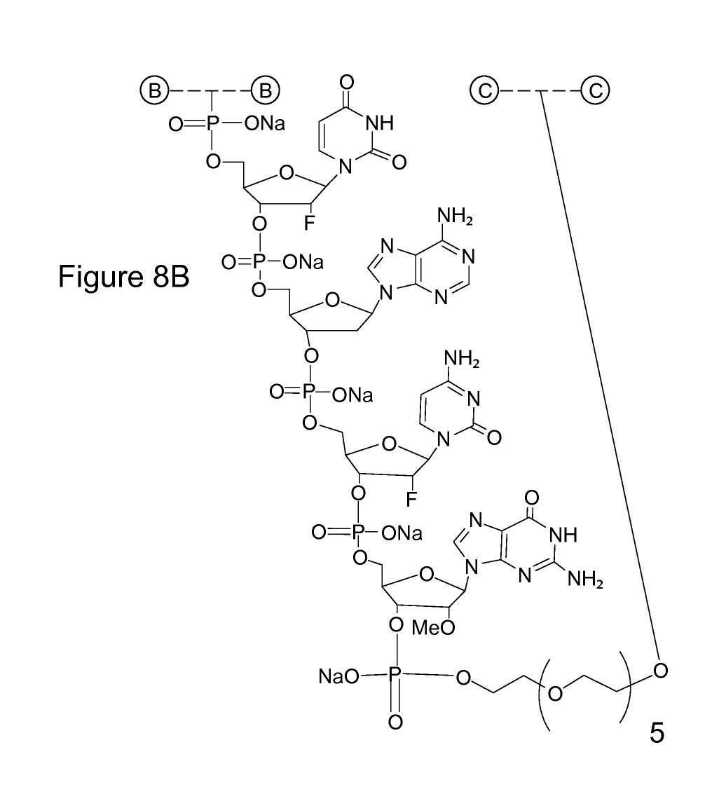

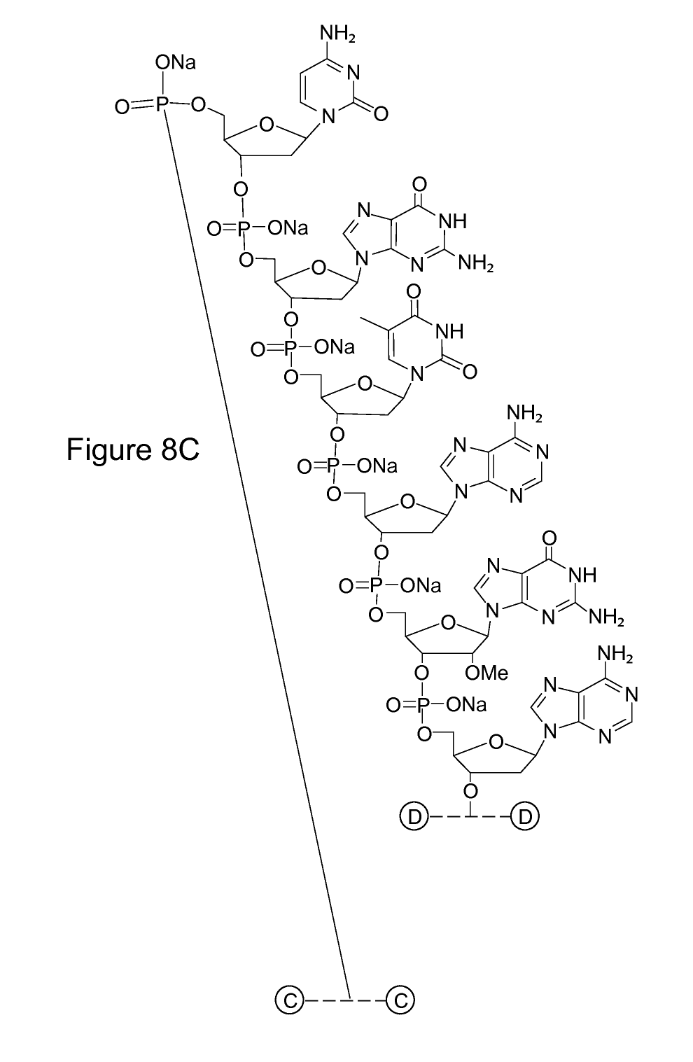

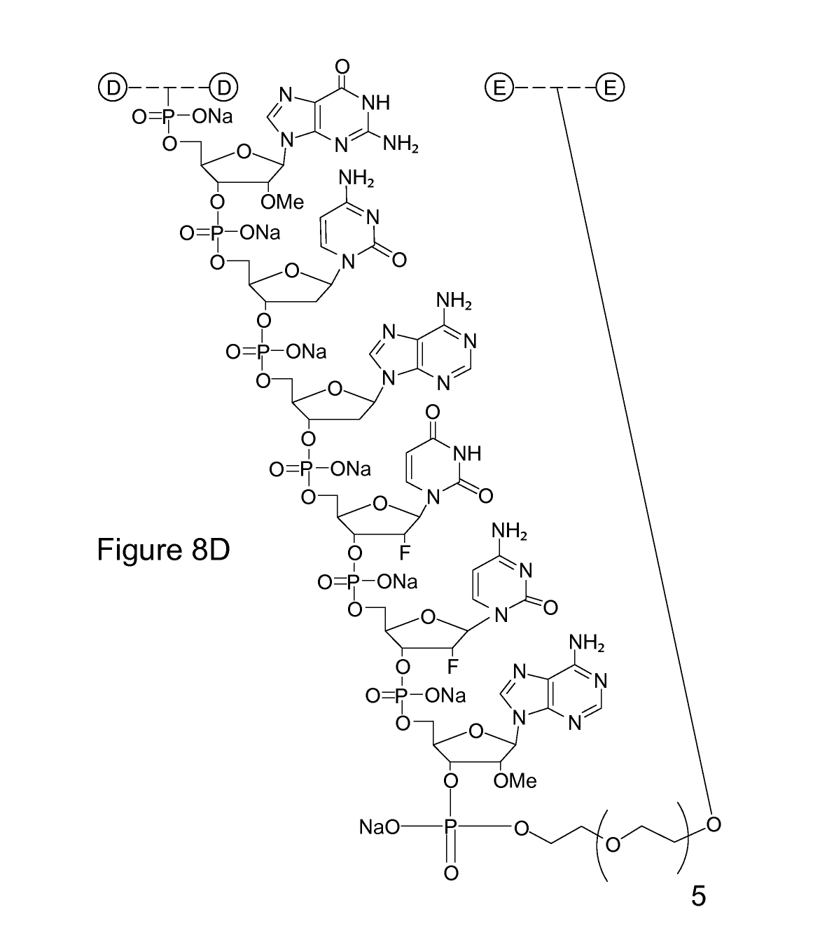

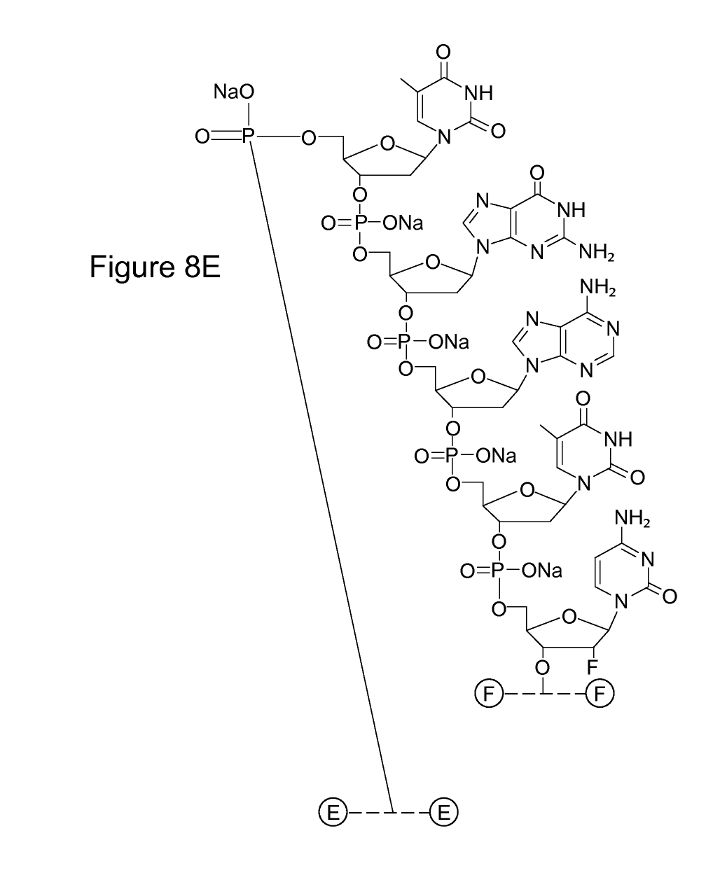

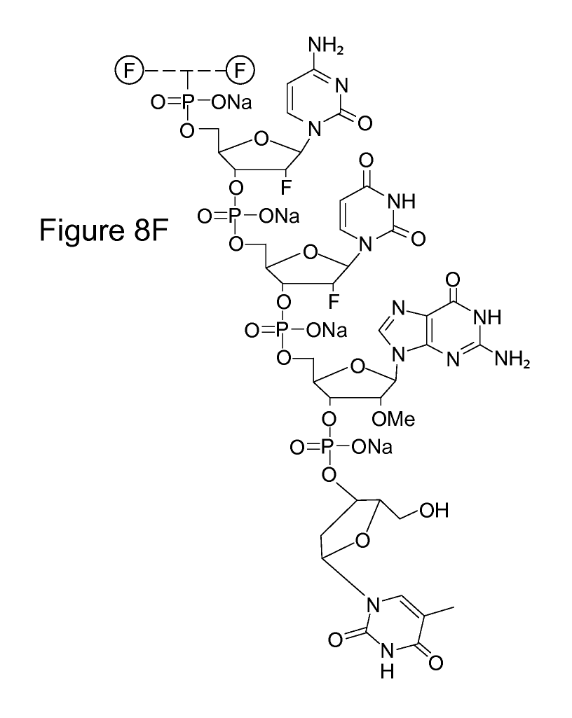

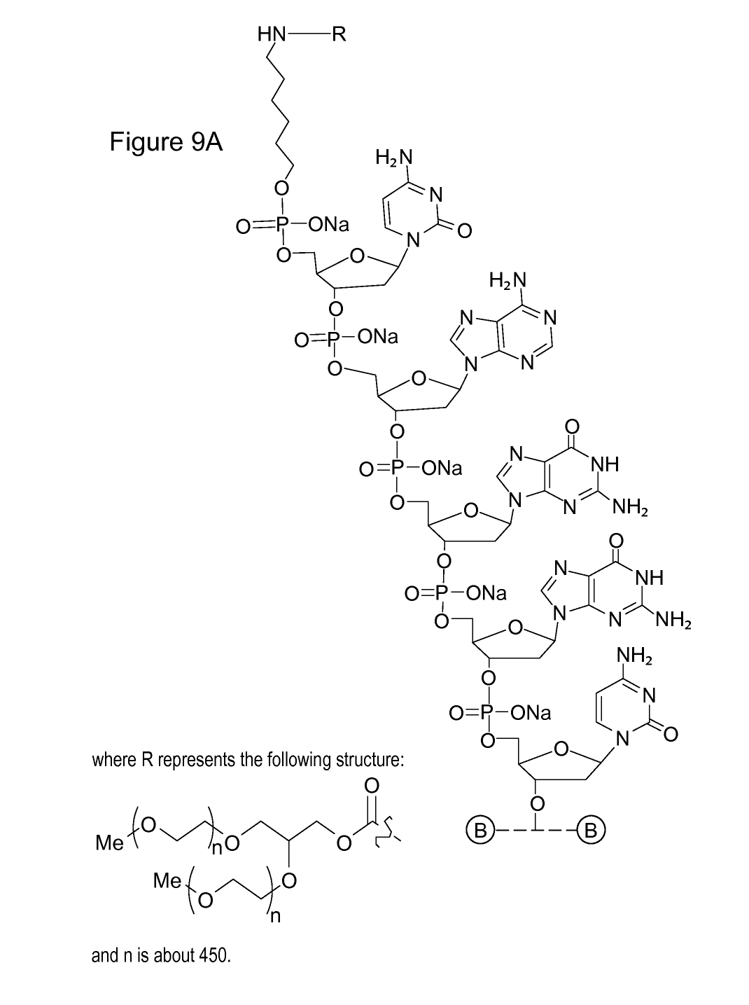

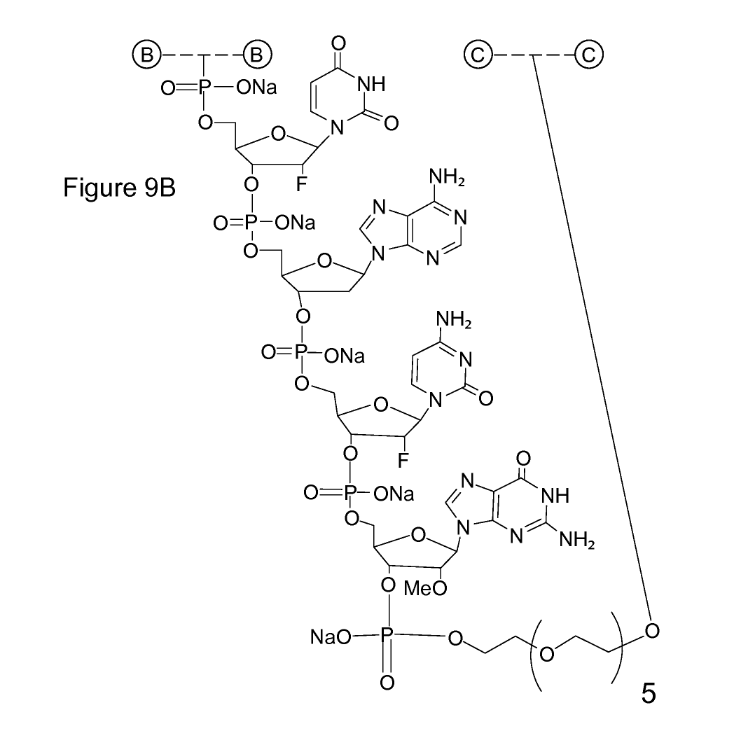

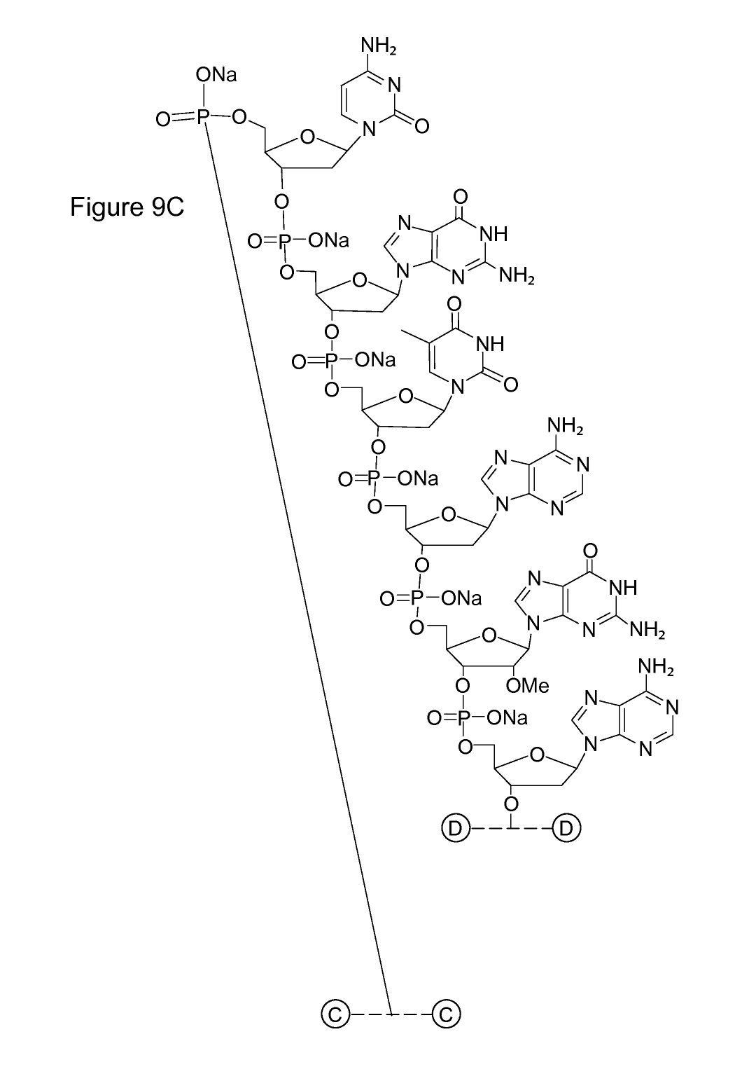

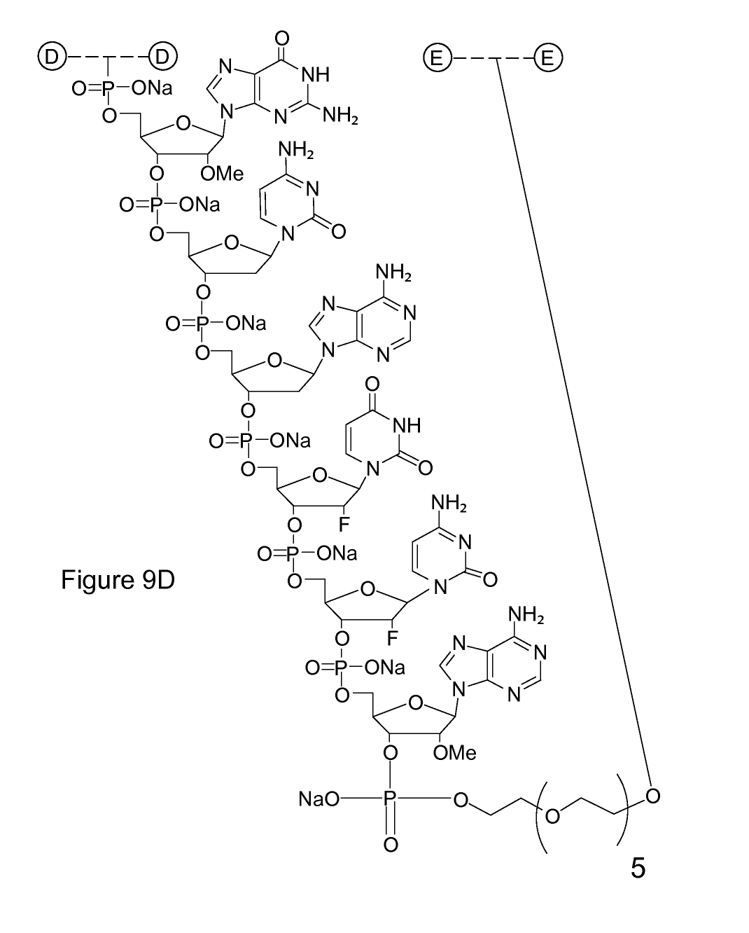

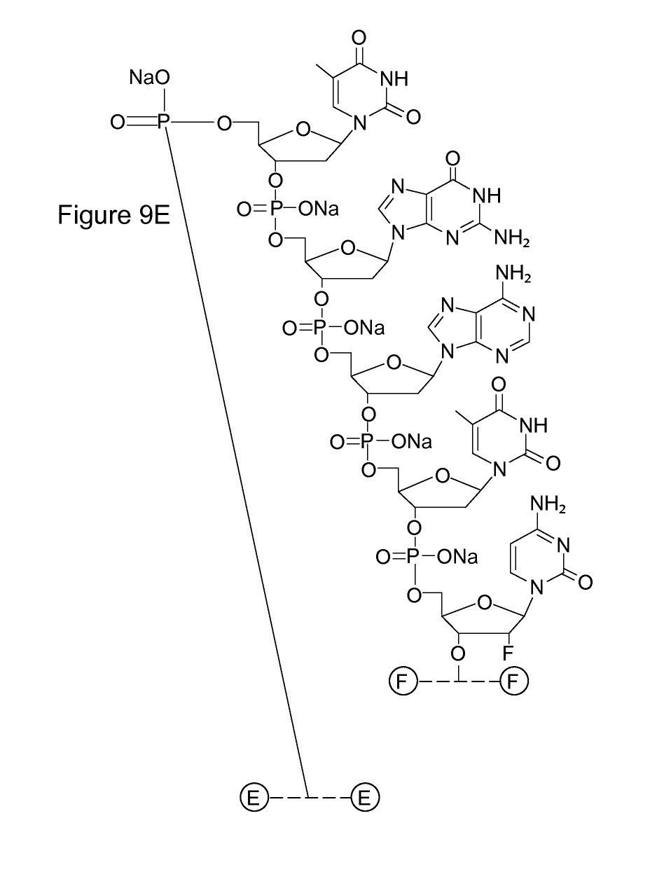

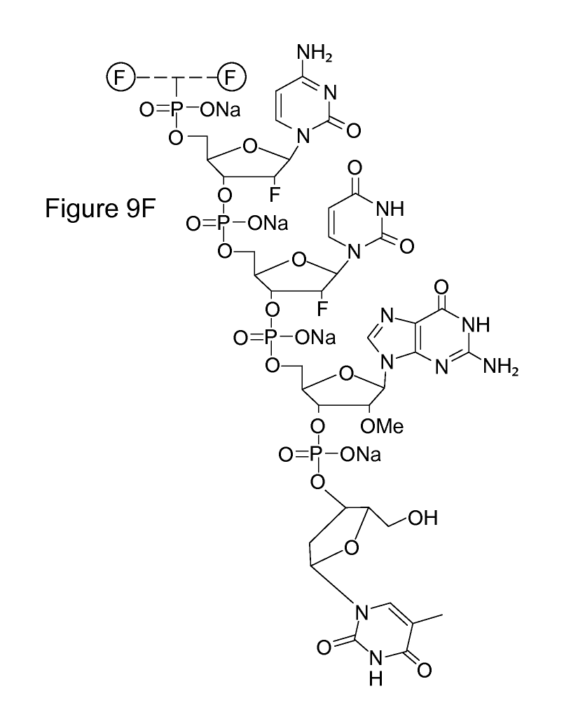

In another embodiment, the PDGF antagonist of Table 1 or 2 is a compound of Formula B (see FIG. 8), wherein w is an integer from 2 to 12. In another embodiment, the PDGF antagonist is a compound of Formula B, wherein w is an integer from 4 to 10. In another embodiment, the PDGF antagonist is a compound of Formula B, wherein w is 5, 6, 7, or 8. In one embodiment, the PDGF antagonist is a compound of Formula B, wherein w is 5. In another embodiment, the PDGF antagonist is a compound of Formula B, wherein w is 6. In another embodiment, the PDGF antagonist is a compound of Formula B, wherein w is 7. In another embodiment, the PDGF antagonist is a compound of Formula B, wherein w is 8. In one embodiment, the PDGF antagonist is a compound of Formula B having two 20 kD polyethylene glycol (PEG) polymer chains. In one embodiment, the PDGF antagonist is a compound of Formula B having an α-hydroxy-ω-amino group. In one embodiment, the α-hydroxy-ω-amino group is attached to the oligonucleotide by a phosphodiester linkage. In one embodiment, the α-hydroxy-ω-amino group is attached at the 5′-end of the oligonucleotide. In one embodiment, the PDGF antagonist is a compound of Formula B having hexaethylene glycol (HEX) moieties that join segments of the oligonucleotide via phosphodiester linkages. In one embodiment, the PDGF antagonist hexaethylene glycol (HEX) moieties join together the 9th and 10th nucleotides and 21st and 22nd nucleotides of the oligonucleotide via phosphodiester linkages between the linker and the respective nucleotides. In one embodiment, the PDGF antagonist has the structure of FIG. 9.

In another embodiment, the PDGF antagonist of Table 1 or 2 is Antagonist B or a pharmaceutically acceptable salt thereof.

The structure of Antagonist B is shown in FIG. 9.

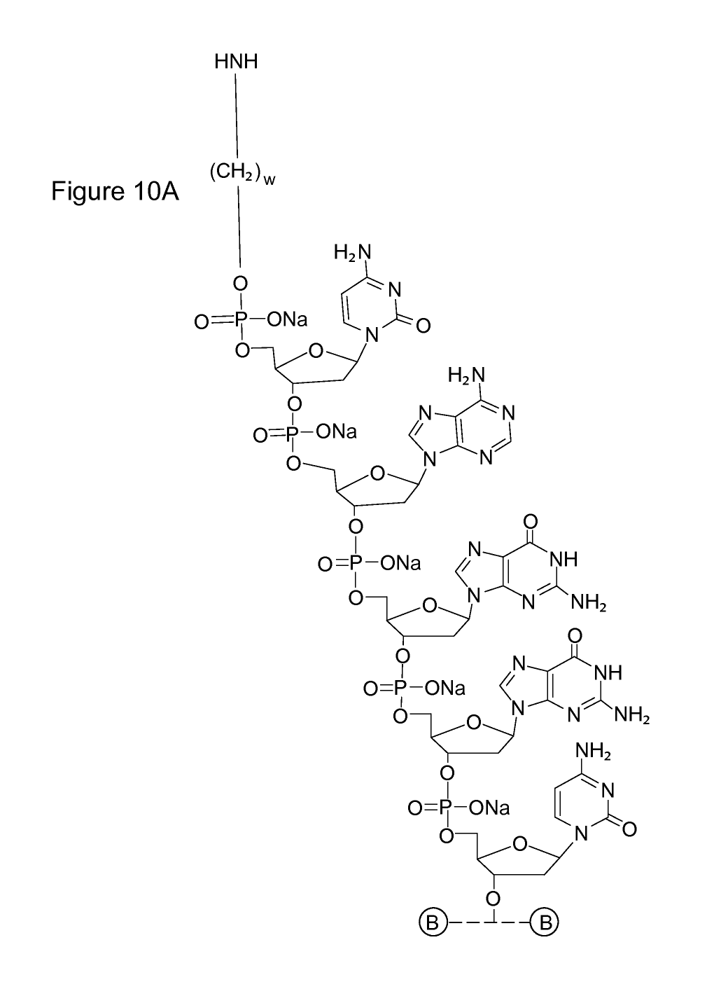

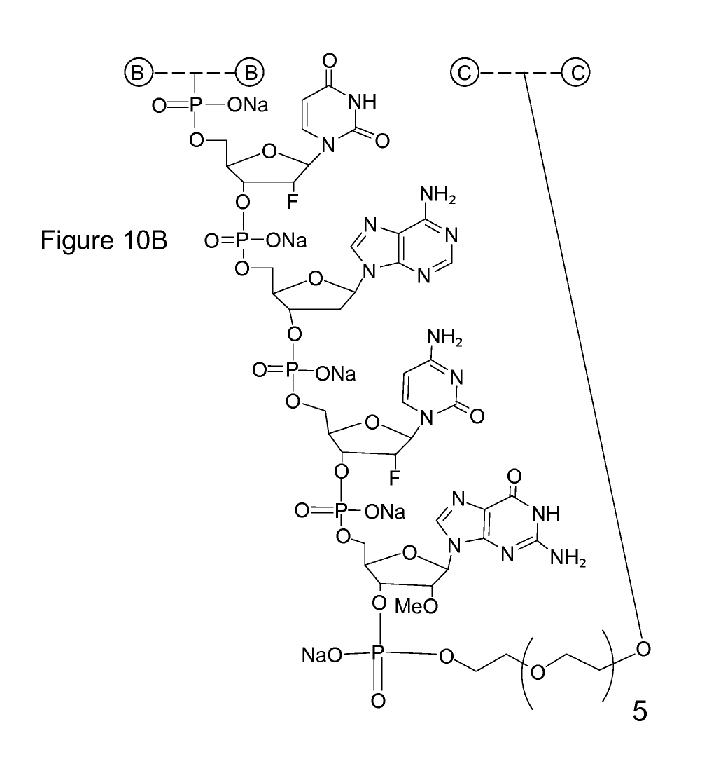

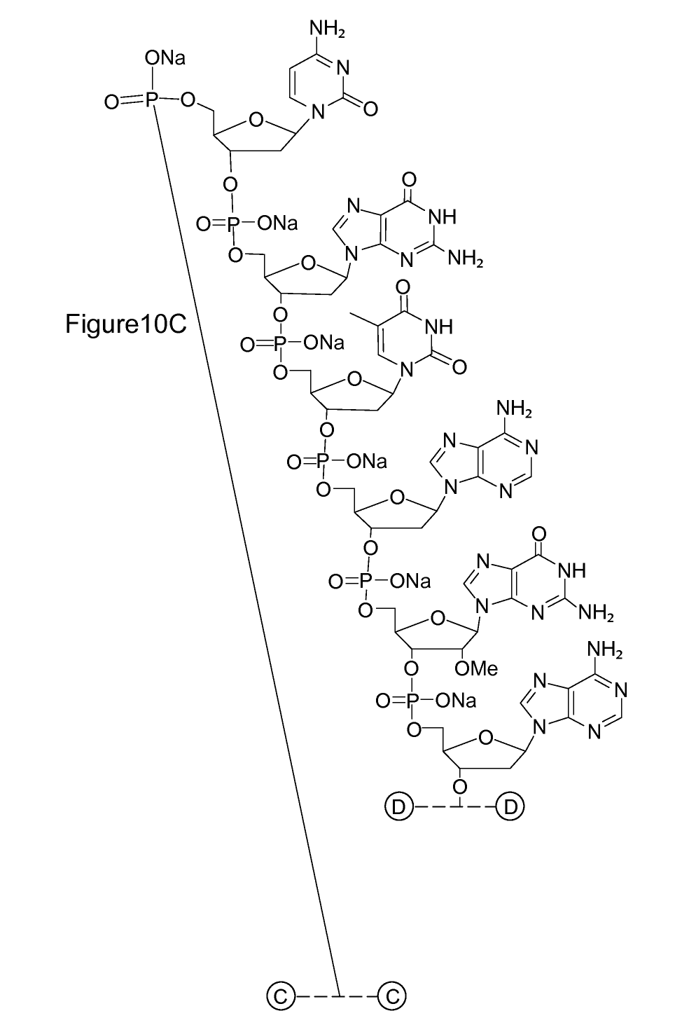

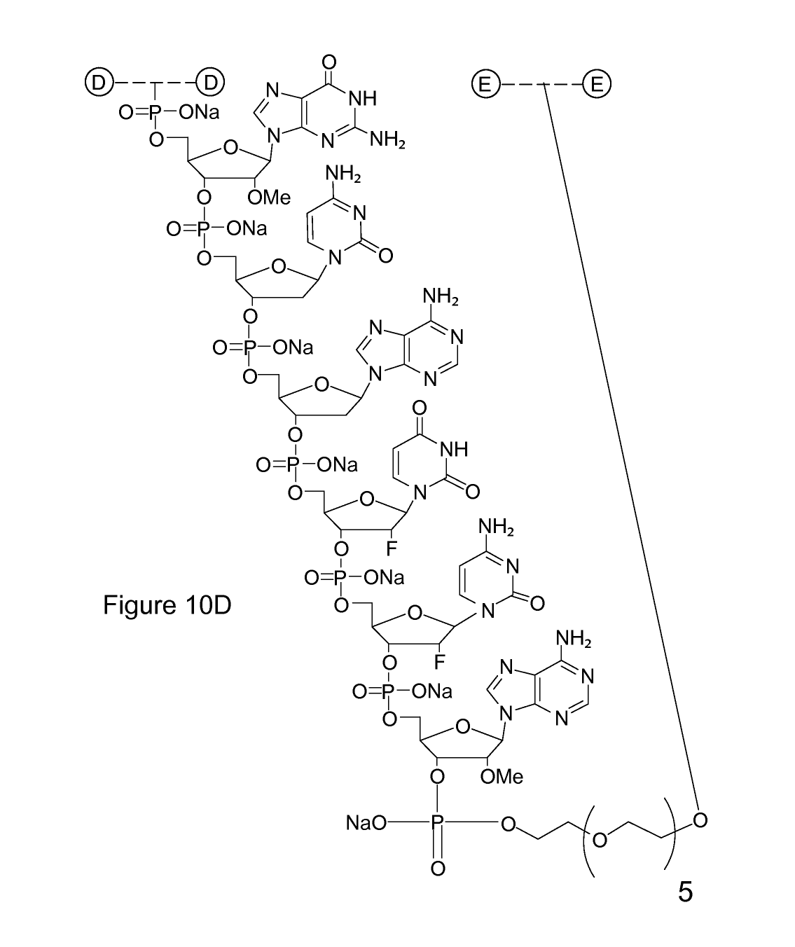

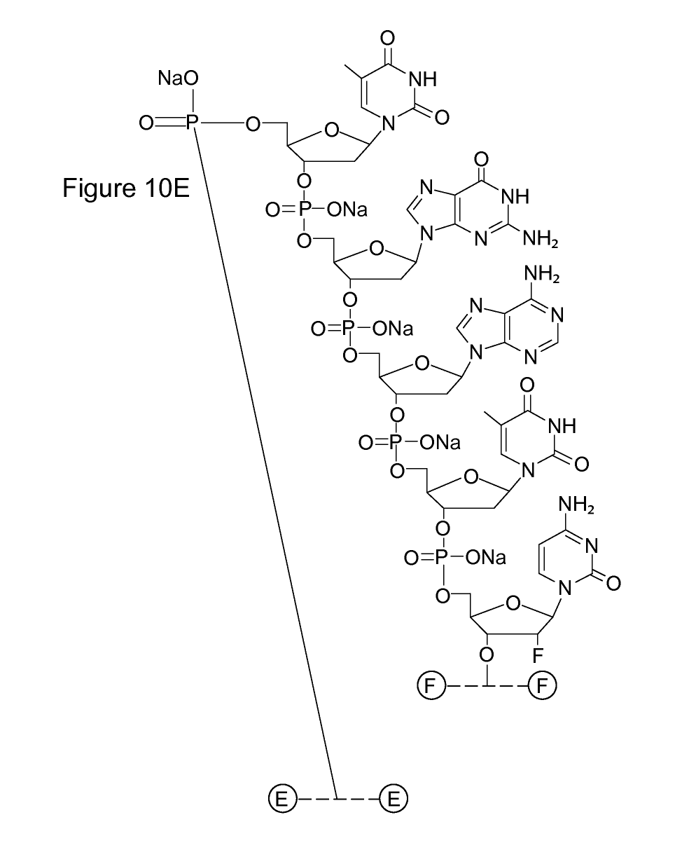

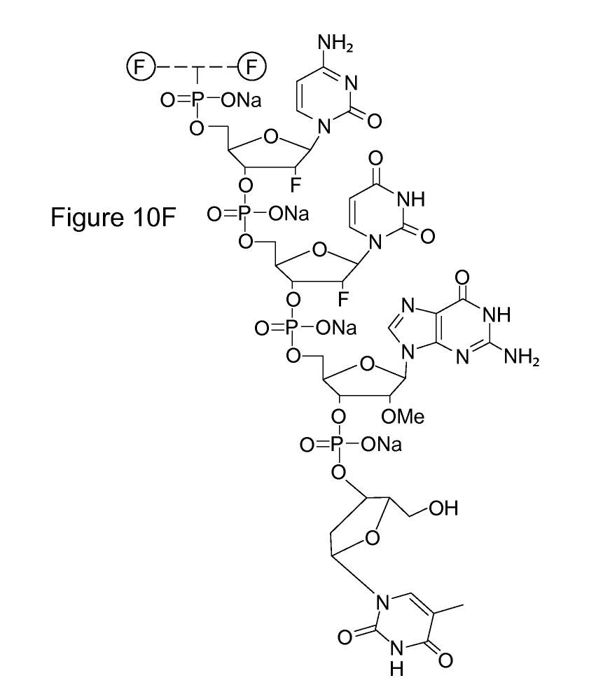

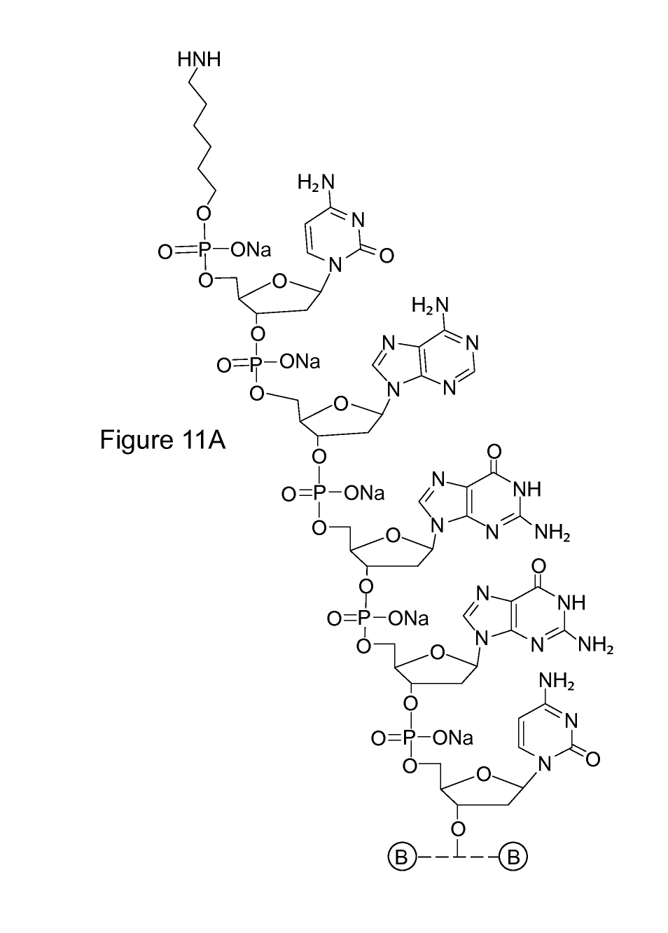

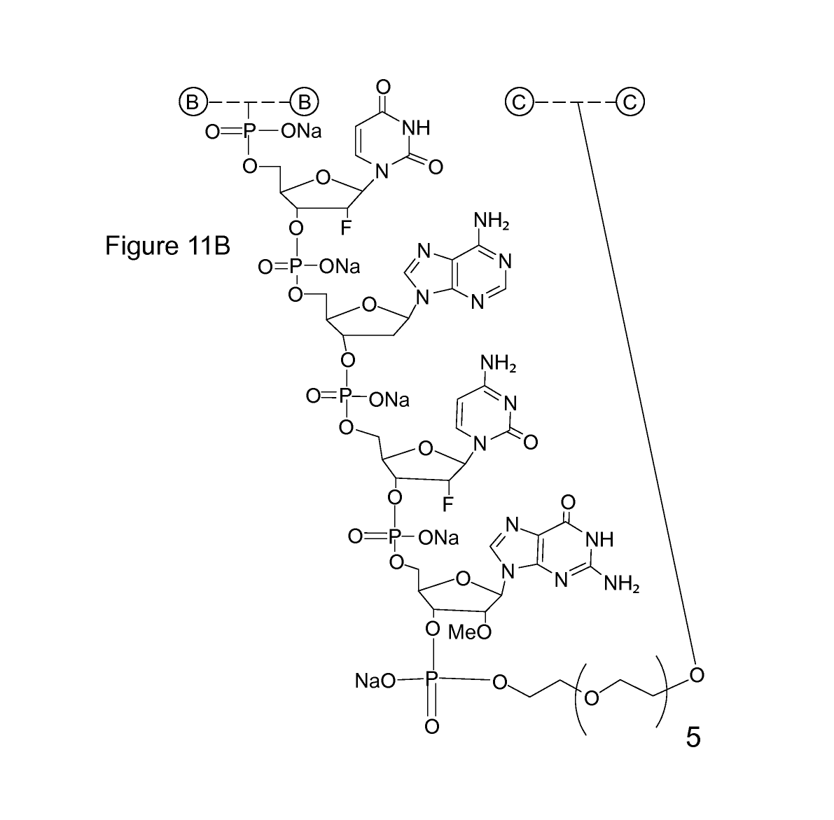

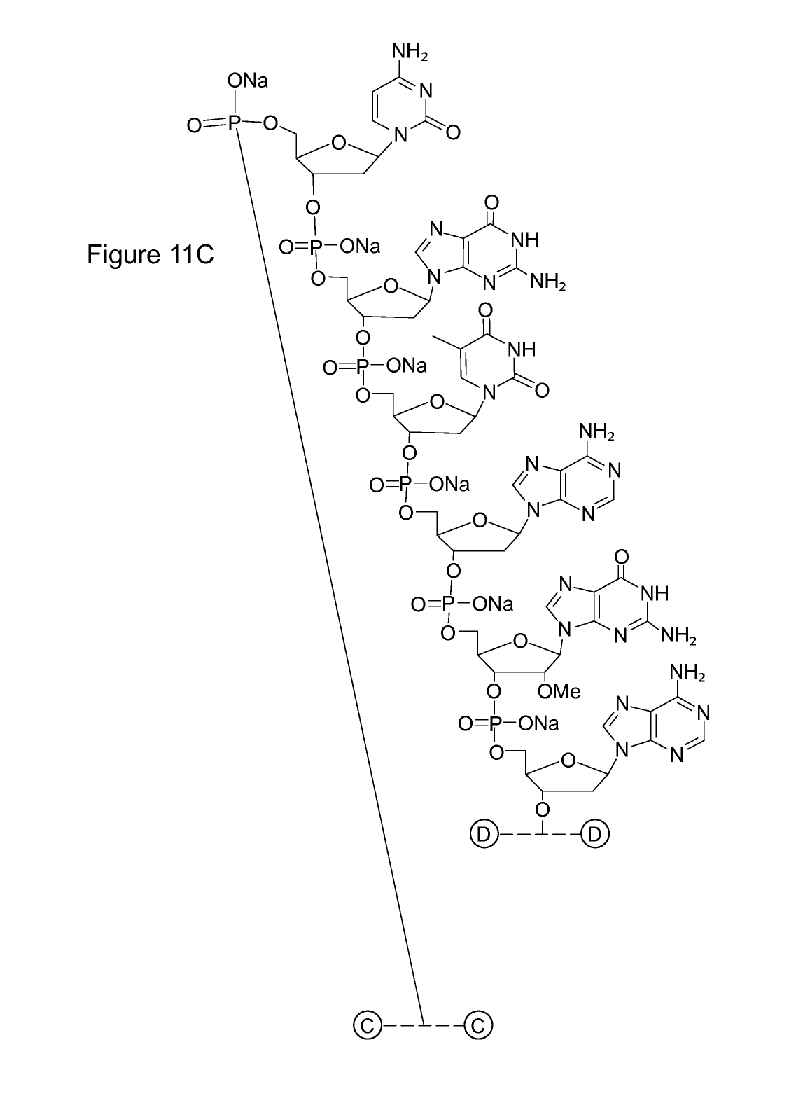

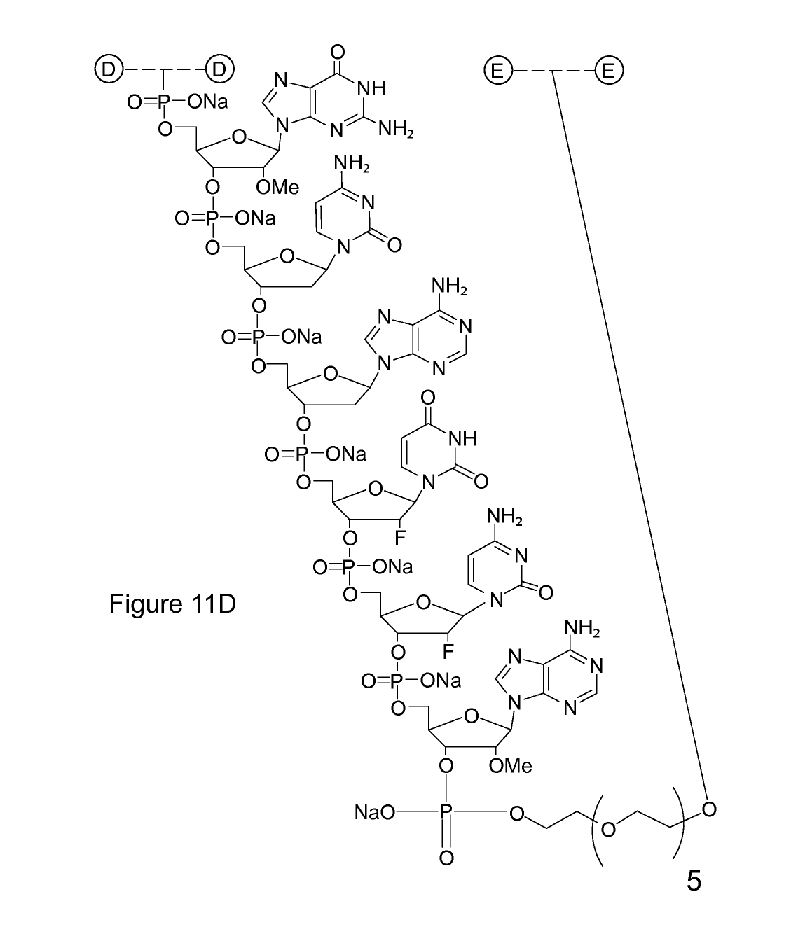

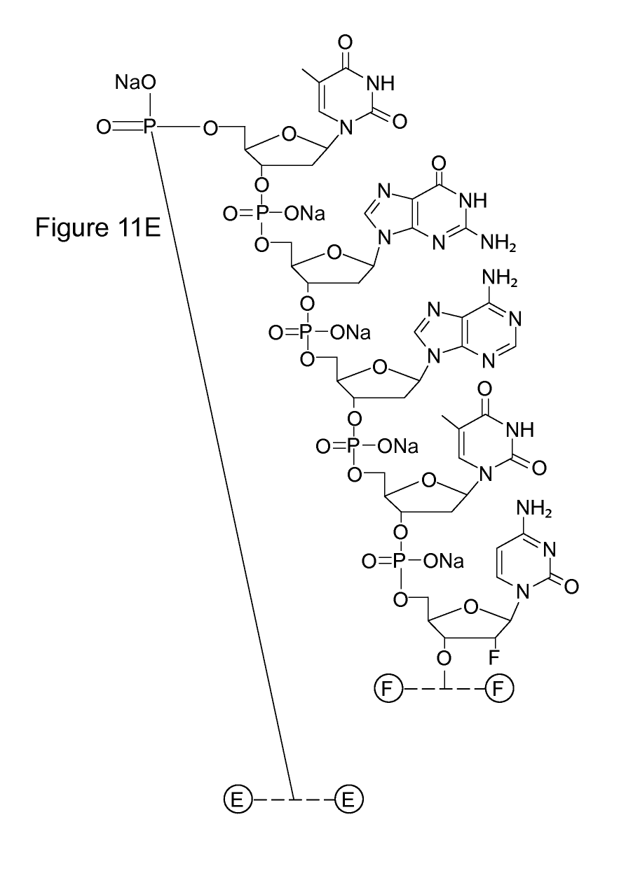

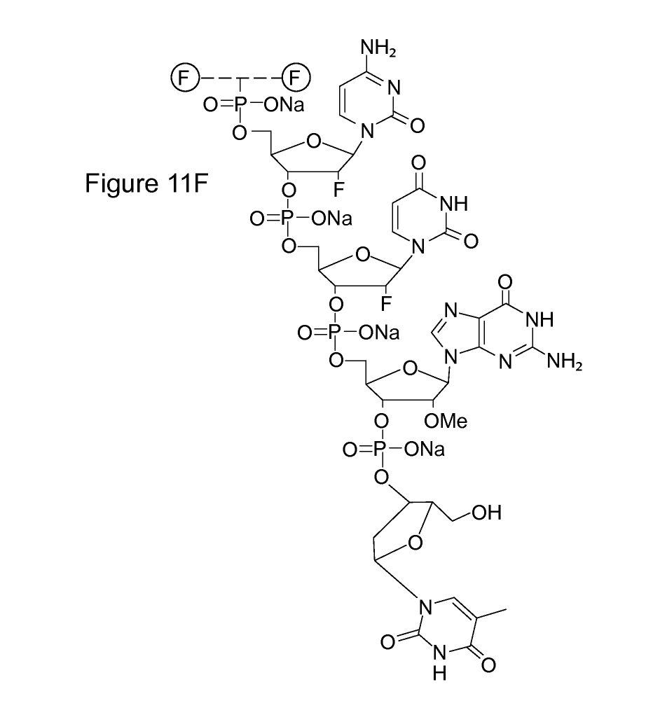

In another embodiment, the PDGF antagonist of Table 1 or 2 is a compound of Formula C (see FIG. 10), wherein w is an integer from 2 to 12. In another embodiment, the PDGF antagonist is a compound of Formula C, wherein w is an integer from 4 to 10. In another embodiment, the PDGF antagonist is a compound of Formula C, wherein w is 5, 6, 7, or 8. In one embodiment, the PDGF antagonist is a compound of Formula C, wherein w is 5. In another embodiment, the PDGF antagonist is a compound of Formula C, wherein w is 6. In another embodiment, the PDGF antagonist is a compound of Formula C, wherein w is 7. In another embodiment, the PDGF antagonist is a compound of Formula C, wherein w is 8. In one embodiment, the PDGF antagonist is a compound of Formula C having an α-hydroxy-ω-amino group. In one embodiment, the α-hydroxy-ω-amino group is attached to the oligonucleotide by a phosphodiester linkage. In one embodiment, the α-hydroxy-ω-amino group is attached at the 5′-end of the oligonucleotide. In one embodiment, the PDGF antagonist is a compound of Formula C having hexaethylene glycol (HEX) moieties that join segments of the oligonucleotide via phosphodiester linkages. In one embodiment, the PDGF antagonist hexaethylene glycol (HEX) moieties join together the 9th and 10th nucleotides and 21st and 22nd nucleotides of the oligonucleotide via phosphodiester linkages between the linker and the respective nucleotides. In one embodiment, the PDGF antagonist has the structure of FIG. 11.

In another embodiment, the PDGF antagonist of Table 1 or 2 is Antagonist C or a pharmaceutically acceptable salt thereof.

The structure of Antagonist C is shown in FIG. 11.

The phosphodiester linkages in the oligonucleotide, with the exception of the 3′-terminus, connect the 5′- and 3′-oxygens of the ribose ring with standard nucleoside phosphodiester linkages. The phosphodiester linkage between the 3′-terminal thymidine and the penultimate Gₘ links their respective 3′-oxygens, which is referred to as the 3′,3′-cap.

In another embodiment, the PDGF antagonist of Table 1 or 2 is Antagonist D or a pharmaceutically acceptable salt thereof.

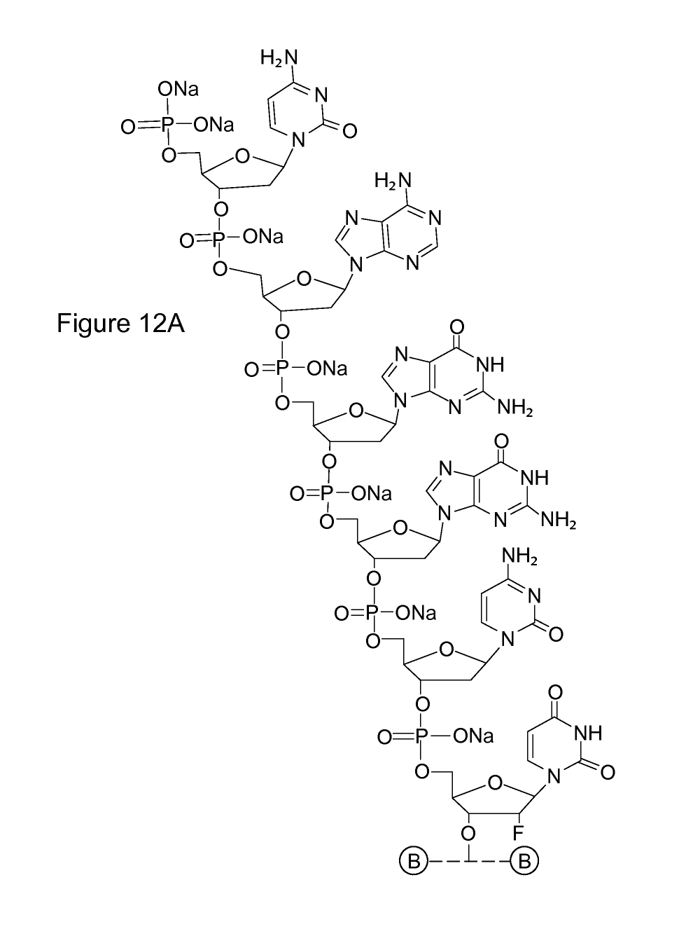

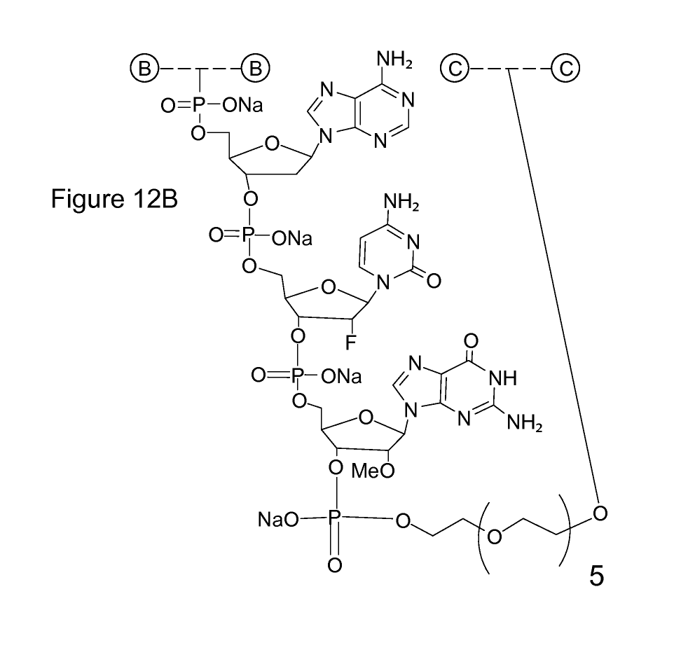

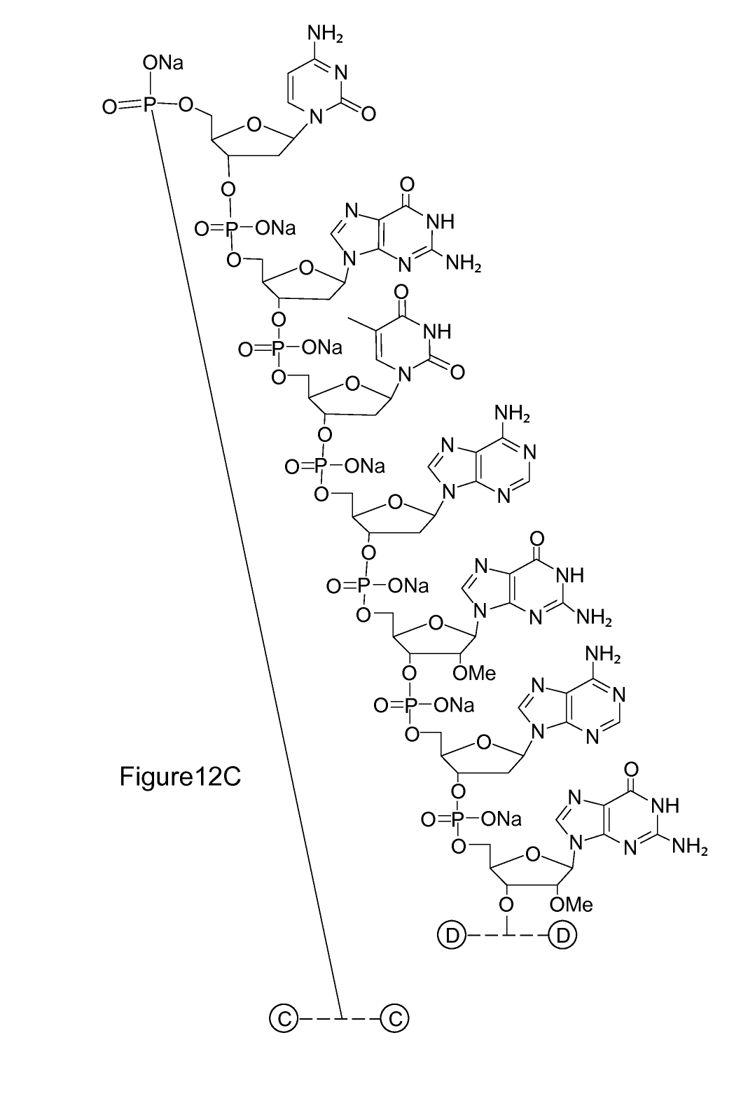

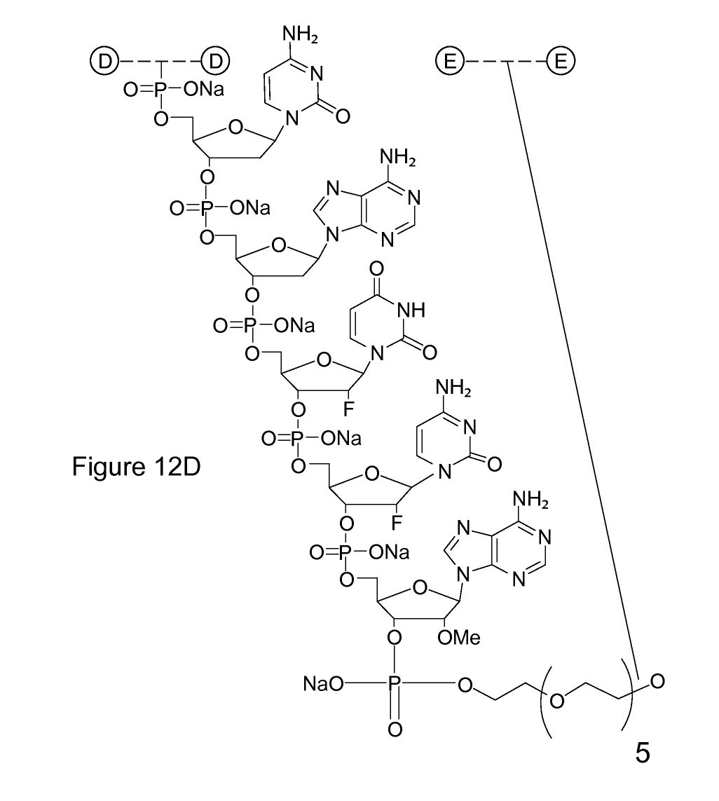

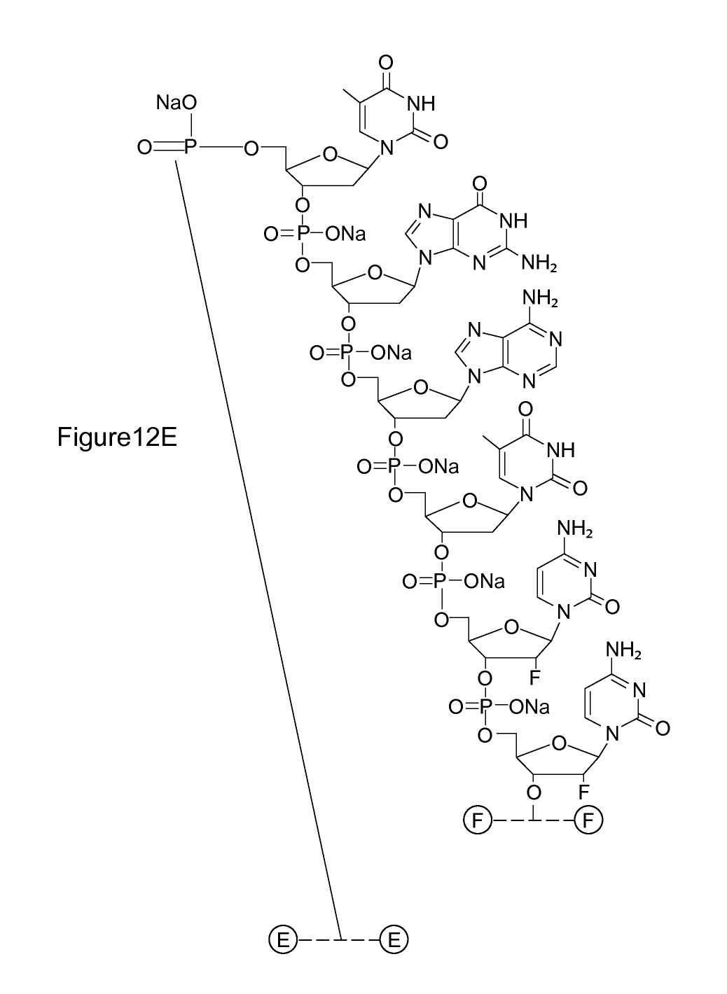

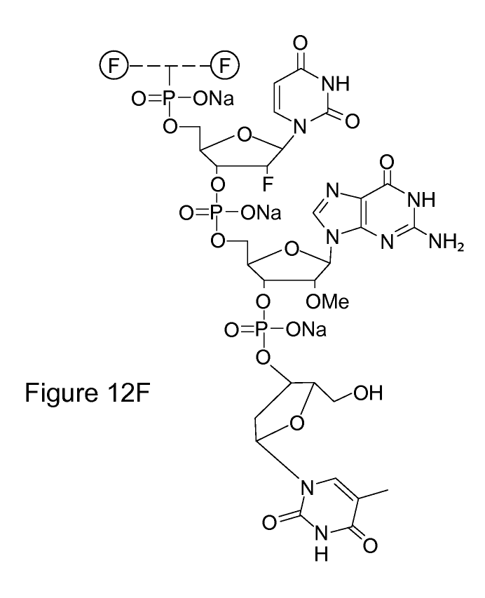

The structure of Antagonist D is shown in FIG. 12.

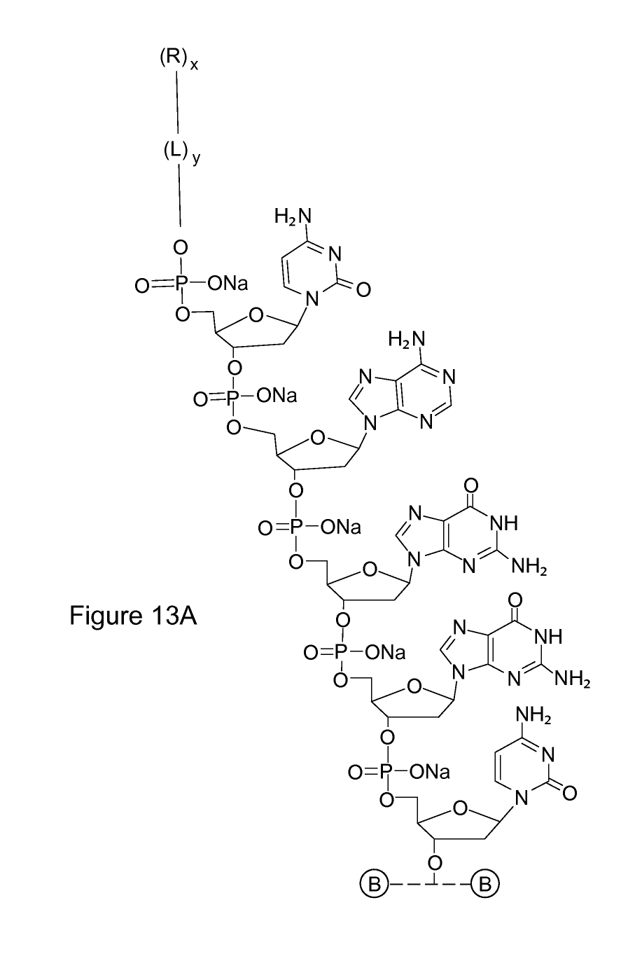

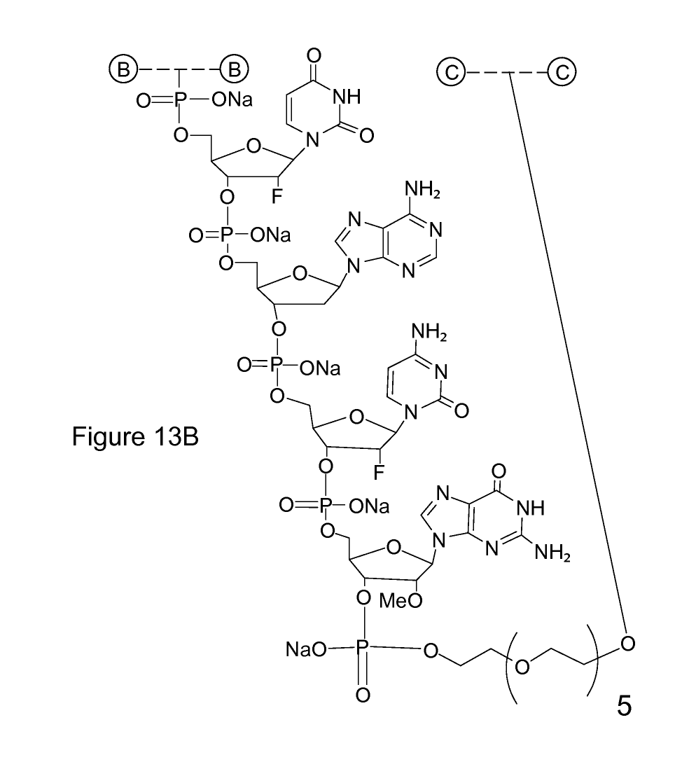

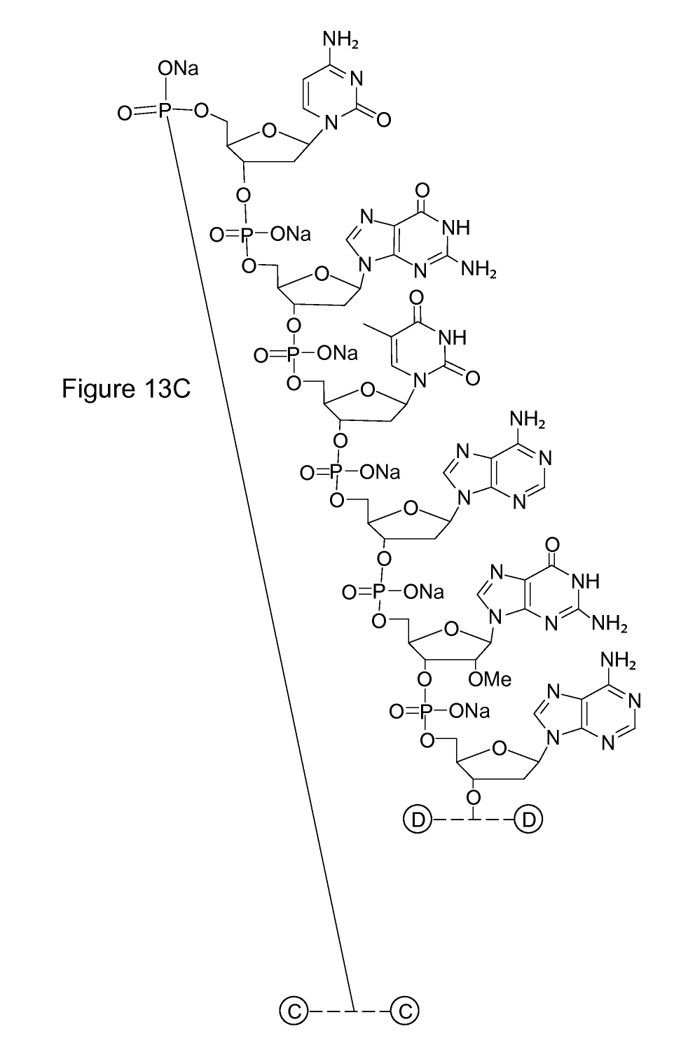

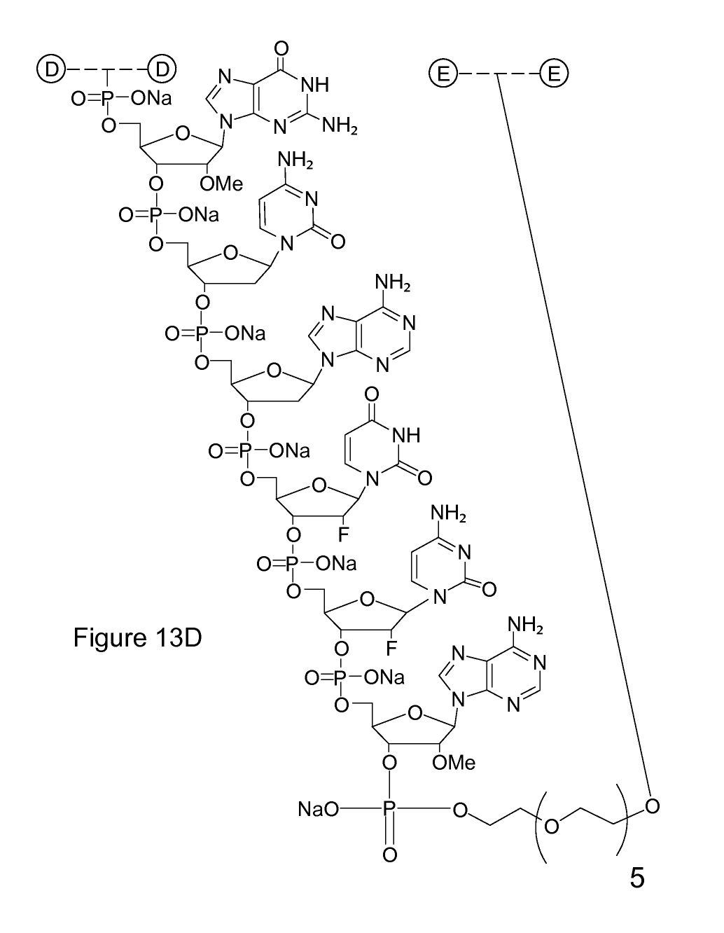

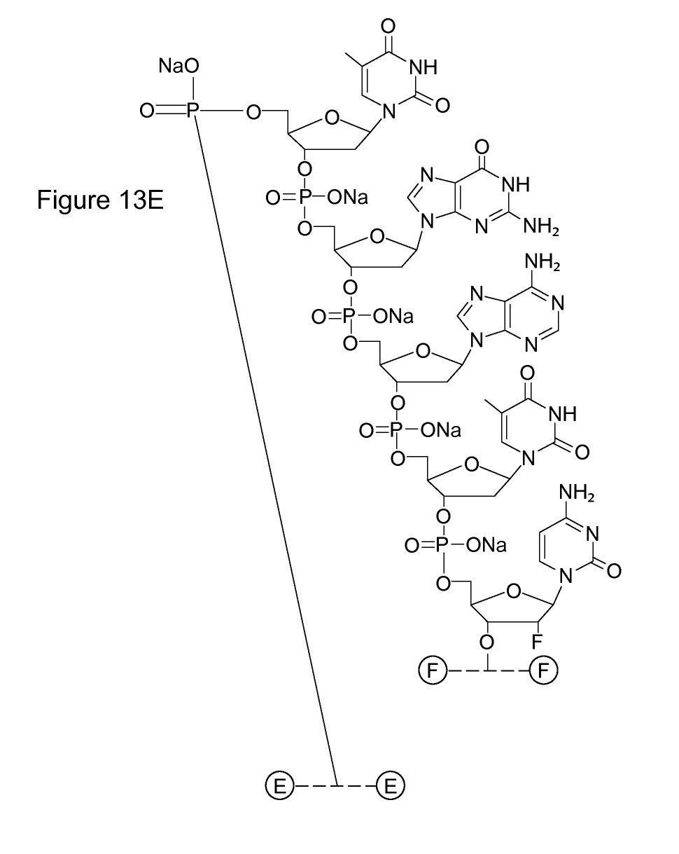

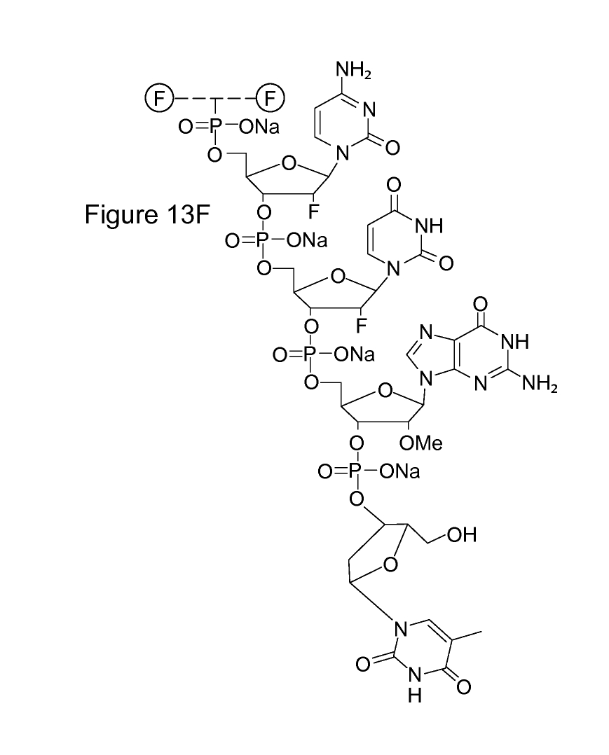

In another embodiment, the PDGF antagonist of Table 1 or 2 is a compound of Formula E (see FIG. 13), wherein L is a linker, Y is 0 or 1, R is a nonphysiologically active group, lipophilic group or High Molecular Weight Compound, and X is an integer ranging from 1 to 4.

In another embodiment, the PDGF antagonist of Table 1 or 2 is the antibody 1B3 or a pharmaceutically acceptable salt thereof (US Patent Publication No. 20090053241 (paragraph 0073 and Table 1), which is hereby incorporated by reference in its entirety).

In another embodiment, the PDGF antagonist of Table 1 or 2 is the antibody CDP860 or a pharmaceutically acceptable salt thereof (Serruys et al. (2003) Int. J. Cardiovasc Intervent. 5:214-22, which is hereby incorporated by reference in its entirety).

In another embodiment, the PDGF antagonist of Table 1 or 2 is the antibody IMC-3G3 or a pharmaceutically acceptable salt thereof (Dolloff et al. (2007) Cancer Res. 67:555-62, which is hereby incorporated by reference in its entirety).

In one embodiment, the PDGF antagonist of Table 1 or 2 is imatinib or a pharmaceutically acceptable salt thereof. A composition comprising imatinib mesylate is commercially available under the trademark Gleevec.

In another embodiment, the PDGF antagonist of Table 1 or 2 is the antibody 162.62 or a pharmaceutically acceptable salt thereof (U.S. Pat. No. 5,976,534, which is hereby incorporated by reference in its entirety).

In another embodiment, the PDGF antagonist of Table 1 or 2 is the antibody 163.31 or a pharmaceutically acceptable salt thereof (U.S. Pat. No. 5,976,534, which is hereby incorporated by reference in its entirety).

In another embodiment, the PDGF antagonist of Table 1 or 2 is the antibody 169.14 or a pharmaceutically acceptable salt thereof (U.S. Pat. No. 5,976,534, which is hereby incorporated by reference in its entirety).

In another embodiment, the PDGF antagonist of Table 1 or 2 is the antibody 169.31 or a pharmaceutically acceptable salt thereof (U.S. Pat. No. 5,976,534, which is hereby incorporated by reference in its entirety).

In another embodiment, the PDGF antagonist of Table 1 or 2 is the antibody αR1 or a pharmaceutically acceptable salt thereof (U.S. Pat. No. 5,833,986 (Column 4, lines 46-51), which is hereby incorporated by reference in its entirety).

In another embodiment, the PDGF antagonist of Table 1 or 2 is the antibody 2A1E2 or a pharmaceutically acceptable salt thereof (U.S. Pat. No. 5,817,310 (Column 11, lines 52-59), which is hereby incorporated by reference in its entirety).

In another embodiment, the PDGF antagonist of Table 1 or 2 is the antibody M4TS.11 or a pharmaceutically acceptable salt thereof (U.S. Pat. No. 5,882,644 (FIG. 7), which is hereby incorporated by reference in its entirety).

In another embodiment, the PDGF antagonist of Table 1 or 2 is the antibody M4TS.22 or a pharmaceutically acceptable salt thereof (U.S. Pat. No. 5,882,644 (FIG. 1), which is hereby incorporated by reference in its entirety).

In another embodiment, the PDGF antagonist of Table 1 or 2 is A10 or a pharmaceutically acceptable salt thereof (U.S. Pat. No. 6,331,555 (FIG. 1), which is hereby incorporated by reference in its entirety).

In another embodiment, the PDGF antagonist of Table 1 or 2 is brefeldin A or a pharmaceutically acceptable salt thereof (U.S. Pat. No. 5,618,837 (Column 2, lines 15-19), which is hereby incorporated by reference in its entirety).

In another embodiment, the PDGF antagonist of Table 1 or 2 is sunitinib or a pharmaceutically acceptable salt thereof. A composition comprising sunitinib malate is commercially available under the trademark Sutent.

In another embodiment, the PDGF antagonist of Table 1 or 2 is the antibody Hyb 120.1.2.1.2 or a pharmaceutically acceptable salt thereof (U.S. Pat. No. 5,094,941 (Example VI, col. 32, lines 1-15), which is hereby incorporated by reference in its entirety).

In another embodiment, the PDGF antagonist of Table 1 or 2 is the antibody Hyb 121.6.1.1.1 or a pharmaceutically acceptable salt thereof (U.S. Pat. No. 5,094,941 (Example VI, col. 32, lines 1-15), which is hereby incorporated by reference in its entirety).

In another embodiment, the PDGF antagonist of Table 1 or 2 is the antibody Hyb 127.5.7.3.1 or a pharmaceutically acceptable salt thereof (U.S. Pat. No. 5,094,941 (Example VII, col. 33, lines 1-15), which is hereby incorporated by reference in its entirety).

In another embodiment, the PDGF antagonist of Table 1 or 2 is the antibody Hyb 127.8.2.2.2 or a pharmaceutically acceptable salt thereof (U.S. Pat. No. 5,094,941 (Example VII, col. 33, lines 1-15), which is hereby incorporated by reference in its entirety).

In another embodiment, the PDGF antagonist of Table 1 or 2 is the antibody Hyb 1.6.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.11.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.17.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.18.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.19.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.23.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.24 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.25 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.29 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.33 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.38 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.39 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.40 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.45 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.46 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.48 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.49 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 1.51 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody Hyb 6.4.1 or a pharmaceutically acceptable salt thereof (U.S. Pat. No. 7,135,174 (col. 32, lines 34-42), which is hereby incorporated by reference in its entirety).

In another embodiment, the PDGF antagonist of Table 1 or 2 is the antibody F3 or a pharmaceutically acceptable salt thereof (US Patent Publication No. 20030219839 (paragraph 144), which is hereby incorporated by reference in its entirety).

In another embodiment, the PDGF antagonist of Table 1 or 2 is the antibody Humanized F3 or a pharmaceutically acceptable salt thereof (US Patent Publication No. 20030219839 (paragraph 153-183), which is hereby incorporated by reference in its entirety).

In another embodiment, the PDGF antagonist of Table 1 or 2 is the antibody C1 or a pharmaceutically acceptable salt thereof (US Patent Publication No. 20030219839 (paragraph 192-196), which is hereby incorporated by reference in its entirety).

In another embodiment, the PDGF antagonist of Table 1 or 2 is the antibody Humanized C1 or a pharmaceutically acceptable salt thereof (US Patent Publication No. 20030219839 (paragraph 197-199), which is hereby incorporated by reference in its entirety).

In another embodiment, the PDGF antagonist of Table 1 or 2 is the antibody 6.4.1 or a pharmaceutically acceptable salt thereof (US Patent Publication No. 20040141969 (Example 4, paragraph 192-197), which is hereby incorporated by reference in its entirety).

In another embodiment, the PDGF antagonist of Table 1 or 2 is the anti-mPDGF-C goat IgG antibody or a pharmaceutically acceptable salt thereof (Crawford et al. (2009) Cancer Cell 15:21-34, which is hereby incorporated by reference in its entirety).

In another embodiment, the PDGF antagonist of Table 1 or 2 is the antibody C3.1 or a pharmaceutically acceptable salt thereof (Kawahara et al. (1987) Biochem. Biophys. Res. Commun. 147:839-845, which is hereby incorporated by reference in its entirety).

In another embodiment, the PDGF antagonist of Table 1 or 2 is 5-methyl-7-diethylamino-s-triazolo (1,5-a) pyrimidine or a pharmaceutically acceptable salt thereof (Ohnishi et al. (1983) Life Sci. 31:2595-2602, which is hereby incorporated by reference in its entirety).

In another embodiment, the PDGF antagonist of Table 1 or 2 is interferon or a pharmaceutically acceptable salt thereof (Zagari et al. (1988) Biochem. Biophys 150:1207-12, which is hereby incorporated by reference in its entirety).

In another embodiment, the PDGF antagonist of Table 1 or 2 is protamine or a pharmaceutically acceptable salt thereof (Huang (1984) J. Cell. Biol. 26:205-220, which is hereby incorporated by reference in its entirety).

In another embodiment, the PDGF antagonist of Table 1 or 2 is the monoclonal antibody PDGFR-B1 or a pharmaceutically acceptable salt thereof (Ronnestrand, L. and Terracio, L. (1988) J. Biol. Chem. 263: 10429-10435, which is hereby incorporated by reference in its entirety).

In another embodiment, the PDGF antagonist of Table 1 or 2 is the monoclonal antibody PDGFR-B2 or a pharmaceutically acceptable salt thereof (Ronnestrand, L. and Terracio, L. (1988) J. Biol. Chem. 263: 10429-10435, which is hereby incorporated by reference in its entirety).

In another embodiment, the PDGF antagonist of Table 1 or 2 is the monoclonal antibody 6D11 or a pharmaceutically acceptable salt thereof (Vassbotn et al. (1990) Biochim. Biophy. Acta, 1054: 246-249, which is hereby incorporated by reference in its entirety).

In another embodiment, the PDGF antagonist of Table 1 or 2 is the monoclonal antibody Sis 1 or a pharmaceutically acceptable salt thereof (La Rochelle et al. (1989) Mol. Cell. Bio., 9: 3538-3542, which is hereby incorporated by reference in its entirety).

In another embodiment, the PDGF antagonist of Table 1 or 2 is the monoclonal antibody PR7212 or a pharmaceutically acceptable salt thereof (Seifert et al. (1989) J. Biol. Chem. 264: 8771-8778, which is hereby incorporated by reference in its entirety).

In another embodiment, the PDGF antagonist of Table 1 or 2 is the monoclonal antibody PR292 or a pharmaceutically acceptable salt thereof (La Rochelle et al. (1993) Cell Growth Differ. 4:547-53, which is hereby incorporated by reference in its entirety).

In another embodiment, the PDGF antagonist of Table 1 or 2 is the monoclonal antibody HYB 9610 or a pharmaceutically acceptable salt thereof (EP0798002 (see para (0023)), which is hereby incorporated by reference in its entirety).

In another embodiment, the PDGF antagonist of Table 1 or 2 is the monoclonal antibody HYB 9611 or a pharmaceutically acceptable salt thereof (EP0798002 (see para (0023)), which is hereby incorporated by reference in its entirety).

In another embodiment, the PDGF antagonist of Table 1 or 2 is the monoclonal antibody HYB 9612 or a pharmaceutically acceptable salt thereof (EP0798002 (see para (0023)), which is hereby incorporated by reference in its entirety).

In another embodiment, the PDGF antagonist of Table 1 or 2 is the monoclonal antibody HYB 9613 or a pharmaceutically acceptable salt thereof (EP0798002 (see para (0023)), which is hereby incorporated by reference in its entirety).

In another embodiment, the PDGF antagonist of Table 1 or 2 is 4-(2-(N-(-2-carboxamidoindole)aminoethyl)-benzenesulfonamide or a pharmaceutically acceptable salt thereof (EP0835115, which is hereby incorporated by reference in its entirety).

In another embodiment, the PDGF antagonist of Table 1 or 2 is 4-(2-(N-(-2-carboxamidoindole)aminoethyl)-sulfonylurea or a pharmaceutically acceptable salt thereof (EP0835115, which is hereby incorporated by reference in its entirety).

In another embodiment, the PDGF antagonist of Table 1 or 2 is CGP 53716 or a pharmaceutically acceptable salt thereof (Buchdunger, et al. (1995) Proc. Natl. Acad. Sci.; 92:2558-2562, which is hereby incorporated by reference in its entirety).

In another embodiment, the PDGF antagonist of Table 1 or 2 is the antibody g162 or a pharmaceutically acceptable salt thereof (WO1998025971 (see Example 7), which is hereby incorporated by reference in its entirety).

In another embodiment, the PDGF antagonist of Table 1 or 2 is pyrazolo[3,4-g]quinoxaline or a pharmaceutically acceptable salt thereof (U.S. Pat. No. 5,476,851, which is hereby incorporated by reference in its entirety).

In another embodiment, the PDGF antagonist of Table 1 or 2 is 6-[2-(methylcarbamoyl)phenylsulphanyl]-3-E-[2-(pyridine-2-yl)ethenyl]-indazole or a pharmaceutically acceptable salt thereof (EP1925941 (see para (0121)), which is hereby incorporated by reference in its entirety).

In another embodiment, the PDGF antagonist of Table 1 or 2 is 1-{2-[5-(2-methoxy-ethoxy)-benzoimidazole-1-yl]-quinoline-8-yl}-piperidine-4-ylamine or a pharmaceutically acceptable salt thereof (EP 1925941 (see para (0121)), which is hereby incorporated by reference in its entirety).

In another embodiment, the PDGF antagonist of Table 1 or 2 is 4-[4-[N-(4-nitrophenyl)carbamoyl]-1-piperazinyl]-6,7-dimethoxyquinazoline or a pharmaceutically acceptable salt thereof (EP1925941 (see para (0121)), which is hereby incorporated by reference in its entirety).

In another embodiment, the PDGF antagonist of Table 1 or 2 is 4-amino-5-fluoro-3-(6-(4-methyl-piperazine-1-yl)-1H-benzimidazole-2-yl)-1H-quinoline-2-one or a pharmaceutically acceptable salt thereof (EP1925941 (see para (0121)), which is hereby incorporated by reference in its entirety).

In another embodiment, the PDGF antagonist of Table 1 or 2 is (4-tert-butylphenyl) {4-[(6,7-dimethoxy-4-quinolyl)oxy]phenyl}methaneone or a pharmaceutically acceptable salt thereof (EP 1925941 (see para (0121)), which is hereby incorporated by reference in its entirety).

In another embodiment, the PDGF antagonist of Table 1 or 2 is 5-methyl-N-[4-(trifluoromethyl)phenyl]-4-isoxazolecarboxamide or a pharmaceutically acceptable salt thereof (EP 1925941 (see para (0121)), which is hereby incorporated by reference in its entirety).

In another embodiment, the PDGF antagonist of Table 1 or 2 is trans-4-[(6,7-dimethoxyquinoxaline-2-yl)amino]cyclohexanol or a pharmaceutically acceptable salt thereof (EP 1925941 (see para (0121)), which is hereby incorporated by reference in its entirety).

In another embodiment, the PDGF antagonist of Table 1 or 2 is (Z)-3-[(2,4-dimethyl-5-(2-oxo-1,2-dihydroindole-3-ylidenemethyl)-1H-pyrrole-3-yl)-propionic acid or a pharmaceutically acceptable salt thereof (EP 1925941 (see para (0121)), which is hereby incorporated by reference in its entirety).

In another embodiment, the PDGF antagonist of Table 1 or 2 is 5-(5-fluoro-2-oxo-1,2-dihydroindole-3-ylidenemethyl)-2,4-dimethyl-1H-pyrrole-3-carboxylic acid or a pharmaceutically acceptable salt thereof (EP1925941 (see para (0121)), which is hereby incorporated by reference in its entirety).

In another embodiment, the PDGF antagonist of Table 1 or 2 is 1-(4-chloroanilino)-4-(4-pyridylmethyl)phthalazine or a pharmaceutically acceptable salt thereof (EP1925941 (see para (0121)), which is hereby incorporated by reference in its entirety).

In another embodiment, the PDGF antagonist of Table 1 or 2 is N-[4-(3-amino-1H-indazole-4-yl)phenyl-N′-(2-fluoro-5-methylphenyl)urea or a pharmaceutically acceptable salt thereof (EP1925941 (see para (0121)), which is hereby incorporated by reference in its entirety).

In another embodiment, the PDGF antagonist of Table 1 or 2 is 1,2-dimethyl-7-(2-thiophene)imidazolo[5,4-g]quinoxaline or a pharmaceutically acceptable salt thereof (U.S. Pat. No. 6,358,954 (see FIG. 4), which is hereby incorporated by reference in its entirety).

In another embodiment, the PDGF antagonist of Table 1 or 2 is 1,2-dimethyl-6-phenyl imidazolo[5,4-g]quinoxaline or a pharmaceutically acceptable salt thereof (U.S. Pat. No. 6,358,954 (see FIG. 6), which is hereby incorporated by reference in its entirety).

In another embodiment, the PDGF antagonist of Table 1 or 2 is 1,2-dimethyl-6-(2-thiophene)imidazolo[5,4-g]quinoxaline or a pharmaceutically acceptable salt thereof (U.S. Pat. No. 6,358,954 (see FIG. 2), which is hereby incorporated by reference in its entirety).

In another embodiment, the PDGF antagonist of Table 1 or 2 is AG1295 or a pharmaceutically acceptable salt thereof (Kovalenko et al. (1994) Cancer Research 54: 6106-6114, which is hereby incorporated by reference in its entirety).

In another embodiment, the PDGF antagonist of Table 1 or 2 is AG1296 or a pharmaceutically acceptable salt thereof (Kovalenko et al. (1994) Cancer Research 54: 6106-6114, which is hereby incorporated by reference in its entirety).

In another embodiment, the PDGF antagonist of Table 1 or 2 is 3-arylquinoline or a pharmaceutically acceptable salt thereof (Dolle et al. (1994) J. Med. Chem. 37, 2627-2629, which is hereby incorporated by reference in its entirety).

In another embodiment, the PDGF antagonist of Table 1 or 2 is 4-pyridyl-2-arylpyrimidine or a pharmaceutically acceptable salt thereof (Buchdunger et al. (1995) Proc. Natl. Acad. Sci. USA. 92: 2558-62, which is hereby incorporated by reference in its entirety).

In another embodiment, the PDGF antagonist of Table 1 or 2 is sorafenib or a pharmaceutically acceptable salt thereof (US2009081709 (see para (0007)), which is hereby incorporated by reference in its entirety).

In another embodiment, the PDGF antagonist of Table 1 or 2 is MLN518 or a pharmaceutically acceptable salt thereof (US2009081709 (see para (0007)), which is hereby incorporated by reference in its entirety).

In another embodiment, the PDGF antagonist of Table 1 or 2 is PKC412 or a pharmaceutically acceptable salt thereof (US2009081709 (see para (0007)), which is hereby incorporated by reference in its entirety).

In another embodiment, the PDGF antagonist of Table 1 or 2 is AMN107 or a pharmaceutically acceptable salt thereof (US2009081709 (see para (0007)), which is hereby incorporated by reference in its entirety).

In another embodiment, the PDGF antagonist of Table 1 or 2 is suramin or a pharmaceutically acceptable salt thereof (Williams et al. (1984) J. Biol. Chem. 259:287-5294, which is hereby incorporated by reference in its entirety).

In another embodiment, the PDGF antagonist of Table 1 or 2 is neomycin or a pharmaceutically acceptable salt thereof (Vassbotn et al. (1992) J. Biol. Chem. 267:15635-15641, which is hereby incorporated by reference in its entirety).

In another embodiment, the PDGF antagonist of Table 1 or 2 is an antibody or an antibody fragment which binds to an epitope PDGF-C (SEQ ID NO:28), PDGF-C (SEQ ID NO:29), PDGF-D (SEQ ID NO:30) or PDGF-D (SEQ ID NO:31), or any portion of the epitopes.

PDGF-C epitope:
(SEQ ID NO: 28)
Arg Lys Ser Arg Val Val Asp Leu Asn Leu Leu Thr Glu
Glu Val Arg Leu Tyr Ser Cys Thr Pro Arg Asn Phe Ser
Val Ser Ile Arg Glu Glu Leu Lys Arg Thr Asp Thr Ile
Phe Trp Pro Gly Cys
PDGF-C epitope:
(SEQ ID NO: 29)
Arg Lys Ser Arg Val Val Asp Leu Asn Leu Leu Thr Glu
Glu Val Arg Leu Tyr Ser Cys
PDGF-D epitope:
(SEQ ID NO: 30)
Arg Lys Ser Lys Val Asp Leu Asp Arg Leu Asn Asp Asp
Ala Lys Arg Tyr Ser Cys Thr Pro Arg Asn Tyr Ser Val
Asn Ile Arg Glu Glu Leu Lys Leu Ala Asn Val Val Phe
Phe Pro Arg Cys
PDGF-D epitope:
(SEQ ID NO: 31)
Cys Lys Ser Lys Val Asp Leu Asp Arg Leu Asn Asp Asp
Ala Lys Arg Tyr Ser Cys

In another embodiment, the PDGF antagonist of Table 1 or 2 is an antibody or an antibody fragment which binds to an epitope of PDGF, such as an epitope of PDGF-A, PDGF-B, PDGF-C, or PDGF-D. In some embodiments, the PDGF antagonist binds to an epitope of PDGF such that binding of PDGF and PDGFR are inhibited. In one embodiment, the epitope encompasses a component of the three dimensional structure of PDGF that is displayed, such that the epitope is exposed on the surface of the folded PDGF molecule. In one embodiment, the epitope is a linear amino acid sequence from PDGF.

7.3.2 VEGF Antagonists

In one embodiment, the VEGF antagonist of Table 1 or 2 is the antibody ranibizumab or a pharmaceutically acceptable salt thereof (see U.S. Pat. No. 7,060,269 (FIG. 1) for the heavy chain and light chain variable region sequences, which is hereby incorporated by reference in its entirety). Ranibizumab is commercially available under the trademark Lucentis.

In another embodiment, the VEGF antagonist of Table 1 or 2 is the antibody bevacizumab or a pharmaceutically acceptable salt thereof (see U.S. Pat. No. 6,054,297 (FIG. 1) for the heavy chain and light chain variable region sequences, which is hereby incorporated by reference in its entirety). Bevacizumab is commercially available under the trademark Avastin.

In another embodiment, the VEGF antagonist of Table 1 or 2 is aflibercept or a pharmaceutically acceptable salt thereof (Do et al. (2009) Br J Ophthalmol. 93:144-9, which is hereby incorporated by reference in its entirety).

In another embodiment, the VEGF antagonist of Table 1 or 2 is KH902 or a pharmaceutically acceptable salt thereof (Zhang et al. (2008) Mol. Vis. 14:37-49, which is hereby incorporated by reference in its entirety).

In another embodiment, the VEGF antagonist of Table 1 or 2 is the antibody 2C3 or a pharmaceutically acceptable salt thereof (U.S. Pat. No. 6,342,221 (Column 8, lines 48-67, Column 9, lines 1-21), which is hereby incorporated by reference in its entirety).

In another embodiment, the VEGF antagonist is ORA102 or a pharmaceutically acceptable salt thereof (Ora Bio, Ltd).

In one embodiment, the VEGF antagonist of Table 1 or 2 is pegaptanib or a pharmaceutically acceptable salt thereof (U.S. Pat. No. 6,051,698 (FIG. 1), which is hereby incorporated by reference in its entirety). A composition comprising pegaptanib is commercially available under the trademark Macugen.

In another embodiment, the VEGF antagonist of Table 1 or 2 is bevasiranib or a pharmaceutically acceptable salt thereof (Dejneka et al. (2008) Mol. Vis. 14:997-1005, which is hereby incorporated by reference in its entirety).

In another embodiment, the VEGF antagonist of Table 1 or 2 is Sirna-027 or a pharmaceutically acceptable salt thereof (Shen et al. (2006) Gene Ther. 13:225-34, which is hereby incorporated by reference in its entirety).

In another embodiment, the VEGF antagonist of Table 1 or 2 is decursin or a pharmaceutically acceptable salt thereof (U.S. Pat. No. 6,525,089 (Column 3, lines 5-16), which is hereby incorporated by reference in its entirety).

In another embodiment, the VEGF antagonist of Table 1 or 2 is decursinol or a pharmaceutically acceptable salt thereof (Ahn et al. (1997) Planta Med. 63:360-1, which is hereby incorporated by reference in its entirety).

In another embodiment, the VEGF antagonist of Table 1 or 2 is picropodophyllin or a pharmaceutically acceptable salt thereof (Economou (2008) Investigative Ophthalmology & Visual Science. 49:2620-6, which is hereby incorporated by reference in its entirety).

In another embodiment, the VEGF antagonist of Table 1 or 2 is guggulsterone or a pharmaceutically acceptable salt thereof (Kim et al. (2008) Oncol. Rep. 20:1321-7, which is hereby incorporated by reference in its entirety).

In another embodiment, the VEGF antagonist of Table 1 or 2 is PLG101 or a pharmaceutically acceptable salt thereof (Ahmadi and Lim (2008) Expert Opin Pharmacother. 9:3045-52, which is hereby incorporated by reference in its entirety).

In another embodiment, the VEGF antagonist of Table 1 or 2 is PLG201 or a pharmaceutically acceptable salt thereof (Ahmadi and Lim (2008) Expert Opin Pharmacother. 9:3045-52, which is hereby incorporated by reference in its entirety).

In another embodiment, the VEGF antagonist of Table 1 or 2 is eicosanoid LXA4 or a pharmaceutically acceptable salt thereof (Baker et al (2009) J Immun. 182:3819-26, which is hereby incorporated by reference in its entirety).

In another embodiment, the VEGF antagonist of Table 1 or 2 is PTK787 or a pharmaceutically acceptable salt thereof (Barakat and Kaiser (2009) Expert Opin Investig Drugs 18:637-46, which is hereby incorporated by reference in its entirety). A composition comprising PTK787 is commercially available under the trademark Vitalanib.

In another embodiment, the VEGF antagonist of Table 1 or 2 is pazopanib or a pharmaceutically acceptable salt thereof (Takahashi et al. (2009) Arch Ophthalmol. 127:494-9, which is hereby incorporated by reference in its entirety).

In another embodiment, the VEGF antagonist of Table 1 or 2 is axitinib or a pharmaceutically acceptable salt thereof (Hu-Lowe et al. (2008) Clin Cancer Res. 14:7272-83, which is hereby incorporated by reference in its entirety).

In another embodiment, the VEGF antagonist of Table 1 or 2 is CDDO-Me or a pharmaceutically acceptable salt thereof (Sogno et al. (2009) Recent Results Cancer Res. 181:209-12, which is hereby incorporated by reference in its entirety).

In another embodiment, the VEGF antagonist of Table 1 or 2 is CDDO-Imm or a pharmaceutically acceptable salt thereof (Sogno et al. (2009) Recent Results Cancer Res. 181:209-12, which is hereby incorporated by reference in its entirety).

In another embodiment, the VEGF antagonist of Table 1 or 2 is shikonin or a pharmaceutically acceptable salt thereof (Hisa et al. (1998) Anticancer Res. 18:783-90, which is hereby incorporated by reference in its entirety).

In another embodiment, the VEGF antagonist of Table 1 or 2 is beta-hydroxyisovalerylshikonin or a pharmaceutically acceptable salt thereof (Hisa et al. (1998) Anticancer Res. 18:783-90, which is hereby incorporated by reference in its entirety).

In another embodiment, the VEGF antagonist of Table 1 or 2 is ganglioside GM3 or a pharmaceutically acceptable salt thereof (Chung et al. (2009) Glycobio. 19:229-39, which is hereby incorporated by reference in its entirety).

In another embodiment, the VEGF antagonist of Table 1 or 2 is the antibody DC101 or a pharmaceutically acceptable salt thereof (U.S. Pat. No. 6,448,077 (Column 2, lines 61-65), which is hereby incorporated by reference in its entirety).

In another embodiment, the VEGF antagonist of Table 1 or 2 is the antibody Mab25 or a pharmaceutically acceptable salt thereof (U.S. Pat. No. 6,448,077 (Column 2, lines 61-65), which is hereby incorporated by reference in its entirety).

In another embodiment, the VEGF antagonist of Table 1 or 2 is the antibody Mab73 or a pharmaceutically acceptable salt thereof (U.S. Pat. No. 6,448,077 (Column 2, lines 61-65), which is hereby incorporated by reference in its entirety).

In another embodiment, the VEGF antagonist of Table 1 or 2 is the antibody 4A5 or a pharmaceutically acceptable salt thereof (U.S. Pat. No. 6,383,484 (Column 12, lines 50-54), which is hereby incorporated by reference in its entirety).

In another embodiment, the VEGF antagonist of Table 1 or 2 is the antibody 4E10 or a pharmaceutically acceptable salt thereof (U.S. Pat. No. 6,383,484 (Column 10, lines 66-67, Column 11, lines 1-2), which is hereby incorporated by reference in its entirety).

In another embodiment, the VEGF antagonist of Table 1 or 2 is the antibody 5F12 or a pharmaceutically acceptable salt thereof (U.S. Pat. No. 6,383,484 (Column 10, lines 62-65), which is hereby incorporated by reference in its entirety).

In another embodiment, the VEGF antagonist of Table 1 or 2 is the antibody VA01 or a pharmaceutically acceptable salt thereof (U.S. Pat. No. 5,730,977 (Column 6, lines 26-30), which is hereby incorporated by reference in its entirety).

In another embodiment, the VEGF antagonist of Table 1 or 2 is the antibody BL2 or a pharmaceutically acceptable salt thereof (U.S. Pat. No. 5,730,977 (Column 6, lines 30-32), which is hereby incorporated by reference in its entirety).

In one embodiment, the VEGF antagonist of Table 1 or 2 is VEGF-related protein or a pharmaceutically acceptable salt thereof (U.S. Pat. No. 6,451,764 (FIG. 1), which is hereby incorporated by reference in its entirety).

In another embodiment, the VEGF antagonist of Table 1 or 2 is sFLT01 or a pharmaceutically acceptable salt thereof (Pechan et al. (2009) Gene Ther. 16:10-6, which is hereby incorporated by reference in its entirety).

In another embodiment, the VEGF antagonist of Table 1 or 2 is sFLT02 or a pharmaceutically acceptable salt thereof (Pechan et al. (2009) Gene Ther. 16:10-6, which is hereby incorporated by reference in its entirety).

In another embodiment, the VEGF antagonist of Table 1 or 2 is Peptide B3 or a pharmaceutically acceptable salt thereof (Lacal et al. (2008) Eur J Cancer 44:1914-21, which is hereby incorporated by reference in its entirety).

In another embodiment, the VEGF antagonist of Table 1 or 2 is TG100801 or a pharmaceutically acceptable salt thereof (Palanki et al. (2008) J Med. Chem. 51:1546-59, which is hereby incorporated by reference in its entirety).

In another embodiment, the VEGF antagonist of Table 1 or 2 is sorafenib or a pharmaceutically acceptable salt thereof (Kernt et al. (2008) Acta Ophthalmol. 86:456-8, which is hereby incorporated by reference in its entirety). A composition comprising sorafenib is commercially available under the trademark Nexavar.

In another embodiment, the VEGF antagonist of Table 1 or 2 is G6-31 antibody or a pharmaceutically acceptable salt thereof (Crawford et al. (2009) Cancer Cell 15:21-34, which is hereby incorporated by reference in its entirety).

In another embodiment, the VEGF antagonist of Table 1 or 2 is an antibody or an antibody fragment which binds to an epitope VEGF-A (SEQ ID NO:32) or VEGF-B (SEQ ID NO:33), or any portion of the epitopes.

VEGF-A epitope:
(SEQ ID NO: 32)
Cys Asn Asp Glu Gly Leu Glu Cys Val Pro Thr Glu Glu
Ser Asn Ile
VEGF-B epitope:
(SEQ ID NO: 33)
Cys Pro Asp Asp Gly Lue Glu Cys Val Pro Thr Gly Gln
His Gln Val

In one embodiment, the PDGF or VEGF antagonist of Table 1 or 2 is an antibody or antibody fragment that binds to one or more of an epitope of PDGF (e.g. SEQ ID NO:28-31) and one or more of an epitope of VEGF (e.g., SEQ ID NO:32-33)

In another embodiment, the VEGF antagonist of Table 1 or 2 is an antibody or an antibody fragment which binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three dimensional structure of VEGF that is displayed, such that the epitope is exposed on the surface of the folded VEGF molecule. In one embodiment, the epitope is a linear amino acid sequence from VEGF.

7.3.3 Other Agents for Treatment or Prevention of an Ophthalmological Disease

In another embodiment, another agent useful for treating or preventing an ophthalmological disease is volociximab or a pharmaceutically acceptable salt thereof (Ramakrishnan et al. (2008) J Exp Ther Oncol. 5:273-86, which is hereby incorporated by reference in its entirety).

### 7.4 Modification of Antagonist Agents

Aptamer Antagonists

Where an antagonist of the present invention is an aptamer, the invention encompasses modified versions thereof, as set forth below. In some embodiments, an aptamer can have chemically modified nucleotides, including 5-X and/or 2′-Y substitutions in pyrimidine bases and 8-X and/or 2′-Y substitutions in purine bases. 2′-Modifications, such as 2′-fluoro and 2′-O-Me, can be utilized for stabilization against nucleases without compromising the aptamer binding interaction with the target. See, e.g., Lin et al., Nucleic Acids Res., 22, 5229-5234 (1994); Jellinek et al., Biochemistry, 34, 11363-1137 (1995); Lin et al., Nucleic Acids Res., 22, 5229-5234 (1994); Kubik et al., J. Immunol., 159(1), 259-267 (1997); Pagratis et al., Nat. Biotechnol., 1, 68-73 (1997); and Wilson et al., Curr Opin Chem Biol, 10(6), 607-614 (2006). In some embodiments, the chemical substitution can be a chemical substitution at a sugar position; a chemical substitution at a base position or a chemical substitution at a phosphate position.

Modifications of aptamers of this invention include, but are not limited to, those which provide other chemical groups that incorporate additional charge, polarizability, hydrophobicity, hydrogen bonding, electrostatic interaction, or fluxionality to the aptamer bases or to the aptamer as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping or modification with sugar moieties. In some embodiments of the instant invention, the aptamers are RNA molecules that are 2′-fluoro (2′-F) modified on the sugar moiety of pyrimidine residues.

The stability of the aptamer can be increased by the introduction of such modifications and as well as by modifications and substitutions along the phosphate backbone of the RNA. In addition, a variety of modifications can be made on the nucleobases themselves which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers can also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer can further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N2-carboxypropyl)uracil, 5-methylaminomethyluracil, 5-propynyl uracil, 6-azouracil, or 4-thiouracil.

Examples of other modified base variants known in the art include, without limitation, 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, wybutosine, 3-(3-amino-3-carboxypropyl)uridine.

Examples of modified nucleoside and nucleotide sugar backbone variants known in the art include, without limitation, those having, e.g., 2′ ribosyl substituents such as F, SH, SCH3, OCN, Cl, Br, CN, CF3, OCF3, SOCH3, SO2, CH3, ONO2, NO2, N3, NH2, OCH2CH2OCH3, O(CH2)2-0N(CH3)2, OCH2OCH2N(CH3)2, O(CH1-10 alkyl), O(C2-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′ OCH2CH2CH2NH2), 2′-allyl (2′-CH2-CH.dbd.CH2), 2′-O-allyl (2′-O-CH2-CH.dbd.CH2), 2′-amino (2′-NH2), and 2′-fluoro (2′-F). The 2′-substituent may be in the arabino (up) position or ribo (down) position.

Examples of modifications include: a purine substitution for a pyrimidine; a 2′-deoxy dihydrouridine substitution for a uridine; a 2′-deoxy-5-methyl cytidine for a cytidine; a 2-amino purine substitution for a purine; a phosphorothioate substituted for a phosphodiester; a phosphorodithioate substituted for a phosphodiester; a deoxynucleotide substituted for a 2′-OH nucleotide; a 2′-OMe nucleotide, a 2′-fluoro nucleotide or a 2′-O-methoxyethyl nucleotide substituted for a 2′-OH or deoxynucleotide; the addition of a PEG or PAG polymer; the addition of a large steric molecule; the addition of a 3′ cap; or any other modification known to block nuclease degradation. See, for example, U.S. Patent Publication No. 20090075342, which is incorporated by reference in its entirety.

The aptamers of the invention may be made up of nucleotides and/or nucleotide analogs such as described above, or a combination of both, or are oligonucleotide analogs. The aptamers of the invention may contain nucleotide analogs at positions which do not affect the function of the oligomer, for example, to bind PDGF or VEGF (or their cognate receptors).

The aptamers described herein can be linked with one or more non-physiologically active groups, such as a lipophilic compound (e.g., cholesterol); non-immunogenic high molecular weight compounds; or attached to or encapsulated in a complex comprising a lipophilic component (eg., a liposome). In one embodiment, the linked aptamers enhance the cellular uptake of the aptamers by a cell for delivery of the aptamers to an intracellular target. U.S. Pat. No. 6,011,020 describes a method for preparing a therapeutic or diagnostic compounds of an aptamer linked with lipophilic compound or a non-immunogenic, high molecular weight compound.

The invention further encompasses linking selected aptamers with one or more non-physiologically active group, such as lipophilic or Non-Immunogenic, High Molecular Weight compounds, in a diagnostic or therapeutic complex as described in U.S. Pat. No. 6,011,020. Aptamers that are linked with a Lipophilic Compound, such as diacyl glycerol or dialkyl glycerol, in a diagnostic or therapeutic complex are described in U.S. Pat. No. 5,859,228. Aptamers that are linked with a Lipophilic Compound, such as a glycerol lipid, or a Non-Immunogenic, High Molecular Weight Compound, such as polyalkylene glycol, are further described in U.S. Pat. No. 6,051,698. Aptamers that are linked with a Non-Immunogenic, High Molecular Weight compound or a lipophilic compound are also further described in PCT/US97/18944, filed Oct. 17, 1997, entitled “Vascular Endothelial Growth Factor (VEGF) Nucleic Acid Ligand Complexes.” Each of the above described patents and patent applications are specifically incorporated by reference herein in its entirety.

Certain embodiments of the present invention provide compounds comprising one or more aptamers covalently linked with a Non-Immunogenic, High Molecular Weight compound or lipophilic compound. A Non-Immunogenic, High Molecular Weight compound can be a compound that has a molecular weight of about 100 Da to 1,000,000 Da, about 1000 Da to 500,000 Da, or about 1000 Da to 200,000 Da, that typically does not generate an immunogenic response. For the purposes of this invention, an immunogenic response is one that causes the organism to make antibody proteins directed to the non-physiologically active group. In one embodiment, the Non-Immunogenic, High Molecular Weight compound can be a polyalkylene glycol. In another embodiment, the polyalkylene glycol can be polyethylene glycol (PEG). In some embodiments, the PEG has a molecular weight of about 10-80K or a molecular weight of about 20-45K. In some embodiments, the Non-Immunogenic, High Molecular Weight compound can be an aptamer.

Another embodiment of the invention is directed to compounds comprising an aptamer linked with lipophilic compound. Lipophilic compounds are compounds that have the propensity to associate with or partition into lipid and/or other materials or phases having a low dielectric constant, including compounds based mostly on lipophilic components. Lipophilic compounds include lipids as well as non-lipid containing compounds that have the propensity to associate with lipids (and/or other materials or phases with low dielectric constants). Cholesterol, phospholipid, and glycerol lipids, such as dialkyl glycerol, diacyl glycerol, and glycerol amide lipids are further examples of lipophilic compounds. In one embodiment, the lipophilic compound is a glycerol lipid.

The Non-Immunogenic, High Molecular Weight compound or lipophilic compound can be covalently bound to a variety of positions on the aptamer, such as to an exocyclic amino group on a nucleotide's base, the 5-position of a pyrimidine nucleotide, the 8-position of a purine nucleotide, the hydroxyl group of a nucleotide's phosphate, or a hydroxyl group or other group at the 5′ or 3′ terminus of the aptamer. In some embodiments where the lipophilic compound is a glycerol lipid, or the Non-Immunogenic, High Molecular Weight compound is polyalkylene glycol or polyethylene glycol, the Non-Immunogenic, High Molecular Weight compound can be bonded to the 5′ or 3′ hydroxyl of the phosphate group thereof. In one embodiment, the lipophilic compound or Non-Immunogenic, High Molecular Weight compound is bonded to the 5′ phosphate group of the aptamer. Attachment of the Non-Immunogenic, High Molecular Weight compound or lipophilic compound to the aptamer can be done directly or with the utilization of one or more linkers that interpose between the aptamer and lipophilic compound or Non-Immunogenic, High Molecular Weight compound. When attachment is done directly, on the other hand, no linker is present.

A linker is a molecular entity that connects two or more molecular entities through covalent bonds or non-covalent interactions, and can allow spatial separation of the molecular entities in a manner that preserves the functional properties of one or more of the molecular entities.

In one embodiment of the invention, the Non-Immunogenic, High Molecular Weight Compound covalently linked with the aptamer is a polyalkylene glycol and has the structure R(O(CH₂)ₓ)ₙO—, where R is independently selected from the group consisting of H and CH₃, x=2-5, and n.apprxeq.MW of the Polyalkylene Glycol/(16+14x). In one embodiment of the present invention, the molecular weight of the polyalkylene glycol is about between 10-80 kDa. In another embodiment, the molecular weight of the polyalkylene glycol is about between 20-45 kDa. In yet another embodiment, x=2 and n=9.times.10². There can be one or more Polyalkylene Glycols attached to the same aptamer.

In one embodiment, a Complex of the present invention is a PDGF aptamer covalently linked with a Non-Immunogenic, High Molecular Weight Compound such as Polyalkylene Glycol or PEG. In this embodiment, the pharmacokinetic properties of the Complex are improved relative to the PDGF aptamer alone. The Polyalkylene Glycol or PEG can be covalently bound to a variety of positions on the PDGF aptamer. In embodiments where Polyalkylene Glycol or PEG are used, the PDGF aptamer can be bonded through the 5′ hydroxyl group via a phosphodiester linkage.

In some embodiments, a plurality of aptamers can be associated with a single Non-Immunogenic, High Molecular Weight Compound, such as Polyalkylene Glycol or PEG, or a Lipophilic Compound, such as a glycerolipid. The aptamers can all be to one target or to different targets. In embodiments where a compound comprises more than one PDGF aptamer, there can be an increase in avidity due to multiple binding interactions with a target, such as PDGF or VEGF. In yet further embodiments, a plurality of Polyalkylene Glycol, PEG, glycerol lipid molecules can be attached to each other. In these embodiments, one or more aptamers can be associated with each Polyalkylene Glycol, PEG, or glycerol lipid. This can result in an increase in avidity of each aptamer to its target. In addition, in embodiments where there are aptamers to PDGF or aptamers to PDGF and different Targets associated with Polyalkylene Glycol, PEG, or glycerol lipid, a drug can also be associated with, e.g., covalently bonded to, Polyalkylene Glycol, PEG, or glycerol lipid. Thus the compound would provide targeted delivery of the drug, with Polyalkylene Glycol, PEG, or glycerol lipid serving as a Linker, optionally, with one or more additional linkers.

Aptamers can be 5′-capped and/or 3′-capped with a 5′-5′ inverted nucleoside cap structure at the 5′ end and/or a 3′-3′ inverted nucleoside cap structure at the 3′ end. In several embodiments, Antagonist A, Antagonist B, Antagonist C, Antagonist D, pegaptanib, bevasiranib and Sirna-027 are 5′ or 3′ end-capped.

Antibody Antagonists

Where the PDGF antagonist or VEGF antagonist of Table 1 or 2 is an antibody, such as for example 1B3, CDP860, 162.62, 163.31, 169.14, 169.31, αR1, 2A1E2, M4TS.11, M4TS.22, Hyb 120.1.2.1.2 antibody, Hyb 121.6.1.1.1 antibody, Hyb 127.5.7.3.1 antibody, Hyb 127.8.2.2.2 antibody, Hyb 1.6.1 antibody, Hyb 1.11.1 antibody, Hyb 1.17.1 antibody, Hyb 1.18.1 antibody, Hyb 1.19.1 antibody, Hyb 1.23.1 antibody, Hyb 1.24 antibody, Hyb 1.25 antibody, Hyb 1.29 antibody, Hyb 1.33 antibody, Hyb 1.38 antibody, Hyb 1.39 antibody, Hyb 1.40 antibody, Hyb 1.45 antibody, Hyb 1.46 antibody, Hyb 1.48 antibody, Hyb 1.49 antibody, Hyb 1.51 antibody, Hyb 6.4.1 antibody, F3 antibody, Humanized F3 antibody, C1 antibody, Humanized C1 antibody, 6.4 antibody, anti-mPDGF-C goat IgG antibody, C3.1 antibody, PDGFR-B1 monoclonal antibody, PDGFR-B2 monoclonal antibody, 6D11 monoclonal antibody, Sis 1 monoclonal antibody, PR7212 monoclonal antibody, PR292 monoclonal antibody, HYB 9610 monoclonal antibody, HYB 9611 monoclonal antibody, HYB 9612 monoclonal antibody, HYB 9613 monoclonal antibody, human antibody g162, ranibizumab, bevacizumab, KH902, DC101, Mab25, Mab73, 4A5, 4E10, 5F12, VA01, BL2, G6-31 antibody, or anti-mPDGF-C goat IgG antibody, the invention also relates to antibody fragments. Unless specified otherwise, the term antibody refers only to whole antibodies.

The antagonist antibodies of the invention include monoclonal inhibitory antibodies. Monoclonal antibodies, or fragments thereof, encompass all immunoglobulin classes such as IgM, IgG, IgD, IgE, IgA, or their subclasses, such as the IgG subclasses or mixtures thereof. IgG and its subclasses are useful, such as IgG₁, IgG₂, IgG₂ₐ, IgG_{2b}, IgG₃ or IgG_{M}. The IgG subtypes IgG₁/kappa and IgG_{2b}/kapp are included as useful embodiments. Fragments of the invention are truncated or modified antibody fragments with an antigen-complementary binding site. In some embodiments, an antibody fragment is formed by light and heavy chains, such as Fv, Fab or F(ab′)₂ fragments, or single-stranded fragments.

The invention further includes derivatives of antibodies of the present invention which retain their antagonist activity while altering one or more other properties related to their use as a pharmaceutical agent, e.g., serum stability or efficiency of production. Examples of such antibody derivatives include peptides, peptidomimetics derived from the antigen-binding regions of the antibodies, and antibodies, antibody fragments or peptides bound to solid or liquid carriers such as polyethylene glycol, glass, synthetic polymers such as polyacrylamide, polystyrene, polypropylene, polyethylene or natural polymers such as cellulose, sepharose or agarose, or conjugates with enzymes, toxins or radioactive or nonradioactive markers such as ³H, ¹²³I, ¹²⁵I, ¹³¹I, ³²P, ³⁵S, ¹⁴C, ⁵¹Cr, ³⁶Cl, ⁵⁷Co, ⁵⁵Fe, ⁵⁹Fe, ⁹⁰Y, ⁹⁹mTc, ⁷⁵Se, or antibodies, fragments, or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

In some embodiments, a monoclonal antibody of the present invention can be modified by splicing a variable (including hypervariable) domain of the antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments, so long as they exhibit the desired biological activity. See, for example, U.S. Pat. No. 4,816,567 and Mage & Lamoyi, in Monoclonal Antibody Production Techniques and Applications, pp. 79-97 (Marcel Dekker, Inc.), New York (1987). Methods for humanizing non-human antibodies are well known in the art.

### 7.5 Treatment or Prevention of an Ophthalmological Disease

In some embodiments of the invention, the ophthalmological disease is a neovascular disorder. In other embodiments of the invention, the ophthalmological disease results in retinal edema. Illustrative ophthalmological disease are listed below.

7.5.1 Treatment or Prevention of Age-Related Macular Degeneration

In one embodiment, the ophthalmological disease is age-related macular degeneration. Examples of age-related macular degeneration are normeovascular (also known as “Dry”) and neovascular (also known as “Wet”) macular degeneration. In one embodiment the dry age-related macular degeneration is associated with the formation of drusen. Treating or preventing dry macular degeneration also encompasses treating or preventing an abnormality of the retinal pigment epithelium. Examples of abnormalities of the retinal pigment epithelium include geographic atrophy, non-geographic atrophy, focal hypopigmentation, and focal hyperpigmentation. Treating or preventing wet age-related macular degeneration also encompasses treating or preventing choroidal neovascularization or pigment epithelial detachment.

7.5.2 Treatment or Prevention of Polypoidal Choroidal Vasculopathy

In one embodiment, the ophthalmological disease is polypoidal choroidal vasculopathy. Polypoidal choroidal vasculopathy is characterized by a lesion from an inner choroidal vascular network of vessels ending in an aneurysmal bulge or outward projection (Ciardella et al. (2004) Surv Ophthalmol. 49:25-37).

7.5.3 Treatment or Prevention of a Condition Associated with Choroidal Neovascularization

In one embodiment, the ophthalmological disease is a condition associated with choroidal neovascularization. Examples of conditions associated with choroidal neovascularization include a degenerative, inflammatory, traumatic or idiopathic condition. Treating or preventing a degenerative disorder associated with choroidal neovascularization also encompasses treating or preventing a heredodegerative disorder. Examples of heredodegerative disorders include vitelliform macular dystrophy, fundus flavimaculatus and optic nerve head drusen. Examples of degenerative conditions associated with choroidal neovascularization include myopic degeneration or angioid streaks. Treating or preventing an inflammatory disorder associated with choroidal neovascularization also encompasses treating or preventing ocular histoplasmosis syndrome, multifocal choroiditis, serpininous choroiditis, toxoplasmosis, toxocariasis, rubella, Vogt-Koyanagi-Harada syndrome, Behcet syndrome or sympathetic ophthalmia. Treating or preventing a traumatic disorder associated with choroidal neovascularization also encompasses treating or preventing choroidal rupture or a traumatic condition caused by intense photocoagulation.

7.5.4 Treatment or Prevention of Hypertensive Retinopathy

In one embodiment, the ophthalmological disease is hypertensive retinopathy.

7.5.5 Treatment or Prevention of Diabetic Retinopathy

In one embodiment, the ophthalmological disease is diabetic retinopathy. Diabetic retinopathy can be nonproliferative or proliferative diabetic retinopathy. Examples of nonproliferative diabetic retinopathy include macular edema and macular ischemia.

7.5.6 Treatment or Prevention of Sickle Cell Retinopathy

In one embodiment, the ophthalmological disease is sickle cell retinopathy.

7.5.7 Treatment or Prevention of a Condition Associated with Peripheral Retinal Neovascularization

In one embodiment, the ophthalmological disease is a condition associated with peripheral retinal neovascularization. Examples of conditions associated with peripheral retinal neovascularization include ischemic vascular disease, inflammatory disease with possible ischemia, incontinentia pigmenti, retinitis pigmentosa, retinoschisis or chronic retinal detachment.

Examples of ischemic vascular disease include proliferative diabetic retinopathy, branch retinal vein occlusion, branch retinal arteriolar occlusion, carotid cavernous fistula, sickling hemoglobinopathy, non-sickling hemoglobinopathy, IRVAN syndrome (retinal vasculitic disorder characterized by idiopathic retinal vasculitis, an aneurysm, and neuroretinitis), retinal embolization, retinopathy of prematurity, familial exudative vitreoretinopathy, hyperviscosity syndrome, aortic arch syndrome or Eales disease. Examples of sickling hemoglobinopathy include SS hemoglobinopathy and SC hemoglobinopathy. Examples of non-sickling hemoglobinopathy include AC hemoglobinopathy and AS hemoglobinopathy. Examples of hyperviscosity syndrome include leukemia, Waldenstrom macroglobulinemia, multiple myeloma, polycythemia or myeloproliferative disorder.

Treating or preventing an inflammatory disease with possible ischemia also encompasses treating or preventing retinal vasculitis associated with systemic disease, retinal vasculitis associated with an infectious agent, uveitis or birdshot retinopathy. Examples of systemic diseases include systemic lupus erythematosis, Behcet's disease, inflammatory bowel disease, sarcoidosis, multiple sclerosis, Wegener's granulomatosis and polyarteritis nodosa. Examples of infectious agents include a bacterial agent that is the causative agent for syphilis, tuberculosis, Lyme disease or cat-scratch disease, a virus such as herpesvirus, or a parasite such as Toxocara canis or Toxoplasma gondii. Examples of uveitis include pars planitis or Fuchs uveitis syndrome.

7.5.8 Treatment or Prevention of Retinopathy of Prematurity

In one embodiment, the ophthalmological disease is retinopathy of prematurity. Retinopathy of prematurity can result from abnormal growth of blood vessels in the vascular bed supporting the developing retina (Pollan C (2009) Neonatal Netw. 28:93-101).

7.5.9 Treatment or Prevention of Venous Occlusive Disease

In one embodiment, the ophthalmological disease is venous occlusive disease. Examples of venous occlusive disease include branch retinal vein occlusion and central retinal vein occlusion. A branch retinal vein occlusion can be a blockage of the portion of the circulation that drains the retina of blood. The blockage can cause back-up pressure in the capillaries, which can lead to hemorrhages and also to leakage of fluid and other constituents of blood.

7.5.10 Treatment or Prevention of Arterial Occlusive Disease

In one embodiment, the ophthalmological disease is arterial occlusive disease. Examples of arterial occlusive disease include branch retinal artery occlusion, central retinal artery occlusion or ocular ischemic syndrome. A branch retinal artery occlusion (BRAO) can occur when one of the branches of the arterial supply to the retina becomes occluded.

7.5.11 Treatment or Prevention of Central Serous Chorioretinopathy

In one embodiment, the ophthalmological disease is central serous chorioretinopathy (CSC). In one embodiment, CSC is characterized by leakage of fluid in the central macula.

7.5.12 Treatment or Prevention of Cystoid Macular Edema

In one embodiment, the ophthalmological disease is cystoid macular edema (CME). In one embodiment, CME affects the central retina or macula. In another embodiment, CME occurs after cataract surgery.

7.5.13 Treatment or Prevention of Retinal Telangiectasia

In one embodiment, the ophthalmological disease is retinal telangiectasia. In one embodiment, retinal telangiectasia is characterized by dilation and tortuosity of retinal vessels and formation of multiple aneurysms. Idiopathic JXT, Leber's miliary aneurysms, and Coats' disease are three types of retinal telangiectasias.

7.5.14 Treatment or Prevention of Arterial Macroaneurysm

In one embodiment, the ophthalmological disease is arterial macroaneurysm.

7.5.15 Treatment or Prevention of Retinal Angiomatosis

In one embodiment, the ophthalmological disease is retinal angiomatosis. In one embodiment, retinal angiomatosis occurs when the ocular vessels form multiple angiomas.

7.5.16 Treatment or Prevention of Radiation-Induced Retinopathy

In one embodiment, the ophthalmological disease is radiation-induced retinopathy (RIRP). In one embodiment, RIRP may display symptoms such as macular edema and nonproliferative and proliferative retinopathy.

7.5.17 Treatment or Prevention of Rubeosis Iridis

In one embodiment, the ophthalmological disease is rubeosis iridis. In another embodiment, rubeosis iridis results in the formation of neovascular glaucoma. In another embodiment, rubeosis iridis is caused by diabetic retinopathy, central retinal vein occlusion, ocular ischemic syndrome, or chronic retinal detachment.

7.5.18 Treatment or Prevention of a Neoplasm

In one embodiment, the ophthalmological disease is a neoplasm. Examples of neoplasm include an eyelid tumor, a conjunctival tumor, a choroidal tumor, an iris tumor, an optic nerve tumor, a retinal tumor, an infiltrative intraocular tumor or an orbital tumor. Examples of an eyelid tumor include basal cell carcinoma, squamous carcinoma, sebaceous carcinoma, malignant melanoma, capillary hemangioma, hydrocystoma, nevus or seborrheic keratosis. Examples of a conjunctival tumor include conjunctival Kaposi's sarcoma, squamous carcinoma, intraepithelial neoplasia of the conjunctiva, epibular dermoid, lymphoma of the conjunctiva, melanoma, pingueculum, or pterygium. Examples of a choroidal tumor include choroidal nevus, choroidal hemangioma, metastatic choroidal tumor, choroidal osteoma, choroidal melanoma, ciliary body melanoma or nevus of Ota. Examples of an iris tumor include anterior uveal metastasis, iris cyst, iris melanocytoma, iris melanoma, or pearl cyst of the iris. Examples of an optic nerve tumor include optic nerve melanocytoma, optic nerve sheath meningioma, choroidal melanoma affecting the optic nerve, or circumpapillary metastasis with optic neuropathy. Examples of a retinal tumor include retinal pigment epithelial (RPE) hypertrophy, RPE adenoma, RPE carcinoma, retinoblastoma, hamartoma of the RPE, or von Hippel angioma. Examples of an infiltrative intraocular tumor include chronic lymphocytic leukemia, infiltrative choroidopathy, or intraocular lymphoma. Examples of an orbital tumor include adenoid cystic carcinoma of the lacrimal gland, cavernous hemangioma of the orbit, lymphangioma of the orbit, orbital mucocele, orbital pseudotumor, orbital rhabdomyosarcoma, periocular hemangioma of childhood, or sclerosing orbital psuedotumor.

### 7.6 Compositions for Therapeutic or Prophylactic Administration

The PDGF antagonist or VEGF antagonist of Table 1 or 2 can be administered as a component of a composition that further comprises a pharmaceutically acceptable carrier or vehicle. In one embodiment, a composition of the invention comprises an effective amount of a PDGF antagonist, a VEGF antagonist of Table 1 or 2 and a pharmaceutically acceptable carrier or vehicle. In another embodiment, a composition comprising a PDGF antagonist and another composition comprising a VEGF antagonist are administered.

Administration of each antagonist may be by any suitable means that results in an amount of PDGF antagonist and VEGF antagonist of Table 1 or 2 that is effective for the treatment or prevention of an ophthalmological disease. Each antagonist, for example, can be admixed with a suitable carrier substance, and is generally present in an amount of 1-95% by weight of the total weight of the composition. The composition may be provided in a dosage form that is suitable for ophthalmic, oral, parenteral (e.g., intravenous, intramuscular, subcutaneous), rectal, transdermal, nasal, or inhalant administration. In one embodiment, the composition is in a form that is suitable for injection directly in the eye. The composition may be in form of, e.g., tablets, capsules, pills, powders, granulates, suspensions, emulsions, solutions, gels including hydrogels, pastes, ointments, creams, plasters, delivery devices, suppositories, enemas, injectables, implants, sprays, drops or aerosols. The compositions comprising one or more antagonists can be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds., J. Swarbrick and J. C. Boylan, 1988-2002, Marcel Dekker, New York).

The compositions are, in one useful aspect, administered parenterally (e.g., by intramuscular, intraperitoneal, intravenous, intraocular, intravitreal, retro-bulbar, subconjunctival, subtenon or subcutaneous injection or implant) or systemically. Formulations for parenteral or systemic administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, gelatin, hydrogels, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to the art for the manufacture of pharmaceutical compositions.

Solid dosage forms for oral administration include capsules, tablets, pills, powders, and granules. Generally, these pharmaceutical preparations contain active ingredients admixed with non-toxic pharmaceutically acceptable excipients. These include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings. The compositions may optionally contain sweetening, flavoring, coloring, perfuming, and preserving agents in order to provide a more palatable preparation.

For example, compositions of the present invention may be administered intraocularly by intravitreal injection into the eye as well as by subconjunctival and subtenon injections. Other routes of administration include transcleral, retrobulbar, intraperitoneal, intramuscular, and intravenous. Alternatively, compositions can be administered using a drug delivery device or an intraocular implant (see below).

Liquid dosage forms for oral administration can include pharmaceutically acceptable emulsions, solutions, suspensions, syrups, and soft gelatin capsules. These forms can contain inert diluents commonly used in the art, such as water or an oil medium, and can also include adjuvants, such as wetting agents, emulsifying agents, and suspending agents.

In some instances, the compositions can also be administered topically, for example, by patch or by direct application to a region, such as the epidermis or the eye, susceptible to or affected by a neovascular disorder, or by iontophoresis.

Compositions useful for ophthalmic use include tablets comprising one or more antagonists in admixture with a pharmaceutically acceptable excipient. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The antagonists of the present invention may be admixed in a tablet or other vehicle, or may be partitioned. In one example, one antagonist is contained on the inside of the tablet, and the other antagonist is on the outside, such that a substantial portion of the other antagonist is released prior to the release of the contained antagonist. If desired, antagonists in a tablet form may be administered using a drug delivery device (see below).

In one embodiment, compositions that comprise a PDGF antagonist can comprise one or more pharmaceutically acceptable excipients. In one embodiment, excipients for compositions that comprise a PDGF antagonist include, but are not limited to, buffering agents, nonionic surfactants, preservatives, tonicity agents, amino acids, and pH-adjusting agents. Suitable buffering agents include, but are not limited to, monobasic sodium phosphate, dibasic sodium phosphate, and sodium acetate. Suitable nonionic surfactants include, but are not limited to, polyoxyethylene sorbitan fatty acid esters such as polysorbate 20 and polysorbate 80. Suitable preservatives include, but are not limited to, benzyl alcohol. Suitable tonicity agents include, but are not limited to sodium chloride, mannitol, and sorbitol. Suitable amino acids include, but are not limited to glycine and histidine. Suitable pH-adjusting agents include, but are not limited to, hydrochloric acid, acetic acid, and sodium hydroxide. In one embodiment, the pH-adjusting agent or agents are present in an amount effective to provide a pH of about 3 to about 8, about 4 to about 7, about 5 to about 6, about 6 to about 7, or about 7 to about 7.5. In one embodiment, a composition comprising a PDGF antagonist does not comprise a preservative. In another embodiment, a composition comprising a PDGF antagonist does not comprise an antimicrobial agent. In another embodiment, a composition comprising a PDGF antagonist does not comprise a bacteriostat.

In one embodiment, a composition comprising a PDGF antagonist is in the form of an aqueous solution that is suitable for injection. In one embodiment, a composition comprises a PDGF antagonist, a buffering agent, a pH-adjusting agent, and water for injection. In another embodiment, a composition comprises a PDGF antagonist, monobasic sodium phosphate, dibasic sodium phosphate, sodium chloride, hydrochloride acid, and sodium hydroxide. In one embodiment, the PDGF antagonist is a pegylated anti-PDGF aptamer. In another embodiment, the pegylated anti-PDGF aptamer is ARC-127. In another embodiment, the pegylated anti-PDGF antagonist is a compound of Formula A. In another embodiment, the pegylated anti-PDGF antagonist is Antagonist A. In another embodiment, the pegylated anti-PDGF antagonist is a compound of Formula B. In another embodiment, the pegylated anti-PDGF antagonist is Antagonist B. In another embodiment, the pegylated anti-PDGF antagonist is a compound of Formula C. In another embodiment, the pegylated anti-PDGF antagonist is a compound of Formula D. In another embodiment, the PDGF antagonist is a non-pegylated anti-PDGF aptamer. In another embodiment, the non-pegylated aptamer is Antagonist C. In another embodiment, the non-pegylated aptamer is Antagonist D.

In one embodiment, compositions that comprise a VEGF antagonist can comprise one or more pharmaceutically acceptable excipients. In one embodiment, excipients for compositions that comprise a VEGF antagonist include, but are not limited to, buffering agents, nonionic surfactants, preservatives, tonicity agents, sugars, amino acids, and pH-adjusting agents. Suitable buffering agents include, but are not limited to, monobasic sodium phosphate, dibasic sodium phosphate, and sodium acetate. Suitable nonionic surfactants include, but are not limited to, polyoxyethylene sorbitan fatty acid esters such as polysorbate 20 and polysorbate 80. Suitable preservatives include, but are not limited to, benzyl alcohol. Suitable tonicity agents include, but are not limited to sodium chloride, mannitol, and sorbitol. Suitable sugars include, but are not limited to, α,α-trehalose. Suitable amino acids include, but are not limited to, glycine and histidine. Suitable pH-adjusting agents include, but are not limited to, hydrochloric acid, acetic acid, and sodium hydroxide. In one embodiment, the pH-adjusting agent or agents are present in an amount effective to provide a pH of about 3 to about 8, about 4 to about 7, about 5 to about 6, about 6 to about 7, or about 7 to about 7.5. In one embodiment, a composition comprising a VEGF antagonist does not comprise a preservative. Suitable excipients for the VEGF antagonist also include those described in U.S. Pat. No. 7,365,166, the contents of which are herein incorporated by reference in their entirety.

In one embodiment, the composition is in the form of an aqueous solution that is suitable for injection. In one embodiment, the composition comprises a VEGF antagonist, a buffering agent, a sugar, a nonionic surfactant, and water for injection. In another embodiment, the composition comprises a VEGF antagonist, monobasic sodium phosphate, dibasic sodium phosphate, α,α-trehalose dehydrate, and polysorbate 20. In one embodiment, the composition comprises a VEGF antagonist, a buffering agent, a pH-adjusting agent, a tonicity agent, and water that is suitable for injection. In another embodiment, the composition comprises a VEGF antagonist, monobasic sodium phosphate, dibasic sodium phosphate, sodium chloride, hydrochloric acid, and sodium hydroxide. In one embodiment, the VEGF antagonist is a pegylated anti-VEGF aptamer.

In another embodiment, the VEGF antagonist is ranibizumab or bevacizumab. This invention includes the pharmaceutically acceptable salts of the antagonists of Table 1 or 2. An antagonist of the present invention can possess a sufficiently basic functional group, which can react with any of a number of inorganic and organic acids, to form a pharmaceutically acceptable salt. A pharmaceutically-acceptable acid addition salt is formed from a pharmaceutically-acceptable acid, as is well known in the art. Such salts include the pharmaceutically acceptable salts listed in Journal of Pharmaceutical Science, 66, 2-19 (1977) and The Handbook of Pharmaceutical Salts; Properties, Selection, and Use. P. H. Stahl and C. G. Wermuth (ED.s), Verlag, Zurich (Switzerland) 2002, which are hereby incorporated by reference in their entirety.

Pharmaceutically acceptable salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” also refers to a salt of an antagonists of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a compound of the invention.

In one embodiment, each of the PDGF and VEGF antagonists of Table 1 or 2 is administered in an amount effective to treat or prevent an ophthalmological disease. The amount of antagonist that is admixed with the carrier materials to produce a single dosage can vary depending upon the mammal being treated and the particular mode of administration.

The dosage of each antagonist can depend on several factors including the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Furthermore, the exact individual dosages can be adjusted somewhat depending on a variety of factors, including the specific combination of antagonists being administered, the time of administration, the route of administration, the nature of the formulation, the rate of excretion, the particular ophthalmological disease being treated, the severity of the disorder, and the anatomical location of the neovascular disorder. Some variations in the dosage can be expected.

Generally, when orally administered to a mammal, the dosage of an antagonist of the present invention is normally 0.001 mg/kg/day to 100 mg/kg/day, 0.01 mg/kg/day to 50 mg/kg/day, or 0.1 mg/kg/day to 10 mg/kg/day. Generally, when orally administered to a human, the dosage of an antagonist of the present invention is normally 0.001 mg to 300 mg per day, 1 mg to 200 mg per day, or 5 mg to 50 mg per day. Dosages up to 200 mg per day may be necessary. For administration of an antagonist of the present invention by parenteral injection, the dosage is normally 0.1 mg to 250 mg per day, 1 mg to 20 mg per day, or 3 mg to 5 mg per day. Injections may be given up to four times daily. Generally, when orally or parenterally administered, the dosage of a PDGF or VEGF antagonist of Table 1 or 2 for use in the present invention is normally 0.1 mg to 1500 mg per day, or 0.5 mg to 10 mg per day, or 0.5 mg to 5 mg per day. A dosage of up to 3000 mg per day can be administered.

When ophthalmologically administered to a human, for example intravitreally, the dosage of an antagonist of Table 1 or 2 is normally 0.003 mg to 5.0 mg per eye per administration, or 0.03 mg to 3.0 mg per eye per administration, or 0.1 mg to 1.0 mg per eye per administration. In one embodiment, the dosage of PDGF antagonist of Table 1 or 2 is 0.03 mg, 0.3 mg, 1.5 mg or 3.0 mg per eye. In another embodiment, the dosage of VEGF antagonist of Table 1 or 2 is 0.5 mg per eye. The dosage can range from 0.01 mL to 0.2 mL administered per eye, or 0.03 mL to 0.15 mL administered per eye, or 0.05 mL to 0.10 mL administered per eye.

For example, the PDGF aptamer Antagonist A, Antagonist B or Antagonist C or a pharmaceutically acceptable salt thereof can be delivered intravitreally at up to 30 mg/ml with injection volumes up to 100 μL.

Administration of each antagonist of Table 1 or 2 can, independently, be one to four times daily or one to four times per month or one to six times per year or once every two, three, four or five years. Administration can be for the duration of one day or one month, two months, three months, six months, one year, two years, three years, and may even be for the life of the patient. In one embodiment, the administration is performed once a month for three months. Chronic, long-term administration will be indicated in many cases. The dosage may be administered as a single dose or divided into multiple doses. In general, the desired dosage should be administered at set intervals for a prolonged period, usually at least over several weeks or months, although longer periods of administration of several months or years or more may be needed.

In addition to treating pre-existing ophthalmological diseases, the compositions can be administered prophylactically in order to prevent or slow the onset of these disorders. In prophylactic applications, the composition can be administered to a patient susceptible to or otherwise at risk of a particular ophthalmological disease.

In one embodiment, the PDGF antagonist and the VEGF antagonist of Table 1 or 2 are administered to a mammal in need of treatment therewith, typically in the form of an injectable pharmaceutical composition. The PDGF antagonist and VEGF antagonist of Table 1 or 2 can be administered either in separate compositions or in a pharmaceutical composition comprising both the PDGF antagonist and VEGF antagonist. The administration can be by injection, for example by intraocular injection, or by using a drug delivery device. Parenteral, systemic, or transdermal administration is also within the scope of the invention.

The administration of the PDGF antagonist and the VEGF antagonist of Table 1 or 2 can be sequential in time or concurrent. When administered sequentially, the administration of each can be by the same or different route. In one embodiment, a PDGF antagonist of Table 1 or 2 is administered within 90 days, 30 days, 10 days, 5 days, 24 hours, 1 hour, 30 minutes, 10 minutes, 5 minutes or one minute of administration of a VEGF antagonist of Table 1 or 2. Where the PDGF antagonist is administered prior to the VEGF antagonist, the VEGF antagonist is administered within a time and in an amount such that the total amount of PDGF antagonist and VEGF antagonist is effective to treat or prevent an ophthalmological disease. Where the VEGF antagonist is administered prior to the PDGF antagonist, the PDGF antagonist is administered within a time and in an amount such that the total amount of PDGF antagonist and VEGF antagonist is effective to treat or prevent an ophthalmological disease.

Pharmaceutical compositions according to the invention may be formulated to release a PDGF or VEGF antagonist of Table 1 or 2 substantially immediately upon administration or at any predetermined time period after administration, using controlled release formulations. For example, a pharmaceutical composition can be provided in sustained-release form. The use of immediate or sustained release compositions depends on the nature of the condition being treated. If the condition consists of an acute disorder, treatment with an immediate release form can be utilized over a prolonged release composition. For certain preventative or long-term treatments, a sustained released composition can also be appropriate.

Administration of one or both of the antagonists of Table 1 or 2 in controlled release formulations can be useful where the antagonist, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of degradation or metabolism of the therapeutic antagonist. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes. Methods for preparing such sustained or controlled release formulations are well known in the art.

The PDGF antagonist or VEGF antagonist can also be delivered using a drug-delivery device such as an implant. Such implants can be biodegradable and/or biocompatible, or can be non-biodegradable. The implants can be permeable to the PDGF antagonist or VEGF antagonist. Ophthalmic drug delivery devices can be inserted into a chamber of the eye, such as the anterior or posterior chamber or can be implanted in or on the sclera, choroidal space, or an avascularized region exterior to the vitreous. In one embodiment, the implant can be positioned over an avascular region, such as on the sclera, so as to allow for transcleral diffusion of the PDGF antagonist or VEGF antagonist to the desired site of treatment, e.g., the intraocular space and macula of the eye. Furthermore, the site of transcleral diffusion can be proximal to a site of neovascularization such as a site proximal to the macula. Suitable drug delivery devices are described, for example, in U.S. Publication Nos. 2008/0286334; 2008/0145406; 2007/0184089; 2006/0233860; 2005/0244500; 2005/0244471; and 2005/0244462, and U.S. Pat. Nos. 6,808,719 and 5,322,691, the contents of each of which is herein incorporated by reference in its entirety.

In one embodiment, the implant comprises a PDGF antagonist and/or VEGF antagonist dispersed in a biodegradable polymer matrix. The matrix can comprise PLGA (polylactic acid-polyglycolic acid copolymer), an ester-end capped polymer, an acid end-capped polymer, or a mixture thereof. In another embodiment, the implant comprises a PDGF antagonist and/or a VEGF antagonist, a surfactant, and lipophilic compound. The lipophilic compound can be present in an amount of about 80-99% by weight of the implant. Suitable lipophilic compounds include, but are not limited to, glyceryl palmitostearate, diethylene glycol monostearate, propylene glycol monostearate, glyceryl monostearate, glyceryl monolinoleate, glyceryl monooleate, glyceryl monopalmitate, glyceryl monolaurate, glyceryl dilaurate, glyceryl monomyristate, glyceryl dimyristate, glyceryl monopalmitate, glyceryl dipalmitate, glyceryl monostearate, glyceryl distearate, glyceryl monooleate, glyceryl dioleate, glyceryl monolinoleate, glyceryl dilinoleate, glyceryl monoarachidate, glyceryl diarachidate, glyceryl monobehenate, glyceryl dibehenate, and mixtures thereof. In another embodiment, the implant comprises a PDGF antagonist and/or a VEGF antagonist housed within a hollow sleeve. The PDGF antagonist or VEGF antagonist, or both, are delivered to the eye by inserting the sleeve into the eye, releasing the implant from the sleeve into the eye, and then removing the sleeve from the eye. An example of this delivery device is described in U.S. Publication No. 2005/0244462, which is hereby incorporated by reference in its entirety.

In one embodiment, the implant is a flexible ocular insert device adapted for the controlled sustained release of a PDGF antagonist and/or a VEGF antagonist into the eye. In one embodiment, the device includes an elongated body of a polymeric material in the form of a rod or tube containing a PDGF antagonist, VEGF antagonist or both, and with at least two anchoring protrusions extending radially outwardly from the body. The device may have a length of at least 8 mm and the diameter of its body portion including the protrusions does not exceed 1.9 mm. The sustained release mechanism can, for example, be by diffusion or by osmosis or bioerosion. The insert device can be inserted into the upper or lower formix of the eye so as to be independent of movement of the eye by virtue of the formix anatomy. The protrusions can be of various shapes such as, for example, ribs, screw threads, dimples or bumps, truncated cone-shaped segments or winding braid segments. In a further embodiment, the polymeric material for the body is selected as one which swells in a liquid environment. Thus a device of smaller initial size can be employed. The insert device can be of a size and configuration such that, upon insertion into the upper or lower formix, the device remains out of the field of vision so as to be well retained in place and imperceptible by a recipient over a prolonged period of use. The device can be retained in the upper or lower formix for 7 to 14 days or longer. An example of this device is described in U.S. Pat. No. 5,322,691, which is hereby incorporated by reference in its entirety.

The invention relates to kits comprising one or more pharmaceutical compositions and instructions for use. At least two antagonists of Table 1 or 2 can be formulated together or in separate compositions and in individual dosage amounts. The antagonists of Table 1 or 2 are also useful when formulated as pharmaceutically acceptable salts. In one embodiment, the kits comprise a composition comprising a PDGF antagonist and a pharmaceutically acceptable carrier or vehicle and another composition comprising a VEGF antagonist and a pharmaceutically acceptable carrier or vehicle. In another embodiment, the kits comprise a composition comprising a VEGF antagonist, a PDGF antagonist and a pharmaceutically acceptable carrier or vehicle. Each of the kits' compositions can be contained in a container.

The kits can comprise (1) an amount of a PDGF antagonist of Table 1 or 2 and a pharmaceutically acceptable carrier, vehicle, or diluent in a first unit dosage form; (2) an amount of a VEGF antagonist of Table 1 or 2 and a pharmaceutically acceptable carrier, vehicle, or diluent in a second unit dosage form; and (3) a container. The container can be used to separate components and include, for example, a divided bottle or a divided foil packet. The separate antagonist compositions may also, if desired, be contained within a single, undivided container. The kits can also comprise directions for the administration of the antagonists. The kits are particularly advantageous when the separate components are administered in different dosage forms, are administered at different dosage levels, or when titration of the individual antagonists is desired.

### 8. EXAMPLES

### Example 1

### Corneal Neovascularization (Corneal NV)

Corneal Neovascularization is a widely used animal model that allows clear visualization of abnormal vascular growth in the eye. The vessels that grow into the normally avascular cornea, can become well established, making this an attractive model to study vessel regression. To induce experimental corneal NV, male C57BL/6 mice (18-20 g; Charles River, Wilmington, Mass.) are anesthetized with intramuscular ketamine hydrochloride (25 mg/kg) and xylazine (10 mg/kg). NaOH (2 ul of 0.2 mM) is applied topically. The corneal and limbal epithelia are removed by applying a rotary motion parallel to the limbus using #21 blade (Feather, Osaka, Japan). After 7 days, mice are treated with intra-peritoneal injections of 2.0 mg/ml of Antagonist A, an anti-PDGF aptamer, agent twice a day or by intra-peritoneal injections of 2.0 mg/mL of ranibizumab (as the commercially available composition Lucentis®, an anti-VEGF antibody agent, twice a day or both for 7 days. At day 14 following corneal NV induction, mice receive 20 ug/g of fluorescein-isothiocyanate coupled concanavalin A lectin (Vector Laboratories, Burlingame, Calif.) intravenously while deeply anesthetized with xylazine hydrochloride and ketamine hydrochloride. Thirty minutes later, mice eyes are enucleated, and the corneas flat-mounted. Corneal NV is visualized using fluorescence microscopy and quantified using Openlab software. The percent of cornea covered by vessels is calculated as a percentage of total corneal area.

The effects of the administration of Antagonist A and ranibizumab are measured for decrease in vessel growth and pictures of the fluorescent microscopic image are taken.

### Example 2

### Administration of Antagonist A and Ranibizumab

The objectives of this study were to assess safety of Antagonist A, an intravitreal anti-PDGF aptamer targeting pericytes, in combination with ranibizumab in subjects with neovascular age-related macular degeneration (NV-AMD).

Dose-escalating, uncontrolled, single- and multiple-dose, multicenter phase I study. Included were subjects with predominantly or minimally classic subfoveal NV-AMD≧5 disc areas in total lesion size. Subjects were enrolled in a dose escalation scheme to a single injection of 0.03 mg/eye and 3 monthly injections of ranibizumab (as the commercially available composition Lucentis®) 0.5 mg/eye (n=3), or to three monthly injections of one of 4 different doses of Antagonist A (0.03, 0.3, 1.5, 3.0 mg/eye) and ranibizumab (as the commercially available composition Lucentis®) (0.5 mg/eye) (n=3-8/dose), administered as separate injections. Assessments included vital signs and clinical lab tests, complete ocular examination with intraocular pressure, standardized ETDRS visual acuity, color fundus photos and fluorescein angiography, and optical coherence tomography.

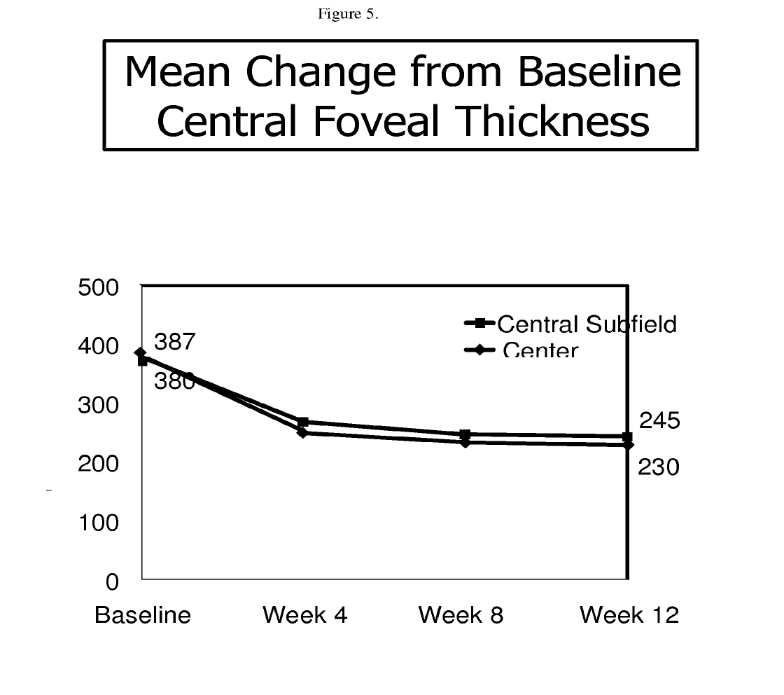

No evidence of drug related adverse events were detected. All of the ocular adverse events were associated with the intravitreal injection. In the combined analysis of 22 subjects over 12 weeks, 36%, 45% and 59% of the subjects gained ≧15 letters at weeks 4, 8, and 12 respectively. The mean change in visual acuity was +11.2, +12.3 and +14.0 ETDRS letters at weeks 4 (n=22), 8 (n=22), and 12 (n=22). The mean center point retinal thickness was 387 μm at baseline and 230 μm at week 12 (see FIG. 5). Analysis of FA by independent readers revealed a mean decrease in CNV area of 86% at Week 12 compared to baseline.

These results suggest potential bioactivity associated with regression of the neovascular membrane.

### Example 3

### Patterns of Choroidal Neovascularization (CNV) Fluorescein and Indocyanine Green Angiographic Regression Responses after Ranihizumab Monotherapy or Ranibizumab and Antagonist A Combotherapy

Anti-VEGF monotherapy for NV-AMD can cause stabilization of CNV lesion size and leakage. The fluorescein angiographic (FA) and dynamic indocyanine green angiographic (ICGA) patterns of CNV regression responses in eyes receiving either ranibizumab only or ranibizumab and Antagonist A were compared.

A retrospective review was performed of 20 cases of NV-AMD in which 2-3 doses of intravitreal ranibizumab (as the commercially available composition Lucentis®) monotherapy successfully induced anatomic improvement by OCT. These eyes were compared with 13 eyes of patients in a study of monthly intravitreal ranibizumab (as the commercially available composition Lucentis®) (up to 3 doses) plus intravitreal Antagonist A (at least one but up to 3 doses). Eyes were imaged by FA pretreatment and at various times post treatment. Eyes could also be imaged by dynamic ICGA (Spectralis, Heidelberg). Angiograms were evaluated to assess the changes in lesion size and vascular perfusion.

Three angiographic patterns of “OCT successful” responses to treatment were observed. (1) Stable inactivity was characterized by FA with stable lesion size and uniform low grade fluorescein hyperfluorescence (staining) of the CNV. ICGA typically demonstrated persistence of feeder arteries with branching arterioles. (2) Vascular regression demonstrated FA with stable CNV area but shrinkage of area of fluorescein staining. ICGA demonstrated disappearance of homogenous capillaries and small branching arterioles. (3) Lesion regression was characterized by partial to nearly complete disappearance of both the CNV lesion and hyperfluorescent staining. Persistent hypofluorescence in the bed of the CNV was often present. ICGA revealed significant disappearance of most vascular components. Partial or extensive lesion regression occurred in 85% (11 of 13 eyes) treated with ranibizumab and Antagonist A, compared with only 20% (4 of 20 eyes) treated with ranibizumab monotherapy. In contrast, stable inactivity was observed in only 15% (2 of 13 eyes) treated with ranibizumab and Antagonist A versus 55% (11 of 20 eyes) treated with ranibizumab monotherapy.

### Example 4

### Synthesis of Antagonist A

The iterative chemical synthesis of the 32-mer oligonucleotide of Antagonist A was performed on a solid phase inverted deoxyribothymidine controlled pore glass (CPG) support using a flow through reactor design. The oligonucleotide synthesis process was comprised of four chemical reactions carried out in the following sequence: (a) deblocking of the dimethyoxytrityl (DMT) protected nucleoside or nascent oligonucleotide (detritylation); (b) activation and coupling of the incoming phosphoramidite (amidite); (c) oxidation of the resultant phosphite triester to the pentavalent phosphate linkage; and (d) capping of oligonucleotide chains that failed to successfully couple.

Starting with an inverted thymidine CPG support (3′-DMT-5′-dT-CPG) the four steps above were repeated to add phosphoramidites in the order of the sequence until the desired oligonucleotide, terminating in the hexylamino linker, was synthesized. The internal hexaethylene glycol spacers were coupled in the same manner as the other phosphoramidites.

The first step in the cycle involved removal of the dimethyoxytrityl protecting group on the terminal hydroxyl group of the nascent oligonucleotide chain. This was achieved by treating the DMT protected oligonucleotide on CPG with a solution of dichloroacetic acid in dichloromethane. This reaction produced the unprotected terminal hydroxyl group. The cleaved DMT group was removed with the dichloroacetic acid/dichloromethane (DCA/DCM) solvent. The CPG was then washed with acetonitrile (ACN).

The second step involved activation of the incoming phosphoramidite with ethylthiotetrazole (ETT) to produce a species that would quickly couple with the terminal hydroxyl group produced in the previous step. The resultant phosphite triester was washed with ACN to remove activator and unreacted phosphoramidite.

The third step is oxidation of the newly formed phosphite triester to the pentavalent phosphate. This was accomplished by reacting the phosphite triester with a mixture of iodine and pyridine in water. Unused oxidant was washed from the CPG with ACN.

The fourth step involved capping of any unreacted hydroxyls that had failed to couple. The CPG was treated with a mixture of CAP NMI (N-methylimidazole in ACN) and CAP ALA (acetic anhydride, 2,6-lutidine, ACN). These reagents were washed from the CPG with ACN.

This cycle of four reactions was repeated until an oligonucleotide of the correct length and sequence was assembled on the solid support. The last phosphoramidite (hexylamino linker at the 5′ terminus of the oligonucleotide) was reacted in the same fashion as the other phosphoramidites used in the synthesis; however, this linker was not capped.

The oligonucleotide was deprotected and cleaved by treating the solid support, containing the crude synthesized oligonucleotide, with a t-butyl amine/ammonium hydroxide solution. The CPG was separated from the deprotected and cleaved oligonucleotide. The purity of the crude fully deprotected oligonucleotide was determined by analytical anion exchange chromatography and met a specification of greater than 50%.

The resultant oligonucleotide from Stage 1 was filtered, diluted and purified by preparative anion exchange chromatography (AX HPLC). Fractions were analyzed for product purity by analytical anion exchange HPLC. Individual fractions with a purity greater than 70% unpegylated aptamer, defined as the full length oligonucleotide that contains the 5′-hexylamino linker, were combined. In preparation for pegylation, the resultant fraction pool was first desalted and then concentrated using ultrafiltration. In some instances, the anion exchange chromatography step was replaced by a step in which diafiltration against sodium chloride was used to remove amine salts prior to Stage3.

In forming a covalent bond between the primary amine on the 5′ end of the oligonucleotide and the pegylation reagent (mPEG2-NHS ester), the reaction was conducted at pH 9 in sodium borate buffer. The reaction has been demonstrated to be site specific to the hexylamino linker at the 5′ end of the oligonucleotide using the pegylation conditions described.

The pegylated oligonucleotide was purified from unconjugated PEG reagent, unpegylated aptamer, and other by-products by the same preparative AX HPLC method described above for Stage 2. The individual fractions were analyzed by analytical AX HPLC. Fractions with greater than 85% full length pegylated oligonucleotide were pooled and the resultant pool was desalted, concentrated, and filtered.

The resultant drug substance was vacuum freeze dried to reduce the water content.

### Example 5

### Choroidal Neovascularization (CNV)

Experimental CNV is useful as a model for Age-related Macular degeneration (AMD). In CNV, vessels of the choroid grow through breaks in Bruch's membrane and into the retina, similar to what is observed in AMD patients. To induce experimental CNV, male C57BL/6 mice (18-20 g; Charles River, Wilmington, Mass.) are anesthetized with intramuscular ketamine hydrochloride (25 mg/kg) and xylazine (10 mg/kg) and the mice pupils are dilated with 1% tropicamide. Four burns are generated using diode laser photocoagulation (75-μm spot size, 0.1-second duration, 90 mW, Oculight SL laser, IRIDEX, Mountain View, Calif.) and a hand-held cover slide as a contact lens. Burns are localized to the 3, 6, 9 and 12 o'clock positions of the posterior pole of the retina. Production of a bubble in the choroid at the time of laser photocoagulation, which indicates rupture of Bruch's membrane, is an important factor in obtaining choroidal neovascularization, so only mice in which a bubble is produced for all four burns are included in the study. After 7 days, mice are treated with (a) an intra-peritoneal injection of 2.0 mg/ml of Antagonist A twice a day for seven days; (b) an intra-peritoneal injection of 2.0 mg/mL of ranibizumab (as the commercially available composition Lucentis®) twice a day for 7 days; or (c) an intra-peritoneal injection of 2.0 mg/ml of Antagonist A and an intra-peritoneal injection of 2.0 mg/mL of ranibizumab (as the commercially available composition Lucentis®, both being administered twice a day for 7 days. The area of choroidal NV lesions is measured in flat-mounted choroid stained with platelet endothelial cell adhesion molecule (PECAM) antibody. Flat-mounts are examined by fluorescence microscopy and quantified using Openlab software.

The effects of the administration of one or more of (a), (b) and (c) are measured for decrease in CNV area compared to untreated controls.

### 9. INCORPORATION BY REFERENCE

All publications and patent applications disclosed in this specification are herein incorporated by reference to the same extent as if each individual publication or patent application was specifically and individually indicated to be incorporated by reference.

What is claimed is:

1. A method for treating wet age-related macular degeneration, comprising administering to a human in need thereof an effective amount of:
(a) a pegylated anti-PDGF-B aptamer having the formula 5′-[mPEG2 40 kD]-[HN—(CH₂)₆O] CAGGCU_{f}AC_{f}Gₘ [PO₃(CH₂CH₂O)₆] CGTAGₘAGₘCAU_{f}C_{f}Aₘ [PO₃(CH₂CH₂O)₆] TGATC_{f}C_{f}U_{f}Gₘ-[3T]-3′ (SEQ ID NO: 23),
where
[3T] refers to an inverted thymidine nucleotide that is attached to the 3′ end of the oligonucleotide at the 3′ position on the ribose sugar;
[mPEG2 40 kD] represents two about 20 kD polyethylene glycol (PEG) polymer chains that are covalently attached to the two amino groups of a lysine residue via carbamate linkages;
the [mPEG2 40 kD]-lysine moiety is covalently attached to the [HN—(CH₂)₆—O] linker via an amide bond;
Gₘ and Aₘ represent 2′-methoxy substituted forms of guanosine and adenosine, respectively; and
C_{f} and U_{f} represent 2′-fluoro substituted forms of cytosine and uridine, respectively,
or a pharmaceutically acceptable salt thereof; and
(b) aflibercept, or a pharmaceutically acceptable salt thereof.

2. The method of claim 1, wherein (a) and (b) are administered within 24 hours of each other.

3. The method of claim 1, wherein (a) and (b) are administered within 60 minutes of each other.

4. The method of claim 1, wherein (a) and (b) are administered concurrently.

5. The method of claim 1, wherein (a) and (b) are present in the same composition.

6. The method of claim 1, wherein (a) or (b) is present in a drug-delivery device.

7. The method of claim 1, wherein (a) and (b) are present in a drug-delivery device.

8. The method of claim 1, wherein (a) and (b) are present in the same drug-delivery device.

9. The method of claim 1, wherein (a) or (b) is administered intraocularly.

10. The method of claim 1, wherein (a) and (b) are administered intraocularly.

11. The method of claim 9, wherein (a) or (b) is administered by intravitreal administration or anterior chamber administration.

12. The method of claim 10, wherein (a) and (b) are administered by intravitreal administration or anterior chamber administration.

13. The method of any one of claims 1-12, wherein the pegylated anti-PDGF-B aptamer has the structure: 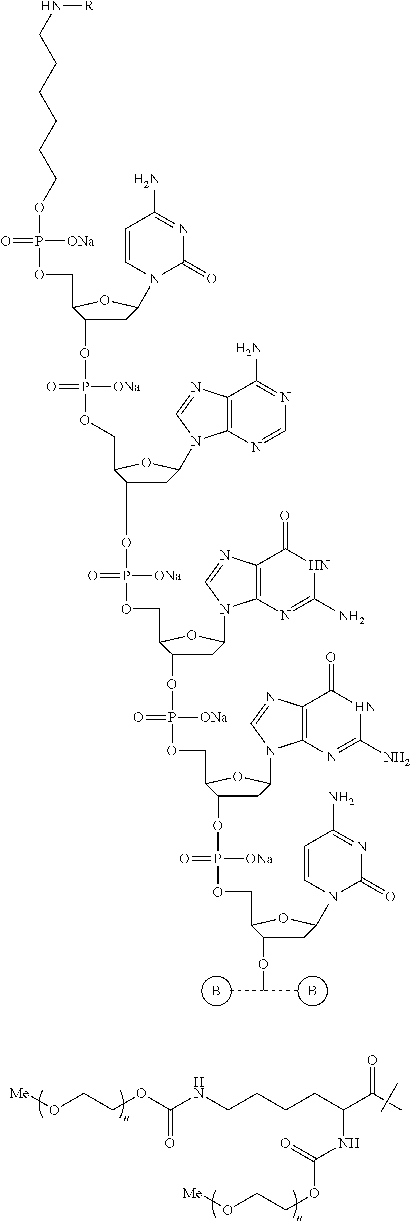
where R represents the following structure:
and n is about 450. 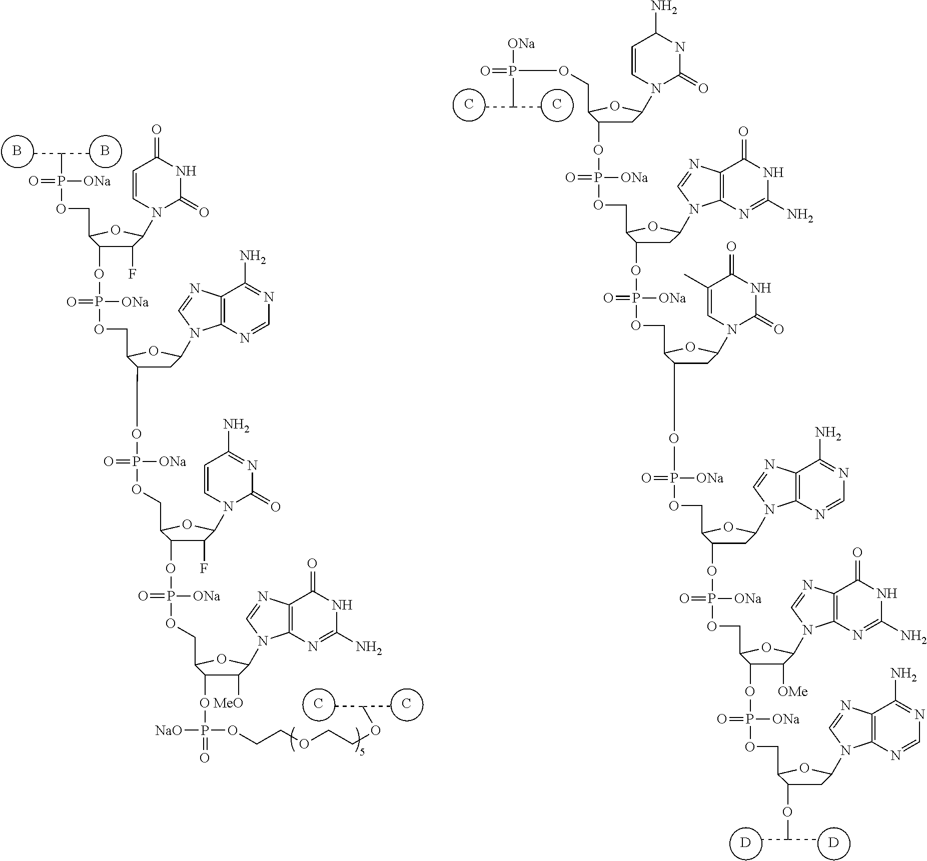 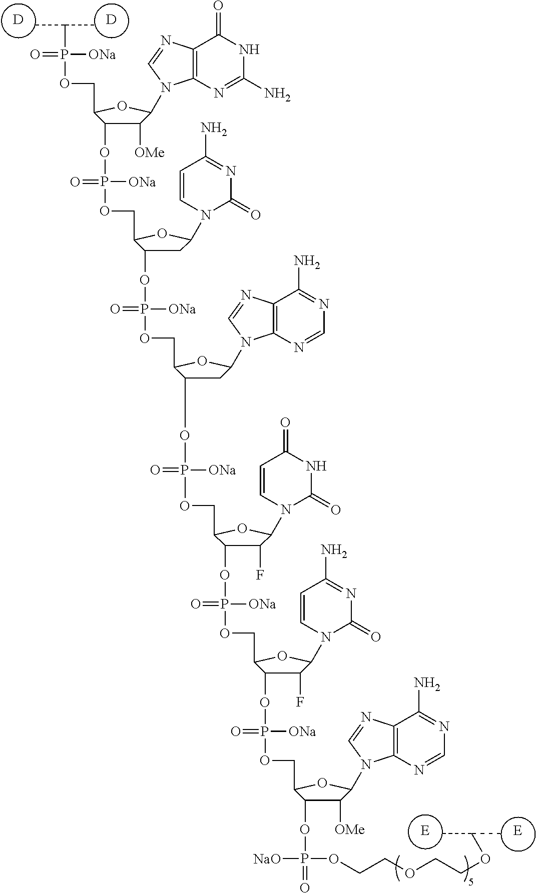 [chemical structure]
